(19) United States
(12) Patent Application Publication
(10) Pub. No.: US 2024/0124408 A1
(43) Pub. Date: Apr. 18, 2024

(54) Substituted Cyclohexanecarboxamides, Their Preparation and Their Therapeutic Application

(71) Applicant: Sanofi, Paris (FR)

(72) Inventors: Paul Justin Mueller, Bridgewater, NJ (US); Florian Auger, Paris (FR); Guillaume Begis, Paris (FR); Yann Foricher, Paris (FR); Christoph Grebner, Frankfurt am Main (DE); Stéphane Hourcade, Paris (FR); Stéphanie Meyfroot, Paris (FR); Sven Ruf, Frankfurt am Main (DE); Klaus Wirth, Frankfurt am Main (DE)

(73) Assignee: Sanofi, Paris (FR)

(21) Appl. No.: 18/267,644

(22) PCT Filed: Dec. 16, 2021
(86) PCT No.: PCT/US2021/063704

§ 371 Date: Jun. 15, 2023

Related U.S. Application Data
(60) Provisional application No. 63/126,184, filed on Dec. 16, 2020.

(30) Foreign Application Priority Data
Feb. 25, 2021 (EP) 21315029.5

(51) Int. Cl. C07D 305/06; A61P 11/04; C07C 235/40; C07C 237/12

(52) U.S. Cl. CPC C07D 305/06; A61P 11/04; C07C 235/40; C07C 237/12

(57) ABSTRACT

Compounds of formula (1):

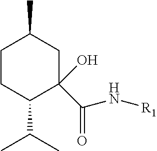

wherein R₁ represents a —C(R₂)(R₃)—[C(R₄)(R₅)]ₘ-L-R₆ group or —R₇; and the preparation and the therapeutic uses of the compounds of formula (1) as agonists of TRPM8 receptors, useful especially in the treatment of oropharyngeal dysphagia.

Provided herein are substituted cyclohexanecarboxamide compounds, processes for their preparation, pharmaceutical compositions containing the compounds, as well as therapeutic uses thereof.

Oropharyngeal dysphagia (OD) is a prevalent medical condition where patients have difficulty swallowing food or liquids, likely due to either a lack of nerve sensitivity or muscle weakness. Although dysphagia is commonly associated with aging, neurological injuries after stroke or trauma, neurological disorders (for example, multiple sclerosis, Parkinson disease, Alzheimer disease), and cancer treatments (for example, radiation/surgery sequalae), dysphagia can arise in many patient populations, including children with acquired brain injury or other neuromuscular disorders, craniofacial or airway malformations, as well as those with respiratory, cardiac, or gastrointestinal disease. Dysphagia may also present in all critically ill patients and large-scale clinical data show that post-extubation dysphagia (PED) is commonly observed in intensive care unit (ICU) patients. Dysphagia is a serious condition as it impairs quality of life and causes nutritional and respiratory complications associated with poor prognosis and high mortality rates. This disabling condition impacts an estimated over 50 million patients worldwide (Nat Rev Gastroenterol Hepatol. 2015, 12(5): 259-270).

There are currently no approved pharmacological therapies for OD. The current standard of care is mostly limited to food modification (e.g., thickeners) and physical rehabilitation (e.g., postural adjustments and behavioral exercises to both strengthen the muscles involved in swallowing and improve swallowing ability).

Transient receptor potential cation channel subfamily M member 8 (TRPM8), also called the cold and menthol receptor 1 (CMR1), is the primary molecular transducer of cold somatosensation in humans and is activated at cool and cold temperatures (Nature. 2002, 416(6876): 52-58). TRPM8 is also activated by a variety of chemical compounds such as icilin or menthol (J Pharm Pharm Sci. 2010, 13(2): 242-253). This activation provokes the intracellular entry of calcium and sodium, leading to the depolarization of the membrane and triggering action potential and activating multiple signaling pathways. TRPM8 is notably expressed in ganglia and peripheral nerve fibers that innervate the skin (BMC Neurol. 2007, 7:11) and also in tongue, pharyngeal and laryngeal tissues (Neurogastroenterol Motil. 2018, (11): e13398), which are associated with swallowing functions. This expression of the channel provides the possibility for therapeutic activation of TRPM8 in various pathologies, such as, for example, chronic cough, dry and pruritic skin, dry eye syndrome, and oropharyngeal dysphagia.

Activation of TRPM8 receptors in sensory nerves in the oral cavity and pharynx can activate swallowing reflexes in dysphagic patients (Journal of GHR. 2014, 3(5): 1066-1072). In particular, cold temperature (ice massage) and local application of menthol can reduce the abnormal delay in initiating the swallow reflex in dysphagic patients through TRPM8 activation (J Stroke Cerebrovasc Dis. 2013, 22(4): 378-382); Br J Clin Pharmacol. 2006, 62 (3): 369-371).

However, both approaches have limitations as therapeutic solutions: ice massage being impractical for daily living, and menthol presenting several disadvantages such as its low potency on TRPM8, poor selectivity (likely responsible for irritations), and its strong smell and taste.

Thus, there continues to be a need for activators of TRPM8 that can be used in the treatment of patients with a disease or condition affected by the activation of TRPM8 receptors.

Provided herein are novel compounds able to activate TRPM8, which may be useful in treating a disease, syndrome, or condition in a subject in which the disease, syndrome, or condition is affected by the activation of TRPM8 receptors, such as oropharyngeal dysphagia, chronic cough, pharyngeal irritation, dry and pruritic skin, and/or dry eye syndrome.

Provided herein is a compound of the formula (I):

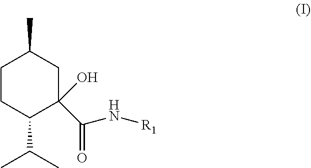

wherein:
R₁ is —C(R₂)(R₃)[C(R₄)(R₅)]ₘ-L-R₆ or —R₇;
m represents 0, 1, 2 or 3;
R₂ and R₃ independently represent a hydrogen atom, a deuterium atom, a —(C₁-C₆)-alkyl, a (C₁-C₆)-alkyl-OH group, a —C(═O)NH₂ group, a —(C₁-C₆)-alkoxyl group, or a —C(═O)O(C₁-C₆)-alkyl group;
R₄ and R₅ independently represent a hydrogen atom, a deuterium atom, a fluorine atom, a —NH₂ group, a —OH group, a —(C₁-C₆)-alkyl group, a —CF₃ group, a carboxyl group, or a —R₈—(C₁-C₆)-alkyl-R₉ group;
   R₈ represents a bond, an —O—, a —OC(═O)— group, a —N(H)C(═O)— group, a —C(═O)O— group, or a —C(═O)N(H)— group;
   R₉ represents a hydrogen atom, a —C(═O)—OH group, a —C(═O)O(C₁-C₃)-alkyl group, a —OH group, a —O(C₁-C₃)-alkyl group, or a —NH₂ group;
   or R₄ and R₅ can form, together with the carbon atom to which they are attached, a heterocycloalkyl group comprising 3 to 5 carbon atoms and comprising from 1 or 2 heteroatoms selected from oxygen and nitrogen;
L represents a bond, a —(C₁-C₆)-alkylene- group, an —O—(C₁-C₆)-alkylene- group, an —O—, a —OC(═O)— group, a —N(H)— group, a —C(═O)— group, a —C(═O)O— group, a —C(═O)—O—(C₁-C₃)-alkyl-group, a —C(═O)—N(H)— or a —CONH(C₁-C₆)-alkyl- group;
R₆ is selected from the group consisting of a —OH group; a —(C₁-C₆)-alkyl group; a phenyl group; a monocyclic heteroaryl group comprising 3 to 5 carbon atoms and comprising from 1 to 2 heteroatoms independently selected from oxygen, nitrogen and sulfur; an ortho-fused bicyclic heteroaryl group comprising 7 to 10 carbon atoms and comprising from 1 to 4 heteroatoms independently selected from oxygen, nitrogen and sulfur; an ortho-fused bicyclic cycloalkyl group comprising 8 to 11 carbon atoms; and an ortho-fused bicyclic heterocycloalkyl group comprising 8 to 9 carbon atoms and comprising 1 or 2 heteroatoms independently selected from oxygen and nitrogen; said phenyl, monocyclic heteroaryl, ortho-fused bicyclic heteroaryl, ortho-fused bicyclic cycloalkyl, ortho-fused bicyclic heterocycloalkyl groups represented by R₆ are unsubstituted or substituted with 1 to 3 substituents independently selected from the group consisting of:
   a halogen atom, a —OH group, an oxo group, an —O—(C₁-C₆)-alkyl group, a —(C₁-C₆)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₆)-alkyl group, or a —C(═O)N(C₁-C₃)-alkyl group, a —(OCH₂CH₂)ₙ—R₁₀ group, and a —R₁₁—(C₁-C₆)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents a —O(C₁-C₄)-alkyl group, a —N⁺—(CH₃)₃ group, or —N⁺H—(CH₃)₂ group; R₁₁ represents a bond, an —O—, or a —C(═O)O— group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, a heterocyclic group or an —O-heterocyclic group, said heterocyclic group and —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group.
R₇ represents a phenyl group; a monocyclic cycloalkyl group comprising 4 to 7 carbon atoms; a monocyclic heterocycloalkyl group comprising 3 to 6 carbon atoms and comprising from 1 to 2 heteroatoms independently selected from oxygen and nitrogen; or an ortho-fused bicyclic heterocycloalkyl group comprising 8 to 9 carbon atoms and comprising 1 or 2 heteroatoms independently selected from oxygen and nitrogen; wherein said phenyl group is unsubstituted or substituted with 1 to 3 substituents independently selected from a halogen atom, a —(C₁-C₃)-alkyl group, an —O—(C₁-C₃)-alkyl group and a morpholine group; said monocyclic cycloalkyl, monocyclic heterocycloalkyl, an ortho-fused bicyclic heterocycloalkyl groups being unsubstituted or substituted with 1 to 3 substituents independently selected from the group consisting of: a halogen atom, an oxo group, a —(C₁-C₆)-alkyl group, a phenyl group, an —O—, a benzyl group, a —OH group, and an —O—(C₁-C₆)-alkyl group;
   or a pharmaceutically acceptable salt thereof.

The compounds of formula (I) contain more than one asymmetric carbon atoms, more particularly one asymmetric carbon atom on the cyclohexyl group. They may therefore exist in the form of enantiomers. The compounds of formula (I) include enantiomers, racemates, and mixtures thereof. In particular, the carbon of the cyclohexyl group linked to the hydroxyl group of the formula (I) may be in the absolute configuration (R) or (S). The carbon of the cyclohexyl group linked to the hydroxyl group is advantageously in the absolute configuration (S).

The compounds of formula (I) also include tautomer forms thereof.

The compounds of formula (I) may exist in the form of bases, acids, or zwitterions.

The compounds of formula (I) can be in the form of addition salts with acids or bases, for example: hydrochloride acid and citric acid. Hence, provided herein inter alia, are compounds of formula (I) or pharmaceutically acceptable salts thereof.

As used herein, the following terms, unless otherwise indicated, shall be understood to have the following meanings:

As used herein, the term “alkyl” means a straight or branched aliphatic hydrocarbon group having 1 to about 12 carbon atoms in the chain. In one aspect, an alkyl has 1 to 6 carbon atoms in the chain. Another aspect, an alkyl has 1 to 3 carbon atoms in the chain. “Lower alkyl” means an alkyl group having 1 to about 4 carbon atoms in an alkyl chain that may be straight or branched. Branched means that one or more lower alkyl groups, such as methyl, ethyl or propyl, are attached to a linear alkyl chain. Additionally, the term “(C₁-C₆)-alkyl” denotes a straight or branched alkyl group having one to six carbon atoms. The term “(C₁-C₄)-alkyl” denotes a straight or branched alkyl group having one to four carbon atoms. The term “(C₁-C₃)-alkyl” denotes a straight or branched alkyl group having one to three carbon atoms. Exemplary alkyl includes methyl, ethyl, i-propyl, t-butyl, and the like.

As used herein, the term “alkoxy” means an alkyl-O— group wherein the alkyl group is as herein described. Exemplary alkoxy groups include methoxy, ethoxy, n-propoxy, i-propoxy, n-butoxy, t-butoxy, heptoxy, and the like.

As used herein, the term “alkylene” means a straight or branched bivalent hydrocarbon chain having from 1 to about 12 carbon atoms. In one aspect, an alkylene has 1 to about 10 carbon atoms in the chain. Another aspect, an alkylene has 1 to about 6 carbon atoms in the chain. A “lower alkylene” is an alkylene having from 1 to about 4 carbon atoms. Exemplary alkylene includes methylene, ethylene, propylene, and butylene.

As used herein, the term “aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

As used herein, the term “aryl” means an aromatic monocyclic or bicyclic ring system of about 5 to about 10 carbon atoms. Exemplary aryl include phenyl and naphthyl.

As used herein, the term “benzyl” means phenyl-CH₂— group.

As used herein, the term “carboxyl” means a substituent of the formula —C(═O)OH.

As used herein, the term “cycloalkyl” means a ring system comprising, unless otherwise mentioned, from 3 to 10 carbon atoms, that is saturated or partially unsaturated and unsubstituted or substituted. Said cycloalkyl group may be monocyclic or bicyclic. The term “monocyclic cycloalkyl” means an unsubstituted or substituted ring system comprising 3 to 7 carbon atoms; a “bicyclic cycloalkyl group” means a two-ring system comprising 8 to 11 carbon atoms. By way of examples, mention may be made of, but not limited to: cyclopropyl, cyclobutyl, cyclopentyl, cyclohexyl, cyclohexenyl, cycloheptyl, spiro[2.2]pentyl, spiro[3.4]octyl, [5.5]undecyl, [4.5]decyl groups, and the like.

As used herein, the term “ortho-fused cycloalkyl group” means an unsubstituted or substituted 8-10 membered bicyclic ring group. Included within the scope of the definition of ortho-fused cycloalkyl group are bicyclic ring systems wherein one of the rings is saturated or partially unsaturated cycloalkyl ring and the other ring is an aromatic ring. By way of examples, mention may be made of, but not limited to: 1,2,3,4-tetrahydronaphthalen-1-yl, indan-1-yl, hydroxy-1,2,3,4-tetrahydronaphthalen-1-yl, 1-hydroxy-2,3-dihydro-1H-inden-1-yl, 5-methoxyindan-1-yl, and 4-chloro-1-hydroxy-indan-1-yl.

As used herein, the terms “halo” or “halogen” mean fluoro, chloro, bromo, or iodo. Particular halogens are fluoro and chloro.

As used herein, the term “heteroaryl” whether used alone or with other terms, such as “heteroaryl group”, means a cyclic aromatic group containing 3 to 10 carbon atoms and containing between 1 and 4 heteroatoms, such as nitrogen, oxygen or sulfur. Said heteroaryl group may be monocyclic or bicyclic. As used herein, the term “monocyclic heteroaryl” means a cyclic aromatic group containing 3 to 5 carbon atoms and containing between 1 and 2 heteroatoms, such as nitrogen, oxygen or sulfur. By way of examples of monocyclic heteroaryl groups, mention may be made of, but not limited to: benzimidazole, benzothiazole, benzothiadiazole, benzofuran, benzotriazole, benzoxazole, furanyl, furazanyl, indole, imidazolyl, isoxazole, isothiazolyl, isoxazolyl, oxadiazolyl, oxazolyl, pyridine, pyridyl, pyridinyl, pyrimidine, pyrimidinyl, pyrrolo[2,3-b]pyridine, pyrazinyl, pyrazolyl, pyridazinyl, pyrrolyl, thienyl, 1,2-oxazolyl, 1,2,4-thiadiazolyl, 1,2,4-triazinyl, 1,3,4-thiadiazolyl, thiazolyl, thienyl, triazolyl, thiophenyl and the like.

As used herein, the term “ortho-fused” means a ring system where the two adjacent rings have two adjacent atoms in common. The term “ortho-fused heteroaryl” means a bicyclic ring system comprising 7 to 10 carbon atoms and comprising from 1 to 4 heteroatoms independently selected from oxygen, nitrogen and sulfur. Included within the scope of the definition of ortho-fused heteroaryl group is a bicyclic ring system wherein one of the rings is heteroaryl and the other ring is aryl ring or both rings are heteroaryl. Examples include indolyl and benzimidazolyl.

The terms “heterocycle” and derivatives thereof such as “heterocyclyl” and “heterocyclic” mean an aromatic, a partially unsaturated or a saturated ring containing one or more carbon atoms and one or more heteroatoms such as nitrogen, oxygen and sulfur, but may be more specifically defined where appropriate in the specification, for example with respect to degree of saturation, number of members (i.e. atoms) in the ring and/or the type and quantity of heteroatoms in the ring. The point of attachment in a compound structure may be via any carbon or nitrogen in the heterocyclic ring which results in the creation of a stable structure, unless specified otherwise. The heterocyclic ring may be substituted on any available carbon or nitrogen in the ring which results in the creation of a stable structure, unless specified otherwise. Exemplary heterocyclyl groups include piperidinyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

As used herein, heterocycloalkyl means a cyclic alkyl group comprising, unless otherwise mentioned, from 3 to 9 carbon atoms and containing 1 or 2 heteroatoms such as oxygen, nitrogen or sulfur. Such heterocycloalkyl group may be saturated or partially saturated and unsubstituted or substituted and may be monocyclic or bicyclic. As used herein, the term “monocyclic heterocycloalkyl” means monocyclic heterocycloalkyl group comprising 3 to 6 carbon atoms and comprising from 1 or 2 heteroatoms selected from oxygen and nitrogen. By way of examples of monocyclic heterocycloalkyl groups, mention may be made of, but not limited to: tetrahydropyridinyl, dihydropyridinyl, dihydropyranyl, 2-oxotetrahydrofuran-3-yl, tetrahydropyranyl groups, and the like.

As used herein, the term “ortho-fused” means a ring system where the two adjacent rings have two adjacent atoms in common. The term “ortho-fused bicyclic heterocycloalkyl” means a bicyclic ring system comprising 8 to 9 carbon atoms and comprising 1 or 2 heteroatoms independently selected from oxygen and nitrogen. Included within the scope of the definition of ortho-fused heterocycloalkyl group are bicyclic ring systems wherein only one of the rings is heterocycloalkyl, the other ring is aryl ring or heteroaryl ring. By way of examples of ortho-fused heterocycloalkyl group, mention may be made of, but not limited to: 2,3-dihydrobenzofuran-3-yl, 6-methoxy-2,3-dihydrobenzofuran-3-yl, chroman-4-yl, isochroman-1-yl, 4-hydroxychroman-4-yl, 3-oxo-1,3-dihydroisobenzofuran-1-yl.

As used herein, the term “patient” includes mammals, especially humans, who use the instant active agents for the prevention or treatment of a medical condition. Administering of the compound of formula (I) to the patient includes both self-administration and administration to the patient by another person. The patient may be in need of treatment for an existing disease or medical condition, or may desire prophylactic treatment to prevent or reduce the risk of medical condition.

As used herein, “treating,” unless otherwise indicated, means to partially or totally alleviate symptoms on a temporary or permanent basis, or to slow the worsening of symptoms of the named disorder or condition. The term “treatment” as used herein, unless otherwise indicated, refers to the act of treating.

As used herein, the phrase “a method of treating” or its equivalent, when applied to, for example, oropharyngeal dysphagia, refers to a procedure or course of action that is designed to reduce, eliminate, or inhibit the progression of medical condition in a patient; and/or to alleviate the symptoms of oropharyngeal dysphagia.

As used herein, the term “therapeutically effective amount” or “effective amount” means the amount of the subject compound, composition or combination that will elicit the biological or medical response of a tissue, system, animal or human that is being sought by the researcher, veterinarian, medical doctor or other clinician.

“Pharmaceutically acceptable salts,” as used herein, refers to the relatively non-toxic, inorganic and organic acid addition salts, and base addition salts, of compounds of Formula (I). These salts can be prepared in situ during the final isolation and purification of the compounds. Some of the compounds of the invention are basic, and such compounds are useful in the form of the free base, or in the form of a pharmaceutically acceptable acid addition salt thereof.

Acid addition salts are a convenient form for use; and in practice, use of the salt form in essence amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial activation effects inherent in the free base are not vitiated by side effects ascribable to the anions. Although pharmaceutically acceptable salts of said basic compounds are preferred, all acid addition salts are useful as sources of the free base form even if the particular salt, per se, is desired only as an intermediate product as, for example, when the salt is formed only for purposes of purification, and identification, or when it is used as intermediate in preparing a pharmaceutically acceptable salt by ion exchange procedures. In particular, acid addition salts can be prepared by separately reacting the purified compound in its free base form with a suitable organic or inorganic acid and isolating the salt thus formed. Pharmaceutically acceptable salts within the scope of the invention include those derived from mineral acids and organic acids. See, for example S. M. Berge, et al., “Pharmaceutical Salts,” J. Pharm. Sci., (66), 1-19 (1977).

Where the compound disclosed is substituted with an acidic moiety, base addition salts may be formed and are simply a convenient form for use; and in practice, use of the salt form in essence amounts to use of the free acid form. The bases which can be used to prepare the base addition salts include preferably those which produce, when combined with the free acid, pharmaceutically acceptable salts, that is, salts whose cations are non-toxic to the patient in pharmaceutical doses of the salts, so that the beneficial activation effects inherent in the free base are not vitiated by side effects ascribable to the cations.

As well as being useful in themselves as active compounds, salts of compounds of the invention are useful for the purposes of purification of the compounds, for example by exploitation of the solubility differences between the salts and the parent compounds, side products and/or starting materials by techniques well known to those skilled in the art.

The compounds disclosed may contain more than one asymmetric center. These asymmetric centers may independently be in either the R or S configuration. It will be apparent to those skilled in the art that certain compounds of formula (I) may also exhibit geometrical isomerism. It is to be understood that the present disclosure includes individual geometrical isomers and stereoisomers and mixtures thereof, including racemic mixtures, of compounds of formula (I) hereinabove. Such isomers can be separated from their mixtures, by the application or adaptation of known methods. Chiral chromatography techniques represent one means for separating isomers from mixtures thereof. Some compounds may be separated by chiral recrystallization techniques as an alternative means for separating isomers from mixtures thereof. Individual isomeric compounds can also be prepared by employing, where applicable, chiral precursors.

One embodiment is a compound of formula (I), wherein R₁ represents —C(R₂)(R₃)—[C(R₄)(R₅)]ₘ-L-R₆.

Another embodiment is a compound of formula (I), wherein R₁ represents —R₇.

Another embodiment is a compound of formula (I), wherein one of R₂ and R₃ independently represents a hydrogen atom.

Another embodiment is a compound of formula (I), wherein both R₂ and R₃ represent a hydrogen atom.

Another embodiment is a compound of formula (I), wherein one of R₂ and R₃ independently represents a deuterium atom.

Another embodiment is a compound of formula (I), wherein one of R₂ and R₃ independently represents a-(C₁-C₆)-alkyl group.

Another embodiment is a compound of formula (I), wherein one of R₂ and R₃ independently represents a-(C₁-C₃)-alkyl group.

Another embodiment is a compound of formula (I), wherein one of R₂ and R₃ independently represents a (C₁-C₆)-alkyl-OH group.

Another embodiment is a compound of formula (I), wherein one of R₂ and R₃ independently represents a (C₁-C₃)-alkyl-OH group.

Another embodiment is a compound of formula (I), wherein one of R₂ and R₃ independently represents a —C(═O)NH₂ group.

Another embodiment is a compound of formula (I), wherein one of R₂ and R₃ independently represents a —(C₁-C₆)-alkoxyl group.

Another embodiment is a compound of formula (I), wherein one of R₂ and R₃ independently represents a —(C₁-C₃)-alkoxyl group.

Another embodiment is a compound of formula (I), wherein one of R₂ and R₃ independently represents a —C(═O)O(C₁-C₆)-alkyl group.

Another embodiment is a compound of formula (I), wherein one of R₂ and R₃ independently represents a —C(═O)O(C₁-C₃)-alkyl group.

Another embodiment is a compound of formula (I), wherein m represents 0.

Another embodiment is a compound of formula (I), wherein m represents 1.

Another embodiment is a compound of formula (I), wherein m represents 2.

Another embodiment is a compound of formula (I), wherein m represents 3.

Another embodiment is a compound of formula (I), wherein one R₄ and R₅ independently represents a hydrogen atom.

Another embodiment is a compound of formula (I), wherein both R₄ and R₅ represent a hydrogen atom.

Another embodiment is a compound of formula (I), wherein one of R₄ and R₅ independently represents a deuterium atom.

Another embodiment is a compound of formula (I), wherein one of R₄ and R₅ independently represents a fluorine atom.

Another embodiment is a compound of formula (I), wherein both R₄ and R₅ represent a fluorine atom.

Another embodiment is a compound of formula (I), wherein one of R₄ and R₅ independently represents a —NH₂ group.

Another embodiment is a compound of formula (I), wherein one of R₄ and R₅ independently represents a —OH group.

Another embodiment is a compound of formula (I), wherein one of R₄ and R₅ independently represents a —(C₁-C₆)-alkyl group.

Another embodiment is a compound of formula (I), wherein one of R₄ and R₅ independently represents a —(C₁-C₃)-alkyl group.

Another embodiment is a compound of formula (I), wherein one of R₄ and R₅ independently represents a —CF₃ group.

Another embodiment is a compound of formula (I), wherein one of R₄ and R₅ independently represents a carboxyl group.

Another embodiment is a compound of formula (I), wherein one of R₄ and R₅ independently represents a —R₈—(C₁-C₆)-alkyl-R₅ group.

Another embodiment is a compound of formula (I), wherein one of R₄ and R₅ independently represents a —R₈—(C₁-C₃)-alkyl-R₅ group.

Another embodiment is a compound of formula (I), wherein R₄ and R₅ can form, together with the carbon atom to which they are attached, a heterocycloalkyl group comprising 3 to 5 carbon atoms and comprising from 1 or 2 heteroatoms selected from oxygen and nitrogen.

Another embodiment is a compound of formula (I), wherein L represents a bond.

Another embodiment is a compound of formula (I), wherein L represents a —(C₁-C₆)-alkylene-group.

Another embodiment is a compound of formula (I), wherein L represents a —(C₁-C₃)-alkylene-group.

Another embodiment is a compound of formula (I), wherein L represents an —O—.

Another embodiment is a compound of formula (I), wherein L represents a —OC(═O)— group.

Another embodiment is a compound of formula (I), wherein L represents a —N(H)— group.

Another embodiment is a compound of formula (I), wherein L represents a —C(═O)— group.

Another embodiment is a compound of formula (I), wherein L represents a —C(═O)O— group.

Another embodiment is a compound of formula (I), wherein L represents a —C(═O)—O—(C₁-C₃)-alkyl- group.

Another embodiment is a compound of formula (I), wherein L represents a —C(═O)—N(H)— group.

Another embodiment is a compound of formula (I), wherein L represents a —CONH(C₁-C₆)-alkyl-group.

Another embodiment is a compound of formula (I), wherein L represents a —CONH(C₁-C₃)-alkyl-group.

Another embodiment is a compound of formula (I), wherein R₅ represents a —OH group.

Another embodiment is a compound of formula (I), wherein R₆ represents a —(C₁-C₆)-alkyl-group.

Another embodiment is a compound of formula (I), wherein R₆ represents a —(C₁-C₃)-alkyl-group.

Another embodiment is a compound of formula (I), wherein R₅ represents a phenyl group; a monocyclic heteroaryl group comprising 3 to 5 carbon atoms and comprising from 1 to 2 heteroatoms independently selected from oxygen, nitrogen and sulfur; an ortho-fused bicyclic heteroaryl group comprising 7 to 10 carbon atoms and comprising from 1 to 4 heteroatoms independently selected from oxygen, nitrogen and sulfur; an ortho-fused bicyclic cycloalkyl group comprising 8 to 11 carbon atoms; and an ortho-fused bicyclic heterocycloalkyl group comprising 8 to 9 carbon atoms and comprising 1 or 2 heteroatoms independently selected from oxygen and nitrogen; said phenyl, monocyclic heterocycloalkyl, monocyclic heteroaryl, ortho-fused bicyclic heteroaryl, ortho-fused bicyclic cycloalkyl, ortho-fused bicyclic heterocycloalkyl groups being unsubstituted or substituted with 1 to 3 substituents independently selected from the group consisting of:

a halogen atom, an oxo group, a —OH group, an —O—(C₁-C₆)-alkyl group, a —(C₁-C₆)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₆)-alkyl group, a —C(═O)N(C₁-C₃)-alkyl group, a —(OCH₂CH₂)ₙ—R₁₀ group, and a —R₁₁—(C₁-C₆)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents a —O(C₁-C₄)-alkyl group, a —N⁺—(CH₃)₃ group, or —N⁺H—(CH₃)₂ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, a heterocyclic group or an —O-heterocyclic group; said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group.

Another embodiment is a compound of formula (I), R₆ represents a phenyl group; a monocyclic heteroaryl group comprising 3 to 5 carbon atoms and comprising from 1 to 2 heteroatoms independently selected from oxygen, nitrogen and sulfur; an ortho-fused bicyclic heteroaryl group comprising 7 to 10 carbon atoms and comprising from 1 to 4 heteroatoms independently selected from oxygen, nitrogen and sulfur; an ortho-fused bicyclic cycloalkyl group comprising 8 to 11 carbon atoms; and an ortho-fused bicyclic heterocycloalkyl group comprising 8 to 9 carbon atoms and comprising 1 or 2 heteroatoms independently selected from oxygen and nitrogen; said phenyl, monocyclic heterocycloalkyl, monocyclic heteroaryl, ortho-fused bicyclic heteroaryl, ortho-fused bicyclic cycloalkyl, and ortho-fused bicyclic heterocycloalkyl groups are unsubstituted or substituted with 1 to 3 substituents independently selected from the group consisting of:

a halogen atom, an oxo group, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, or a —C(═O)N(C₁-C₆)-alkyl group, a —(OCH₂CH₂)ₙ—R₁₀ group, and a —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents a —O(C₁-C₄)-alkyl group, a —N⁺—(CH₃)₃ group, or a-N⁺H—(CH₃)₂ group; R₁₁ represents a bond, an —O— or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, a heterocyclic group or an —O-heterocyclic group, said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group.

Another embodiment is a compound of formula (I), wherein R₆ represents a phenyl group, said phenyl being unsubstituted.

Another embodiment is a compound of formula (I), wherein R₆ represents a phenyl group, said phenyl being substituted with 1 to 3 substituents independently selected from the group consisting of: a halogen atom, a —OH group, an —O—(C₁-C₆)-alkyl group, a —(C₁-C₆)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₆)-alkyl group, and a —C(═O)N(C₁-C₃)-alkyl group.

Another embodiment is a compound of formula (I), wherein R₆ represents a phenyl group, said phenyl being substituted with 1 to 2 substituents independently selected from the group consisting of: a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, and a —C(═O)N(C₁-C₃)-alkyl group.

Another embodiment is a compound of formula (I), wherein R₆ represents a phenyl group, said phenyl being substituted with 1 to 2 —(OCH₂CH₂)ₙ—R₁₀ groups, wherein n independently represents 1, 2 or 3; and R₁₀ independently represents an —O—(C₁-C₄)-alkyl group, a —N⁺—(CH₃)₃ group, or a —N⁺H—(CH₃)₂ group.

Another embodiment is a compound of formula (I), wherein R₆ represents a phenyl group, wherein said phenyl is substituted with 1 to 3 —R₁₁—(C₁-C₆)-alkyl-R₁₂ group which is unsubstituted or substituted on the (C₁-C₆)-alkyl with 1 to 3 substituents independently selected from a —OH group, a —NH₂ group and —OCH₃ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, a heterocyclic group or an —O-heterocyclic group, said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group.

Another embodiment is a compound of formula (I), wherein R₆ represents a phenyl group, wherein said phenyl is substituted with 1 to 2 —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, a heterocyclic group or an —O-heterocyclic group, said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group.

Another embodiment is a compound of formula (I), wherein R₆ represents a monocyclic heteroaryl group comprising 3 to 5 carbon atoms and comprising from 1 to 2 heteroatoms independently selected from oxygen, nitrogen and sulfur, said monocyclic heteroaryl group being unsubstituted or substituted with 1 to 3 substituents independently selected from the group consisting of: a halogen atom, a —OH group, an —O—(C₁-C₆)-alkyl group, a —(C₁-C₆)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₆)-alkyl group, and a —C(═O)N(C₁-C₃)-alkyl group.

Another embodiment is a compound of formula (I), wherein R₆ represents a monocyclic heteroaryl group comprising 3 to 5 carbon atoms and comprising from 1 to 2 heteroatoms independently selected from oxygen, nitrogen and sulfur, said monocyclic heteroaryl group being unsubstituted or substituted with 1 to 2 substituents independently selected from the group consisting of: a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, and a —C(═O)N(C₁-C₆)-alkyl group.

Another embodiment is a compound of formula (I), wherein R₅ represents a monocyclic heteroaryl group comprising 3 to 5 carbon atoms and comprising from 1 to 2 heteroatoms independently selected from oxygen, nitrogen and sulfur, said monocyclic heteroaryl group being substituted with 1 to 3 —(OCH₂CH₂)ₙ—R₁₀ groups, wherein n independently represents 1, 2 or 3; R₁₀ independently represents a —O(C₁-C₄)-alkyl group, a —N⁺—(CH₃)₃ group, or a —N⁺H—(CH₃)₂ group.

Another embodiment is a compound of formula (I), wherein R₆ represents a monocyclic heteroaryl group comprising 3 to 5 carbon atoms and comprising from 1 to 2 heteroatoms independently selected from oxygen, nitrogen and sulfur, said monocyclic heteroaryl group being substituted with 1 to 3 —R₁₁—(C₁-C₆)-alkyl-R₁₂ group which is unsubstituted or substituted on the (C₁-C₆)-alkyl with 1 to 3 substituents independently selected from a —OH group, a —NH₂ group and —OCH₃ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, or a heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group.

Another embodiment is a compound of formula (I), wherein R₆ represents a monocyclic heteroaryl group comprising 3 to 5 carbon atoms and comprising from 1 to 2 heteroatoms independently selected from oxygen, nitrogen and sulfur, said monocyclic heteroaryl group being substituted with 1 to 2 —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group, R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, or a heterocyclic group comprising 3 to 9 carbon atoms and comprising from 1 to 3 heteroatoms independently selected from oxygen, nitrogen and sulfur being unsubstituted or substituted with 1 to 2 substituents independently selected from an oxo group and a —(C₁-C₃)-alkyl group.

Another embodiment is a compound of formula (I), wherein R₆ is selected from a monocyclic heteroaryl group comprising 3 to 5 carbon atoms and comprising from 1 to 2 heteroatoms independently selected from oxygen, nitrogen and sulfur, said monocyclic heteroaryl group being unsubstituted or substituted with 1 to 2 substituents independently selected from: a halogen atom, a —OH group, an —O—(C₁-C₃)-alkyl group, and a —(C₁-C₄)-alkyl-OH group.

Another embodiment is a compound of formula (I), wherein R₆ represents a monocyclic heteroaryl group selected from the following list:

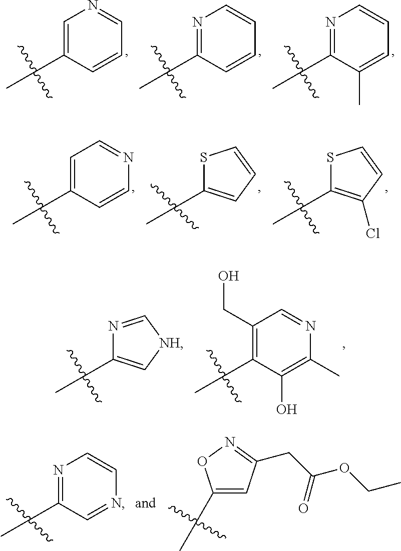

Another embodiment is a compound of formula (I), wherein R₆ represents an ortho-fused bicyclic heteroaryl group comprising 7 to 10 carbon atoms and comprising from 1 to 4 heteroatoms independently selected from oxygen, nitrogen and sulfur, said ortho-fused bicyclic heteroaryl group being unsubstituted or substituted with 1 to 3 substituents independently selected from the group consisting of: a halogen atom, an oxo group, a —OH group, an —O—(C₁-C₆)-alkyl group, a —(C₁-C₆)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₆)-alkyl group, and a —C(═O)N(C₁-C₃)-alkyl group.

Another embodiment is a compound of formula (I), wherein R₆ represents an ortho-fused bicyclic heteroaryl group comprising 7 to 10 carbon atoms and comprising from 1 to 4 heteroatoms independently selected from oxygen, nitrogen and sulfur, said ortho-fused bicyclic heteroaryl group being unsubstituted or substituted with 1 to 2 substituents independently selected from the group consisting of: a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, and a —C(═O)N(C₁-C₃)-alkyl group.

Another embodiment is a compound of formula (I), wherein R₆ represents an ortho-fused bicyclic heteroaryl group comprising 7 to 10 carbon atoms and comprising from 1 to 4 heteroatoms independently selected from oxygen, nitrogen and sulfur, said ortho-fused bicyclic heteroaryl group being substituted with a —(OCH₂CH₂)ₙ—R₁₀ group, wherein n represents 1, 2 or 3; R₁₀ represents a —O(C₁-C₄)-alkyl group, a —N⁺—(CH₃)₃ group, or a —N⁺H—(CH₃)₂ group.

Another embodiment is a compound of formula (I), wherein R₆ represents an ortho-fused bicyclic heteroaryl group comprising 7 to 10 carbon atoms and comprising from 1 to 4 heteroatoms independently selected from oxygen, nitrogen and sulfur, said ortho-fused bicyclic heteroaryl group being substituted with 1 to 3 —R₁₁—(C₁-C₆)-alkyl-R₁₂ group which is unsubstituted or substituted on the (C₁-C₆)-alkyl with 1 to 3 substituents independently selected from a —OH group, a —NH₂ group and —OCH₃ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, or a heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group.

Another embodiment is a compound of formula (I), wherein R₆ represents an ortho-fused bicyclic heteroaryl group comprising 7 to 10 carbon atoms and comprising from 1 to 4 heteroatoms independently selected from oxygen, nitrogen and sulfur, said ortho-fused bicyclic heteroaryl group being substituted with 1 to 2 —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, or a heterocyclic group comprising 3 to 9 carbon atoms and comprising from 1 to 3 heteroatoms independently selected from oxygen, nitrogen and sulfur being unsubstituted or substituted with 1 to 2 substituents independently selected from an oxo group and a —(C₁-C₃)-alkyl group.

Another embodiment is a compound of formula (I), wherein R₆ is selected from a bicyclic heteroaryl group comprising 7 to 10 carbon atoms and comprising from 1 to 4 heteroatoms independently selected from oxygen, nitrogen and sulfur, said bicyclic heteroaryl group being unsubstituted or substituted with 1 to 2 substituents independently selected from: a halogen atom, an oxo group, a —OH group, an —O—(C₁-C₃)-alkyl group, a —(C₁-C₃)-alkyl group and a —(C₁-C₄)-alkyl-OH group.

Another embodiment is a compound of formula (I), wherein R₆ represents a bicyclic heteroaryl group selected from the following list:

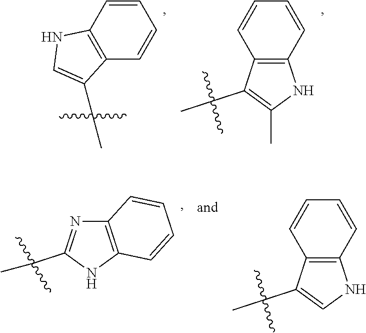 [SMILES: *c1c[nH]c2ccccc12 ; *c1c(C)[nH]c2ccccc12 ; *c1nc2ccccc2[nH]1 ; *c1c[nH]c2ccccc12]

Another embodiment is a compound of formula (I), wherein R₆ represents an ortho-fused bicyclic cycloalkyl group comprising 8 to 11 carbon atoms, or an ortho-fused bicyclic heterocycloalkyl group comprising 8 to 9 carbon atoms and comprising 1 or 2 heteroatoms independently selected from oxygen and nitrogen; said ortho-fused bicyclic cycloalkyl group or ortho-fused bicyclic heterocycloalkyl group being unsubstituted or substituted with 1 to 3 substituents independently selected from the group consisting of: a halogen atom, an oxo group, a —OH group, an —O—(C₁-C₆)-alkyl group, a —(C₁-C₆)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₆)-alkyl group, and a —C(═O)N(C₁-C₃)-alkyl group.

Another embodiment is a compound of formula (I), wherein R₆ represents an ortho-fused bicyclic cycloalkyl group comprising 8 to 11 carbon atoms, or an ortho-fused bicyclic heterocycloalkyl group comprising 8 to 9 carbon atoms and comprising 1 or 2 heteroatoms independently selected from oxygen and nitrogen; said ortho-fused bicyclic cycloalkyl group or ortho-fused bicyclic heterocycloalkyl group being unsubstituted or substituted with 1 to 2 substituents independently selected from the group consisting of: a halogen atom, an oxo group, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, and a —C(═O)N(C₁-C₃)-alkyl group.

Another embodiment is a compound of formula (I), wherein R₆ represents an ortho-fused bicyclic cycloalkyl group comprising 8 to 11 carbon atoms, or an ortho-fused bicyclic heterocycloalkyl group comprising 8 to 9 carbon atoms and comprising 1 or 2 heteroatoms independently selected from oxygen and nitrogen; said ortho-fused bicyclic cycloalkyl group or ortho-fused bicyclic heterocycloalkyl group being substituted with a —(OCH₂CH₂)ₙ—R₁₀ group wherein n represents 1, 2 or 3; R₁₀ represents a —O(C₁-C₄)-alkyl group, a —N⁺—(CH₃)₃ group, or a —N⁺H—(CH₃)₂ group.

Another embodiment is a compound of formula (I), wherein R₆ represents an ortho-fused bicyclic cycloalkyl group comprising 8 to 11 carbon atoms, or an ortho-fused bicyclic heterocycloalkyl group comprising 8 to 9 carbon atoms and comprising 1 or 2 heteroatoms independently selected from oxygen and nitrogen; said ortho-fused bicyclic cycloalkyl group or ortho-fused bicyclic heterocycloalkyl group being substituted with 1 to 3 —R₁₁—(C₁-C₆)-alkyl-R₁₂ group which is unsubstituted or substituted on the (C₁-C₆)-alkyl with 1 to 3 substituents independently selected from a —OH group, a —NH₂ group and —OCH₃ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NHC(═O)(C₁-C₃)-alkyl group, a —C(═O)H group, or a heterocyclic group comprising 3 to 9 carbon atoms and comprising from 1 to 3 heteroatoms independently selected from oxygen, nitrogen and sulfur that is unsubstituted or substituted with 1 to 3 substituents independently selected from an oxo group and a —(C₁-C₃)-alkyl group.

Another embodiment is a compound of formula (I), wherein R₆ is selected from an ortho-fused bicyclic cycloalkyl group comprising 8 to 11 carbon atoms, said ortho-fused bicyclic cycloalkyl group being unsubstituted or substituted with 1 to 3 substituents independently selected from: a halogen atom; a methyl group; a hydroxy group, an —O-methyl group, and an oxo group.

Another embodiment is a compound of formula (I), wherein R₆ is selected from a bicyclic cycloalkyl group selected from the following list:

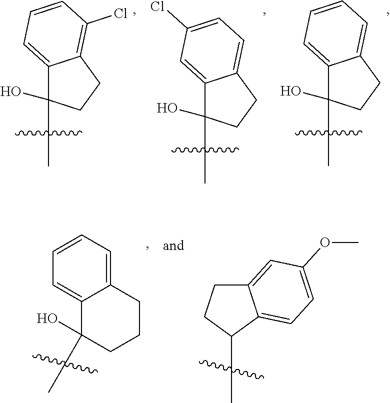

Another embodiment is a compound of formula (I), wherein R₆ is selected from a ortho-fused bicyclic heterocycloalkyl group comprising 8 to 9 carbon atoms and comprising 1 or 2 heteroatoms independently selected from oxygen and nitrogen, said bicyclic heterocycloalkyl group being unsubstituted or substituted with 1 to 3 substituents independently selected from: a halogen atom, a methyl group, an —O-methyl group, and an oxo group.

Another embodiment is a compound of formula (I), wherein R₆ is selected from a bicyclic heterocycloalkyl group selected from the following list:

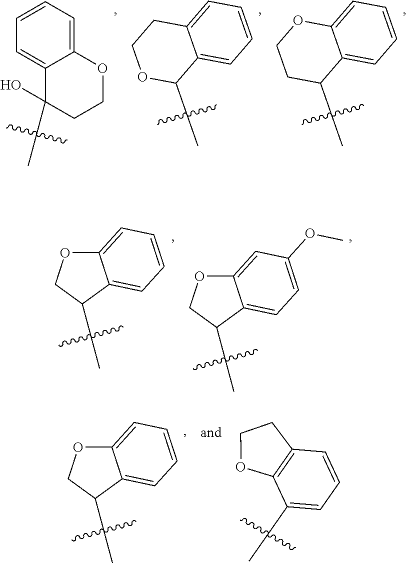

Another embodiment is a compound of formula (I), wherein R₇ represents a phenyl group, said phenyl group being unsubstituted or substituted with 1 to 3 substituents independently selected from the group consisting of a halogen atom, a —(C₁-C₃)-alkyl group, an —O—(C₁-C₃)-alkyl group and a morpholine group.

Another embodiment is a compound of formula (I), wherein R₇ represents a monocyclic cycloalkyl group comprising 4 to 7 carbon atoms, or a monocyclic heterocycloalkyl group comprising 3 to 6 carbon atoms and comprising from 1 to 2 heteroatoms independently selected from oxygen and nitrogen, said cycloalkyl and heterocycloalkyl group being unsubstituted or substituted with 1 to 3 substituents independently selected from the group consisting of: a halogen atom, an oxo group, a —(C₁-C₆)-alkyl group, a phenyl group, an —O—, a benzyl group, a —OH group, and an —O—(C₁-C₆)-alkyl group.

Another embodiment is a compound of formula (I), wherein R₇ represents a monocyclic heterocycloalkyl group comprising 3 to 6 carbon atoms and comprising from 1 to 2 heteroatoms independently selected from oxygen and nitrogen and being unsubstituted or substituted with 1 or 2 substituents independently selected from an —O— group, a phenyl group and a benzyl group.

Another embodiment is a compound of formula (I), wherein R₇ represents an ortho-fused bicyclic heterocycloalkyl group comprising 8 to 9 carbon atoms and comprising 1 or 2 heteroatoms independently selected from oxygen and nitrogen; wherein said ortho-fused bicyclic heterocycloalkyl groups being unsubstituted or substituted with 1 to 3 substituents independently selected from the group consisting of: a halogen atom, an oxo group, a —(C₁-C₆)-alkyl group, a phenyl group, an —O— group, a benzyl group, a —OH group, and an —O—(C₁-C₆)-alkyl group.

Another embodiment is a compound of formula (I), wherein R₇ represents an ortho-fused bicyclic cycloalkyl group comprising 8 to 11 carbon atoms, said bicyclic cycloalkyl group being unsubstituted or substituted with 1 to 3 substituents independently selected from the group consisting of: a halogen atom; a methyl group; a —OH group, an —O—(C₁-C₃)alkyl group, and an oxo group.

Another embodiment is a compound of formula (I), wherein R₇ is selected from an ortho-fused bicyclic heterocycloalkyl group comprising 8 to 9 carbon atoms and comprising 1 or 2 heteroatoms independently selected from oxygen and nitrogen, said bicyclic heterocycloalkyl group being unsubstituted or substituted with 1 to 3 substituents independently selected from: a halogen atom; a methyl group; a —OH group, an —O—(C₁-C₃)alkyl group, and an oxo group.

Another embodiment is a compound of formula (I), wherein R₇ represents a group selected from the following list:

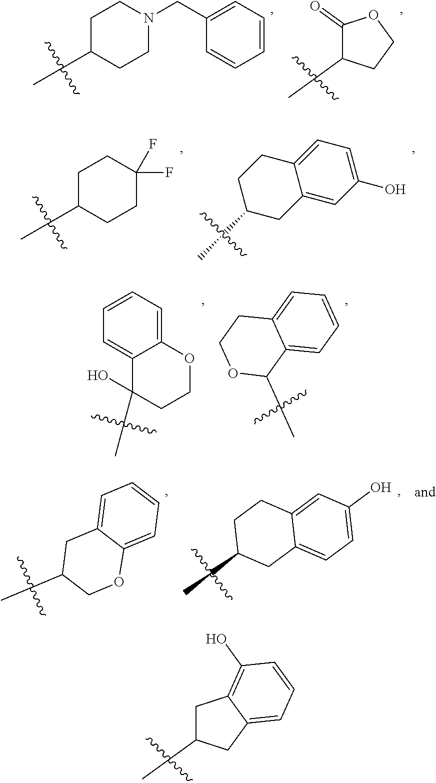

Another embodiment is a compound of formula (I), wherein R₃ represents a bond.

Another embodiment is a compound of formula (I), wherein R₃ represents an —O—.

Another embodiment is a compound of formula (I), wherein R₃ represents a —OC(═O)— group.

Another embodiment is a compound of formula (I), wherein R₃ represents a —N(H)C(═O)— group.

Another embodiment is a compound of formula (I), wherein R₃ represents a —C(═O)O— group.

Another embodiment is a compound of formula (I), wherein R₃ represents a —C(═O)N(H)— group.

Another embodiment is a compound of formula (I), wherein R₉ represents a hydrogen atom.

Another embodiment is a compound of formula (I), wherein R₉ represents a —C(═O)—OH group.

Another embodiment is a compound of formula (I), wherein R₉ represents a —C(═O)O(C₁-C₃)-alkyl group.

Another embodiment is a compound of formula (I), wherein R₉ represents a —OH group.

Another embodiment is a compound of formula (I), wherein R₉ represents a —O(C₁-C₃)-alkyl group.

Another embodiment is a compound of formula (I), wherein R₉ represents a —NH₂ group.

Another embodiment is a compound of formula (I), wherein R₁₀ represents an —O—(C₁-C₄)-alkyl group.

Another embodiment is a compound of formula (I), wherein R₁₀ represents a —N⁺—(CH₃)₃ group.

Another embodiment is a compound of formula (I), wherein R₁₀ represents a —N⁺H—(CH₃)₂ group.

Another embodiment is a compound of formula (I), wherein R₁₁ represents a bond.

Another embodiment is a compound of formula (I), wherein R₁₁ represents an —O—.

Another embodiment is a compound of formula (I), wherein R₁₁ represents a —C(═O)O— group.

Another embodiment is a compound of formula (I), wherein R₁₂ represents a —OH group.

Another embodiment is a compound of formula (I), wherein R₁₂ represents a —C(═O)OH group.

Another embodiment is a compound of formula (I), wherein R₁₂ represents a —C(═O)O(C₁-C₃)-alkyl group.

Another embodiment is a compound of formula (I), wherein R₁₂ represents a —C(═O)N(C₁-C₃)-alkyl group.

Another embodiment is a compound of formula (I), wherein R₁₂ represents a —NH₂ group.

Another embodiment is a compound of formula (I), wherein R₁₂ represents a —NHC(═O) (C₁-C₃)-alkyl group.

Another embodiment is a compound of formula (I), wherein R₁₂ represents a —C(═O)H group.

Another embodiment is a compound of formula (I), wherein R₁₂ represents or a heterocyclic group comprising 3 to 9 carbon atoms and comprising from 1 to 3 heteroatoms independently selected from oxygen, nitrogen and sulfur that is unsubstituted or substituted with 1 to 3 oxo group or —(C₁-C₃)-alkyl group.

Another embodiment is a compound of formula (I), wherein R₁₂ represents or an —O-heterocyclic group comprising 3 to 9 carbon atoms and comprising from 1 to 3 heteroatoms independently selected from oxygen, nitrogen and sulfur being unsubstituted or substituted with 1 to 3 oxo group or —(C₁-C₃)-alkyl group.

One embodiment is a compound of formula (I):

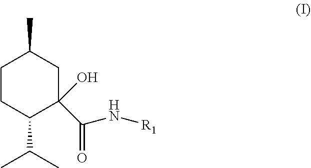

wherein:
R₁ is —C(R₂)(R₃)—[C(R₄)(R₅)]ₘ-L-R₆ or —R₇
m represents 0, 1, 2 or 3;
R₂ and R₃ independently represent a hydrogen atom, a deuterium atom, a —(C₁-C₆)-alkyl group, a (C₁-C₆)-alkyl-OH group, a —C(═O)NH₂ group, a —(C₁-C₆)-alkoxyl group, or a —C(═O)O(C₁-C₆)-alkyl group;
R₄ and R₅ independently represent a hydrogen atom, a deuterium atom, a fluorine atom, a —NH₂ group, a —OH group, a —(C₁-C₆)-alkyl group, a —CF₃ group, a carboxyl group, or a —R₈—(C₁-C₆)-alkyl-R₉ group wherein:
R₈ represents a bond, an —O—, a —OC(═O)— group, a —N(H)C(═O)— group, a —C(═O)O— group, or a —C(═O)N(H)— group;
R₉ represents a hydrogen atom, a —C(═O)—OH group, a —C(═O)O(C₁-C₃)-alkyl group, a —OH group, an —O—(C₁-C₃)-alkyl group, or a —NH₂ group;
or R₄ and R₅ can form, together with the carbon atom to which they are attached, a heterocycloalkyl group comprising 3 to 5 carbon atoms and comprising from 1 or 2 heteroatoms selected from oxygen and nitrogen;
L represents a bond, a —(C₁-C₆)-alkylene- group, an —O—(C₁-C₆)-alkylene- group, an —O—, a —OC(═O)— group, a —N(H)— group, a —C(═O)— group, a —C(═O)O— group, a —C(═O)—O—(C₁-C₃)-alkyl- group, a —C(═O)—N(H)— or a —CONH(C₁-C₆)-alkyl- group;
R₆ is selected from the group consisting of a —OH group; a —(C₁-C₆)-alkyl group; a phenyl group, a monocyclic heteroaryl group comprising 3 to 5 carbon atoms and comprising from 1 to 2 heteroatoms independently selected from oxygen, nitrogen and sulfur, an ortho-fused bicyclic heteroaryl group comprising 7 to 10 carbon atoms and comprising from 1 to 4 heteroatoms independently selected from oxygen, nitrogen and sulfur, an ortho-fused bicyclic cycloalkyl group comprising 8 to 11 carbon atoms, and an ortho-fused bicyclic heterocycloalkyl group comprising 8 to 9 carbon atoms and comprising 1 or 2 heteroatoms independently selected from oxygen and nitrogen; said phenyl, monocyclic heteroaryl, ortho-fused bicyclic heteroaryl, ortho-fused bicyclic cycloalkyl, ortho-fused bicyclic heterocycloalkyl groups being unsubstituted or substituted with 1 to 3 substituents independently selected from the group consisting of:
   a halogen atom, a —OH group, an oxo group, an —O—(C₁-C₆)-alkyl group, a —(C₁-C₆)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₆)-alkyl group, a —C(═O)N(C₁-C₃)-alkyl group, a —(OCH₂CH₂)ₙ—R₁₀ group, and a —R₁₁—(C₁-C₆)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents an —O—(C₁-C₄)-alkyl group, a —N⁺—(CH₃)₃ group, or —N⁺H—(CH₃)₂ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, a —C(═O)H group, a heterocyclic group or an —O-heterocyclic group, said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group.
R₇ represents a phenyl group, a monocyclic cycloalkyl group comprising 4 to 7 carbon atoms, a monocyclic heterocycloalkyl group comprising 3 to 6 carbon atoms and comprising from 1 to 2 heteroatoms independently selected from oxygen and nitrogen, an ortho-fused bicyclic cycloalkyl group comprising 8 to 11 carbon atoms, or an ortho-fused bicyclic heterocycloalkyl group comprising 8 to 9 carbon atoms and comprising 1 or 2 heteroatoms independently selected from oxygen and nitrogen; wherein said phenyl group being unsubstituted or substituted with 1 to 3 substituents independently selected from a halogen atom, a —(C₁-C₃)-alkyl group, an —O—(C₁-C₃)-alkyl group and a morpholine group; said monocyclic cycloalkyl, monocyclic heterocycloalkyl, an ortho-fused bicyclic heterocycloalkyl groups being unsubstituted or substituted with 1 to 3 substituents independently selected from the group consisting of: a halogen atom, an oxo group, a —(C₁-C₆)-alkyl group, a phenyl group, an —O— group, a benzyl group, a —OH group, and an —O—(C₁-C₆)-alkyl group;
   or a pharmaceutically acceptable salt thereof.

Another embodiment is a compound of formula (I), wherein R₁ represents a —C(R₂)(R₃)—[C(R₄)(R₅)]ₘ-L-R₆ group; m represents 0 or 1; or a pharmaceutically acceptable salt thereof.

In one embodiment, the compound of formula (I) has the absolute configuration corresponding to a compound of formula (Ia):

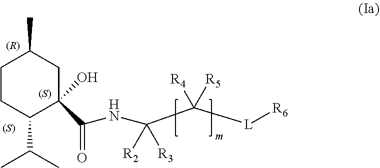

wherein:
m represents 0, 1, 2 or 3;
R₂ and R₃ independently represent a hydrogen atom, a deuterium atom, a —(C₁-C₃)-alkyl group, a (C₁-C₃)-alkyl-OH group, a —C(═O)NH₂ group, a —(C₁-C₃)-alkoxyl group, or a —C(═O)O(C₁-C₃)-alkyl group;
R₄ and R₅ independently represent a hydrogen atom, a deuterium atom, a fluorine atom, a —NH₂ group, a —OH group, a —(C₁-C₈)-alkyl group, a —CF₃ group, a carboxyl group, or a —R₈—(C₁-C₈)-alkyl-R₉ group wherein:
R₈ represents a bond, an —O—, a —OC(═O)— group, a —N(H)C(═O)— group, a —C(═O)O— group, or a —C(═O)N(H)— group;
R₉ represents a hydrogen atom, a —C(═O)—OH group, a —C(═O)O(C₁-C₃)-alkyl group, a —OH group, an —O—(C₁-C₃)-alkyl group, or a —NH₂ group;
or R₄ and R₅ can form, together with the carbon atom to which they are attached, a heterocycloalkyl group comprising 3 to 5 carbon atoms and comprising from 1 or 2 heteroatoms selected from oxygen and nitrogen;
L represents a bond, a —(C₁-C₃)-alkylene- group, an —O—(C₁-C₃)-alkylene- group, an —O—, a —OC(═O)— group, a —N(H)— group, a —C(═O)— group, a —C(═O)O— group, a —C(═O)—O—(C₁-C₃)-alkyl- group, a —C(═O)—N(H)— or a —CONH(C₁-C₃)-alkyl- group;
R₆ is selected from the group consisting of a —OH group; a —(C₁-C₃)-alkyl group; a phenyl group, a monocyclic heteroaryl group comprising 3 to 5 carbon atoms and comprising from 1 to 2 heteroatoms independently selected from oxygen, nitrogen and sulfur, an ortho-fused bicyclic heteroaryl group comprising 7 to 10 carbon atoms and comprising from 1 to 4 heteroatoms independently selected from oxygen, nitrogen and sulfur, an ortho-fused bicyclic cycloalkyl group comprising 8 to 11 carbon atoms, and an ortho-fused bicyclic heterocycloalkyl group comprising 8 to 9 carbon atoms and comprising 1 or 2 heteroatoms independently selected from oxygen and nitrogen; said phenyl, monocyclic heteroaryl, ortho-fused bicyclic heteroaryl, ortho-fused bicyclic cycloalkyl, ortho-fused bicyclic heterocycloalkyl groups being unsubstituted or substituted with 1 to 3 substituents independently selected from the group consisting of:
   a halogen atom, a —OH group, an oxo group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, a —C(═O)N(C₁-C₃)-alkyl group, a —(OCH₂CH₂)ₙ—R₁₀ group, and a —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents an —O—(C₁-C₃)-alkyl group, a —N⁺—(CH₃)₃ group, or —N⁺H—(CH₃)₂ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, a —C(═O)H group, a heterocyclic group or an —O-heterocyclic group, said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group;
      or a pharmaceutically acceptable salt thereof.

In one embodiment, the compound of formula (I) has the absolute configuration corresponding to a compound of formula (Ia):

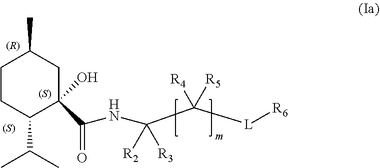

wherein:
m represents 0, or 1;
R₂ and R₃ independently represent a hydrogen atom, a deuterium atom, a —(C₁-C₃)-alkyl group, a (C₁-C₃)-alkyl-OH group, a —C(═O)NH₂ group, a —(C₁-C₃)-alkoxyl group, or a —C(═O)O(C₁-C₃)-alkyl group;
R₄ and R₅ independently represent a hydrogen atom, a deuterium atom, a fluorine atom, a —NH₂ group, a —OH group, a —(C₁-C₃)-alkyl group, a —CF₃ group, a carboxyl group, or a —R₈—(C₁-C₄)-alkyl-R₉ group wherein:
R₈ represents a bond, an —O—, a —OC(═O)— group, a —N(H)C(═O)— group, a —C(═O)O— group, or a —C(═O)N(H)— group;
R₉ represents a hydrogen atom, a —C(═O)—OH group, a —C(═O)O(C₁-C₃)-alkyl group, a —OH group, an —O—(C₁-C₃)-alkyl group, or a —NH₂ group;
or R₄ and R₅ can form, together with the carbon atom to which they are attached, a heterocycloalkyl group comprising 3 to 5 carbon atoms and comprising from 1 or 2 heteroatoms selected from oxygen and nitrogen;
L represents a bond, a —(C₁-C₃)-alkylene- group, an —O—(C₁-C₃)-alkylene- group, an —O—, a —OC(═O)— group, a —N(H)— group, a —C(═O)— group, a —C(═O)O— group, a —C(═O)—O—(C₁-C₃)-alkyl- group, a —C(═O)—N(H)— or a —CONH(C₁-C₃)-alkyl- group;
R₆ represents a phenyl group, wherein said phenyl group being unsubstituted or substituted with 1 to 3 substituents independently selected from the group consisting of:
   a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, a —C(═O)N(C₁-C₃)-alkyl group, a —(OCH₂CH₂)ₙ—R₁₀ group, and a —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents an —O—(C₁-C₃)-alkyl group, a —N⁺—(CH₃)₃ group, or —N⁺H—(CH₃)₂ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, a —C(═O)H group, a heterocyclic group or an —O-heterocyclic group, said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group;
      or a pharmaceutically acceptable salt thereof.

In one embodiment, the compound of formula (I) has the absolute configuration corresponding to a compound of formula (Ia):

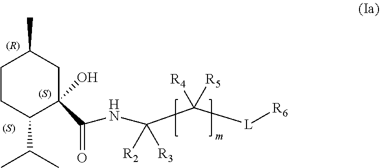

wherein:
m represents 1;
R₂ and R₃ independently represent a hydrogen atom, a —(C₁-C₃)-alkyl group, a —(C₁-C₃)-alkyl-OH group
R₄ and R₅ independently represent a hydrogen atom, a deuterium atom, a fluorine atom, a —NH₂ group, a —OH group, a —(C₁-C₃)-alkyl group, a —CF₃ group,
L represents a bond or a —C(═O)— group;
R₆ represents a phenyl group, wherein said phenyl group being unsubstituted or substituted with 1 to 2 substituents independently selected from the group consisting of:
   a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, a —C(═O)N(C₁-C₃)-alkyl group, a —(OCH₂CH₂)ₙ—R₁₀ group, and a —R₁₁—(C₁-C₃)-alkyl-R₁₂ group which is unsubstituted or substituted on the (C₁-C₆)-alkyl with a —OH group or a —NH₂ group; wherein n represents 1, 2 or 3; R₁₀ represents an —O—(C₁-C₃)-alkyl group, or a —N⁺—(CH₃)₃ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, a —C(═O)H group;
      or a pharmaceutically acceptable salt thereof.

In one embodiment, the compound of formula (I) has the absolute configuration corresponding to a compound of formula (Ia):

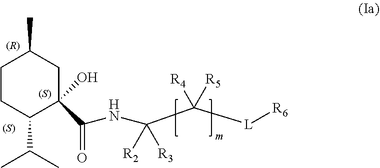

wherein:
m represents 0, or 1;
R₂ and R₃ independently represent a hydrogen atom, a —(C₁-C₃)-alkyl group, a —(C₁-C₃)-alkyl-OH group, a —(C₁-C₃)-alkoxyl group
R₄ and R₅ independently represent a hydrogen atom, a deuterium atom, a fluorine atom, a —NH₂ group, a —OH group, a —(C₁-C₃)-alkyl group, a —CF₃ group, or a —R₈—(C₁-C₄)-alkyl-R₉ group wherein:
R₈ represents a bond, an —O—, a —OC(═O)— group, a —N(H)C(═O)— group, a —C(═O)O— group, or a —C(═O)N(H)— group;
R₉ represents a hydrogen atom, a —C(═O)—OH group, a —C(═O)O(C₁-C₃)-alkyl group, a —OH group, an —O—(C₁-C₃)-alkyl group, or a —NH₂ group;
L represents a bond or a —C(═O)— group;
R₆ represents a phenyl group, wherein said phenyl group being unsubstituted or substituted with 1 to 3 substituents independently selected from the group consisting of:
   a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, a —C(═O)N(C₁-C₃)-alkyl group, a —(OCH₂CH₂)ₙ—R₁₀ group, and a —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents an —O—(C₁-C₃)-alkyl group, a —N⁺—(CH₃)₃ group, or —N⁺H—(CH₃)₂ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, a —C(═O)H group;
      or a pharmaceutically acceptable salt thereof.

Another embodiment is a compound of formula (I), wherein R₁ represents —R₇.

In one embodiment, the compound of formula (I) is (1S,2S,5R)-1-hydroxy-N-(3-hydroxyphenethyl)-2-isopropyl-5-methylcyclohexane-1-carboxamide; or a pharmaceutically acceptable salt thereof.

In one embodiment, the compound of formula (I) is 2-hydroxyethyl 2-(2-((1S,2S,5R)-1-hydroxy-2-isopropyl-5-methylcyclohexane-1-carboxamido)ethyl) benzoate; or a pharmaceutically acceptable salt thereof.

In one embodiment, the compound of formula (I) is (1S,2S,5R)-1-hydroxy-N-(2-(2-hydroxyethyl)phenethyl)-2-isopropyl-5-methylcyclohexane-1-carboxamide; or a pharmaceutically acceptable salt thereof.

In one embodiment, the compound of formula (I) is methyl-3-(2-((1S,2S,5R)-1-hydroxy-2-isopropyl-5-methylcyclohexane-1-carboxamido)ethyl)benzoate; or a pharmaceutically acceptable salt thereof.

In one embodiment, the compound of formula (I) is (1S,2S,5R)—N-(2-(2-amino-2-oxoethoxy)phenethyl)-1-hydroxy-2-isopropyl-5-methylcyclohexane-1-carboxamide; or a pharmaceutically acceptable salt thereof.

In one embodiment, the compound of formula (I) is (1S,2S,5R)-1-hydroxy-N—((S)-2-hydroxy-2-phenylethyl)-2-isopropyl-5-methylcyclohexane-1-carboxamide; or a pharmaceutically acceptable salt thereof.

In one embodiment, the compound of formula (I) is (1S,2S,5R)-1-hydroxy-N—((S)-2-hydroxy-2-phenylethyl)-2-isopropyl-5-methylcyclohexane-1-carboxamide; or a pharmaceutically acceptable salt thereof.

In one embodiment, the compound of formula (I) is 2-hydroxyethyl 3-(2-((1S,2S,5R)-1-hydroxy-2-isopropyl-5-methylcyclohexane-1-carboxamido)ethyl) benzoate; or a pharmaceutically acceptable salt thereof.

In one embodiment, the compound of formula (I) is (1S,2S,5R)-1-hydroxy-2-isopropyl-5-methyl-N-((3-phenyloxetan-3-yl)methyl)cyclohexane-1-carboxamide; or a pharmaceutically acceptable salt thereof.

Another embodiment is a process for preparing a compound of formula (I), comprising submitting a compound (intermediate 4):

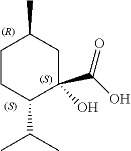

to a coupling reaction with a compound of formula (Ic) or a compound of formula (Id)

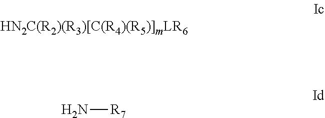

wherein m, R₂, R₃, R₄, R₅, R₆, L, R₇ are as defined for a compound of formula (I).

In one embodiment, the compound of formula (I) is selected from the group consisting of:
(1S,2S,5R)-1-hydroxy-N—((S)-2-hydroxy-2-phenylethyl)-2-isopropyl-5-methylcyclohexane-1-carboxamide:
2-hydroxyethyl 2-(2-((1S,2S,5R)-1-hydroxy-2-isopropyl-5-methylcyclohexane-1-carboxamido)ethyl) benzoate;
(1S,2S,5R)-1-hydroxy-N-(3-hydroxyphenethyl)-2-isopropyl-5-methylcyclohexane-1-carboxamide;
2-hydroxyethyl 3-(2-((1S,2S,5R)-1-hydroxy-2-isopropyl-5-methylcyclohexane-1-carboxamido)ethyl) benzoate;
(1S,2S,5R)-1-hydroxy-N-(2-(2-hydroxyethyl)phenethyl)-2-isopropyl-5-methylcyclohexane-1-carboxamide;
Methyl-3-(2-((1S,2S,5R)-1-hydroxy-2-isopropyl-5-methylcyclohexane-1-carboxamido)ethyl)benzoate;
(1S,2S,5R)-1-hydroxy-2-isopropyl-5-methyl-N-((3-phenyloxetan-3-yl)methyl)cyclohexane-1-carboxamide;
(1S,2S,5R)—N-(2-(2-amino-2-oxoethoxy)phenethyl)-1-hydroxy-2-isopropyl-5-methylcyclohexane-1-carboxamide;
   or a pharmaceutically acceptable salt thereof.

The compounds of formula (I) include the compounds having any combination of the above-defined embodiments for R₁, R₂, R₃, R₄, R₅, R₆, R₇, R₃, R₉, R₁₀, R₁₁, R₁₂, m, n, and L with each other.

In one embodiment, the compound of formula (I) is selected from the group consisting of:
(1S,2S,5R)-1-hydroxy-N—((S)-2-hydroxy-2-phenylethyl)-2-isopropyl-5-methylcyclohexane-1-carboxamide;
(1S,2S,5R)-1-hydroxy-2-isopropyl-5-methyl-N-(2-oxo-2-phenylethyl)cyclohexane-1-carboxamide;
(S)-2-((1S,2S,5R)-1-hydroxy-2-isopropyl-5-methylcyclohexane-1-carboxamido)-1-phenylethyl acetate;
(1S,2S,5R)—N-(2,2-difluoro-2-phenylethyl)-1-hydroxy-2-isopropyl-5-methylcyclohexane-1-carboxamide;
(1S,2S,5R)—N-(2-fluoro-2-phenylethyl)-1-hydroxy-2-isopropyl-5-methylcyclohexane-1-carboxamide;
(1S,2S,5R)—N-(2-(2-aminoethoxy)phenethyl)-1-hydroxy-2-isopropyl-5-methylcyclohexane-1-carboxamide:
(1S,2S,5R)-1-hydroxy-N-(2-(2-hydroxyethoxy)phenethyl)-2-isopropyl-5-methylcyclohexane-1-carboxamide;
(1S,2S,5R)—N-(2-(1H-benzo[d]imidazol-2-yl)ethyl)-1-hydroxy-2-isopropyl-5-methylcyclohexane-1-carboxamide 2-hydroxypropane-1,2,3-tricarboxylate;
(1S,2S,5R)—N-(2-(2,3-dihydroxypropoxy)phenethyl)-1-hydroxy-2-isopropyl-5-methylcyclohexane-1-carboxamide;
(1S,2S,5R)—N-(3-(2,3-dihydroxypropoxy)phenethyl)-1-hydroxy-2-isopropyl-5-methylcyclohexane-1-carboxamide;
2-(2-(2-((1S,2S,5R)-1-hydroxy-2-isopropyl-5-methylcyclohexane-1-carboxamido)ethyl)phenoxy)-N,N,N-trimethylethan-1-aminium iodide;
(1S,2S,5R)-1-hydroxy-N-(2-hydroxy-2-(m-tolyl)ethyl)-2-isopropyl-5-methylcyclohexane-1-carboxamide;
2-aminoethyl 2-(2-((1S,2S,5R)-1-hydroxy-2-isopropyl-5-methylcyclohexane-1-carboxamido)ethyl) benzoate hydrochloride;
(1S,2S,5R)-1-hydroxy-N-(2-(hydroxymethyl)phenethyl)-2-isopropyl-5-methylcyclohexane-1-carboxamide;
(1S,2S,5R)-1-hydroxy-N-(3-(hydroxymethyl)phenethyl)-2-isopropyl-5-methylcyclohexane-1-carboxamide;
2-aminoethyl 3-(2-((1S,2S,5R)-1-hydroxy-2-isopropyl-5-methylcyclohexane-1-carboxamido)ethyl) benzoate hydrochloride;
(1S,2S,5R)-1-hydroxy-2-isopropyl-5-methyl-N-((1RS)-(3-oxo-1,3-dihydroisobenzofuran-1-yl)methyl) cyclohexane-1-carboxamide;
(1S,2S,5R)-1-hydroxy-2-isopropyl-5-methyl-N-((1RS)-(3-oxo-1,3-dihydroisobenzofuran-1-yl)methyl) cyclohexane-1-carboxamide;
methyl (S)-2-amino-3-(2-(2-((1S,2S,5R)-1-hydroxy-2-isopropyl-5-methylcyclohexane-1-carboxamido) ethyl)phenyl)propanoate;
methyl (S)-2-amino-3-(3-(2-((1S,2S,5R)-1-hydroxy-2-isopropyl-5-methylcyclohexane-1carboxamido) ethyl)phenyl)propanoate;
2-hydroxyethyl 3-(2-((1S,2S,5R)-1-hydroxy-2-isopropyl-5-methylcyclohexane-1-carboxamido)ethyl) benzoate;
2-hydroxyethyl 2-(2-((1S,2S,5R)-1-hydroxy-2-isopropyl-5-methylcyclohexane-1-carboxamido)ethyl) benzoate;
(1S,2S,5R)-1-hydroxy-N-((1-hydroxy-2,3-dihydro-1H-inden-1-yl)methyl)-2-isopropyl-5-methylcyclohexane-1-carboxamide;
(1S,2S,5R)—N-(2-((R)-2,3-dihydroxypropoxy)phenethyl)-1-hydroxy-2-isopropyl-5-methylcyclohexane-1-carboxamide;
(1S,2S,5R)—N-(2-((S)-2,3-dihydroxypropoxy)phenethyl)-1-hydroxy-2-isopropyl-5-methylcyclohexane-1-carboxamide;
methyl O-(3-(2-((1S,2S,5R)-1-hydroxy-2-isopropyl-5-methylcyclohexane-1-carboxamido)ethyl)phenyl)-D-serinate;
methyl O-(3-(2-((1S,2S,5R)-1-hydroxy-2-isopropyl-5-methylcyclohexane-1-carboxamido)ethyl)phenyl)-L-serinate;
ethyl 3-(2-((1S,2S,5R)-1-hydroxy-2-isopropyl-5-methylcyclohexane-1-carboxamido)ethyl) benzoate;
(5-methyl-2-oxo-1,3-dioxol-4-yl)methyl 3-(2-((1S,2S,5R)-1-hydroxy-2-isopropyl-5-methylcyclohexane-1-carboxamido)ethyl) benzoate;
(1S,2S,5R)—N-(2-(benzylamino)-2-oxoethyl)-1-hydroxy-2-isopropyl-5-methylcyclohexane-1-carboxamide;
(1S,2S,5R)-1-hydroxy-2-isopropyl-5-methyl-N-((3-phenyloxetan-3-yl)methyl)cyclohexane-1-carboxamide;
(1S,2S,5R)-1-hydroxy-N-(2-hydroxy-2-(3-hydroxyphenyl)ethyl)-2-isopropyl-5-methylcyclohexane-1-carboxamide;
methyl 2-(3-(1-hydroxy-2-((1S,2S,5R)-1-hydroxy-2-isopropyl-5-methylcyclohexane-1-carboxamido)ethyl) phenoxy)acetate;
methyl 3-(1-hydroxy-2-((1S,2S,5R)-1-hydroxy-2-isopropyl-5-methylcyclohexane-1-carboxamido)ethyl) benzoate;
(1S,2S,5R)-1-hydroxy-N-(2-hydroxy-2-(2-hydroxyphenyl)ethyl)-2-isopropyl-5-methylcyclohexane-1-carboxamide;
methyl 2-(2-(1-hydroxy-2-((1S,2S,5R)-1-hydroxy-2-isopropyl-5-methylcyclohexane-1-carboxamido)ethyl) phenoxy)acetate;
(1S,2S,5R)-1-hydroxy-N-(2-hydroxy-2-(3-(hydroxymethyl) phenyl)ethyl)-2-isopropyl-5-methylcyclohexane-1-carboxamide;
(1S,2S,5R)-1-hydroxy-N-(2-(2-hydroxyphenyl)-2-oxoethyl)-2-isopropyl-5-methylcyclohexane-1-carboxamide;
(1S,2S,5R)-1-hydroxy-N-(2-(3-hydroxyphenyl)-2-oxoethyl)-2-isopropyl-5-methylcyclohexane-1-carboxamide;
(1S,2S,5R)-1-hydroxy-N-(3-hydroxyphenethyl)-2-isopropyl-5-methylcyclohexane-1-carboxamide;
(1S,2S,5R)-1-hydroxy-N-((2R)-hydroxy-2-(3-hydroxyphenyl)ethyl)-2-isopropyl-5-methylcyclohexane-1-carboxamide;
(1S,2S,5R)-1-hydroxy-N-((2S)-hydroxy-2-(3-hydroxyphenyl)ethyl)-2-isopropyl-5-methylcyclohexane-1-carboxamide;
(1S,2S,5R)-1-hydroxy-N-((2R)-hydroxy-2-(3-(hydroxymethyl)phenyl)ethyl)-2-isopropyl-5-methylcyclohexane-1-carboxamide;
(1S,2S,5R)-1-hydroxy-N-((2S)-hydroxy-2-(3-(hydroxymethyl)phenyl)ethyl)-2-isopropyl-5-methylcyclohexane-1-carboxamide;
(1S,2S,5R)-1-hydroxy-N-((2R)-hydroxy-2-(m-tolyl)ethyl)-2-isopropyl-5-methylcyclohexane-1-carboxamide;
(1S,2S,5R)-1-hydroxy-N-((2S)-hydroxy-2-(m-tolyl)ethyl)-2-isopropyl-5-methylcyclohexane-1-carboxamide;
(1S,2S,5R)-1-hydroxy-N-((2R)-1-hydroxy-3-phenylpropan-2-yl)-2-isopropyl-5-methylcyclohexane-1-carboxamide;
(1S,2S,5R)—N-(3-cyanophenethyl)-1-hydroxy-2-isopropyl-5-methylcyclohexane-1-carboxamide;
3-(2-((1S,2S,5R)-1-hydroxy-2-isopropyl-5-methylcyclohexane-1-carboxamido)ethyl) benzamide;
(1S,2S,5R)—N-((2S)-amino-2-phenylethyl)-1-hydroxy-2-isopropyl-5-methylcyclohexane-1-carboxamide;
(1S,2S,5R)-1-hydroxy-2-isopropyl-5-methyl-N-phenethylcyclohexane-1-carboxamide;
(1S,2S,5R)-1-hydroxy-N-(2-(2-hydroxyethyl)phenethyl)-2-isopropyl-5-methylcyclohexane-1-carboxamide;
(1S,2S,5R)—N-(2-(2-aminoethyl)phenethyl)-1-hydroxy-2-isopropyl-5-methylcyclohexane-1-carboxamide;
isopropyl ((1S,2S,5R)-1-hydroxy-2-isopropyl-5-methylcyclohexane-1-carbonyl)glycinate;
2-((S)-2-((1S,2S,5R)-1-hydroxy-2-isopropyl-5-methylcyclohexane-1-carboxamido)-1-phenylethoxy)acetic acid;
(1S,2S,5R)-1-hydroxy-N—((S)-2-(2-hydroxyethoxy)-2-phenylethyl)-2-isopropyl-5-methylcyclohexane-1-carboxamide;
methyl 2-((S)-2-((1S,2S,5R)-1-hydroxy-2-isopropyl-5-methylcyclohexane-1-carboxamido)-1-phenylethoxy)acetate;
(1S,2S,5R)-1-hydroxy-2-isopropyl-5-methyl-N-(3-methylphenethyl)cyclohexane-1-carboxamide;
(1S,2S,5R)-1-hydroxy-2-isopropyl-5-methyl-N-(2-methylphenethyl)cyclohexane-1-carboxamide;
(1S,2S,5R)-1-hydroxy-2-isopropyl-5-methyl-N-(4-methylphenethyl)cyclohexane-1-carboxamide;
methyl 2-(2-(2-((1S,2S,5R)-1-hydroxy-2-isopropyl-5-methylcyclohexane-1-carboxamido)ethyl)phenoxy)acetate;
(1S,2S,5R)—N—((S)-2-((R)-2-aminopropanamido)-2-phenylethyl)-1-hydroxy-2-isopropyl-5-methylcyclohexane-1-carboxamide;
(1S,2S,5R)—N—((S)-2-((S)-2-aminopropanamido)-2-phenylethyl)-1-hydroxy-2-isopropyl-5-methylcyclohexane-1-carboxamide;
methyl 2-(3-(2-((1S,2S,5R)-1-hydroxy-2-isopropyl-5-methylcyclohexane-1-carboxamido)ethyl)phenoxy)acetate;
methyl 4-((1S,2S,5R)-1-hydroxy-2-isopropyl-5-methylcyclohexane-1-carboxamido)-3-phenyl butanoate;
(1S,2S,5R)-1-hydroxy-N-(4-hydroxy-2-phenylbutyl)-2-isopropyl-5-methylcyclohexane-1-carboxamide;
phenyl ((1S,2S,5R)-1-hydroxy-2-isopropyl-5-methylcyclohexane-1-carbonyl)glycinate;
4-formyl-2-methoxyphenyl ((1S,2S,5R)-1-hydroxy-2-isopropyl-5-methylcyclohexane-1-carbonyl)glycinate;
methyl 4-((1S,2S,5R)-1-hydroxy-2-isopropyl-5-methylcyclohexane-1-carboxamido)-3-phenyl butanoate;
(1S,2S,5R)-1-hydroxy-N-(3-hydroxy-2-phenylpropyl)-2-isopropyl-5-methylcyclohexane-1-carboxamide;
2-aminoethyl 3-((1S,2S,5R)-1-hydroxy-2-isopropyl-5-methylcyclohexane-1-carboxamido)-2-phenylpropanoate hydrochloride;
(1S,2S,5R)-1-hydroxy-N-(2-hydroxy-2-phenylethyl-2-d)-2-isopropyl-5-methylcyclohexane-1-carboxamide:
2-amino-2-methylpropyl 3-((1S,2S,5R)-1-hydroxy-2-isopropyl-5-methylcyclohexane-1-carboxamido)-2-phenylpropanoate hydrochloride;
methyl 2-(3-(2-((1S,2S,5R)-1-hydroxy-2-isopropyl-5-methylcyclohexane-1-carboxamido)ethyl)phenyl)acetate;
methyl 3-(2-(2-((1S,2S,5R)-1-hydroxy-2-isopropyl-5-methylcyclohexane-1-carboxamido)ethyl)phenoxy)propanoate;
3,5-dihydroxyphenyl ((1S,2S,5R)-1-hydroxy-2-isopropyl-5-methylcyclohexane-1-carbonyl)glycinate;
methyl 2-(2-(2-((1S,2S,5R)-1-hydroxy-2-isopropyl-5-methylcyclohexane-1-carboxamido)ethyl)phenyl)acetate;
2-(3-(2-((1S,2S,5R)-1-hydroxy-2-isopropyl-5-methylcyclohexane-1-carboxamido)ethyl)phenyl)acetic acid;
4-(hydroxymethyl)-2-methoxyphenyl ((1S,2S,5R)-1-hydroxy-2-isopropyl-5-methylcyclohexane-1-carbonyl)glycinate;
(1S,2S,5R)-1-hydroxy-N-(2-(3-hydroxypropoxy)phenethyl)-2-isopropyl-5-methylcyclohexane-1-carboxamide;
(1S,2S,5R)—N-(2-(2-amino-2-oxoethoxy)phenethyl)-1-hydroxy-2-isopropyl-5-methyl cyclohexane-1-carboxamide;
(1S,2S,5R)-1-hydroxy-2-isopropyl-5-methyl-N-((3RS)-3,3,3-trifluoro-2-hydroxy-2-phenylpropyl) cyclohexane-1-carboxamide;
(1S,2S,5R)-1-hydroxy-2-isopropyl-5-methyl-N-((3RS)-3,3,3-trifluoro-2-hydroxy-2-phenylpropyl) cyclohexane-1-carboxamide;
(1S,2S,5R)—N-(2-(2-acetamidoethoxy)phenethyl)-1-hydroxy-2-isopropyl-5-methylcyclohexane-1-carboxamide;
(1S,2S,5R)—N-(2-(2-fluorophenyl)-2-oxoethyl)-1-hydroxy-2-isopropyl-5-methylcyclohexane-1-carboxamide;
(1S,2S,5R)-1-hydroxy-N—((R)-3-hydroxy-1-phenylpropyl)-2-isopropyl-5-methylcyclohexane-1-carboxamide;
3-(3-(2-((1S,2S,5R)-1-hydroxy-2-isopropyl-5-methylcyclohexane-1-carboxamido)ethyl) phenyl)propanoic acid;
(1S,2S,5R)-1-hydroxy-N-(2-((4-hydroxyphenyl)amino)-2-oxoethyl)-2-isopropyl-5-methylcyclohexane-1-carboxamide;
(1S,2S,5R)-1-hydroxy-N-((2RS)-3-hydroxy-2-phenylpropyl)-2-isopropyl-5-methylcyclohexane-1-carboxamide;
(1S,2S,5R)-1-hydroxy-N-((2RS)-3-hydroxy-2-phenylpropyl)-2-isopropyl-5-methylcyclohexane-1-carboxamide;
(1S,2S,5R)-1-hydroxy-N-((2RS)-2-hydroxy-2-phenylethyl-2-d)-2-isopropyl-5-methylcyclohexane-1-carboxamide;
(1S,2S,5R)-1-hydroxy-N-((2RS)-2-hydroxy-2-phenylethyl-2-d)-2-isopropyl-5-methylcyclohexane-1-carboxamide;
methyl 3-(2-(2-((1S,2S,5R)-1-hydroxy-2-isopropyl-5-methylcyclohexane-1-carboxamido) ethyl)phenyl)propanoate;
(1S,2S,5R)—N-((3S)-chroman-3-yl)-1-hydroxy-2-isopropyl-5-methylcyclohexane-1-carboxamide;
(1S,2S,5R)—N-((3R)-chroman-3-yl)-1-hydroxy-2-isopropyl-5-methylcyclohexane-1-carboxamide;
(1S,2S,5R)-1-hydroxy-N-((4-hydroxychroman-4-yl)methyl)-2-isopropyl-5-methylcyclohexane-1-carboxamide;
(1S,2S,5R)-1-hydroxy-N-((1RS)-isochroman-1-ylmethyl)-2-isopropyl-5-methylcyclohexane-1-carboxamide;
(1S,2S,5R)-1-hydroxy-N-((1RS)-isochroman-1-ylmethyl)-2-isopropyl-5-methylcyclohexane-1-carboxamide;
(1S,2S,5R)—N-(2-(2,4-dimethoxyphenyl)-2-oxoethyl)-1-hydroxy-2-isopropyl-5-methylcyclohexane-1-carboxamide;
(1S,2S,5R)—N-(2-(3,4-dihydroxyphenyl)-2-oxoethyl)-1-hydroxy-2-isopropyl-5-methylcyclohexane-1-carboxamide;
(1S,2S,5R)-1-hydroxy-2-isopropyl-5-methyl-N-((3-(3-(((tetrahydro-2H-pyran-2-yl)oxy)methyl)phenyl)oxetan-3-yl)methyl)cyclohexane-1-carboxamide;
(1S,2S,5R)—N-(2-(2,4-dihydroxyphenyl)-2-oxoethyl)-1-hydroxy-2-isopropyl-5-methylcyclohexane-1-carboxamide;
(1S,2S,5R)—N-(chroman-4-ylmethyl)-1-hydroxy-2-isopropyl-5-methylcyclohexane-1-carboxamide;
(1S,2S,5R)—N-(2-(2,3-dihydroxyphenyl)-2-oxoethyl)-1-hydroxy-2-isopropyl-5-methylcyclohexane-1-carboxamide;
(S)-2-hydroxypropyl 3-(2-((1S,2S,5R)-1-hydroxy-2-isopropyl-5-methylcyclohexane-1-carboxamido)ethyl)benzoate;
methyl 2-(2-((1S,2S,5R)-1-hydroxy-2-isopropyl-5-methylcyclohexane-1-carboxamido)ethyl)benzoate;
methyl 3-(2-((1S,2S,5R)-1-hydroxy-2-isopropyl-5-methylcyclohexane-1-carboxamido)ethyl)benzoate;
methyl 4-(2-((1S,2S,5R)-1-hydroxy-2-isopropyl-5-methylcyclohexane-1-carboxamido) ethyl)benzoate;
(1S,2S,5R)-1-hydroxy-N-(4-(hydroxymethyl)phenethyl)-2-isopropyl-5-methylcyclohexane-1-carboxamide;
(1S,2S,5R)-1-hydroxy-N-(2-hydroxyethyl)-2-isopropyl-5-methylcyclohexane-1-carboxamide;
2-((1S,2S,5R)-1-hydroxy-2-isopropyl-5-methylcyclohexane-1-carboxamido)ethyl benzoate;
(1S,2S,5R)-1-hydroxy-2-isopropyl-5-methyl-N-(2-oxotetrahydrofuran-3-yl)cyclohexane-1-carboxamide;
(1S,2S,5R)—N-[2-(3,4-dimethoxyphenyl)ethyl]-1-hydroxy-2-isopropyl-5-methyl-cyclohexanecarboxamide;
(1S,2S,5R)-1-hydroxy-N-[2-(4-hydroxy-3-methoxy-phenyl)ethyl]-2-isopropyl-5-methyl-cyclohexanecarboxamide;
(1S,2S,5R)—N-[2-(2,3-dimethoxyphenyl)ethyl]-1-hydroxy-2-isopropyl-5-methyl-cyclohexanecarboxamide;
(1S,2S,5R)-1-hydroxy-N-[2-(2-hydroxyphenyl)ethyl]-2-isopropyl-5-methyl-cyclohexanecarboxamide;
(1S,2S,5R)-1-hydroxy-N-[2-(4-hydroxyphenyl)ethyl]-2-isopropyl-5-methyl-cyclohexanecarboxamide;
(1S,2S,5R)-1-hydroxy-2-isopropyl-5-methyl-N-[2-(4-sulfamoylphenyl)ethyl]cyclohexanecarboxamide;
(1S,2S,5R)-1-hydroxy-2-isopropyl-5-methyl-N-[2-(4-pyridyl)ethyl]cyclohexanecarboxamide;
(1S,2S,5R)-1-hydroxy-2-isopropyl-5-methyl-N-(2-phenoxyethyl)cyclohexanecarboxamide;
(1S,2S,5R)-1-hydroxy-2-isopropyl-N-[2-(4-methoxyphenyl)-2-oxo-ethyl]-5-methyl-cyclohexanecarboxamide;
(1S,2S,5R)-1-hydroxy-N-[(1S,2S)-2-hydroxy-1-(methoxymethyl)-2-phenyl-ethyl]-2-isopropyl-5-methyl-cyclohexanecarboxamide;
(1S,2S,5R)—N-[2-(3,5-dimethoxyphenyl)ethyl]-1-hydroxy-2-isopropyl-5-methyl-cyclohexanecarboxamide;
(1S,2S,5R)-1-hydroxy-2-isopropyl-5-methyl-N-[2-(3-pyridyl)ethyl]cyclohexanecarboxamide;
(1S,2S,5R)-1-hydroxy-2-isopropyl-5-methyl-N-[2-(2-pyridyl)ethyl]cyclohexanecarboxamide;
(1S,2S,5R)-1-hydroxy-N-[2-(3-hydroxy-4-methoxy-phenyl)ethyl]-2-isopropyl-5-methyl-cyclohexanecarboxamide;
(1S,2S,5R)-1-hydroxy-2-isopropyl-5-methyl-N-[2-(3-methyl-2-pyridyl)ethyl]cyclohexanecarboxamide;
(1S,2S,5R)—N-[2-(2,5-dimethoxyphenyl)ethyl]-1-hydroxy-2-isopropyl-5-methyl-cyclohexanecarboxamide;
(1S,2S,5R)—N-(2-anilino-2-oxo-ethyl)-1-hydroxy-2-isopropyl-5-methyl-cyclohexanecarboxamide;
(1S,2S,5R)-1-hydroxy-N-[2-(4-hydroxy-3,5-dimethoxy-phenyl)ethyl]-2-isopropyl-5-methyl-cyclohexanecarboxamide;
(1S,2S,5R)-1-hydroxy-2-isopropyl-5-methyl-N-(2-pyrazin-2-ylethyl)cyclohexanecarboxamide;
benzyl 2-[[(1S,2S,5R)-1-hydroxy-2-isopropyl-5-methyl-cyclohexanecarbonyl]amino]acetate;
(1S,2S,5R)-1-hydroxy-2-isopropyl-5-methyl-N-[2-(3-sulfamoylphenyl)ethyl]cyclohexanecarboxamide;
(1S,2S,5R)—N-[2-(4-chlorophenyl)-2-oxo-ethyl]-1-hydroxy-2-isopropyl-5-methyl-cyclohexanecarboxamide;
(1S,2S,5R)—N-[2-(4-fluorophenyl)-2-oxo-ethyl]-1-hydroxy-2-isopropyl-5-methyl-cyclohexanecarboxamide;
(1S,2S,5R)—N-[2-(3,4-difluorophenyl)-2-oxo-ethyl]-1-hydroxy-2-isopropyl-5-methyl-cyclohexanecarboxamide;
(1S,2S,5R)—N-[2-(2,4-dichlorophenyl)-2-oxo-ethyl]-1-hydroxy-2-isopropyl-5-methyl-cyclohexanecarboxamide;
(1S,2S,5R)—N-[2-(3,5-difluorophenyl)-2-oxo-ethyl]-1-hydroxy-2-isopropyl-5-methyl-cyclohexanecarboxamide;
(1S,2S,5R)—N-[2-(2,5-difluorophenyl)-2-oxo-ethyl]-1-hydroxy-2-isopropyl-5-methyl-cyclohexanecarboxamide;
(1S,2S,5R)—N-[2-(2-chlorophenyl)-2-oxo-ethyl]-1-hydroxy-2-isopropyl-5-methyl-cyclohexanecarboxamide;
(1S,2S,5R)-1-hydroxy-2-isopropyl-5-methyl-N-[2-(m-tolyl)-2-oxo-ethyl]cyclohexanecarboxamide;
(1S,2S,5R)—N-[2-(2,3-difluorophenyl)-2-oxo-ethyl]-1-hydroxy-2-isopropyl-5-methyl-cyclohexanecarboxamide;
(1S,2S,5R)-1-hydroxy-N-[2-(4-hydroxyphenyl)-2-oxo-ethyl]-2-isopropyl-5-methyl-cyclohexanecarboxamide;
(1S,2S,5R)—N-[(4-chloro-1-hydroxy-indan-1-yl)methyl]-1-hydroxy-2-isopropyl-5-methyl-cyclohexanecarboxamide;
(1S,2S,5R)—N-[(6-chloro-1-hydroxy-indan-1-yl)methyl]-1-hydroxy-2-isopropyl-5-methyl-cyclohexanecarboxamide;
(1S,2S,5R)—N-[(1S)-1-benzyl-2-hydroxy-ethyl]-1-hydroxy-2-isopropyl-5-methyl-cyclohexanecarboxamide;
(1S,2S,5R)—N-(4,4-difluorocyclohexyl)-1-hydroxy-2-isopropyl-5-methyl-cyclohexanecarboxamide;
(1S,2S,5R)—N-[2-(3-chloro-2-thienyl)-2-oxo-ethyl]-1-hydroxy-2-isopropyl-5-methyl-cyclohexanecarboxamide;
(1S,2S,5R)-1-hydroxy-2-isopropyl-5-methyl-N-[2-oxo-2-(2-thienyl)ethyl]cyclohexanecarboxamide;
(1S,2S,5R)-1-hydroxy-N-[2-(1H-indol-3-yl)-2-oxo-ethyl]-2-isopropyl-5-methyl-cyclohexanecarboxamide;
(1S,2S,5R)-1-hydroxy-N-[(1R)-3-hydroxy-1-(2-thienylmethyl)propyl]-2-isopropyl-5-methyl-cyclohexanecarboxamide;
(1S,2S,5R)-1-hydroxy-2-isopropyl-N-[(5-methoxyindan-1-yl)methyl]-5-methyl-cyclohexanecarboxamide;
(1S,2S,5R)-1-hydroxy-2-isopropyl-5-methyl-N-[2-(2-methyl-1H-indol-3-yl)ethyl]cyclohexanecarboxamide;
(1S,2S,5R)-1-hydroxy-2-isopropyl-N-[(6-methoxy-2,3-dihydrobenzofuran-3-yl)methyl]-5-methyl-cyclohexanecarboxamide;
(1S,2S,5R)-1-hydroxy-2-isopropyl-N-[2-(2-methoxyphenyl)ethyl]-5-methyl-cyclohexanecarboxamide;
(1S,2S,5R)-1-hydroxy-2-isopropyl-5-methyl-N-(2-phenylpropyl)cyclohexanecarboxamide;
(1S,2S,5R)—N-[2-(2-fluorophenyl)-2-hydroxy-ethyl]-1-hydroxy-2-isopropyl-5-methyl-cyclohexanecarboxamide;
(1S,2S,5R)—N-[2-(4-fluorophenyl)-2-hydroxy-ethyl]-1-hydroxy-2-isopropyl-5-methyl-cyclohexanecarboxamide;
(1S,2S,5R)—N-[2-(2,3-dihydrobenzofuran-7-yl)ethyl]-1-hydroxy-2-isopropyl-5-methyl-cyclohexanecarboxamide;
(1S,2S,5R)—N-[2-(2-chlorophenyl)-2-hydroxy-ethyl]-1-hydroxy-2-isopropyl-5-methyl-cyclohexanecarboxamide;
(1S,2S,5R)-1-hydroxy-N-[[3-(hydroxymethyl)phenyl]methyl]-2-isopropyl-5-methyl-cyclohexanecarboxamide;
(1S,2S,5R)—N-(2,3-dihydrobenzofuran-3-ylmethyl)-1-hydroxy-2-isopropyl-5-methyl-cyclohexanecarboxamide;
(1S,2S,5R)-1-hydroxy-N-[3-hydroxy-1-(3-hydroxyphenyl)propyl]-2-isopropyl-5-methyl-cyclohexanecarboxamide;
(1S,2S,5R)-1-hydroxy-N-(2-hydroxy-2-phenyl-propyl)-2-isopropyl-5-methyl-cyclohexanecarboxamide;
(1S,2S,5R)-1-hydroxy-N-[(3-hydroxyphenyl)methyl]-2-isopropyl-5-methyl-cyclohexanecarboxamide;
(1S,2S,5R)-1-hydroxy-N-[2-(4-hydroxy-3-nitro-phenyl)ethyl]-2-isopropyl-5-methyl-cyclohexanecarboxamide;
(1S,2S,5R)-1-hydroxy-N-[3-(4-hydroxyphenyl)propyl]-2-isopropyl-5-methyl-cyclohexanecarboxamide;
(1S,2S,5R)-1-hydroxy-N-[(1S)-3-(4-hydroxyphenyl)-1-methyl-propyl]-2-isopropyl-5-methyl-cyclohexanecarboxamide;
(1S,2S,5R)-1-hydroxy-N-[2-(5-hydroxy-1H-indol-3-yl)ethyl]-2-isopropyl-5-methyl-cyclohexanecarboxamide;
(1S,2S,5R)-1-hydroxy-N-[(1R)-1-(hydroxymethyl)-2-(4-hydroxyphenyl)ethyl]-2-isopropyl-5-methyl-cyclohexanecarboxamide;
(1S,2S,5R)-1-hydroxy-N-[(2R)-7-hydroxytetralin-2-yl]-2-isopropyl-5-methyl-cyclohexanecarboxamide;
(1S,2S,5R)—N-[2-(2-bromo-5-hydroxy-phenyl)ethyl]-1-hydroxy-2-isopropyl-5-methyl-cyclohexanecarboxamide;
(1S,2S,5R)—N-[2-(2,4-dihydroxyphenyl)ethyl]-1-hydroxy-2-isopropyl-5-methyl-cyclohexanecarboxamide;
(1S,2S,5R)-1-hydroxy-N-(((1RS)-hydroxy-2,3-dihydro-1H-inden-1-yl)methyl)-2-isopropyl-5-methylcyclohexane-1-carboxamide;
(1S,2S,5R)-1-hydroxy-N-(((1RS)-hydroxy-2,3-dihydro-1H-inden-1-yl)methyl)-2-isopropyl-5-methylcyclohexane-1-carboxamide;
(1S,2S,5R)-1-hydroxy-N-(((1RS)-hydroxy-1,2,3,4-tetrahydronaphthalen-1-yl)methyl)-2-isopropyl-5-methylcyclohexane-1-carboxamide;
(1S,2S,5R)-1-hydroxy-N-((2R)-2-hydroxy-2-phenylethyl)-2-isopropyl-5-methylcyclohexane-1-carboxamide;
(1S,2S,5R)—N-((2R)-2-fluoro-2-phenylethyl)-1-hydroxy-2-isopropyl-5-methylcyclohexane-1-carboxamide;
(1S,2S,5R)—N-((2S)-2-fluoro-2-phenylethyl)-1-hydroxy-2-isopropyl-5-methylcyclohexane-1-carboxamide;
(1S,2S,5R)—N-((2RS)-2-((S)-2-aminopropanamido)-2-phenylethyl)-1-hydroxy-2-isopropyl-5-methylcyclohexane-1-carboxamide;
(1S,2S,5R)—N-((2RS)-2-((S)-2-aminopropanamido)-2-phenylethyl)-1-hydroxy-2-isopropyl-5-methylcyclohexane-1-carboxamide;
(1S,2S,5R)—N-((2RS)-(2-aminoacetamido)-2-phenylethyl)-1-hydroxy-2-isopropyl-5-methylcyclohexane-1-carboxamide;
(1S,2S,5R)—N-((2RS)-2-((S)-2-aminopropanamido)-2-phenylethyl)-1-hydroxy-2-isopropyl-5-methylcyclohexane-1-carboxamide;
(1S,2S,5R)-1-hydroxy-2-isopropyl-N-[2-[3-[2-[2-(2-methoxyethoxy)ethoxy]ethoxy]phenyl]ethyl]-5-methyl-cyclohexanecarboxamide;
(1S,2S,5R)—N-[2,2-difluoro-2-(2-methoxyphenyl)ethyl]-1-hydroxy-2-isopropyl-5-methyl-cyclohexanecarboxamide;
(1S,2S,5R)—N-[2,2-difluoro-2-(3-methylphenyl)ethyl]-1-hydroxy-2-isopropyl-5-methyl-cyclohexanecarboxamide;
(1S,2S,5R)—N-[2,2-difluoro-2-(3-methoxyphenyl)ethyl]-1-hydroxy-2-isopropyl-5-methyl-cyclohexanecarboxamide; and
(1S,2S,5R)-1-hydroxy-N-(4-methoxyphenyl)-5-methyl-2-propan-2-ylcyclohexane-1-carboxamide;
   or a pharmaceutically acceptable salt thereof.

Another embodiment is a compound of formula (I) or a pharmaceutically acceptable salt thereof, for use in therapy, especially as an agonist of TRPM8 receptors or an activator of TRPM8 receptors.

Another embodiment is a compound of formula (I) or a pharmaceutically acceptable salt thereof, for use as medicament.

Another embodiment is a compound of formula (I), or a pharmaceutically acceptable salt thereof, for use in the treatment of oropharyngeal dysphagia.

Another embodiment is a compound of formula (I), or a pharmaceutically acceptable salt thereof, for use in the treatment of pediatric dysphagia.

Another embodiment is a compound of formula (I), or a pharmaceutically acceptable salt thereof, for use in the treatment of post-extubation dysphagia.

Another embodiment is a method of treating oropharyngeal dysphagia, comprising administering to a subject in need thereof, in particular a human, a therapeutically effective amount of a compound of formula (I), or a pharmaceutically acceptable salt thereof.

Another embodiment is a method of treating pediatric dysphagia, comprising administering to a subject in need thereof, in particular a human, a therapeutically effective amount of a compound of formula (I), or a pharmaceutically acceptable salt thereof.

Another embodiment is a method of treating post-extubation dysphagia, comprising administering to a subject in need thereof, in particular a human, a therapeutically effective amount of a compound of formula (I), or a pharmaceutically acceptable salt thereof.

Another embodiment is a pharmaceutical composition comprising as active principle an effective dose of a compound of formula (I), or a pharmaceutically acceptable salt thereof, and at least one pharmaceutically acceptable excipient.

One embodiment is a pharmaceutical composition comprising a compound of formula (I) or a pharmaceutically acceptable salt and at least one pharmaceutically acceptable excipient.

One embodiment is a pharmaceutical composition comprising a compound of formula (I) or a pharmaceutically acceptable salt thereof and at least one pharmaceutically acceptable excipient, wherein the compound of formula (I) is (1S,2S,5R)-1-hydroxy-N—((S)-2-hydroxy-2-phenylethyl)-2-isopropyl-5-methylcyclohexane-1-carboxamide; or a pharmaceutically acceptable salt thereof.

One embodiment is a pharmaceutical composition comprising a compound of formula (I) or a pharmaceutically acceptable salt thereof and at least one pharmaceutically acceptable excipient, wherein the compound of formula (I) is 2-hydroxyethyl 2-(2-((1S,2S,5R)-1-hydroxy-2-isopropyl-5-methylcyclohexane-1-carboxamido)ethyl) benzoate; or a pharmaceutically acceptable salt thereof.

One embodiment is a pharmaceutical composition comprising a compound of formula (I) or a pharmaceutically acceptable salt thereof and at least one pharmaceutically acceptable excipient, wherein the compound of formula (I) is (1S,2S,5R)-1-hydroxy-N-(3-hydroxyphenethyl)-2-isopropyl-5-methylcyclohexane-1-carboxamide; or a pharmaceutically acceptable salt thereof.

One embodiment is a pharmaceutical composition comprising a compound of formula (I) or a pharmaceutically acceptable salt thereof and at least one pharmaceutically acceptable excipient, wherein the compound of formula (I) is 2-hydroxyethyl 3-(2-((1S,2S,5R)-1-hydroxy-2-isopropyl-5-methylcyclohexane-1-carboxamido)ethyl) benzoate; or a pharmaceutically acceptable salt thereof.

One embodiment is a pharmaceutical composition comprising a compound of formula (I) or a pharmaceutically acceptable salt thereof and at least one pharmaceutically acceptable excipient, wherein the compound of formula (I) is (1S,2S,5R)-1-hydroxy-N-(2-(2-hydroxyethyl)phenethyl)-2-isopropyl-5-methylcyclohexane-1-carboxamide; or a pharmaceutically acceptable salt thereof.

One embodiment is a pharmaceutical composition comprising a compound of formula (I) or a pharmaceutically acceptable salt thereof and at least one pharmaceutically acceptable excipient, wherein the compound of formula (I) is Methyl-3-(2-((1S,2S,5R)-1-hydroxy-2-isopropyl-5-methylcyclohexane-1-carboxamido)ethyl)benzoate; or a pharmaceutically acceptable salt thereof.

One embodiment is a pharmaceutical composition comprising a compound of formula (I) or a pharmaceutically acceptable salt thereof and at least one pharmaceutically acceptable excipient, wherein the compound of formula (I) is (1S,2S,5R)-1-hydroxy-2-isopropyl-5-methyl-N-((3-phenyloxetan-3-yl)methyl)cyclohexane-1-carboxamide; or a pharmaceutically acceptable salt thereof.

One embodiment is a pharmaceutical composition comprising a compound of formula (I) or a pharmaceutically acceptable salt thereof and at least one pharmaceutically acceptable excipient, wherein the compound of formula (I) is (1S,2S,5R)—N-(2-(2-amino-2-oxoethoxy)phenethyl)-1-hydroxy-2-isopropyl-5-methylcyclohexane-1-carboxamide; or a pharmaceutically acceptable salt thereof.

One embodiment is a method of treating a disease involving activation of TRPM8 receptors, comprising administering to a subject in need thereof a therapeutically effective amount of a compound of formula (I), or a pharmaceutically acceptable salt thereof.

One embodiment is a method of treating oropharyngeal dysphagia, comprising administering to a subject in need thereof a therapeutically effective amount of a compound of formula (I), or a pharmaceutically acceptable salt thereof.

The compounds of the formula (I) can be prepared by the following processes.

The compounds of the formula (I) are synthesized using techniques and materials described below or otherwise known by the skilled person in the art. In addition, solvents, temperatures and other reaction conditions presented below may vary as deemed appropriate to the skilled person in the art.

General below methods for the preparation of compounds of formula (I) are optionally modified by the use of appropriate reagents and conditions for the introduction of the various moieties found in the compound of formula (I) as described below.

As used above, and throughout the description of the invention, the following abbreviations, unless otherwise indicated, shall be understood to have the following meanings:
BBr₃ boron bromide
BH₃-THF borane tetrahydrofuran
BOC-BETA-IODO-ALA-OME N-(tert-butoxycarbonyl)-3-iodo-L-alanine methyl ester
BOC-D-SER-OME N-(tert-butoxycarbonyl)-L-serine methyl ester,
BOC-D-ALA-OH N-(tert-butoxycarbonyl)-D-alanine
BOC-L-ALA-OH N-(tert-butoxycarbonyl)-L-alanine
BOC-GLY-OH N-(tert-butoxycarbonyl)glycine
CaCl₂ calcium chloride
CDI 1,1′-carbonyldiimidazole
CH₃CN methyl cyanide
CHO Chinese hamster ovary
CO₂ carbon dioxide
DBU 1,8-diazabicyclo[5.4.0]undec-7-ene
DCC dicyclohexylcarbodiimide
DCM dichloromethane
Dess-Martin periodinane 1, 1, 1-tris(acetyloxy)-1,1-dihydro-1,2-benziodoxol-3-(1H)-one
DMAP 4-(dimethylamino)pyridine
DME 1,2-dimethoxyethane
DM N,N-dimethylformamide
DMSO dimethyl sulfoxide
MeOH methanol
EDAC N-ethyl-N′-(3-dimethylaminopropyl)carbodiimide hydrochloride
EGTA ethylene glycol-bis(2-aminoethylether)-N,N,N′,N′-tetraacetic acid
Eq equivalent
Et2O diethyl ether
EtOAc ethyl acetate
ES+: electrospray positive ionization
FA formic acid
FCS fetal calf serum
FLIP FLIP assay results on human cell lines
FLIPR FLIP assay results on pig cell lines
Gr grams
HCl hydrogen chloride
H₂ hydrogen
HBSS Hank's balanced salt solution
HCOOH formic acid
HEPES 4-(2-hydroxyethyl)piperazine-1-ethanesulfonic acid
HNMR Proton NMR spectrum
HOBT 1-hydroxybenzotriazole hydrate
HPLC High Performance Liquid Chromatography
hr hour
K₂CO₃ potassium carbonate
KCl potassium chloride
KF potassium fluoride
KOH potassium hydroxide
LCMS liquid chromatography mass spectrometry
LDA lithium diisopropylamide
LiBH₄ lithium borohydride
M molar
MeCN methyl cyanide
MeOH methanol
MTBE methyl tert-butyl ether
Mg magnesium
MgCl₂ magnesium chloride
ml or mL milliliter
N normal
N-Boc N-tert-butoxycarbonyl
NaCl sodium chloride
NaN₃ sodium azide
NaH₂PO₄ sodium dihydrogen phosphate
NaOClO sodium chlorite
NaOH sodium hydroxide
Na₂SO₃ sodium sulfite
Na₂S₂O₃ sodium thiosulfate
Na₂SO₄ sodium sulfate
NH₄Cl ammonium chloride
N₂ nitrogen
Pd(Ph)₄ tetrakis(triphenylphosphine)palladium(0)
PPh₃ triphenylphosphine
PPTS pyridinium p-toluenesulfonate
PtO₂ platinum oxide
Pd palladium
RP-HPLC reversed-phase high performance liquid chromatography
rt room temperature
RT retention time
TBAF tetra-n-butylammonium fluoride
t-BuOH tert-butyl hydroxide
TEA triethylamine
TFA trifluoroacetic acid
THF tetrahydrofuran
UPLC ultra performance liquid chromatography
V volume
XPhos 2-dicyclohexylphosphino-2′,4′,6′-triisopropylbiphenyl
XPhos Pd chloro(2-dicyclohexylphosphino-2′,4′,6′-triisopropyl-1,1′-biphenyl)[2-(2′-amino-1,1′-biphenyl)]palladium(II)

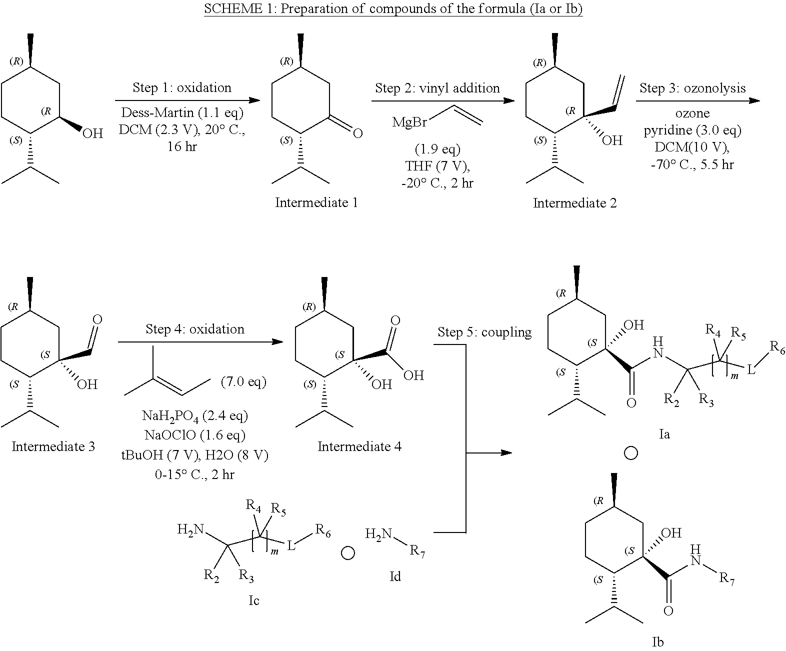

According to SCHEME 1, in which R₁, R₂, R₃, R₄, R₅, R₆ and R₇ are defined as described above, L-Menthol is oxidized in Step 1 into corresponding ketone L-menthone (intermediate 1) for instance by treatment with Dess-Martin reagent in a solvent such as dichloromethane (DCM). This intermediate 1 is subjected in Step 2 to a vinyl addition by treatment for example with bromomagnesium vinyl in a solvent such as tetrahydrofuran (THF) to give intermediate 2.

The intermediate 2 is then oxidized in Step 3 to give the aldehyde intermediate 3, for instance with ozone in a solvent such as dichloromethane (DCM) in the presence of a base, like pyridine. The intermediate 3 is oxidized in Step 4 to generate the acid intermediate 4 such as by treatment with sodium chlorite and sodium dihydrogen phosphate in the presence of 2-methyl-2-butene at room temperature.

The intermediate 4 is then subjected in Step 5 to a coupling reaction with an amino compound H₂N—C(R₂)(R₃)—[C(R₄)(R₅)]ₘ-L-R₆ (1c) or H₂N—R₇ (1d), wherein R₂, R₃, R₄, R₅, m, L, R₆ and R₇ are as above defined, using for example CDI in EtOAc, at room temperature or by heating up to reflux, to provide amide (Ia or Ib).

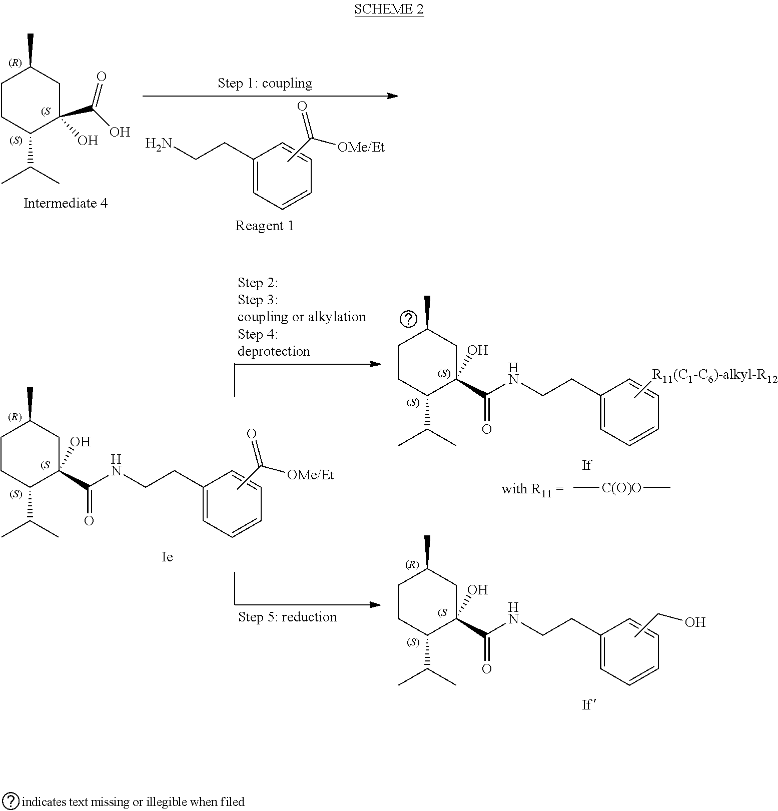

According to SCHEME 2, the intermediate 4 can be coupled with an amine compound (Reagent 1) in Step 1 under condition B as described below in REACTION 1 to give an amid derivative (Ie). The amid derivative (Ie) can be transformed into compound of formula (If or If′) by subjecting the amid derivative (Ie) to further reactions such as alkylation under condition D, E& F as described below in REACTION 3, coupling under condition C as described below in REACTION 1, saponification under conditions as described below in REACTION 5, or reduction under conditions A-C as described below in REACTION 6.

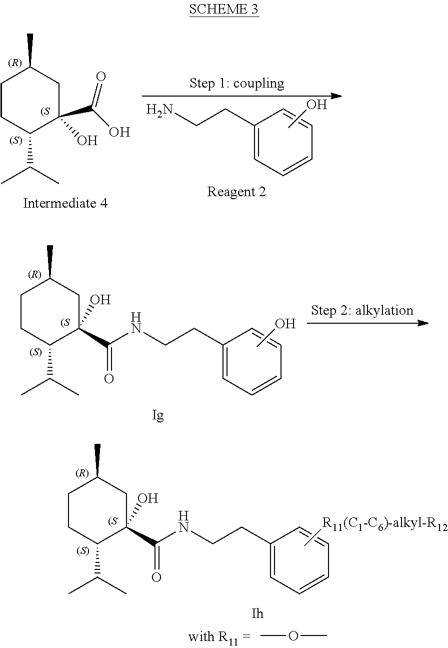

According to SCHEME 3, the intermediate 4 is coupled with an amine compound (Reagent 2) in Step 1 under condition A or B as described below in REACTION 1 to give an amide derivative (Ig). The amide derivative (Ig) is transformed into compound of formula (Ih) in Step 2 by subjecting the amide derivative (Ig) to alkylation reaction under condition A, B or C as described below in REACTION 3.

According to SCHEME 4, potassium tert-butyl N-[2-(trifluoroboranuidyl)ethyl]carbamate and a bromo compound of formula (1i) is subjected to a Suzuki-miyaura aminoethylation reaction as shown in Step 1 with a suitable palladium reagent, for example 1,1′bis(diphenylphosphino)ferrocene-palladium(II) dichloride dichloromethane complex, in a mixture of toluene and water and in the presence of a base, for example cesium carbonate. The intermediate obtained is then subjected to a deprotection reaction as shown in Step 2 of SCHEME 4 in the presence of a suitable acid for example HCl to give an amine compound of formula (Ij). The amine compound obtained (Ij) is then subjected in Step 3 to a coupling reaction or a coupling reaction under condition B as described below in REACTION 1 and a saponification under condition as described below in REACTION 5 to give compound of formula (Ik or Il).

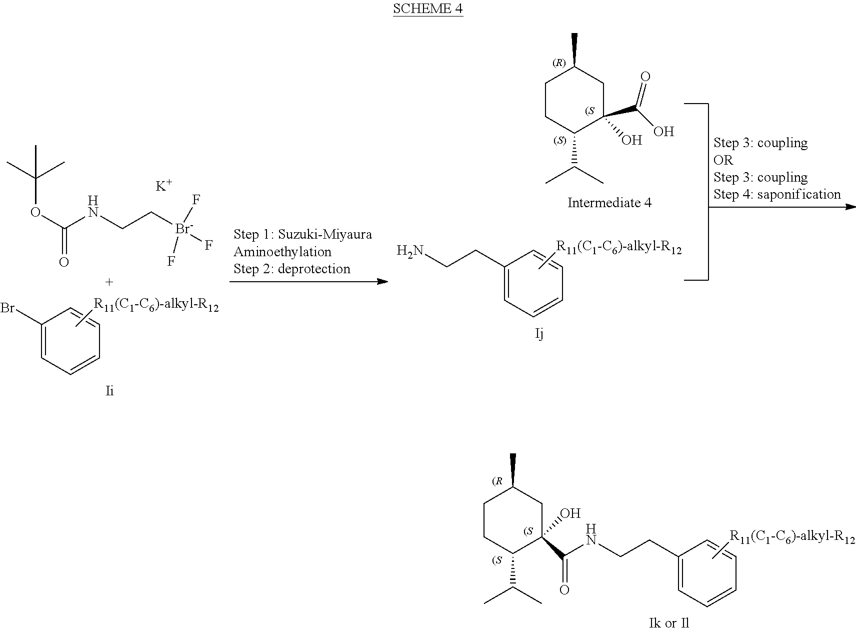

An ester or amide can be obtained through a coupling reaction (step 5 in SCHEME 1; step 1 in SCHEME 2, step 1 in SCHEME 3, step 3 in SCHEME 4) of an acid (Intermediate 4) with an alcohol or an amine compound under conditions A & B described in REACTION 1 as shown below:

Reaction 1: Coupling Reactions: Conversion of Acid into an Ester or Amide

Coupling Conditions A

(1S,2S,5R)-1-hydroxy-2-isopropyl-5-methylcyclohexane-1-carboxylic acid is dissolved in EtOAc (0.1M) under inert atmosphere, and CDI (1eq) is added portionwise to the resulting solution. The reaction mixture is then stirred at rt for 2 hrs, and the amine corresponding to the desired amide (1.11 eq) is added portionwise. The reaction mixture is then heated at 60-70° C. for 16 hrs (The reaction temperature and time may vary depending on the amine and specific temperature and time is noted in example if it is different from 60-70° C. for 16 hrs). Water (0.5V) is then poured into the reaction mixture as well as an 1N aqueous solution of hydrochloric acid and the resulting mixture was stirred. The organic phase is separated and washed with an 1N aqueous solution of sodium hydroxide (1V), then with brine (1V). The organic phase is then dried over Na₂SO₄, filtrated and concentrated under vacuum yielding a residue which is purified by flash chromatography. Reprecipitation is carried out using DCM (0.12V) and n-pentane (0.12V) and leads to the pure desired amide.

Coupling Conditions B

In a round bottom flask, (1S,2S,5R)-1-hydroxy-2-isopropyl-5-methylcyclohexane-1-carboxylic acid is dissolved in acetonitrile (0.3M) under inert atmosphere and CDI (0.94eq) is added portionwise to the resulting solution. The reaction mixture is then stirred at rt for 50 min and the amine corresponding to the desired amide (1.05 eq) is added portionwise followed by addition of pyridine (1.1eq). The reaction mixture is then stirred at rt for 16 hrs (The reaction temperature and time may vary depending on the amine and specific temperature and time is noted in example if it is different from rt for 16 hrs). After concentration under vacuum of the reaction mixture, the resulting residue is dissolved in EtOAc (4V), and water (4V) was added. The aqueous solution is then acidified to pH 1-2 by addition of an 1N aqueous solution of hydrochloric acid, and, after extraction, the organic phase is recovered. The aqueous phase is brought to pH 11-12 by addition of drops of 35% aqueous solution of sodium hydroxide. The combined organic phases are then dried over Na₂SO₄, filtrated and concentrated under vacuum yielding a residue which is purified by flash chromatography. Reprecipitation is carried out using DCM (0.12V) and n-pentane (0.12V) and leads to the pure desired amide

Coupling Conditions C

An acid (such as for example 2-(2-((1S,2S,5R)-1-hydroxy-2-isopropyl-5-methylcyclohexane-1-carboxamido)ethyl)benzoic acid, 3-(2-((1S,2S,5R)-1-hydroxy-2-isopropyl-5-methylcyclohexane-1-carboxamido)ethyl)benzoic acid or ((1S,2S,5R)-1-hydroxy-2-isopropyl-5-methylcyclohexane-1-carbonyl)glycine) is dissolved in THF (0.2M) under inert atmosphere and the alcohol (such as for example tert-butyl N-(2-hydroxyethyl)carbamate or phenol) (1.29eq) is then added. DCC (1.33eq) and DMAP (0.21eq) are added to the resulting solution. The reaction mixture is then stirred at rt during 50 min and the amine corresponding to the desired amide (1.05 eq) is added portionwise followed by addition of pyridine (1.1eq). The reaction mixture is then stirred at rt over 16 hrs. After concentration under vacuum of the reaction mixture, the resulting residue is dissolved in EtOAc (2V) and water (2V) is added. The aqueous solution is then acidified to pH 1-2 by addition of an 1N aqueous solution of hydrochloric acid. The organic phases are dried over Na₂SO₄, filtrated and concentrated under vacuum yielding the pure desired amide with or without flash chromatography.

Coupling Conditions D

The acid (such as for example ((1S,2S,5R)-1-hydroxy-2-isopropyl-5-methylcyclohexane-1-carbonyl)glycine OR 3-((1S,2S,5R)-1-hydroxy-2-isopropyl-5-methylcyclohexane-1-carboxamido)-2-phenylpropanoic acid) is dissolved in DCM (0.2M) under inert atmosphere and an alcohol/amine compound (such as for example vanillin, tert-butyl N-(2-hydroxyethyl)carbamate or N-Boc-2-amino-2-methyl-1-propanol) (1.1eq) is then added. DCC (1.1eq) and DMAP (0.1eq) are added to the resulting solution. The reaction mixture is then stirred at rt during 20 hrs. After filtration of the reaction mixture, the resulting organic solution is washed with an 1N aqueous solution of sodium hydroxide (10V), an 1N aqueous solution of hydrochloric acid (10V), brine (10V), dried over Na₂SO₄, filtrated and concentrated under vacuum to provide the desired ester/amide with or without flash chromatography.

Coupling Conditions E

An acid (such as for example 3-(2-((1S,2S,5R)-1-hydroxy-2-isopropyl-5-methylcyclohexane-1-carboxamido)ethyl)benzoic acid or ((1S,2S,5R)-1-hydroxy-2-isopropyl-5-methylcyclohexane-1-carbonyl)glycine) and an alcohol/amine (such as for example ethanol, phloroglucinol, vanillin, 4-aminophenol or (S)-2-((tert-butyldimethylsilyl)oxy) propan-1-ol) (3.0eq) are dissolved in DMF (0.17M) and the resulting solution is cooled down to 0° C. EDAC (1.0eq) as well as DMAP (0.2eq) are added and once at room temperature, the reaction mixture is stirred for 4 hr30. Water (5V) is added and the resulting solution is extracted with EtOAc (5V×2). The combined organic phases are washed with brine (5V), dried over Na₂SO₄, filtrated and concentrated under vacuum. The residue is purified by flash chromatography to provide the desired ester/amide.

Coupling Conditions F

(1S,2S,5R)—N—((S)-2-amino-2-phenylethyl)-1-hydroxy-2-isopropyl-5-methylcyclohexane-1-carboxamide is dissolved in THF (0.16M) under inert atmosphere and an alcohol/amine (such as for example BOC-D-ALA-OH, BOC-L-ALA-OH, BOC-ALA-OH or BOC-GLY-OH) (1.1eq) is then added. EDAC (1.1eq), HOBT (1.1eq) and triethylamine (1.2eq) are added to the resulting solution. The reaction mixture is then stirred at rt during 16 hrs. After addition of EtOAc (2V), the resulting organic solution is washed with an 1N aqueous solution of sodium hydroxide (5V), an 1N aqueous solution of hydrochloric acid (5V), brine (5V), dried over Na₂SO₄, filtrated and concentrated under vacuum yielding the pure desired amide with or without flash chromatography.

An alcohol or amine compound can be converted into a corresponding ester or amide by reacting the alcohol or amine compound with acetic anhydride are described in REACTION 2 below.

Reaction 2: Acetylation Reaction: Conversion of an Alcohol or an Amine into Corresponding Ester or Amide

Acetylation Conditions

(1S,2S,5R)-1-hydroxy-N—((S)-2-hydroxy-2-phenylethyl)-2-isopropyl-5-methylcyclohexane-1-carboxamide is dissolved in pyridine (0.18M) and acetic anhydride (2.8eq) is added. The resulting solution is stirred at rt. EtOAc (10V) and water (10V) are added to the reaction mixture and this biphasic solution is acidified to pH 1-2 by addition of an 1N aqueous solution of hydrochloric acid. After separation, the organic phase is then extracted with water (10V) after pH of the aqueous phase is basified to pH 11-12 by addition of drops of 35% aqueous solution of sodium hydroxide. The resulting organic phase is then dried over Na₂SO₄, filtrated and concentrated under vacuum yielding the pure desired acylated product.

A phenol, an alcohol or an acid compound can be converted into a corresponding alkylated compound by reacting the phenol, the alcohol, or the acid compound with an alkylating agent (such as for example step 3 in SCHEME 2, step2 in SCHEME 3) under condition A-F ad described in REACTION 3 as shown below:

Reaction 3: Alkylation Reaction: Conversion of an Alcohol into Corresponding Alkylated Compound.

Alkylation Conditions A

In a microwave tube, an phenol/alcohol (such as for example (1S,2S,5R)-1-hydroxy-N-(2-hydroxyphenethyl)-2-isopropyl-5-methylcyclohexane-1-carboxamide) is dissolved in acetonitrile (0.04M) and cesium carbonate (3.94eq) is then added followed by a bromo alkylating agent (such as for example tert-butyl N-(2-bromoethyl)carbamate, 2-(2-bromoethoxy)tetrahydro-2H-pyran, 2-bromoethyldimethylamine or tert-butyl N-(2-bromoethyl)carbamate) (1.64eq). The resulting reaction mixture is irradiated for 1 hr at 100° C. After concentration under vacuum of the reaction mixture, the resulting residue is dissolved in EtOAc (2V) and water (2V) is added. The aqueous solution is brought to pH 11-12 by addition of drops of 35% aqueous solution of sodium hydroxide and washed once with EtOAc (2V). The combined organic phases are dried over Na₂SO₄, filtrated and concentrated under vacuum. The residue is purified by flash chromatography to yield the desired alkylated product (such as for example, compound of formula (If), compound of formula (Ih)).

Alkylation Conditions B

(1S,2S,5R)-1-hydroxy-N-(2-hydroxyphenethyl)-2-isopropyl-5-methylcyclohexane-1-carboxamide was dissolved in ethanol (0.06M) and an alkylating agent (such as for example glycidol, (R)-glycidol or (S)-glycidol) (3.8eq) is then added followed by triethylamine (1.7eq). The resulting reaction mixture is heated at reflux over 5 days. After concentration under vacuum of the reaction mixture, the resulting residue is purified by flash chromatography to yield compound of formula (Ih).

Alkylation Conditions C

An alcohol (such as for example (1S,2S,5R)-1-hydroxy-N-(2-hydroxy-2-(3-hydroxyphenyl)ethyl)-2-isopropyl-5-methylcyclohexane-1-carboxamide, (1S,2S,5R)-1-hydroxy-N-(3-hydroxyphenethyl)-2-isopropyl-5-methylcyclohexane-1-carboxamide or (1S,2S,5R)-1-hydroxy-N-(2-hydroxy-2-(2-hydroxyphenyl)ethyl)-2-isopropyl-5-methylcyclohexane-1-carboxamide) is dissolved in acetone (0.2M), and K₂CO₃ (2.5eq) was added followed by addition of an alkylating agent (such as for example methyl bromoacetate, 3-bromo-1-propanol, 2-bromoacetamide or 1-bromo-2-[2-(2-methoxyethoxy)ethoxy]ethane) (1.1eq). The reaction is then heated at reflux during 20 min. The reaction mixture is filtrated and concentrated under vacuum. The resulting residue is dissolved in diethyl ether and the resulting organic solution is washed with water (10V), an 1N aqueous solution of sodium hydroxide (10V), with brine (10V), dried over Na₂SO₄, filtrated and concentrated under vacuum. The residue is purified by flash chromatography to yield the desired alkylated product.

Alkylation Conditions D

An acid (such as for example 3-(2-((1S,2S,5R)-1-hydroxy-2-isopropyl-5-methylcyclohexane-1-carboxamido)ethyl)benzoic acid or 2-(2-((1S,2S,5R)-1-hydroxy-2-isopropyl-5-methylcyclohexane-1-carboxamido)ethyl)benzoic acid) is dissolved in acetonitrile (0.09M) and DBU (1.06eq) is then added as well as the alkylating agent (1.1eq). The reaction mixture is heated at 80° C. during 7 hr and then stirred at room temperature during 16 hrs. After concentration under vacuum of the reaction mixture, the resulting residue is dissolved in EtOAc (2V) and water (2V) is added. The aqueous solution is brought to pH 11-12 by addition of drops of 35% aqueous solution of sodium hydroxide and washed another time with EtOAc (2V). The combined organic phases are washed with brine, dried over Na₂SO₄, filtrated and concentrated under vacuum. The residue is purified by flash chromatography to yield the desired alkylated product.

Alkylation Conditions E

(1S,2S,5R)-1-((tert-butyldimethylsilyl)oxy)-N—((S)-2-hydroxy-2-phenylethyl)-2-isopropyl-5-methylcyclohexane-1-carboxamide is dissolved in DMF (0.4M) and the resulting solution is cooled down to 0° C. Sodium hydride (6.0eq) is added, and the reaction mixture is warmed up to room temperature. After cooling down to 0° C., an alkylating agent (methyl bromoacetate or 2-(2-bromoethoxy)tetrahydro-2H-pyran) (6.0eq) is added and is warmed up to room temperature. The reaction mixture is stirred during 24 hrs and is then quenched with water (6V). The resulting mixture is extracted with diethyl ether (6V×2). The organic phase is washed with brine, dried over Na₂SO₄, filtrated and concentrated under vacuum. The residue is purified by flash chromatography with cyclohexane and EtOAc to provide the desired alkylated product.

Alkylation Conditions F

3-(2-((1S,2S,5R)-1-hydroxy-2-isopropyl-5-methylcyclohexane-1-carboxamido)ethyl)benzoic acid is dissolved in acetonitrile (0.07M) and K₂CO₃ (3.00eq) is then added as well as an alkylating agent (such as for example 4-chloromethyl-5-methyl-1,3-dioxol-2-one)(1.19eq). The reaction mixture is heated at 40° C. for 1 hr30 mins, and then sodium iodide (0.11eq) is added. The reaction mixture is heated at 50° C. for 16 hr. After concentration under vacuum of the reaction mixture, the resulting residue is dissolved in EtOAc (4V) and water (4V) is added. The aqueous phase is again washed with EtOAc (4V). The combined organic phases are washed with brine, dried over Na₂SO₄, filtrated and concentrated under vacuum. The residue is purified by flash chromatography to yield the desired alkylated product

The reaction of removing the protecting group (step 2 in SCHEME 4), for instance a carbamate compound, can be performed in accordance with method A or method B as described in REACTION 4 as shown below:

Reaction 4: Deprotection Reaction

Deprotection Conditions A

The carbamate/silylated alcohol was dissolved in DCM (0.08M) and HCl (15.4eq, 4N in dioxane) was added. The reaction mixture was stirred at room temperature over 3 hr30. After concentration under vacuum, the residue was dissolved in MeOH and concentrated under vacuum to yield the pure desired amine.

Deprotection Conditions B

The carbamate was dissolved in DCM (0.08M) and TFA (16.2eq) was added. The reaction mixture was stirred at room temperature over 21 hr. Water (1V) was added followed by portionwise addition of sodium carbonate until pH is 9. DCM (1V) and water (1V) were added. The resulting mixture was filtered on hydrophobic Radley cartridge and the organic phase was concentrated under vacuum. The residue was purified by flash chromatography to yield the desired amine.

The saponification reaction (step 2 in SCHEME 2, step 4 in SCHEME 4) of an ester can be performed in an appropriate solvent in the presence of a base as described in REACTION 5.

Reaction 5: Saponification: Conversion of an Ester Compound into Corresponding Acid Compound.

Saponification Conditions

An ester (such as for example methyl 2-(2-((1S,2S,5R)-1-hydroxy-2-isopropyl-5-methylcyclohexane-1-carboxamido)ethyl)benzoate, methyl 3-(2-((1S,2S,5R)-1-hydroxy-2-isopropyl-5-methylcyclohexane-1-carboxamido)ethyl)benzoate, methyl ((1S,2S,5R)-1-hydroxy-2-isopropyl-5-methylcyclohexane-1-carbonyl)glycinate or methyl 4-((1S,2S,5R)-1-hydroxy-2-isopropyl-5-methylcyclohexane-1-carboxamido)-3-phenyl butanoate) is dissolved in methanol/THF mixture (50/50, 0.06M), and sodium hydroxide (1.0eq) is then added. The resulting reaction mixture is stirred at room temperature over 18 hrs. After concentration under vacuum of the reaction mixture, the resulting residue is dissolved in EtOAc (2V), washed with water, and acidified until obtention of biphasic mixture. The organic phase is dried over Na₂SO₄, filtrated and concentrated under vacuum to yield the desired acid.

The reduction reaction of an ester or ketone (step 5 in SCHEME 2) can be performed in accordance with condition A, B, or C in an appropriate solvent in the presence of a reducing reagent such as a borohydride agent as described in REACTION 6.

Reaction 6: Reduction: Conversion of an Ester or Ketone Compound into Corresponding Alcohol Compound.

Reduction Conditions A

methyl 3-(1-hydroxy-2-((1S,2S,5R)-1-hydroxy-2-isopropyl-5-methylcyclohexane-1-carboxamido)ethyl)benzoate is dissolved in 1,4-dioxane/water mixture (50/50, 0.07M) and sodium borohydride (39-49eq) is then added. The resulting reaction mixture is stirred at room temperature over 20 hrs. Hydrolysis of the reaction is carried out by addition of saturated solution of ammonium chloride at 0° C. and the resulting mixture is concentrated under vacuum. The resulting residue is dissolved in EtOAc (2V) and water (2V). The organic phase is dried over Na₂SO₄, filtrated and concentrated under vacuum to yield the desired acid after flash chromatography or preparative HPLC.

Reduction Conditions B

methyl 4-((1S,2S,5R)-1-hydroxy-2-isopropyl-5-methylcyclohexane-1-carboxamido)-3-phenylbutanoate is dissolved in THF (0.15M) and LiBH4 (11eq) is added, followed by addition of dried MeOH (11eq). The reaction mixture is stirred at room temperature over 72 hrs. An 1N aqueous solution of sodium hydroxide (3V) is then added and the resulting mixture is stirred at 50° C. during 30 min. MeOH (1.5V) is added and the resulting solution is stirred at 50° C. during 22 hrs. After being concentrated under vacuum, the remaining aqueous phase is extracted with EtOAc (3V×2). The combined organic phase is washed with brine (3V), dried over Na₂SO₄, filtrated and concentrated under vacuum. The residue is purified by flash chromatography with cyclohexane and EtOAc to yield the desired alcohol.

Reduction Conditions C

An ester/ketone (such as for example (1S,2S,5R)-1-hydroxy-N-(2-(2-hydroxyphenyl)-2-oxoethyl)-2-isopropyl-5-methylcyclohexane-1-carboxamide, (1S,2S,5R)-1-hydroxy-2-isopropyl-5-methyl-N-(2-oxo-2-phenylethyl)cyclohexane-1-carboxamide or 4-formyl-2-methoxyphenyl ((1S,2S,5R)-1-hydroxy-2-isopropyl-5-methylcyclohexane-1-carbonyl)glycinate) is dissolved in MeOH (0.2M), and sodium borohydride (1eq) is then added portionwise. The resulting reaction mixture is stirred at room temperature over 45 minutes. Water (1V) and EtOAc (1V) are added to the reaction mixture at 0° C. as well as Et₂O (2V), and an 1N aqueous solution of hydrochloric acid. The organic phase is washed with brine (2V), dried over Na₂SO₄, filtrated and concentrated under vacuum. The residue is purified by flash chromatography.

### EXPERIMENTAL PROCEDURES

The starting materials and intermediates may be prepared by the methods described in the present application or are available commercially or are described in the literature, or else may be prepared by methods which are described therein or which are known to a person skilled in the art. By appropriate selection of suitable starting materials, compounds of the formula (I) may be prepared according to the procedures described in the foregoing examples.

The 1H NMR data were performed at 400, 500 and 600 MHz with the chemical shifts (5 in ppm) in the solvent dimethyl sulfoxide-d6 (d6-DMSO) referenced at 2.5 ppm at a temperature of 303 K. Coupling constants (J) are given in Hertz.

The examples which follow describe the preparation of certain compounds in accordance with the disclosure. These examples are not limitative, and merely illustrate the present disclosure. Compounds of formula (I) are identified, for example, by the following analytical methods.

High Pressure Liquid Chromatography-Mass Spectrometry (LCMS) experiments to determine retention times (R_{T}) and associated mass ions are performed using one of the following analytical methods.

Method A: Acquity UPLC CSH C18 1.6 μm, dimension 2.1×50 mm, mobile phase H₂O+0.1% Formic acid/Acetonitrile+0.1% AF. Gradient (3 min): 2 to 100% of B in 2.0 min; 2.6 min: 100% of B; 2.70 min: 2% of B; 3.0 min: 2% B.

Method B: Acquity UPLC CSH C18 1.6 μm, dimension 2.1×50 mm, mobile phase H₂O+0.1% Formic acid/Acetonitrile+0.1% Formic acid. Gradient (10 min): 2 to100% of B in 7.5 min; 9.2 min: 100% of B; 9.3 min: 2% of B; 10.0 min: 2% B.

Method C: Waters ACQUITY UPLC BEH C18 1.7 um 2.1×50 mm; [H₂O+0.05% Formic acid]: [Acetonitrile+0.035% Formic acid] 98:2 (0 min) to 98:2 (0.2 min) to 2:98 (3.8 min) to 2:98 (4.3 min) to 98:2 (4.5 min), 1 ml/min 55° C.; Ionization method: ES+; MS-Type: UPLCesi; MS-Method: Waters SQD2 Single Quadrupol, 0.25s scantime for mass 100-2000; UV detection wavelength: 220 Nm.

Method D: ACQUITY CSH C18 −1.7 μm-2.1×50 mm; Solvants: A: H₂O (0.1% formic acid) B: CH₃CN (0.1% formic acid); Gradient (2.5 min): 3 to100% of B in 2.1 min; 2.45 min: 100% of B; 2.50 min: 3% of B; 1 ml/min 60° C.; UPLC-SQD2 Water apparatus; Ionization method: ES+.

Method E: Acquity UPLC CSH C18 (2.1×50 mm), mobile phase A=H₂O+0.02% HCOOH & B=CH₃CN+0.02% HCOOH, 1 mL/min, 55° C. Gradient: t0 2% B, t4 min 98% B, t4.5 min 98% B, t4.6 min 2% B, t5.0 min 2% B.

Method F: Acquity UPLC CSH C18 (2.1×50 mm), mobile phase A=H₂O+0.05% TFA Eluent B=CH₃CN+0.035% TFA, 1 mL/min, 55° C. Gradient: t0 2% B, t4 min 98% B, t4.5 min 98% B, t4.6 min 2% B, t5.0 min 2% B.

The following Reverse phase preparative chromatography (RP-HPLC) parameters were used for purification:

Method G: Reverse phase preparative chromatography: Waters Sunfire Prep C₁₈ OBD 5 μm 50×50 mm; Solvent A: H₂O+0.1% trifluoroacetic acid; Solvent B: Acetonitrile, Flow rate: 120 mL/min. Gradient (5 minutes) A:B=90:10 or 70:30 at t=0 min; A:B=5:95 at t=3.7 min and A:B=90:10 or 70:30 at t=4.9 min.

Method H: Waters Sunfire Prep C18 OBD 5 μm 50×50 mm; Solvent A: H₂O+0.1% trifluoroacetic acid; Solvent B: Acetonitrile, Flow rate: 120 mL/min. Gradient (7 minutes): A:B=75:25 at t=0 min, A:B=10:90 or 5:95 at t=5.7 min and A:B=25:75 or 95:5 at t=6.9 min.

The following intermediates describe the procedures used for the preparation of various starting materials employed in the preparation of the compounds of formula (I).

### INTERMEDIATES

### Intermediate 1: L-menthone

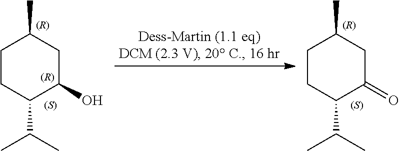

Three batches of the same reaction were carried out in parallel.

To a solution of (R)-menthol (300 g) in DCM (2.7M) was added Dess-martin reagent (1.10eq) at 20° C. under N₂. The reaction was stirred at 20° C. for 16 hrs. The three reactions were combined for workup. The resulting mixture was poured into water (4.3V). The aqueous phase was extracted with DCM (1.4V×3). The combined organic phases were washed with a saturated aqueous solution of Na₂SO₃ (1.4V×2). The organic phase was checked by potassium iodide-starch test paper: the test paper did not change to blue. The organic phase was washed with brine (1.4V×2), dried with anhydrous Na₂SO₄, filtered and concentrated under vacuum. The residue was purified by flash chromatography with petroleum ether & ethyl acetate to give L-menthone (836 g) as colorless oil.

¹H NMR: 400 MHz DMSO-d₆ (δ ppm): 1.98-2.18 (m, 5H), 1.72-1.86 (m, 2H), 1.24-1.40 (m, 2H), 0.95 (d, J=6.4 Hz, 3H), 0.85 (d, J=6.8 Hz, 3H), 0.79 (d, J=6.8 Hz, 3H).

### Intermediate 2: (1R,2S,5R)-2-isopropyl-5-methyl-1-vinylcyclohexan-1-ol

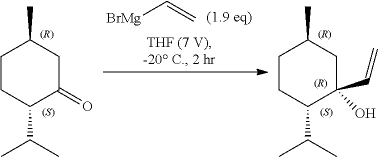

Six batches of the same reaction were carried out in parallel.

To a solution of L-menthone (148 g) in dry THF (0.93M) at −20° C. was added dropwise bromomagnesium vinyl (1.90 eq) in THF under N₂. The reaction mixture was allowed to slowly warm to 15° C. and stirred for 2 hrs. The six reactions were combined for workup. The resulting mixture was poured into a saturated aqueous solution of NH₄Cl (3V) slowly at 10° C. and stirred for 30 mins. The aqueous phase was extracted with MTBE (4V×2). The combined organic phases were washed with brine (2V×2), dried with anhydrous Na₂SO₄, filtered and concentrated under vacuum. The residue was purified by flash chromatography with petroleum ether and ethyl acetate to yield (1R,2S,5R)-2-isopropyl-5-methyl-1-vinylcyclohexan-1-ol (910 g) as colorless oil.

¹H NMR: 400 MHz DMSO-d₆ (δ ppm): 5.75-5.83 (m, 1H), 5.20 (dd, J=17.2 Hz, 2.0 Hz, 1H), 4.97 (dd, J=10.8 Hz, 2.0 Hz, 1H), 3.99 (s, 1H), 1.67-1.92 (m, 3H), 1.36-1.52 (m, 3H), 1.00-1.10 (m, 2H), 0.78-0.88 (m, 10H).

### Intermediate 3: (1S,2S,5R)-1-hydroxy-2-isopropyl-5-methylcyclohexane-1-carbaldehyde

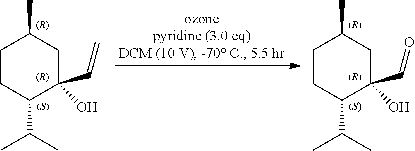

Two batches of same reactions were carried out in parallel.

To a solution of (1R,2S,5R)-2-isopropyl-5-methyl-1-vinylcyclohexan-1-ol (100 g) in DCM (0.55M) and pyridine (3.00 eq) cooled at −70° C. was bubbled ozone until the reaction turned yellow (˜5 hrs). The flow of ozone was stopped, and the solution was degassed using N₂ for 0.5 hr. The two reactions were combined for work up. The combined reaction mixture was poured into water (2V). The aqueous phase was extracted with DCM (1V×3). The combined organic phase was washed with saturated aqueous Na₂SO₃ (1V×2) and the organic phase was checked by potassium iodide-starch test paper: the test paper did not change to blue. The combined organic phase was washed saturated citric acid solution (0.50V×2). The combined organic phase was washed with brine (0.50 V), dried with anhydrous Na₂SO₄, filtered and concentrated under vacuum to (1S,2S,5R)-1-hydroxy-2-isopropyl-5-methylcyclohexane-1-carbaldehyde (180 g) as colorless oil was used into the next step without further purification.

¹H NMR: 400 MHz DMSO-d₆ (δ ppm): 9.63 (s, 1H), 1.98-2.16 (m, 2H), 1.30-1.57 (m, 7H), 0.82-0.86 (m, 9H).

### Intermediate 4: (1S,2S,5R)-1-hydroxy-2-isopropyl-5-methylcyclohexane-1-carboxylic acid

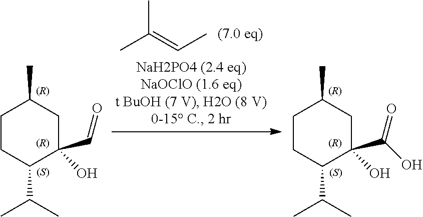

Two batches of the same reaction were carried out in parallel.

To a suspension of (1S,2S,5R)-1-hydroxy-2-isopropyl-5-methylcyclohexane-1-carbaldehyde (90.0 g) in t-BuOH (0.8M) was added 2-methyl-2-butene (7.0 eq) and a solution of NaH₂PO₄ (2.40 eq) in H₂O (1.2V). The reaction was cooled to 0° C. and NaOClO (1.60 eq) was added portion wise. The reaction was stirred for 2 hrs at 15° C. The two reactions were worked up separately. Each reaction mixture was poured into an 1M aqueous solution of NaOH (1V) and MTBE (0.5V) and the resulting mixture was stirred for 10 min. The organic layer was extracted with an 1M aqueous solution of NaOH (0.5V×5). All aqueous phases were combined and MTBE (0.5V) was added. The pH value of the mixture was adjusted to ˜2 with 12N HCl. The resulting solution was extracted with MTBE (0.5V×3). The combined organic phase was washed with saturated aqueous Na₂SO₃ (0.5V) and checked by potassium iodide-starch test paper: the test paper did not change to blue. An aqueous solution of Na₂SO₃ was added and stirring until the test paper did not changed to blue. The organic layer was washed with brine (0.50V), dried with anhydrous Na₂SO₄, filtered and concentrated under vacuum. The other reaction was worked up as described above. The product from two reactions were combined for analysis to yield (1S,2S,5R)-1-hydroxy-2-isopropyl-5-methylcyclohexane-1-carboxylic acid (55.0 g).

¹H NMR: 400 MHz DMSO-d₆ (δ ppm) 12.5 (br.s, 1H), 4.38 (br.s, 1H), 1.67-1.70 (m, 2H), 1.25-1.58 (m, 6H), 0.80-0.90 (m, 10H). LCMS: RT=2.344 min, M-17=183.1.

### EXAMPLES

### Example 1

### (1S,2S,5R)-1-hydroxy-N—((S)-2-hydroxy-2-phenylethyl)-2-isopropyl-5-methylcyclohexane-1-carboxamide

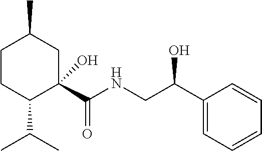 [SMILES: CC(C)[C@@H]1CC[C@@H](C)C[C@@]1(O)C(=O)NC[C@@H](O)c1ccccc1]

Under coupling conditions A, (1S,2S,5R)-1-hydroxy-2-isopropyl-5-methylcyclohexane-1-carboxylic acid (2 gr) and (S)-2-amino-1-phenylethanol (1.11eq) led to (1S,2S,5R)-1-hydroxy-N—((S)-2-hydroxy-2-phenylethyl)-2-isopropyl-5-methylcyclohexane-1-carboxamide (1.62 gr) as white powder after flash chromatography with a gradient between DCM & MeOH and reprecipitation.

### Example 2

### (1S,2S,5R)-1-hydroxy-2-isopropyl-5-methyl-N-(2-oxo-2-phenylethyl)cyclohexane-1-carboxamide

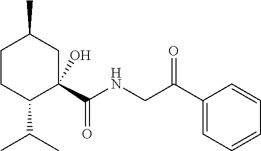 [SMILES: CC(C)[C@@H]1CC[C@@H](C)C[C@@]1(O)C(=O)NCC(=O)c1ccccc1]

Under coupling conditions B, (1S,2S,5R)-1-hydroxy-2-isopropyl-5-methylcyclohexane-1-carboxylic acid (400 mg) and 2-aminoacetophenone hydrochloride (1.05eq) led to (1S,2S,5R)-1-hydroxy-2-isopropyl-5-methyl-N-(2-oxo-2-phenylethyl)cyclohexane-1-carboxamide (320 mg) as white powder after flash chromatography with a gradient between DCM & MeOH and reprecipitation.

### Example 3

### (S)-2-((1S,2S,5R)-1-hydroxy-2-isopropyl-5-methylcyclohexane-1-carboxamido)-1-phenylethyl acetate

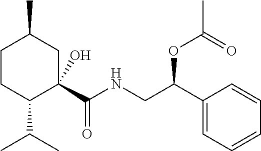 [SMILES: CC(=O)O[C@H](CNC(=O)[C@]1(O)C[C@H](C)CC[C@H]1C(C)C)c1ccccc1]

Acetylation conditions A with (1S,2S,5R)-1-hydroxy-N—((S)-2-hydroxy-2-phenylethyl)-2-isopropyl-5-methylcyclohexane-1-carboxamide (115 mg) led to (S)-2-((1S,2S,5R)-1-hydroxy-2-isopropyl-5-methylcyclohexane-1-carboxamido)-1-phenylethyl acetate (91 mg).

### Example 4

### (1S,2S,5R)—N-(2,2-difluoro-2-phenylethyl)-1-hydroxy-2-isopropyl-5-methylcyclohexane-1-carboxamide

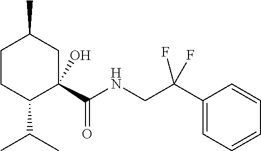 [SMILES: CC(C)[C@@H]1CC[C@@H](C)C[C@@]1(O)C(=O)NCC(F)(F)c1ccccc1]

Under coupling conditions B, (1S,2S,5R)-1-hydroxy-2-isopropyl-5-methylcyclohexane-1-carboxylic acid (200 mg) and 2,2-difluoro-2-phenylethan-1-amine hydrochloride (1.06eq) led to (1S,2S,5R)—N-(2,2-difluoro-2-phenylethyl)-1-hydroxy-2-isopropyl-5-methylcyclohexane-1-carboxamide (160 mg) as white powder without flash chromatography and reprecipitation.

### Example 5

### (1S,2S,5R)—N-(2-fluoro-2-phenylethyl)-1-hydroxy-2-isopropyl-5-methylcyclohexane-1-carboxamide

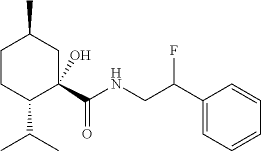 [SMILES: CC(C)[C@@H]1CC[C@@H](C)C[C@@]1(O)C(=O)NCC(F)c1ccccc1]

Under coupling conditions B, (1S,2S,5R)-1-hydroxy-2-isopropyl-5-methylcyclohexane-1-carboxylic acid (200 mg) and 2-fluoro-2-phenylethan-1-amine (1.1eq) [after amine addition, the reaction was heated at 55° C. during 16 hrs] led to (1S,2S,5R)—N-(2-fluoro-2-phenylethyl)-1-hydroxy-2-isopropyl-5-methylcyclohexane-1-carboxamide (175 mg) as white powder after flash chromatography with cyclohexane/EtOAc and without reprecipitation.

### Example 6

### (1S,2S,5R)—N-(2-(2-aminoethoxy)phenethyl)-1-hydroxy-2-isopropyl-5-methylcyclohexane-1-carboxamide

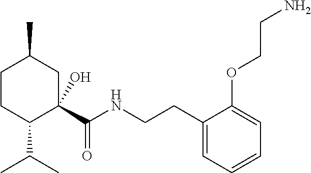 [SMILES: CC(C)[C@@H]1CC[C@@H](C)C[C@@]1(O)C(=O)NCCc1ccccc1OCCN]

Under coupling conditions B, (1S,2S,5R)-1-hydroxy-2-isopropyl-5-methylcyclohexane-1-carboxylic acid (750 mg) and 2-(2-aminoethyl)phenol (1.05eq) [after amine addition, the reaction was heated at 50° C. during 16 hrs] led to (1S,2S,5R)-1-hydroxy-N-(2-hydroxyphenethyl)-2-isopropyl-5-methylcyclohexane-1-carboxamide (702 mg) as yellow powder after flash chromatography with cyclohexane/EtOAc and without reprecipitation.

### (1S,2S,5R)-1-hydroxy-N-(2-hydroxyphenethyl)-2-isopropyl-5-methylcyclohexane-1-carboxamide

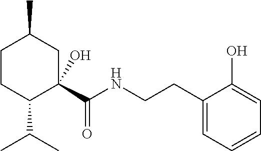 [SMILES: CC(C)[C@@H]1CC[C@@H](C)C[C@@]1(O)C(=O)NCCc1ccccc1O]

Under alkylation conditions A, (1S,2S,5R)-1-hydroxy-N-(2-hydroxyphenethyl)-2-isopropyl-5-methylcyclohexane-1-carboxamide (150 mg) in the presence of tert-butyl N-(2-bromoethyl)carbamate (1.24eq) and followed by treatment with TFA (180 μL) in DCM (6 ml) over 14 hrs led to (1S,2S,5R)—N-(2-(2-aminoethoxy)phenethyl)-1-hydroxy-2-isopropyl-5-methylcyclohexane-1-carboxamide (40 mg) as a yellow wax.

### Example 7

### (1S,2S,5R)-1-hydroxy-N-(2-(2-hydroxyethoxy)phenethyl)-2-isopropyl-5-methylcyclohexane-1-carboxamide

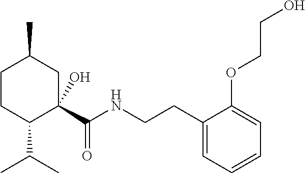 [SMILES: CC(C)[C@@H]1CC[C@@H](C)C[C@@]1(O)C(=O)NCCc1ccccc1OCCO]

Under coupling conditions B, (1S,2S,5R)-1-hydroxy-2-isopropyl-5-methylcyclohexane-1-carboxylic acid (750 mg) and (S)-2-amino-1-phenylethanol (1.05eq) [after amine addition, the reaction was heated at 50° C. during 16 hrs] led to (1S,2S,5R)-1-hydroxy-N-(2-hydroxyphenethyl)-2-isopropyl-5-methylcyclohexane-1-carboxamide (702 mg) as yellow powder after flash chromatography with a gradient between cyclohexane and EtOAc and without reprecipitation.

Under alkylation conditions A, (1S,2S,5R)-1-hydroxy-N-(2-hydroxyphenethyl)-2-isopropyl-5-methylcyclohexane-1-carboxamide (150 mg) in the presence of 2-(2-bromoethoxy)tetrahydro-2H-pyran (1.62eq) and followed by treatment with HCl (4N in dioxane, 5eq) in dioxane (2V) over 14 hrs led to (1S,2S,5R)-1-hydroxy-N-(2-(2-hydroxyethoxy)phenethyl)-2-isopropyl-5-methylcyclohexane-1-carboxamide (40 mg) as a yellow wax.

### Example 8

### (1S,2S,5R)—N-(2-(1H-benzo[d]imidazol-2-yl)ethyl)-1-hydroxy-2-isopropyl-5-methylcyclohexane-1-carboxamide 2-hydroxypropane-1,2,3-tricarboxylate

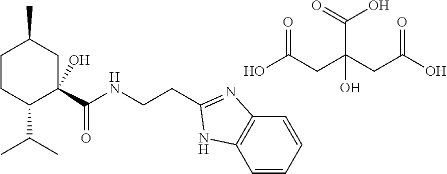

Under coupling conditions A, (1S,2S,5R)-1-hydroxy-2-isopropyl-5-methylcyclohexane-1-carboxylic acid (500 mg) and 2-(1H-benzo[D]imidazol-2-yl)ethanamine (1.12eq) [after amine addition, the reaction was heated at 85° C. during 16 hrs] led to (1S,2S,5R)—N-(2-(1H-benzo[d]imidazol-2-yl)ethyl)-1-hydroxy-2-isopropyl-5-methylcyclohexane-1-carboxamide 2-hydroxypropane-1,2,3-tricarboxylate (210 mg) as white powder and as a citric acid salt due to a wash with a 10% aqueous solution of citric acid after usual sodium hydroxide wash. No flash chromatography & reprecipitation were needed.

### Example 9

### (1S,2S,5R)—N-(2-(2,3-dihydroxypropoxy)phenethyl)-1-hydroxy-2-isopropyl-5-methylcyclohexane-1-carboxamide

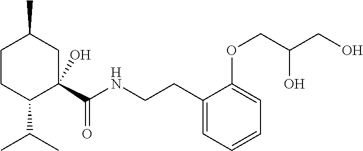 [SMILES: CC(C)[C@@H]1CC[C@@H](C)C[C@@]1(O)C(=O)NCCc1ccccc1OCC(O)CO]

Under coupling conditions B, (1S,2S,5R)-1-hydroxy-2-isopropyl-5-methylcyclohexane-1-carboxylic acid (750 mg) and (S)-2-amino-1-phenylethanol (1.05eq) [after amine addition, the reaction was heated at 75° C. during 16 hrs] led to (1S,2S,5R)-1-hydroxy-N-(2-hydroxyphenethyl)-2-isopropyl-5-methylcyclohexane-1-carboxamide (770 mg) as yellow powder after flash chromatography with a gradient between cyclohexane and EtOAc and without reprecipitation. Under alkylation conditions B, (1S,2S,5R)-1-hydroxy-N-(2-hydroxyphenethyl)-2-isopropyl-5-methylcyclohexane-1-carboxamide (100 mg) in the presence of glycidol (0.95eq) led to (1S,2S,5R)—N-(2-(2,3-dihydroxypropoxy)phenethyl)-1-hydroxy-2-isopropyl-5-methylcyclohexane-1-carboxamide (36 mg) as yellow wax after flash chromatography with DCM & MeOH.

### Example 10

### (1S,2S,5R)—N-(3-(2,3-dihydroxypropoxy)phenethyl)-1-hydroxy-2-isopropyl-5-methylcyclohexane-1-carboxamide

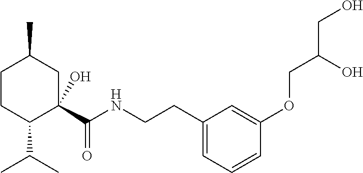 [SMILES: CC(C)[C@@H]1CC[C@@H](C)C[C@@]1(O)C(=O)NCCc1cccc(OCC(O)CO)c1]

Under coupling conditions B, (1S,2S,5R)-1-hydroxy-2-isopropyl-5-methylcyclohexane-1-carboxylic acid (750 mg) and 3-hydroxyphenethylamine hydrochloride (0.67eq) [after amine addition, the reaction was heated at 55° C. during 16 hrs] led to (1S,2S,5R)-3-hydroxy-N-(3-hydroxyphenethyl)-2-isopropyl-5-methylcyclohexane-1-carboxamide (450 mg) as yellow powder after flash chromatography with between cyclohexane and EtOAc and without reprecipitation.

### (1S,2S,5R)-3-hydroxy-N-(3-hydroxyphenethyl)-2-isopropyl-5-methylcyclohexane-1-carboxamide

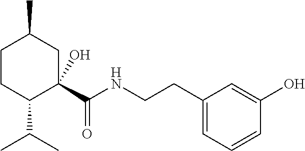 [SMILES: CC(C)[C@@H]1CC[C@@H](C)C[C@@]1(O)C(=O)NCCc1cccc(O)c1]

Under alkylation conditions B, (1S,2S,5R)-1-hydroxy-N-(3-hydroxyphenethyl)-2-isopropyl-5-methylcyclohexane-1-carboxamide (100 mg) in the presence of glycidol (1.82eq) led to (1S,2S,5R)—N-(3-(2,3-dihydroxypropoxy)phenethyl)-1-hydroxy-2-isopropyl-5-methylcyclohexane-1-carboxamide (48 mg) as white solid after flash chromatography with DCM & MeOH.

### Example 11

### 2-(2-(2-((1S,2S,5R)-1-hydroxy-2-isopropyl-5-methylcyclohexane-1-carboxamido)ethyl)phenoxy)-N,N,N-trimethylethan-1-aminium iodide

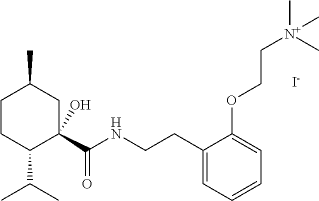 [SMILES: CC(C)[C@@H]1CC[C@@H](C)C[C@@]1(O)C(=O)NCCc1ccccc1OCC[N+](C)(C)C ; [I-]]

Under coupling conditions B, (1S,2S,5R)-1-hydroxy-2-isopropyl-5-methylcyclohexane-1-carboxylic acid (750 mg) and (S)-2-amino-1-phenylethanol (1.05eq) [after amine addition, the reaction was heated at 50° C. during 16 hrs] led to (1S,2S,5R)-1-hydroxy-N-(2-hydroxyphenethyl)-2-isopropyl-5-methylcyclohexane-1-carboxamide (702 mg) as yellow powder after flash chromatography with a gradient between cyclohexane and EtOAc and without reprecipitation.

Under alkylation conditions A, (1S,2S,5R)-1-hydroxy-N-(2-hydroxyphenethyl)-2-isopropyl-5-methylcyclohexane-1-carboxamide (100 mg) in the presence of 2-bromoethyldimethylamine (1.5eq) led to (1S,2S,5R)—N-(2-(2-(dimethylamino)ethoxy)phenethyl)-1-hydroxy-2-isopropyl-5-methylcyclohexane-1-carboxamide (31 mg) as yellow wax.

(1S,2S,5R)—N-(2-(2-(Dimethylamino)ethoxy)phenethyl)-1-hydroxy-2-isopropyl-5-methylcyclohexane-1-carboxamide (30 mg) was dissolved in THF (0.04M) and iodomethane (4.16eq) was added. The reaction mixture was stirred at room temperature over 72 hrs. After concentration under vacuum, the residue was dissolved in EtOAc (5V) and water (5V) was then added. The organic phase was dried over Na₂SO₄, filtrated and concentrated under vacuum to yield an orange wax which was dissolved with MeOH and concentrated under vacuum to provide 2-(2-(2-((1S,2S,5R)-1-hydroxy-2-isopropyl-5-methylcyclohexane-1-carboxamido)ethyl)phenoxy)-N,N,N-trimethylethan-1-aminium iodide (34 mg) as an orange powder.

### Example 12

### (1S,2S,5R)-1-hydroxy-N-(2-hydroxy-2-(m-tolyl)ethyl)-2-isopropyl-5-methylcyclohexane-1-carboxamide

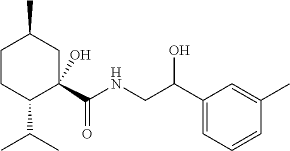 [SMILES: Cc1cccc(C(O)CNC(=O)[C@]2(O)C[C@H](C)CC[C@H]2C(C)C)c1]

Under coupling conditions B, (1S,2S,5R)-1-hydroxy-2-isopropyl-5-methylcyclohexane-1-carboxylic acid (100 mg) and 2-amino-1-(3-methylphenyl)ethan-1-ol (1.07eq) [after amine addition, the reaction was heated at 50° C. during 22 hrs] led to (1S,2S,5R)-1-hydroxy-N-(2-hydroxy-2-(m-tolyl)ethyl)-2-isopropyl-5-methylcyclohexane-1-carboxamide (99 mg) as white powder without flash chromatography and reprecipitation.

### Example 13

### 2-aminoethyl 2-(2-((1S,2S,5R)-1-hydroxy-2-isopropyl-5-methylcyclohexane-1-carboxamido)ethyl) benzoate hydrochloride

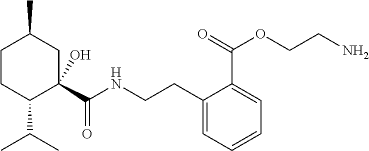 [SMILES: CC(C)[C@@H]1CC[C@@H](C)C[C@@]1(O)C(=O)NCCc1ccccc1C(=O)OCCN]

Under coupling conditions B, (1S,2S,5R)-1-hydroxy-2-isopropyl-5-methylcyclohexane-1-carboxylic acid (654 mg) and methyl 2-(2-aminoethyl)benzoate hydrochloride (1.0eq) [after amine addition, the reaction was heated at 50° C. during 22 hrs] led to methyl 2-(2-((1S,2S,5R)-1-hydroxy-2-isopropyl-5-methylcyclohexane-1-carboxamido)ethyl)benzoate (699 mg) as white powder after flash chromatography with cyclohexane and EtOAc.

### methyl 2-(2-((1S,2S,5R)-1-hydroxy-2-isopropyl-5-methylcyclohexane-1-carboxamido)ethyl) benzoate

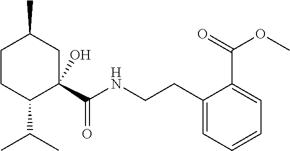 [SMILES: COC(=O)c1ccccc1CCNC(=O)[C@]1(O)C[C@H](C)CC[C@H]1C(C)C]

Under saponification conditions, methyl 2-(2-((1S,2S,5R)-1-hydroxy-2-isopropyl-5-methylcyclohexane-1-carboxamido)ethyl)benzoate (250 mg) in the presence of NaOH (1.0eq) led to 2-(2-((1S,2S,5R)-1-hydroxy-2-isopropyl-5-methylcyclohexane-1-carboxamido)ethyl)benzoic acid (247 mg).

### 2-(2-((1S,2S,5R)-1-hydroxy-2-isopropyl-5-methylcyclohexane-1-carboxamido)ethyl)benzoic acid

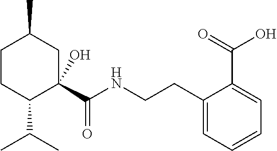 [SMILES: CC(C)[C@@H]1CC[C@@H](C)C[C@@]1(O)C(=O)NCCc1ccccc1C(=O)O]

Under coupling conditions C, with 2-(2-((1S,2S,5R)-1-hydroxy-2-isopropyl-5-methylcyclohexane-1-carboxamido)ethyl)benzoic acid (247 mg) and tert-butyl N-(2-hydroxyethyl)carbamate (1.29eq) led to 2-((tert-butoxycarbonyl)amino)ethyl 2-(2-((1S,2S,5R)-1-hydroxy-2-isopropyl-5-methylcyclohexane-1-carboxamido)ethyl)benzoate (118 mg) after flash chromatography with cyclohexane/EtOAc.

Under deprotection conditions A, 2-((tert-butoxycarbonyl)amino)ethyl 2-(2-((1S,2S,5R)-1-hydroxy-2-isopropyl-5-methylcyclohexane-1-carboxamido)ethyl)benzoate (113 mg) in the presence of HCl (4N in dioxane, 10.4eq) led to 2-aminoethyl 2-(2-((1S,2S,5R)-1-hydroxy-2-isopropyl-5-methylcyclohexane-1-carboxamido)ethyl) benzoate hydrochloride (96 mg).

### Example 14

### (1S,2S,5R)-1-hydroxy-N-(2-(hydroxymethyl)phenethyl)-2-isopropyl-5-methylcyclohexane-1-carboxamide

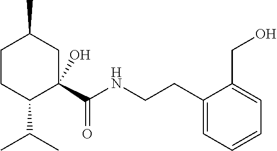 [SMILES: CC(C)[C@@H]1CC[C@@H](C)C[C@@]1(O)C(=O)NCCc1ccccc1CO]

Under coupling conditions B, (1S,2S,5R)-1-hydroxy-2-isopropyl-5-methylcyclohexane-1-carboxylic acid (654 mg) and methyl 2-(2-aminoethyl)benzoate hydrochloride (1.0eq) [after amine addition, the reaction was heated at 50° C. during 22 hrs] led to methyl 2-(2-((1S,2S,5R)-1-hydroxy-2-isopropyl-5-methylcyclohexane-1-carboxamido)ethyl)benzoate (699 mg) as white powder after flash chromatography with cyclohexane and EtOAc.

Under reduction condition A, methyl 2-(2-((1S,2S,5R)-1-hydroxy-2-isopropyl-5-methylcyclohexane-1-carboxamido)ethyl)benzoate (345 mg) in the presence of NaBH4 (39.5eq) led to (1S,2S,5R)-1-hydroxy-N-(2-(hydroxymethyl)phenethyl)-2-isopropyl-5-methylcyclohexane-1-carboxamide (170 mg) after preparative HPLC using Xselect CSH Prep C18 5 μm OBD 50×250 mm column with water+0.1% formic acid and acetonitrile (with this last eluent from 30% to 100%).

### Example 15

### (1S,2S,5R)-1-hydroxy-N-(3-(hydroxymethyl)phenethyl)-2-isopropyl-5-methylcyclohexane-1-carboxamide

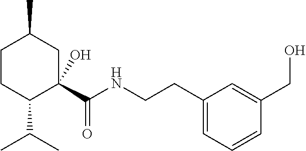 [SMILES: CC(C)[C@@H]1CC[C@@H](C)C[C@@]1(O)C(=O)NCCc1cccc(CO)c1]

Under coupling conditions B, (1S,2S,5R)-1-hydroxy-2-isopropyl-5-methylcyclohexane-1-carboxylic acid (808 mg) and methyl 2-(2-aminoethyl)benzoate hydrochloride (1.05eq) [after amine addition, the reaction was heated at 50° C. during 1 hr20] led to methyl 3-(2-((1S,2S,5R)-1-hydroxy-2-isopropyl-5-methylcyclohexane-1-carboxamido)ethyl)benzoate (916 mg) as white powder after flash chromatography with cyclohexane and EtOAc.

### methyl 3-(2-((1S,2S,5R)-1-hydroxy-2-isopropyl-5-methylcyclohexane-1-carboxamido)ethyl) benzoate

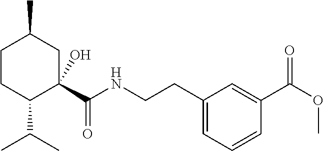 [SMILES: COC(=O)c1cccc(CCNC(=O)[C@]2(O)C[C@H](C)CC[C@H]2C(C)C)c1]

Under reduction condition A, methyl 3-(2-((1S,2S,5R)-1-hydroxy-2-isopropyl-5-methylcyclohexane-1-carboxamido)ethyl)benzoate (645 mg) in the presence of NaBH4 (38.8eq) led to (1S,2S,5R)-1-hydroxy-N-(3-(hydroxymethyl)phenethyl)-2-isopropyl-5-methylcyclohexane-1-carboxamide (175 mg) after preparative HPLC using Xselect CSH Prep C18 5 μm OBD 50×250 mm column with water+0.1% formic acid and acetonitrile (with this last eluent from 29% to 100%).

### Example 16

### 2-aminoethyl 3-(2-((1S,2S,5R)-1-hydroxy-2-isopropyl-5-methylcyclohexane-1-carboxamido)ethyl) benzoate hydrochloride

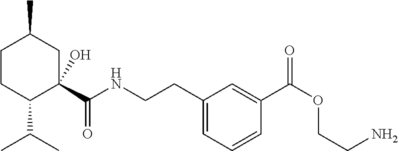 [SMILES: CC(C)[C@@H]1CC[C@@H](C)C[C@@]1(O)C(=O)NCCc1cccc(C(=O)OCCN)c1]

Under coupling conditions B, (1S,2S,5R)-1-hydroxy-2-isopropyl-5-methylcyclohexane-1-carboxylic acid (808 mg) and methyl 2-(2-aminoethyl)benzoate hydrochloride (1.05eq) [after amine addition, the reaction was heated at 50° C. during 1 hr20] led to methyl 3-(2-((1S,2S,5R)-1-hydroxy-2-isopropyl-5-methylcyclohexane-1-carboxamido)ethyl)benzoate (916 mg) as white powder after flash chromatography with cyclohexane and EtOAc.

Under saponification conditions, methyl 3-(2-((1S,2S,5R)-1-hydroxy-2-isopropyl-5-methylcyclohexane-1-carboxamido)ethyl)benzoate (259.2 mg) in the presence of sodium hydroxide (4N in dioxane, 1.0eq) led to 3-(2-((1S,2S,5R)-1-hydroxy-2-isopropyl-5-methylcyclohexane-1-carboxamido)ethyl)benzoic acid (260 mg).

Under coupling conditions C, with 3-(2-((1S,2S,5R)-1-hydroxy-2-isopropyl-5-methylcyclohexane-1-carboxamido)ethyl)benzoic acid (260 mg) and tert-butyl N-(2-hydroxyethyl)carbamate (1.3eq) led to 2-((tert-butoxycarbonyl)amino)ethyl 3-(2-((1S,2S,5R)-1-hydroxy-2-isopropyl-5-methylcyclohexane-1-carboxamido)ethyl)benzoate (221 mg) as white powder after flash chromatography with cyclohexane/EtOAc.

Under deprotection conditions A, 2-((tert-butoxycarbonyl)amino)ethyl 3-(2-((1S,2S,5R)-1-hydroxy-2-isopropyl-5-methylcyclohexane-1-carboxamido)ethyl)benzoate (220 mg) in the presence of HCl (4N in dioxane, 15.2eq) led to 2-aminoethyl 3-(2-((1S,2S,5R)-1-hydroxy-2-isopropyl-5-methylcyclohexane-1-carboxamido)ethyl) benzoate hydrochloride (105 mg) after preparative HPLC using Xselect CSH Prep C18 5 μm OBD 50×250 mm column with water+0.1% formic acid and acetonitrile (with this last eluent from 9% to 100%).

### Examples 17 & 18

### (1S,2S,5R)-1-hydroxy-2-isopropyl-5-methyl-N-(((1RS)-3-oxo-1,3-dihydroisobenzofuran-1-yl)methyl) cyclohexane-1-carboxamide (17)

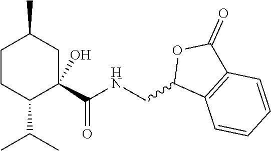 [SMILES: CC(C)[C@@H]1CC[C@@H](C)C[C@@]1(O)C(=O)NCC1OC(=O)c2ccccc21]

### (1S,2S,5R)-1-hydroxy-2-isopropyl-5-methyl-N-(((1RS)-3-oxo-1,3-dihydroisobenzofuran-1-yl)methyl) cyclohexane-1-carboxamide (18)

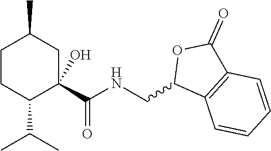 [SMILES: CC(C)[C@@H]1CC[C@@H](C)C[C@@]1(O)C(=O)NCC1OC(=O)c2ccccc21]

Under coupling conditions B, (1S,2S,5R)-1-hydroxy-2-isopropyl-5-methylcyclohexane-1-carboxylic acid (75 mg) and 3-(aminomethyl)isobenzofuran-1 (3H)-one hydrochloride (1.11eq) [after amine addition, the reaction was heated at 55° C. during 1 hr20] led to (1S,2S,5R)-1-hydroxy-2-isopropyl-5-methyl-N-(((1RS)-3-oxo-1,3-dihydroisobenzofuran-1-yl)methyl) cyclohexane-1-carboxamide (example 17, 22 mg) as white powder after flash chromatography with cyclohexane and EtOAc. After flash chromatography, a mixture of both diastereomers was also isolated and purification of this mixture by preparative HPLC using Xselect CSH C18 OBD 5 μm 250×50 mm column with water+0.1% formic acid and acetonitrile (with this last eluent from 29% to 100%) led to (1S,2S,5R)-1-hydroxy-2-isopropyl-5-methyl-N-(((1RS)-3-oxo-1,3-dihydroisobenzofuran-1-yl)methyl) cyclohexane-1-carboxamide (example 18, 11 mg).

### Example 19

### methyl (S)-2-amino-3-(2-(2-((1S,2S,5R)-1-hydroxy-2-isopropyl-5-methylcyclohexane-1-carboxamido) ethyl)phenyl)propanoate

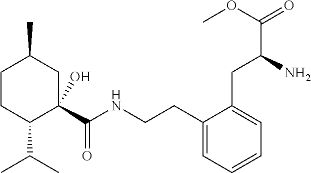 [SMILES: COC(=O)[C@@H](N)Cc1ccccc1CCNC(=O)[C@]1(O)C[C@H](C)CC[C@H]1C(C)C]

Under coupling conditions B, (1S,2S,5R)-1-hydroxy-2-isopropyl-5-methylcyclohexane-1-carboxylic acid (1.55 g) and methyl 2-bromophenethylamine (1.06eq) [here no pyridine was added and after amine addition, the reaction was heated at 55° C. during 16 hr] led to (1S,2S,5R)—N-(2-bromophenethyl)-1-hydroxy-2-isopropyl-5-methylcyclohexane-1-carboxamide (1.05 g) as white powder after flash chromatography with cyclohexane and EtOAc.

Zinc (3.19eq) was added to dried DMF (0.67M). Iodine (0.12eq) was then added followed by addition of BOC-BETA-IODO-ALA-OME (230 mg). Iodine (0.12eq) was again added and after stirring at room temperature during 5 minutes, tris(dibenzylideneacetone)dipalladium(0) (16 mg) and 2-dicyclohexylphosphino-2′,6′-dimethoxybiphenyl (0.03eq) were added to the reaction mixture followed by (1S,2S,5R)—N-(2-bromophenethyl)-1-hydroxy-2-isopropyl-5-methylcyclohexane-1-carboxamide (1.36eq). The resulting was heated at 50° C. during 4 hrs and then at room temperature over 16 hrs. Water (2V) was added as well as EtOAc (2V). The organic phase was dried over Na₂SO₄, filtrated and concentrated under vacuum to yield the N-Boc amine as orange residue.

This residue was dissolved in DCM (5V) and TFA (350 μl) was added to this solution. The resulting reaction mixture was stirred at room temperature over 5 hrs. DCM (5V) and a saturated solution of sodium bicarbonate (5V). The resulting biphasic solution was filtrated over a hydrophobic Radely cartridge. The organic phase was concentrated under vacuum to provide an orange residue. This residue was purified by preparative HPLC using Xselect CSH C18 OBD 5 μm 250×50 mm column with water+0.1% formic acid and acetonitrile (with this last eluent from 29% to 100%) to provide methyl (S)-2-amino-3-(2-(2-((1S,2S,5R)-1-hydroxy-2-isopropyl-5-methylcyclohexane-1-carboxamido)ethyl)phenyl)propanoate (42 mg).

### Example 20

### methyl (S)-2-amino-3-(3-(2-((1S,2S,5R)-1-hydroxy-2-isopropyl-5-methylcyclohexane-1-carboxamido)ethyl)phenyl)propanoate

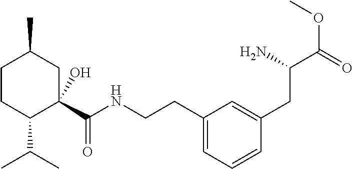 [SMILES: COC(=O)[C@@H](N)Cc1cccc(CCNC(=O)[C@]2(O)C[C@H](C)CC[C@H]2C(C)C)c1]

Under coupling conditions B, (1S,2S,5R)-1-hydroxy-2-isopropyl-5-methylcyclohexane-1-carboxylic acid (1.55 g) and methyl 3-bromophenethylamine (1.06eq) [here no pyridine was added and after amine addition, the reaction was heated at 55° C. during 16 hr] led to (1S,2S,5R)—N-(3-bromophenethyl)-1-hydroxy-2-isopropyl-5-methylcyclohexane-1-carboxamide (1.77 g) as white powder after flash chromatography with cyclohexane and EtOAc.

Zinc (3.19eq) was added to dried DMF (0.67M) and iodine (0.12eq) was added followed by addition of BOC-BETA-IODO-ALA-OME (230 mg). Iodine (0.12eq) was again added and after stirring at room temperature during 5 minutes, tris(dibenzylideneacetone)dipalladium(0) (0.03eq) and 2-dicyclohexylphosphino-2′,6′-dimethoxybiphenyl (0.05eq) were added to the reaction mixture followed by (1S,2S,5R)—N-(3-bromophenethyl)-1-hydroxy-2-isopropyl-5-methylcyclohexane-1-carboxamide (350 mg). The resulting was heated at 50° C. during 4 hrs and then at room temperature over 16 hrs. Water (2V) was added as well as EtOAc (2V). The organic phase was dried over Na2SO4, filtrated and concentrated under vacuum to yield the N-Boc amine as orange residue.

This residue was dissolved in DCM (5V) and TFA (350 μl) was added to this solution. The resulting reaction mixture was stirred at room temperature over 5 hrs. DCM (5V) and a saturated solution of sodium bicarbonate (5V). The resulting biphasic solution was filtrated over a hydrophobic Radely cartridge. The organic phase was concentrated under vacuum to provide an orange residue. This residue was purified by preparative HPLC using Xselect CSH C18 OBD 5 μm 250×50 mm column with water+0.1% formic acid and acetonitrile (with this last eluent from 29% to 100%) to give methyl (S)-2-amino-3-(3-(2-((1S,2S,5R)-1-hydroxy-2-isopropyl-5-methylcyclohexane-1-carboxamido)ethyl)phenyl)propanoate (98 mg) as a white solid.

### Example 21

### 2-hydroxyethyl 3-(2-((1S,2S,5R)-1-hydroxy-2-isopropyl-5-methylcyclohexane-1-carboxamido)ethyl) benzoate

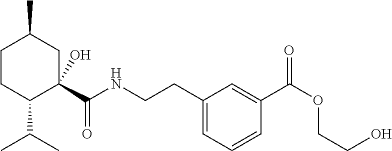 [SMILES: CC(C)[C@@H]1CC[C@@H](C)C[C@@]1(O)C(=O)NCCc1cccc(C(=O)OCCO)c1]

Under coupling conditions B, (1S,2S,5R)-1-hydroxy-2-isopropyl-5-methylcyclohexane-1-carboxylic acid (1.5 g) and methyl 3-(2-aminoethyl)benzoate hydrochloride (1.0eq) [after amine addition, the reaction was heated at 55° C. during 16 hr] led to methyl 3-(2-((1S,2S,5R)-1-hydroxy-2-isopropyl-5-methylcyclohexane-1-carboxamido)ethyl)benzoate (1.86 g) as beige powder after reprecipitation in DCM/n-pentane.

Under saponification conditions, methyl 3-(2-((1S,2S,5R)-1-hydroxy-2-isopropyl-5-methylcyclohexane-1-carboxamido)ethyl)benzoate (1.0 g) in the presence of sodium hydroxide (1N in water, 3.6eq) led to 3-(2-((1S,2S,5R)-1-hydroxy-2-isopropyl-5-methylcyclohexane-1-carboxamido)ethyl)benzoic acid (920 mg).

Under alkylation conditions D, 3-(2-((1S,2S,5R)-1-hydroxy-2-isopropyl-5-methylcyclohexane-1-carboxamido)ethyl)benzoic acid (150 mg), in the presence of 2-bromoethanol (1.11eq) led to 2-hydroxyethyl 3-(2-((1S,2S,5R)-1-hydroxy-2-isopropyl-5-methylcyclohexane-1-carboxamido)ethyl) benzoate (31 mg) as yellow wax after flash chromatography with DCM/MeOH.

### Example 22

### 2-hydroxyethyl 2-(2-((1S,2S,5R)-1-hydroxy-2-isopropyl-5-methylcyclohexane-1-carboxamido)ethyl) benzoate

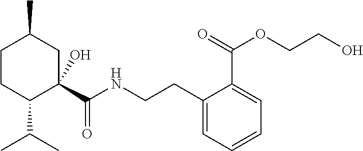 [SMILES: CC(C)[C@@H]1CC[C@@H](C)C[C@@]1(O)C(=O)NCCc1ccccc1C(=O)OCCO]

Under coupling conditions B, (1S,2S,5R)-1-hydroxy-2-isopropyl-5-methylcyclohexane-1-carboxylic acid (1.5 g) and methyl 2-(2-aminoethyl)benzoate hydrochloride (1.06eq) [after amine addition, the reaction was heated at 55° C. during 16 hr] led to methyl 2-(2-((1S,2S,5R)-1-hydroxy-2-isopropyl-5-methylcyclohexane-1-carboxamido)ethyl)benzoate (1.49 g) as colorless wax after flash chromatography with cyclohexane/EtOAc.

Under saponification conditions, methyl 2-(2-((1S,2S,5R)-1-hydroxy-2-isopropyl-5-methylcyclohexane-1-carboxamido)ethyl)benzoate (1.05 g) in the presence of sodium hydroxide (1N in water, 3.4eq) led to 2-(2-((1S,2S,5R)-1-hydroxy-2-isopropyl-5-methylcyclohexane-1-carboxamido)ethyl)benzoic acid (785 mg).

Under alkylation conditions D, 2-(2-((1S,2S,5R)-1-hydroxy-2-isopropyl-5-methylcyclohexane-1-carboxamido)ethyl)benzoic acid (150 mg), in the presence of 2-bromoethanol (1.1eq) led to 2-hydroxyethyl 2-(2-((1S,2S,5R)-1-hydroxy-2-isopropyl-5-methylcyclohexane-1-carboxamido)ethyl) benzoate (101 mg) as yellow wax after flash chromatography with DCM/MeOH.

### Example 23

### (1S,2S,5R)-1-hydroxy-N-((1-hydroxy-2,3-dihydro-1H-inden-1-yl)methyl)-2-isopropyl-5-methylcyclohexane-1-carboxamide

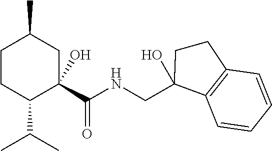 [SMILES: CC(C)[C@@H]1CC[C@@H](C)C[C@@]1(O)C(=O)NCC1(O)CCc2ccccc21]

Under coupling conditions A, (1S,2S,5R)-1-hydroxy-2-isopropyl-5-methylcyclohexane-1-carboxylic acid (500 mg) and 1-(aminomethyl)-2,3-dihydro-1H-inden-1-ol (1.05eq) led to (1S,2S,5R)-1-hydroxy-N-((1-hydroxy-2,3-dihydro-1H-inden-1-yl)methyl)-2-isopropyl-5-methylcyclohexane-1-carboxamide (390 mg) as white powder after flash chromatography with a gradient between cyclohexane & EtOAc.

### Example 24

### (1S,2S,5R)—N-(2-((R)-2,3-dihydroxypropoxy)phenethyl)-1-hydroxy-2-isopropyl-5-methylcyclohexane-1-carboxamide

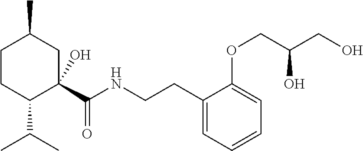 [SMILES: CC(C)[C@@H]1CC[C@@H](C)C[C@@]1(O)C(=O)NCCc1ccccc1OC[C@H](O)CO]

Under coupling conditions A, (1S,2S,5R)-1-hydroxy-2-isopropyl-5-methylcyclohexane-1-carboxylic acid (3 gr) and 2-(2-aminoethyl)phenol (1.06eq) [the reaction mixture was heated at 80° C. during 16 hr] led to (1S,2S,5R)-1-hydroxy-N-(2-hydroxyphenethyl)-2-isopropyl-5-methylcyclohexane-1-carboxamide (3.01 g) as orange powder after flash chromatography with cyclohexane & EtOAc.

Under alkylation conditions B, (1S,2S,5R)-1-hydroxy-N-(2-hydroxyphenethyl)-2-isopropyl-5-methylcyclohexane-1-carboxamide (600 mg) in the presence of (R)-glycidol (0.98eq) [the reaction mixture was heated at 80° C. during 40 hr] led to (1S,2S,5R)—N-(2-((R)-2,3-dihydroxypropoxy)phenethyl)-1-hydroxy-2-isopropyl-5-methylcyclohexane-1-carboxamide (492 mg) as white powder after flash chromatography with DCM & MeOH.

### Example 25

### (1S,2S,5R)—N-(2-((S)-2,3-dihydroxypropoxy)phenethyl)-1-hydroxy-2-isopropyl-5-methylcyclohexane-1-carboxamide

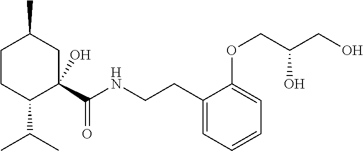 [SMILES: CC(C)[C@@H]1CC[C@@H](C)C[C@@]1(O)C(=O)NCCc1ccccc1OC[C@@H](O)CO]

Under coupling conditions A, (1S,2S,5R)-1-hydroxy-2-isopropyl-5-methylcyclohexane-1-carboxylic acid (3 gr) and 2-(2-aminoethyl)phenol (1.06eq) [the reaction mixture was heated at 80° C. during 16 hr] led to (1S,2S,5R)-1-hydroxy-N-(2-hydroxyphenethyl)-2-isopropyl-5-methylcyclohexane-1-carboxamide (3.01 g) as orange powder after flash chromatography with cyclohexane & EtOAc.

Under alkylation conditions B, (1S,2S,5R)-1-hydroxy-N-(2-hydroxyphenethyl)-2-isopropyl-5-methylcyclohexane-1-carboxamide (600 mg) in the presence of (S)-glycidol (1.0eq) [the reaction mixture was heated at 80° C. during 40 hr] led to (1S,2S,5R)—N-(2-((S)-2,3-dihydroxypropoxy)phenethyl)-1-hydroxy-2-isopropyl-5-methylcyclohexane-1-carboxamide (563 mg) as white powder after flash chromatography with DCM & MeOH.

### Example 26

### methyl O-(3-(2-((1S,2S,5R)-1-hydroxy-2-isopropyl-5-methylcyclohexane-1-carboxamido)ethyl)phenyl)-D-serinate

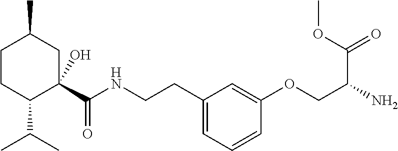 [SMILES: COC(=O)[C@H](N)COc1cccc(CCNC(=O)[C@]2(O)C[C@H](C)CC[C@H]2C(C)C)c1]

Under coupling conditions A, (1S,2S,5R)-1-hydroxy-2-isopropyl-5-methylcyclohexane-1-carboxylic acid (1.55 gr) and 3-bromophenethylamine (1.06eq) led to (1S,2S,5R)—N-(3-bromophenethyl)-1-hydroxy-2-isopropyl-5-methylcyclohexane-1-carboxamide (1.77 gr) as white powder after flash chromatography with cyclohexane & EtOAc.

In photochemistry vial, (1S,2S,5R)—N-(3-bromophenethyl)-1-hydroxy-2-isopropyl-5-methylcyclohexane-1-carboxamide (380 mg) was dissolved in dried acetonitrile (0.5M) and quinuclidine (0.11eq) was added as well as potassium carbonate (0.99eq) and (IR[DF(CF3)PPY]2(DTBPY))PF6 complex (0.01eq). A solution of nickel(II) chloride ethylene glycol dimethyl ether complex (0.05eq) and 4,4′-di-tert-butyl-2,2′-bipyridine (0.05eq) in acetonitrile (1V) was added dropwise. Under argon atmosphere, a solution of BOC-D-SER-OME (1.47eq) in acetonitrile (0.5V) was added to the reaction mixture. The resulting was stirred under irradiation over 12 hr. After concentration under vacuum, the residue was dissolved in EtOAc (10V) and water (10V) was added. The aqueous phase was washed with EtOAc. The combined organic phases were washed with brine, dried over Na₂SO₄, filtrated and concentrated under vacuum. The residue was purified by flash chromatography first with cyclohexane and EtOAc, and secondly with DCM and methanol to yield methyl N-(tert-butoxycarbonyl)-O-(3-(2-((1S,2S,5R)-1-hydroxy-2-isopropyl-5-methylcyclohexane-1-carboxamido)ethyl)phenyl)-D-serinate (93 mg).

Under deprotection conditions B, methyl N-(tert-butoxycarbonyl)-O-(3-(2-((1S,2S,5R)-1-hydroxy-2-isopropyl-5-methylcyclohexane-1-carboxamido)ethyl)phenyl)-D-serinate (90 mg) in the presence of TFA (5.4eq) led to methyl O-(3-(2-((1S,2S,5R)-1-hydroxy-2-isopropyl-5-methylcyclohexane-1-carboxamido)ethyl)phenyl)-D-serinate (49 mg) after flash chromatography with DCM/MeOH.

### Example 27

### methyl O-(3-(2-((1S,2S,5R)-1-hydroxy-2-isopropyl-5-methylcyclohexane-1-carboxamido)ethyl)phenyl)-L-serinate

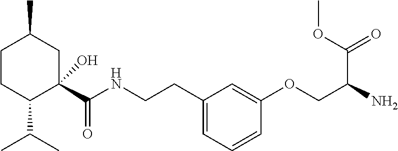 [SMILES: COC(=O)[C@@H](N)COc1cccc(CCNC(=O)[C@]2(O)C[C@H](C)CC[C@H]2C(C)C)c1]

Under coupling conditions A, (1S,2S,5R)-1-hydroxy-2-isopropyl-5-methylcyclohexane-1-carboxylic acid (3 gr) and 3-bromophenethylamine (1.1eq) led to (1S,2S,5R)—N-(3-bromophenethyl)-1-hydroxy-2-isopropyl-5-methylcyclohexane-1-carboxamide (3.75 gr) as white powder after flash chromatography with cyclohexane & EtOAc.

In photochemistry vial, (1S,2S,5R)—N-(3-bromophenethyl)-1-hydroxy-2-isopropyl-5-methylcyclohexane-1-carboxamide (380 mg) was dissolved in dried acetonitrile (0.5M) and quinuclidine (0.11eq) was added as well as potassium carbonate (0.99eq) and (IR[DF(CF3)PPY]2(DTBPY))PF6 complex (0.01eq). A solution of nickel(II) chloride ethylene glycol dimethyl ether complex (0.05eq) and 4,4′-di-tert-butyl-2,2′-bipyridine (0.05eq) in acetonitrile (1V) was added dropwise. Under argon atmosphere, a solution of BOC-L-SER-OME (1.5eq) in acetonitrile (0.5V) was added to the reaction mixture. The resulting was stirred under irradiation over 12 hr. After concentration under vacuum, the residue was dissolved in EtOAc (10V) and water (10V) was added. The aqueous phase was washed with EtOAc. The combined organic phase was washed with brine, dried over Na₂SO₄, filtrated and concentrated under vacuum. The residue was purified by flash chromatography first with cyclohexane and EtOAc, and secondly with DCM and methanol to yield methyl N-(tert-butoxycarbonyl)-O-(3-(2-((1S,2S,5R)-1-hydroxy-2-isopropyl-5-methylcyclohexane-1-carboxamido)ethyl)phenyl)-L-serinate (176 mg) as white powder.

Under deprotection conditions B, methyl N-(tert-butoxycarbonyl)-O-(3-(2-((1S,2S,5R)-1-hydroxy-2-isopropyl-5-methylcyclohexane-1-carboxamido)ethyl)phenyl)-L-serinate (173 mg) in the presence of TFA (20.06eq) led to methyl O-(3-(2-((1S,2S,5R)-1-hydroxy-2-isopropyl-5-methylcyclohexane-1-carboxamido)ethyl)phenyl)-L-serinate (104 mg) after flash chromatography with DCM/MeOH as colorless wax.

### Example 28

### ethyl 3-(2-((1S,2S,5R)-1-hydroxy-2-isopropyl-5-methylcyclohexane-1-carboxamido)ethyl)benzoate

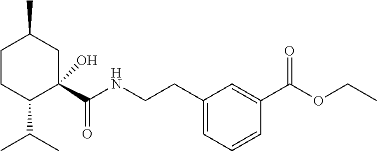 [SMILES: CCOC(=O)c1cccc(CCNC(=O)[C@]2(O)C[C@H](C)CC[C@H]2C(C)C)c1]

Under coupling conditions B, (1S,2S,5R)-1-hydroxy-2-isopropyl-5-methylcyclohexane-1-carboxylic acid (1.5 g) and methyl 3-(2-aminoethyl)benzoate hydrochloride (1.0eq) [after amine addition, the reaction was heated at 55° C. during 16 hr] led to methyl 3-(2-((1S,2S,5R)-1-hydroxy-2-isopropyl-5-methylcyclohexane-1-carboxamido)ethyl)benzoate (1.86 g) as beige powder after reprecipitation in DCM/n-pentane.

Under saponification conditions, methyl 3-(2-((1S,2S,5R)-1-hydroxy-2-isopropyl-5-methylcyclohexane-1-carboxamido)ethyl)benzoate (1.0 g) in the presence of sodium hydroxide (1N in water, 3.6eq) led to 3-(2-((1S,2S,5R)-1-hydroxy-2-isopropyl-5-methylcyclohexane-1-carboxamido)ethyl)benzoic acid (920 mg).

Under coupling conditions E, with 3-(2-((1S,2S,5R)-1-hydroxy-2-isopropyl-5-methylcyclohexane-1-carboxamido)ethyl)benzoic acid (150 mg) and ethanol (2.70eq) led to ethyl 3-(2-((1S,2S,5R)-1-hydroxy-2-isopropyl-5-methylcyclohexane-1-carboxamido)ethyl) benzoate (69 mg) after flash chromatography with cyclohexane/EtOAc.

### Example 29

### (5-methyl-2-oxo-1,3-dioxol-4-yl)methyl 3-(2-((1S,2S,5R)-1-hydroxy-2-isopropyl-5-methylcyclohexane-1-carboxamido)ethy) benzoate

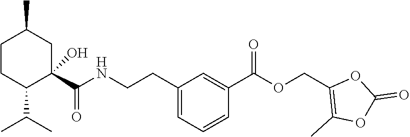 [SMILES: Cc1oc(=O)oc1COC(=O)c1cccc(CCNC(=O)[C@]2(O)C[C@H](C)CC[C@H]2C(C)C)c1]

Under coupling conditions B, (1S,2S,5R)-1-hydroxy-2-isopropyl-5-methylcyclohexane-1-carboxylic acid (1.5 g) and methyl 3-(2-aminoethyl)benzoate hydrochloride (1.0eq) [after amine addition, the reaction was heated at 55° C. during 16 hr] led to methyl 3-(2-((1S,2S,5R)-1-hydroxy-2-isopropyl-5-methylcyclohexane-1-carboxamido)ethyl)benzoate (1.86 g) as beige powder after reprecipitation in DCM/n-pentane.

Under saponification conditions, methyl 3-(2-((1S,2S,5R)-1-hydroxy-2-isopropyl-5-methylcyclohexane-1-carboxamido)ethyl)benzoate (1.0 g) in the presence of sodium hydroxide (1N in water, 3.6eq) led to 3-(2-((1S,2S,5R)-1-hydroxy-2-isopropyl-5-methylcyclohexane-1-carboxamido)ethyl)benzoic acid (920 mg).

Under alkylation conditions F, 3-(2-((1S,2S,5R)-1-hydroxy-2-isopropyl-5-methylcyclohexane-1-carboxamido)ethyl)benzoic acid (150 mg) in the presence of 4-chloromethyl-5-methyl-1,3-dioxol-2-one (1.19eq) led to (5-methyl-2-oxo-1,3-dioxol-4-yl)methyl 3-(2-((1S,2S,5R)-1-hydroxy-2-isopropyl-5-methylcyclohexane-1-carboxamido)ethyl)benzoate (140 mg).

### Example 30

### (1S,2S,5R)—N-(2-(benzylamino)-2-oxoethyl)-1-hydroxy-2-isopropyl-5-methylcyclohexane-1-carboxamide

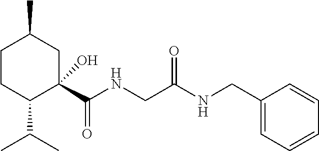 [SMILES: CC(C)[C@@H]1CC[C@@H](C)C[C@@]1(O)C(=O)NCC(=O)NCc1ccccc1]

Under coupling conditions A, (1S,2S,5R)-1-hydroxy-2-isopropyl-5-methylcyclohexane-1-carboxylic acid (80 mg) and 2-amino-N-benzylacetamide (1.05eq) [after amine addition, the reaction was heated at 75° C. during 16 hr] led to (1S,2S,5R)—N-(2-(benzylamino)-2-oxoethyl)-1-hydroxy-2-isopropyl-5-methylcyclohexane-1-carboxamide (65 mg) as white powder.

### Example 31

### (1S,2S,5R)-1-hydroxy-2-isopropyl-5-methyl-N-((3-phenyloxetan-3-yl)methyl)cyclohexane-1-carboxamide

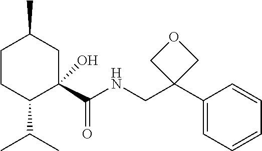 [SMILES: CC(C)[C@@H]1CC[C@@H](C)C[C@@]1(O)C(=O)NCC1(c2ccccc2)COC1]

Under coupling conditions A, (1S,2S,5R)-1-hydroxy-2-isopropyl-5-methylcyclohexane-1-carboxylic acid (100 mg) and (3-phenyloxetan-3-yl)methanamine (1.3eq) [after amine addition, the reaction was heated at 70° C. during 3 hr] led to (1S,2S,5R)-1-hydroxy-2-isopropyl-5-methyl-N-((3-phenyloxetan-3-yl)methyl)cyclohexane-1-carboxamide (86 mg) as white powder.

### Example 32

### (1S,2S,5R)-1-hydroxy-N-(2-hydroxy-2-(3-hydroxyphenyl)ethyl)-2-isopropyl-5-methylcyclohexane-1-carboxamide

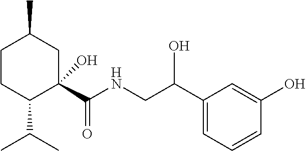 [SMILES: CC(C)[C@@H]1CC[C@@H](C)C[C@@]1(O)C(=O)NCC(O)c1cccc(O)c1]

Under coupling conditions A, (1S,2S,5R)-1-hydroxy-2-isopropyl-5-methylcyclohexane-1-carboxylic acid (507 mg) and norfenefrine (1.05eq) [after amine addition, the reaction was heated at 70° C. during 3 hr] led to (1S,2S,5R)-1-hydroxy-N-(2-hydroxy-2-(3-hydroxyphenyl)ethyl)-2-isopropyl-5-methylcyclohexane-1-carboxamide (455 mg) as white solid.

### Example 33

### methyl 2-(3-(1-hydroxy-2-((1S,2S,5R)-1-hydroxy-2-isopropyl-5-methylcyclohexane-1-carboxamido)ethyl)phenoxy)acetate

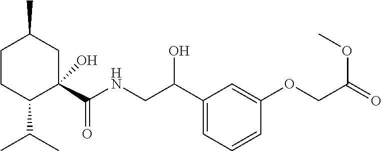 [SMILES: COC(=O)COc1cccc(C(O)CNC(=O)[C@]2(O)C[C@H](C)CC[C@H]2C(C)C)c1]

Under alkylation conditions C, (1S,2S,5R)-1-hydroxy-N-(2-hydroxy-2-(3-hydroxyphenyl)ethyl)-2-isopropyl-5-methylcyclohexane-1-carboxamide (302 mg) in the presence of methyl bromoacetate (1.1eq) led to methyl 2-(3-(1-hydroxy-2-((1S,2S,5R)-1-hydroxy-2-isopropyl-5-methylcyclohexane-1-carboxamido)ethyl)phenoxy)acetate (256 mg) as a colorless wax.

### Example 34

### methyl 3-(1-hydroxy-2-((1S,2S,5R)-1-hydroxy-2-isopropyl-5-methylcyclohexane-1-carboxamido)ethyl)benzoate

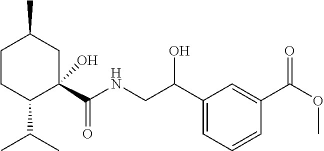 [SMILES: COC(=O)c1cccc(C(O)CNC(=O)[C@]2(O)C[C@H](C)CC[C@H]2C(C)C)c1]

Under coupling conditions A, (1S,2S,5R)-1-hydroxy-2-isopropyl-5-methylcyclohexane-1-carboxylic acid (400 mg) and methyl 3-(2-amino-1-hydroxyethyl)benzoate (1.05eq) [after amine addition, the reaction was heated at 75° C. during 3 hr] led to methyl 3-(1-hydroxy-2-((1S,2S,5R)-1-hydroxy-2-isopropyl-5-methylcyclohexane-1-carboxamido)ethyl)benzoate (205 mg) as white solid after preparative HPLC using CSH 250×50 MM-5 μM column with water and acetonitrile containing 0.1% formic acid.

### Example 35

### (1S,2S,5R)-1-hydroxy-N-(2-hydroxy-2-(2-hydroxyphenyl)ethyl)-2-isopropyl-5-methylcyclohexane-1-carboxamide

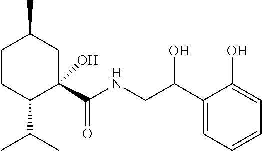 [SMILES: CC(C)[C@@H]1CC[C@@H](C)C[C@@]1(O)C(=O)NCC(O)c1ccccc1O]

Under coupling conditions A, (1S,2S,5R)-1-hydroxy-2-isopropyl-5-methylcyclohexane-1-carboxylic acid (300 mg) and 2-amino-1-(2-hydroxyphenyl)ethan-1-one hydrobromide (1.05eq) [after amine addition, the reaction was heated at 50° C. during 2 hr] led to (1S,2S,5R)-1-hydroxy-N-(2-(2-hydroxyphenyl)-2-oxoethyl)-2-isopropyl-5-methylcyclohexane-1-carboxamide (376 mg) as orange solid after flash chromatography with cyclohexane and EtOAc.

Under reduction conditions C, (1S,2S,5R)-1-hydroxy-N-(2-(2-hydroxyphenyl)-2-oxoethyl)-2-isopropyl-5-methylcyclohexane-1-carboxamide (374 mg) in the presence of NaBH4 (1.05eq) led to (1S,2S,5R)-1-hydroxy-N-(2-hydroxy-2-(2-hydroxyphenyl)ethyl)-2-isopropyl-5-methylcyclohexane-1-carboxamide (46 mg) after flash chromatography with cyclohexane & EtOAc followed by trituration DCM/pentane.

### Example 36

### methyl 2-(2-(1-hydroxy-2-((1S,2S,5R)-1-hydroxy-2-isopropyl-5-methylcyclohexane-1-carboxamido)ethyl)phenoxy)acetate

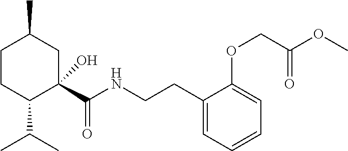 [SMILES: COC(=O)COc1ccccc1CCNC(=O)[C@]1(O)C[C@H](C)CC[C@H]1C(C)C]

Under alkylation conditions C, (1S,2S,5R)-1-hydroxy-N-(2-hydroxy-2-(2-hydroxyphenyl)ethyl)-2-isopropyl-5-methylcyclohexane-1-carboxamide (135 mg) in the presence of methyl bromoacetate (1.1eq) led to methyl 2-(2-(1-hydroxy-2-((1S,2S,5R)-1-hydroxy-2-isopropyl-5-methylcyclohexane-1-carboxamido)ethyl)phenoxy)acetate (116 mg) as a white wax after flash chromatography with cyclohexane and EtOAc.

### Example 37

### (1S,2S,5R)-1-hydroxy-N-(2-hydroxy-2-(3-(hydroxymethyl)phenyl)ethyl)-2-isopropyl-5-methylcyclohexane-1-carboxamide

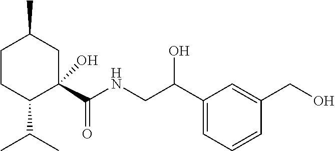 [SMILES: CC(C)[C@@H]1CC[C@@H](C)C[C@@]1(O)C(=O)NCC(O)c1cccc(CO)c1]

Methyl 3-formylbenzoate (1.64 g) was dissolved in THF (1.0M) and the resulting solution was cooled down to 0° C. Nitromethane (10.0eq) was added followed by slow addition of DBU (0.1eq). The reaction mixture was stirred at room temperature during 2 hr30. Diethyl ether (1.5V) was added and the resulting solution was washed with a 0.1N aqueous solution of hydrochloric acid (1.5V), brine (1.5V) dried over Na₂SO₄, filtrated and concentrated under vacuum. The residue was purified by flash chromatography with cyclohexane and EtOAc to provide methyl 3-(1-hydroxy-2-nitroethyl)benzoate (2.11 g) as orange oil.

Methyl 3-(1-hydroxy-2-nitroethyl)benzoate (2.11 g) was dissolved in ethanol (0.31M) and PtO₂ (0.05eq) was added. The reaction mixture was put under hydrogen atmosphere (H₂ 1 bar) and stirred at room temperature under H₂ during 4 hr. The reaction mixture was filtrated on GF/F Whatman filter paper and the column was washed with ethanol (2V). The combined solutions were concentrated under vacuum to give methyl 3-(2-amino-1-hydroxyethyl)benzoate (1.46 g) as yellow oil.

Under coupling conditions A, (1S,2S,5R)-1-hydroxy-2-isopropyl-5-methylcyclohexane-1-carboxylic acid (400 mg) and methyl 3-(2-amino-1-hydroxyethyl)benzoate (1.05eq) [after amine addition, the reaction was heated at 50° C. during 2 hr] led to methyl 3-(1-hydroxy-2-((1S,2S,5R)-1-hydroxy-2-isopropyl-5-methylcyclohexane-1-carboxamido)ethyl)benzoate (316 mg) as orange solid after flash chromatography with cyclohexane and EtOAc.

Under reduction conditions A, methyl 3-(1-hydroxy-2-((1S,2S,5R)-1-hydroxy-2-isopropyl-5-methylcyclohexane-1-carboxamido)ethyl)benzoate (316 mg) in the presence of NaBH₄ (5.0eq) led to (1S,2S,5R)-1-hydroxy-N-(2-hydroxy-2-(3-(hydroxymethyl)phenyl)ethyl)-2-isopropyl-5-methylcyclohexane-1-carboxamide (155 mg) as colorless oil after flash chromatography with cyclohexane & EtOAc.

### Example 38

### (1S,2S,5R)-1-hydroxy-N-(2-(2-hydroxyphenyl)-2-oxoethyl)-2-isopropyl-5-methylcyclohexane-1-carboxamide

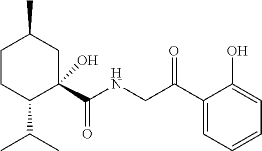 [SMILES: CC(C)[C@@H]1CC[C@@H](C)C[C@@]1(O)C(=O)NCC(=O)c1ccccc1O]

Under coupling conditions A, (1S,2S,5R)-1-hydroxy-2-isopropyl-5-methylcyclohexane-1-carboxylic acid (400 mg) and 2-amino-1-(2-hydroxyphenyl)ethan-1-one hydrobromide (1.05eq) [after CDI addition, pyridine (1.1eq) was added and after amine addition, the reaction was heated at 50° C. during 2 hr] led to (1S,2S,5R)-1-hydroxy-N-(2-(2-hydroxyphenyl)-2-oxoethyl)-2-isopropyl-5-methylcyclohexane-1-carboxamide (417 mg) as yellow solid after flash chromatography with cyclohexane and EtOAc and trituration with n-pentane.

### Example 39

### (1S,2S,5R)-1-hydroxy-N-(2-(3-hydroxyphenyl)-2-oxoethyl)-2-isopropyl-5-methylcyclohexane-1-carboxamide

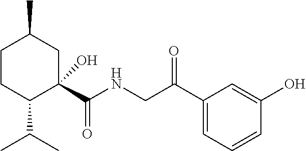 [SMILES: CC(C)[C@@H]1CC[C@@H](C)C[C@@]1(O)C(=O)NCC(=O)c1cccc(O)c1]

Under coupling conditions A, (1S,2S,5R)-1-hydroxy-2-isopropyl-5-methylcyclohexane-1-carboxylic acid (300 mg) and 2-amino-1-(3-hydroxyphenyl)ethan-1-one hydrochloride (1.05eq) [after CDI addition, pyridine (1.1eq) was added and after amine addition, the reaction was heated at 50° C. during 2 hr] led to (1S,2S,5R)-1-hydroxy-N-(2-(3-hydroxyphenyl)-2-oxoethyl)-2-isopropyl-5-methylcyclohexane-1-carboxamide (62 mg) as white solid after flash chromatography with cyclohexane and EtOAc followed by preparative HPLC using CSH 250×50 MM-5 μM column with water and acetonitrile containing 0.1% formic acid.

### Example 40

### (1S,2S,5R)-1-hydroxy-N-(3-hydroxyphenethyl)-2-isopropyl-5-methylcyclohexane-1-carboxamide

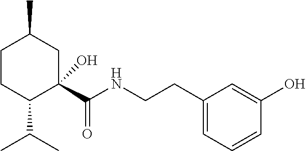 [SMILES: CC(C)[C@@H]1CC[C@@H](C)C[C@@]1(O)C(=O)NCCc1cccc(O)c1]

Under coupling conditions B, (1S,2S,5R)-1-hydroxy-2-isopropyl-5-methylcyclohexane-1-carboxylic acid (1.5 g) and 3-(2-aminoethyl)phenol hydrobromide (1.05eq) [after amine addition, the reaction was heated at 50° C. during 4 hr and at room temperature during 16 hr] led to (1S,2S,5R)-1-hydroxy-N-(3-hydroxyphenethyl)-2-isopropyl-5-methylcyclohexane-1-carboxamide (1.4g, 93% purity) as yellow solid after flash chromatography with cyclohexane and EtOAc. A small portion of this amide (260 mg) is further purified by preparative HPLC using CSH 250×50 MM-5 μM column with water and acetonitrile containing 0.1% formic acid to give (1S,2S,5R)-1-hydroxy-N-(3-hydroxyphenethyl)-2-isopropyl-5-methylcyclohexane-1-carboxamide (180 mg) as white solid.

### Examples 41 & 42

### (1S,2S,5R)-1-hydroxy-N-((2R)-hydroxy-2-(3-hydroxyphenyl)ethyl)-2-isopropyl-5-methylcyclohexane-1-carboxamide (41)

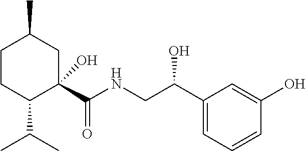 [SMILES: CC(C)[C@@H]1CC[C@@H](C)C[C@@]1(O)C(=O)NC[C@H](O)c1cccc(O)c1]

### (1S,2S,5R)-1-hydroxy-N-((2S)-hydroxy-2-(3-hydroxyphenyl)ethyl)-2-isopropyl-5-methylcyclohexane-1-carboxamide (42)

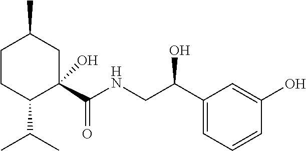 [SMILES: CC(C)[C@@H]1CC[C@@H](C)C[C@@]1(O)C(=O)NC[C@@H](O)c1cccc(O)c1]

Chiral separation of (1S,2S,5R)-1-hydroxy-N-(2-hydroxy-2-(3-hydroxyphenyl)ethyl)-2-isopropyl-5-methylcyclohexane-1-carboxamide (142.8 mg) carried out by liquid chromatography using CHIRALPAK IA with heptane and ethanol led to (1S,2S,5R)-1-hydroxy-N-((2R)-hydroxy-2-(3-hydroxyphenyl)ethyl)-2-isopropyl-5-methylcyclohexane-1-carboxamide (example 41, 83 mg) and (1S,2S,5R)-1-hydroxy-N-((2S)-hydroxy-2-(3-hydroxyphenyl)ethyl)-2-isopropyl-5-methylcyclohexane-1-carboxamide (example 42, 64 mg).

### Examples 43 & 44

### (1S,2S,5R)-1-hydroxy-N-((2R)-hydroxy-2-(3-(hydroxymethyl)phenyl)ethyl)-2-isopropyl-5-methylcyclohexane-1-carboxamide (43)

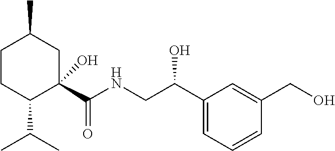 [SMILES: CC(C)[C@@H]1CC[C@@H](C)C[C@@]1(O)C(=O)NC[C@H](O)c1cccc(CO)c1]

### (1S,2S,5R)-1-hydroxy-N-((2S)-hydroxy-2-(3-(hydroxymethyl)phenyl)ethyl)-2-isopropyl-5-methylcyclohexane-1-carboxamide (44)

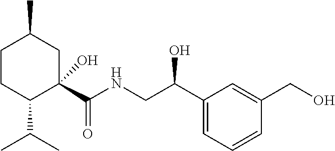 [SMILES: CC(C)[C@@H]1CC[C@@H](C)C[C@@]1(O)C(=O)NC[C@@H](O)c1cccc(CO)c1]

Chiral separation of (1S,2S,5R)-1-hydroxy-N-(2-hydroxy-2-(3-(hydroxymethyl)phenyl)ethyl)-2-isopropyl-5-methylcyclohexane-1-carboxamide (142.3 mg) carried out by liquid chromatography using CHIRALPAK IA with heptane and ethanol led to (1S,2S,5R)-1-hydroxy-N-((2R)-hydroxy-2-(3-(hydroxymethyl)phenyl)ethyl)-2-isopropyl-5-methylcyclohexane-1-carboxamide (example 43, 67 mg) and (1S,2S,5R)-1-hydroxy-N-((2S)-hydroxy-2-(3-(hydroxymethyl)phenyl)ethyl)-2-isopropyl-5-methylcyclohexane-1-carboxamide (example 44, 65 mg).

### Examples 45 and 46

### (1S,2S,5R)-1-hydroxy-N-((2R)-hydroxy-2-(m-tolyl)ethyl)-2-isopropyl-5-methylcyclohexane-1-carboxamide (45)

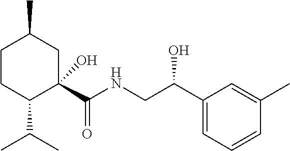 [SMILES: Cc1cccc([C@@H](O)CNC(=O)[C@]2(O)C[C@H](C)CC[C@H]2C(C)C)c1]

### (1S,2S,5R)-1-hydroxy-N-((2S)-hydroxy-2-(m-tolyl)ethyl)-2-isopropyl-5-methylcyclohexane-1-carboxamide (46)

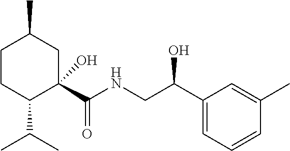 [SMILES: Cc1cccc([C@H](O)CNC(=O)[C@]2(O)C[C@H](C)CC[C@H]2C(C)C)c1]

Chiral separation of (1S,2S,5R)-1-hydroxy-N-(2-hydroxy-2-(m-tolyl)ethyl)-2-isopropyl-5-methylcyclohexane-1-carboxamide (85.8 mg) carried out by liquid chromatography using CHIRALPAK IA with heptane and ethanol to (1S,2S,5R)-1-hydroxy-N-((2R)-hydroxy-2-(m-tolyl)ethyl)-2-isopropyl-5-methylcyclohexane-1-carboxamide (example 45, 45 mg) and (1S,2S,5R)-1-hydroxy-N-((2S)-hydroxy-2-(m-tolyl)ethyl)-2-isopropyl-5-methylcyclohexane-1-carboxamide (example 46, 49 mg).

### Example 47

### (1S,2S,5R)-1-hydroxy-N-((2R)-1-hydroxy-3-phenylpropan-2-yl)-2-isopropyl-5-methylcyclohexane-1-carboxamide

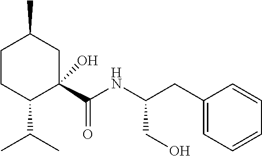 [SMILES: CC(C)[C@@H]1CC[C@@H](C)C[C@@]1(O)C(=O)N[C@@H](CO)Cc1ccccc1]

Under coupling conditions A, (1S,2S,5R)-1-hydroxy-2-isopropyl-5-methylcyclohexane-1-carboxylic acid (300 mg) and D-phenylalaninol (1.1eq) [after amine addition, the reaction was heated at 50° C. during 3 hr] led to (1S,2S,5R)-1-hydroxy-N-((2R)-1-hydroxy-3-phenylpropan-2-yl)-2-isopropyl-5-methylcyclohexane-1-carboxamide (172 mg) as white solid after flash chromatography with cyclohexane and EtOAc followed by trituration with DCM and n-pentane.

### Example 48

### (1S,2S,5R)—N-(3-cyanophenethyl)-1-hydroxy-2-isopropyl-5-methylcyclohexane-1-carboxamide

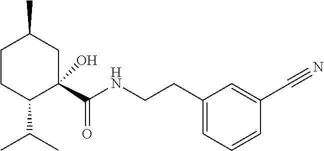 [SMILES: CC(C)[C@@H]1CC[C@@H](C)C[C@@]1(O)C(=O)NCCc1cccc(C#N)c1]

Under coupling conditions B, (1S,2S,5R)-1-hydroxy-2-isopropyl-5-methylcyclohexane-1-carboxylic acid (300 mg) and 3-(2-aminoethyl)benzonitrile hydrochloride (1.05eq) [after amine addition, the reaction was heated at 50° C. during 6 hr and at room temperature during 16 hrs] led to (1S,2S,5R)—N-(3-cyanophenethyl)-1-hydroxy-2-isopropyl-5-methylcyclohexane-1-carboxamide (226 mg) as white solid after flash chromatography with cyclohexane and EtOAc followed by trituration with DCM and n-pentane.

### Example 49

### 3-(2-((1S,2S,5R)-1-hydroxy-2-isopropyl-5-methylcyclohexane-1-carboxamido)ethyl) benzamide

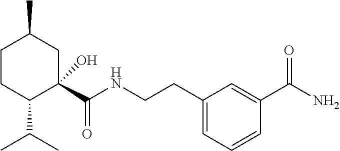 [SMILES: CC(C)[C@@H]1CC[C@@H](C)C[C@@]1(O)C(=O)NCCc1cccc(C(N)=O)c1]

(1S,2S,5R)—N-(3-Cyanophenethyl)-1-hydroxy-2-isopropyl-5-methylcyclohexane-1-carboxamide (115 mg) was dissolved in DMSO (2M) and cooled down to 5-10° C. K₂CO₃ (0.5eq) was added followed by dropwise addition of hydrogen peroxide (4eq). The reaction was then vigorously stirred over 15 minutes. EtOAc (15V) was added and after first extraction, the aqueous phase was washed again with EtOAc (5V). The combined organic phases were washed with brine, dried over Na₂SO₄, filtrated and concentrated under vacuum to provide 3-(2-((1S,2S,5R)-1-hydroxy-2-isopropyl-5-methylcyclohexane-1-carboxamido)ethyl)benzamide (50 mg) as white solid.

### Example 50

### (1S,2S,5R)—N-((2S)-amino-2-phenylethyl)-1-hydroxy-2-isopropyl-5-methylcyclohexane-1-carboxamide

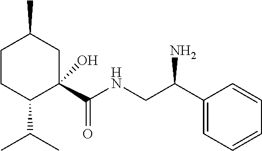 [SMILES: CC(C)[C@@H]1CC[C@@H](C)C[C@@]1(O)C(=O)NC[C@@H](N)c1ccccc1]

Under coupling conditions A, (1S,2S,5R)-1-hydroxy-2-isopropyl-5-methylcyclohexane-1-carboxylic acid (500 mg) and (S)-tert-butyl (2-amino-1-phenylethyl)carbamate (1.2eq) [after amine addition, the reaction was stirred at room temperature during 16 hr] led to tert-butyl ((S)-2-((1S,2S,5R)-1-hydroxy-2-isopropyl-5-methylcyclohexane-1-carboxamido)-1-phenylethyl)carbamate (482 mg) as white solid after flash chromatography with cyclohexane and acetone.

Under deprotection conditions B, tert-butyl ((S)-2-((1S,2S,5R)-1-hydroxy-2-isopropyl-5-methylcyclohexane-1-carboxamido)-1-phenylethyl)carbamate (440 mg) in the presence of TFA (810 μl) led to (1S,2S,5R)—N-((2S)-amino-2-phenylethyl)-1-hydroxy-2-isopropyl-5-methylcyclohexane-1-carboxamide (304 mg) as white solid.

### Example 51

### (1S,2S,5R)-1-hydroxy-2-isopropyl-5-methyl-N-phenethylcyclohexane-1-carboxamide

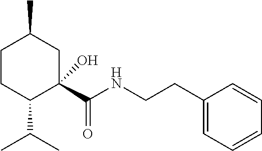 [SMILES: CC(C)[C@@H]1CC[C@@H](C)C[C@@]1(O)C(=O)NCCc1ccccc1]

Under coupling conditions A, (1S,2S,5R)-1-hydroxy-2-isopropyl-5-methylcyclohexane-1-carboxylic acid (75 mg) and phenethylamine (2.1eq) [after amine addition, the reaction was heated at 70° C. during 2 hr] led to (1S,2S,5R)-1-hydroxy-2-isopropyl-5-methyl-N-phenethylcyclohexane-1-carboxamide (83 mg) as white solid after flash chromatography with cyclohexane and EtOAc.

### Example 52

### (1S,2S,5R)-1-hydroxy-N-(2-(2-hydroxyethyl)phenethyl)-2-isopropyl-5-methylcyclohexane-1-carboxamide

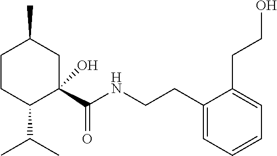 [SMILES: CC(C)[C@@H]1CC[C@@H](C)C[C@@]1(O)C(=O)NCCc1ccccc1CCO]

2-Bromophenethylamine (1.438 gr) was dissolved in THF (0.2M) under argon and K₂CO₃ (1.3eq) was added followed by addition of benzyl chloroformate (1.1eq). The reaction mixture was stirred at room temperature during 22 hr and was then filtrated. The resulting organic phase was washed with water (1V), an 1N aqueous solution of HCl (1V), brine (1V), dried over Na₂SO₄, filtrated and concentrated under vacuum. The residue was purified by flash chromatography with cyclohexane and EtOAc to provide benzyl (2-bromophenethyl)carbamate (719 mg) as white solid.

Benzyl (2-bromophenethyl)carbamate (719 mg) was dissolved in DME (0.13M) and the solution was degassed with argon. Pd(Ph₃)₄ (0.04eq) was added and the resulting mixture was stirred at room temperature during 20 minutes. K₂CO₃ (1.23eq) and water (0.33V) were added followed by addition of 2,4,6-trivinylboroxine pyridine complex (1.2eq). The reaction mixture was heated at reflux over 16 hr. After concentration under vacuum, the residue was dissolved in Et₂O (1V) and water (1V). The organic phase was washed with brine (1V), dried over Na₂SO₄, filtrated and concentrated under vacuum. The residue was purified by flash chromatography with cyclohexane and EtOAc to yield benzyl (2-vinylphenethyl)carbamate (513 mg) as yellow oil.

Benzyl (2-vinylphenethyl)carbamate (512 mg) was dissolved in THF (1M) and the resulting solution was cooled down to 0° C. before dropwise addition of BH₃-THF complex (1.21eq, 1M). Once at room temperature, the reaction mixture was stirred during 2 hr. An 1N aqueous solution of sodium hydroxide (1.0eq) was added dropwise at 0° C. followed by addition of 30% H₂O₂ (5eq) and the resulting solution was then warmed up to room temperature. Water (5V) and Et₂O (10V) were added and the organic phase was then washed with brine (5V), dried over Na₂SO₄, filtrated and concentrated under vacuum. The residue was purified by flash chromatography with cyclohexane/EtOAc to yield benzyl 2-(2-hydroxyethyl)phenethyl)carbamate (294 mg) as colorless oil.

Benzyl 2-(2-hydroxyethyl)phenethyl)carbamate (333 mg) was dissolved in MeOH (0.08M) and the resulting solution was degassed with argon. 10% Palladium on charcoal (0.1eq) was added and the reaction mixture was stirred under 3 bars of hydrogen during 5 hr. The solution was filtrated on GF/F Whatman filter paper and concentrated under vacuum to provide 2-(2-(2-aminoethyl)phenylethan-1-ol (179 mg) as white solid.

Under coupling conditions A, (1S,2S,5R)-1-hydroxy-2-isopropyl-5-methylcyclohexane-1-carboxylic acid (217 mg) and 2-(2-(2-aminoethyl)phenylethan-1-ol (1.0eq) [after amine addition, the reaction was heated at 70° C. during 2 hr and stirred at room temperature during 1-hr] led to (1S,2S,5R)-1-hydroxy-N-(2-(2-hydroxyethyl)phenethyl)-2-isopropyl-5-methylcyclohexane-1-carboxamide (151 mg) as colorless wax after flash chromatography with cyclohexane and acetone.

### Example 53

### (1S,2S,5R)—N-(2-(2-aminoethyl)phenethyl)-1-hydroxy-2-isopropyl-5-methylcyclohexane-1-carboxamide

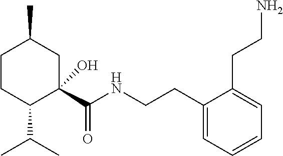 [SMILES: CC(C)[C@@H]1CC[C@@H](C)C[C@@]1(O)C(=O)NCCc1ccccc1CCN]

(1S,2S,5R)-1-Hydroxy-N-(2-(2-hydroxyethyl)phenethyl)-2-isopropyl-5-methylcyclohexane-1-carboxamide (98 mg) was dissolved in DCM (0.11M) and cooled down to 0° C. under argon. Mesyl chloride (1.1eq) and triethylamine (1.53eq) were then added. Once at room temperature, the reaction mixture was stirred over 5 hr and water (1V) was added as well as DCM (1V). The organic phase was dried over Na₂SO₄, filtrated and concentrated under vacuum to yield the desired mesylate (127 mg).

2-(2-((1S,2S,5R)-1-Hydroxy-2-isopropyl-5-methylcyclohexane-1-carboxamido)ethyl)phenethyl methanesulfonate (119 mg) was dissolved in DMF and NaN₃ (1.5eq) was added. The reaction mixture was heated at 60° C. during 3 hr. Once at room temperature, water (20V) and EtOAc (20V) were added. The organic phase was washed with brine (10V), dried over Na₂SO₄, filtrated and concentrated under vacuum.

The residue was then dissolved in THF (2V) and water (6.0eq) was added as well as PPh₃ (1.5eq). The reaction mixture was then stirred at room temperature over 16 hr. After addition of EtOAc (2V), the solution was washed with an 1N aqueous solution of NaOH (4V) and brine (4V). The organic phase was dried over Na₂SO₄, filtrated and concentrated under vacuum. The residue was purified by preparative HPLC using CSH 250×50 MM-5 μM column with water and acetonitrile containing 0.1% formic acid to give (1S,2S,5R)—N-(2-(2-aminoethyl)phenethyl)-1-hydroxy-2-isopropyl-5-methylcyclohexane-1-carboxamide (59 mg).

### Example 54

### isopropyl ((1S,2S,5R)-1-hydroxy-2-isopropyl-5-methylcyclohexane-1-carbonyl)glycinate

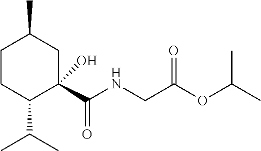 [SMILES: CC(C)OC(=O)CNC(=O)[C@]1(O)C[C@H](C)CC[C@H]1C(C)C]

Under coupling conditions B, (1S,2S,5R)-1-hydroxy-2-isopropyl-5-methylcyclohexane-1-carboxylic acid (300 mg) and isopropyl 2-aminoacetate hydrochloride (1.1eq) [after amine addition, the reaction was heated at 50-55° C. during 16 hr] led to isopropyl ((1S,2S,5R)-1-hydroxy-2-isopropyl-5-methylcyclohexane-1-carbonyl)glycinate (155 mg) as white solid after flash chromatography with cyclohexane and EtOAc followed by trituration with DCM and n-pentane.

### Example 55

### 2-((S)-2-((1S,2S,5R)-1-hydroxy-2-isopropyl-5-methylcyclohexane-1-carboxamido)-1-phenylethoxy)acetic acid

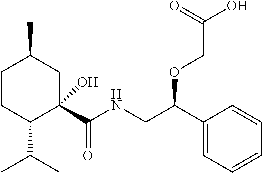 [SMILES: CC(C)[C@@H]1CC[C@@H](C)C[C@@]1(O)C(=O)NC[C@@H](OCC(=O)O)c1ccccc1]

Under coupling conditions A, (1S,2S,5R)-1-hydroxy-2-isopropyl-5-methylcyclohexane-1-carboxylic acid (700 mg) and (S)-2-amino-1-phenylethanol (1.05eq) [after amine addition, the reaction was heated at 60° C. during 6 hr and stirred at room temperature during 72 hr] led to (1S,2S,5R)-1-hydroxy-N—((S)-2-hydroxy-2-phenylethyl)-2-isopropyl-5-methylcyclohexane-1-carboxamide (754 mg) as ecru solid after flash chromatography with cyclohexane and acetone.

Under Acetylation conditions A, (1S,2S,5R)-1-hydroxy-N—((S)-2-hydroxy-2-phenylethyl)-2-isopropyl-5-methylcyclohexane-1-carboxamide (724 mg) in the presence of acetic anhydride (1.6eq) led to (S)-2-((1S,2S,5R)-1-hydroxy-2-isopropyl-5-methylcyclohexane-1-carboxamido)-1-phenylethyl acetate (825 mg) as pale yellow wax.

(S)-2-((1S,2S,5R)-1-Hydroxy-2-isopropyl-5-methylcyclohexane-1-carboxamido)-1-phenylethyl acetate (778 mg) was dissolved in DCM (0.61M) and the resulting solution was cooled down to 0° C. Triethylamine (4.0eq) was added followed by addition of tert-butyldimethylsilyl trilfuoromethanesulfonate (4.0eq). The reaction mixture was stirred at room temperature during 16 hrs. After addition of DCM (1V), the organic phase was washed with an 1N aqueous solution of HCl (2V), with brine (2V), dried over Na₂SO₄, filtrated and concentrated under vacuum. The residue was purified by flash chromatography with cyclohexane and EtOAc to provide (S)-2-((1S,2S,5R)-1-((tert-butyldimethylsilyl)oxy)-2-isopropyl-5-methylcyclohexane-1-carboxamido)-1-phenylethyl acetate (694 mg) as colorless oil.

(S)-2-((1S,2S,5R)-1-((tert-Butyldimethylsilyl)oxy)-2-isopropyl-5-methylcyclohexane-1-carboxamido)-1-phenylethyl acetate (693 mg) was dissolved in methanol (0.2M) and K₂CO₃ (2.0eq) was added. The reaction mixture was stirred at room temperature during 16 hrs. after filtration and concentration under vacuum, the residue was dissolved in diethyl ether, washed with water (1.4V), with brine (1.4V), dried over Na₂SO₄, filtrated and concentrated under vacuum to yield (1S,2S,5R)-1-((tert-butyldimethylsilyl)oxy)-N—((S)-2-hydroxy-2-phenylethyl)-2-isopropyl-5-methylcyclohexane-1-carboxamide (595 mg) as colorless wax.

Under alkylation conditions E, (1S,2S,5R)-1-((tert-butyldimethylsilyl)oxy)-N—((S)-2-hydroxy-2-phenylethyl)-2-isopropyl-5-methylcyclohexane-1-carboxamide (295 mg) in the presence of methyl bromoacetate (6eq) led to methyl 2-((S)-2-((1S,2S,5R)-1-((tert-butyldimethylsilyl)oxy)-2-isopropyl-5-methylcyclohexane-1-carboxamido)-1-phenylethoxy)acetate (107 mg) as colorless oil after flash chromatography with cyclohexane and EtOAc.

Methyl 2-((S)-2-((1S,2S,5R)-1-((tert-butyldimethylsilyl)oxy)-2-isopropyl-5-methylcyclohexane-1-carboxamido)-1-phenylethoxy)acetate (106 mg) was dissolved in THF (0.5M) and cooled down to 0° C. TBAF (15.5eq) was added dropwise at 0° C. and the reaction mixture was warmed up to room temperature. The reaction mixture was stirred during 46 hrs at this temperature. After concentration under vacuum, the residue was dissolved in Et2O (10V) and the organic phase was washed with a saturated aqueous solution of ammonium chloride. The aqueous phase was acidified with HCl 1N up to pH 1 and was extracted with diethyl ether (10 v×3). The combined organic phase was dried over Na₂SO₄, filtrated and concentrated under vacuum.

The residue was dissolved in DCM (0.5M) and HCl (1N, 3.82eq) was added. The reaction mixture was stirred at room temperature during 5 hrs. After concentration under vacuum, the residue was dissolved in Et₂O (10V), was washed with water (10V) and brine (10V). The organic phase was dried over Na₂SO₄, filtrated and concentrated under vacuum. The residue was purified by flash chromatography with DCM and methanol to yield 2-((S)-2-((1S,2S,5R)-1-hydroxy-2-isopropyl-5-methylcyclohexane-1-carboxamido)-1-phenylethoxy)acetic acid (25 mg) as white solid.

### Example 56

### (1S,2S,5R)-1-hydroxy-N—((S)-2-(2-hydroxyethoxy)-2-phenylethyl)-2-isopropyl-5-methylcyclohexane-1-carboxamide

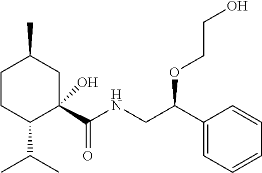 [SMILES: CC(C)[C@@H]1CC[C@@H](C)C[C@@]1(O)C(=O)NC[C@@H](OCCO)c1ccccc1]

Under coupling conditions A, (1S,2S,5R)-1-hydroxy-2-isopropyl-5-methylcyclohexane-1-carboxylic acid (700 mg) and (S)-2-amino-1-phenylethanol (1.05eq) [after amine addition, the reaction was heated at 60° C. during 6 hr and stirred at room temperature during 72 hr] led to (1S,2S,5R)-1-hydroxy-N—((S)-2-hydroxy-2-phenylethyl)-2-isopropyl-5-methylcyclohexane-1-carboxamide (754 mg) as ecru solid after flash chromatography with cyclohexane and acetone.

Under Acetylation conditions A, (1S,2S,5R)-1-hydroxy-N—((S)-2-hydroxy-2-phenylethyl)-2-isopropyl-5-methylcyclohexane-1-carboxamide (724 mg) in the presence of acetic anhydride (1.6eq) led to (S)-2-((1S,2S,5R)-1-hydroxy-2-isopropyl-5-methylcyclohexane-1-carboxamido)-1-phenylethyl acetate (825 mg) as pale yellow wax.

(S)-2-((1S,2S,5R)-1-Hydroxy-2-isopropyl-5-methylcyclohexane-1-carboxamido)-1-phenylethyl acetate (778 mg) was dissolved in DCM (0.61M) and the resulting solution was cooled down to 0° C. Triethylamine (4.0eq) was added followed by addition of tert-butyldimethylsilyl trilfuoromethanesulfonate (4.0eq). The reaction mixture was stirred at room temperature during 16 hrs. After addition of DCM (1V), the organic phase was washed with an 1N aqueous solution of HCl (2V), with brine (2V), dried over Na₂SO₄, filtrated and concentrated under vacuum. The residue was purified by flash chromatography with cyclohexane and EtOAc to provide (S)-2-((1S,2S,5R)-1-((tert-butyldimethylsilyl)oxy)-2-isopropyl-5-methylcyclohexane-1-carboxamido)-1-phenylethyl acetate (694 mg) as colorless oil.

(S)-2-((1S,2S,5R)-1-((tert-Butyldimethylsilyl)oxy)-2-isopropyl-5-methylcyclohexane-1-carboxamido)-1-phenylethyl acetate (693 mg) was dissolved in methanol (0.2M) and K₂CO₃ (2.0eq) was added. The reaction mixture was stirred at room temperature during 16 hrs. after filtration and concentration under vacuum, the residue was dissolved in diethyl ether, washed with water (1.4V), with brine (1.4V), dried over Na₂SO₄, filtrated and concentrated under vacuum to yield (1S,2S,5R)-1-((tert-butyldimethylsilyl)oxy)-N—((S)-2-hydroxy-2-phenylethyl)-2-isopropyl-5-methylcyclohexane-1-carboxamide (595 mg) as colorless wax.

Under alkylation conditions E, (1S,2S,5R)-1-((tert-butyldimethylsilyl)oxy)-N—((S)-2-hydroxy-2-phenylethyl)-2-isopropyl-5-methylcyclohexane-1-carboxamide (291 mg) in the presence of 2-(2-bromoethoxy)tetrahydro-2H-pyran (3eq) [3eq 60% NaH was used] led to (1S,2S,5R)-1-((tert-butyldimethylsilyl)oxy)-2-isopropyl-5-methyl-N-((2S)-2-phenyl-2-(2-((tetrahydro-2H-pyran-2-yl)oxy)ethoxy)ethyl)cyclohexane-1-carboxamide (287 mg) as colorless oil after flash chromatography with cyclohexane and EtOAc.

Under deprotection conditions A, (1S,2S,5R)-1-((tert-butyldimethylsilyl)oxy)-2-isopropyl-5-methyl-N-((2S)-2-phenyl-2-(2-((tetrahydro-2H-pyran-2-yl)oxy)ethoxy)ethyl)cyclohexane-1-carboxamide (271 mg) in the presence of HCl (49.8eq) led to (1S,2S,5R)-1-hydroxy-N—((S)-2-(2-hydroxyethoxy)-2-phenylethyl)-2-isopropyl-5-methylcyclohexane-1-carboxamide (85 mg) as white solid after flash chromatography with cyclohexane and EtOAc.

### Example 57

### methyl 2-((S)-2-((1S,2S,5R)-1-hydroxy-2-isopropyl-5-methylcyclohexane-1-carboxamido)-1-phenylethoxy)acetate

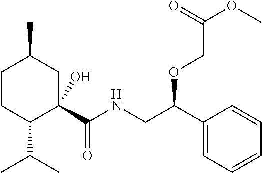 [SMILES: COC(=O)CO[C@H](CNC(=O)[C@]1(O)C[C@H](C)CC[C@H]1C(C)C)c1ccccc1]

Isopropyl ((1S,2S,5R)-1-hydroxy-2-isopropyl-5-methylcyclohexane-1-carbonyl)glycinate (79 mg) was dissolved in DCM/methanol (2/1, 0.1M) and the resulting solution was cooled down to 0° C. A 2M trimethylsilyl-diazomethane solution in hexane (1.6eq) was added and after warming up to room temperature, the reaction mixture was stirred during 3 hr30. The reaction was quenched by dropwise addition of acetic acid (1.0eq) and the reaction mixture was then concentrated under vacuum. The residue was dissolved in diethyl ether (10V), washed with an 1N aqueous solution of sodium hydroxide (10V), with brine (10V), dried over Na₂SO₄ and concentrated under vacuum. The residue was purified by flash chromatography with cyclohexane and EtOAc to provide methyl 2-((S)-2-((1S,2S,5R)-1-hydroxy-2-isopropyl-5-methylcyclohexane-1-carboxamido)-1-phenylethoxy)acetate (46.9 mg) as colorless oil.

### Example 58

### (1S,2S,5R)-1-hydroxy-2-isopropyl-5-methyl-N-(3-methylphenethyl)cyclohexane-1-carboxamide

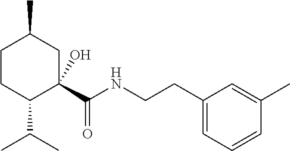 [SMILES: Cc1cccc(CCNC(=O)[C@]2(O)C[C@H](C)CC[C@H]2C(C)C)c1]

Under coupling conditions A, (1S,2S,5R)-1-hydroxy-2-isopropyl-5-methylcyclohexane-1-carboxylic acid (130 mg) and 3-methylphenethylamine (1.05eq) [after amine addition, the reaction was heated at 50-55° C. during 16 hr] led to (1S,2S,5R)-1-hydroxy-2-isopropyl-5-methyl-N-(3-methylphenethyl)cyclohexane-1-carboxamide (149 mg) as white solid after flash chromatography with cyclohexane and EtOAc.

### Example 59

### (1S,2S,5R)-1-hydroxy-2-isopropyl-5-methyl-N-(2-methylphenethyl)cyclohexane-1-carboxamide

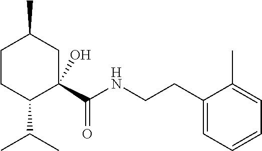 [SMILES: Cc1ccccc1CCNC(=O)[C@]1(O)C[C@H](C)CC[C@H]1C(C)C]

Under coupling conditions B, (1S,2S,5R)-1-hydroxy-2-isopropyl-5-methylcyclohexane-1-carboxylic acid (110 mg) and 2-methylphenethylamine (1.05eq) [after amine addition, the reaction was heated at 50-55° C. during 16 hr] led to (1S,2S,5R)-1-hydroxy-2-isopropyl-5-methyl-N-(2-methylphenethyl)cyclohexane-1-carboxamide (61 mg) as white solid after flash chromatography with cyclohexane and EtOAc.

### Example 60

### (1S,2S,5R)-1-hydroxy-2-isopropyl-5-methyl-N-(4-methylphenethyl)cyclohexane-1-carboxamide

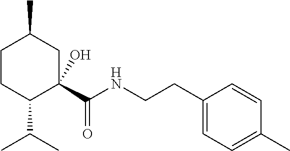 [SMILES: Cc1ccc(CCNC(=O)[C@]2(O)C[C@H](C)CC[C@H]2C(C)C)cc1]

Under coupling conditions B, (1S,2S,5R)-1-hydroxy-2-isopropyl-5-methylcyclohexane-1-carboxylic acid (110 mg) and 4-methylphenethylamine (1.05eq) [after amine addition, the reaction was heated at 50-55° C. during 16 hr] led to (1S,2S,5R)-1-hydroxy-2-isopropyl-5-methyl-N-(4-methylphenethyl)cyclohexane-1-carboxamide (95 mg) as white solid after flash chromatography with cyclohexane and EtOAc.

### Example 61

### methyl 2-(2-(2-((1S,2S,5R)-1-hydroxy-2-isopropyl-5-methylcyclohexane-1-carboxamido)ethyl)phenoxy)acetate

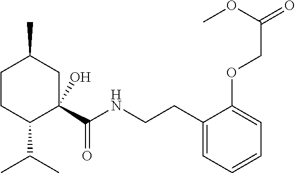 [SMILES: COC(=O)COc1ccccc1CCNC(=O)[C@]1(O)C[C@H](C)CC[C@H]1C(C)C]

Under coupling conditions B, (1S,2S,5R)-1-hydroxy-2-isopropyl-5-methylcyclohexane-1-carboxylic acid (750 mg) and (S)-2-amino-1-phenylethanol (570 mg) [after amine addition, the reaction was heated at 50° C. during 16 hrs] led to (1S,2S,5R)-1-hydroxy-N-(2-hydroxyphenethyl)-2-isopropyl-5-methylcyclohexane-1-carboxamide (702 mg) as yellow powder after flash chromatography with cyclohexane and EtOAc, and without reprecipitation.

Under alkylation conditions C, (1S,2S,5R)-1-hydroxy-N-(2-hydroxyphenethyl)-2-isopropyl-5-methylcyclohexane-1-carboxamide (90 mg) in the presence of methyl bromoacetate (1.5eq) [NB the reaction mixture was irradiated at 100° C. in a microwave] led to methyl 2-(2-(2-((1S,2S,5R)-1-hydroxy-2-isopropyl-5-methylcyclohexane-1-carboxamido)ethyl)phenoxy)acetate (92 mg) as white solid after flash chromatography with cyclohexane/EtOAc.

### Example 62

### (1S,2S,5R)—N—((S)-2-((R)-2-aminopropanamido)-2-phenylethyl)-1-hydroxy-2-isopropyl-5-methylcyclohexane-1-carboxamide

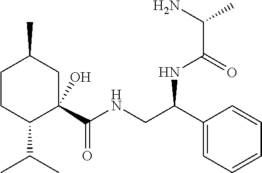 [SMILES: CC(C)[C@@H]1CC[C@@H](C)C[C@@]1(O)C(=O)NC[C@@H](NC(=O)[C@@H](C)N)c1ccccc1]

Under coupling conditions A, (1S,2S,5R)-1-hydroxy-2-isopropyl-5-methylcyclohexane-1-carboxylic acid (1.7 g) and (S)-tert-butyl (2-amino-1-phenylethyl)carbamate (1.05eq) [after amine addition, the reaction was stirred at 50-55° C. during 16 hr] led to tert-butyl ((S)-2-((1S,2S,5R)-1-hydroxy-2-isopropyl-5-methylcyclohexane-1-carboxamido)-1-phenylethyl)carbamate (2.26 g) as white solid after flash chromatography with cyclohexane and acetone.

Under deprotection conditions B, tert-butyl ((S)-2-((1S,2S,5R)-1-hydroxy-2-isopropyl-5-methylcyclohexane-1-carboxamido)-1-phenylethyl)carbamate (2.26 g) in the presence of TFA (10eq) led to (1S,2S,5R)—N—((S)-2-amino-2-phenylethyl)-1-hydroxy-2-isopropyl-5-methylcyclohexane-1-carboxamide (1.11 g) as ecru solid.

### (1S,2S,5R)—N—((S)-2-amino-2-phenylethyl)-1-hydroxy-2-isopropyl-5-methylcyclohexane-1-carboxamide

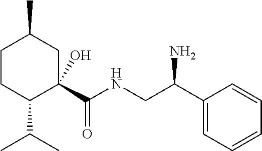 [SMILES: CC(C)[C@@H]1CC[C@@H](C)C[C@@]1(O)C(=O)NC[C@@H](N)c1ccccc1]

Under coupling conditions F, with (1S,2S,5R)—N—((S)-2-amino-2-phenylethyl)-1-hydroxy-2-isopropyl-5-methylcyclohexane-1-carboxamide (100 mg) and BOC-D-ALA-OH (1.1eq) led to tert-butyl ((R)-1-(((S)-2-((1S,2S,5R)-1-hydroxy-2-isopropyl-5-methylcyclohexane-1-carboxamido)-1-phenylethyl)amino)-1-oxopropan-2-yl)carbamate (143 mg) [no purification by flash chromatography was needed] as white foam.

Under deprotection conditions B, tert-butyl ((R)-1-(((S)-2-((1S,2S,5R)-1-hydroxy-2-isopropyl-5-methylcyclohexane-1-carboxamido)-1-phenylethyl)amino)-1-oxopropan-2-yl)carbamate (140 mg) in the presence of TFA (8.2eq) led to (1S,2S,5R)—N—((S)-2-((R)-2-aminopropanamido)-2-phenylethyl)-1-hydroxy-2-isopropyl-5-methylcyclohexane-1-carboxamide (95 mg) after flash chromatography with DCM and methanol.

### Example 63

### (1S,2S,5R)—N—((S)-2-((S)-2-aminopropanamido)-2-phenylethyl)-1-hydroxy-2-isopropyl-5-methylcyclohexane-1-carboxamide

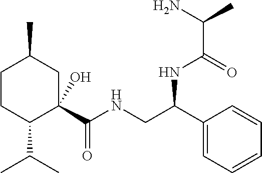 [SMILES: CC(C)[C@@H]1CC[C@@H](C)C[C@@]1(O)C(=O)NC[C@@H](NC(=O)[C@H](C)N)c1ccccc1]

Under coupling conditions A, (1S,2S,5R)-1-hydroxy-2-isopropyl-5-methylcyclohexane-1-carboxylic acid (1.7 g) and (S)-tert-butyl (2-amino-1-phenylethyl)carbamate (1.05eq) [after amine addition, the reaction was stirred at 50-55° C. during 16 hr] led to tert-butyl ((S)-2-((1S,2S,5R)-1-hydroxy-2-isopropyl-5-methylcyclohexane-1-carboxamido)-1-phenylethyl)carbamate (2.26 g) as white solid after flash chromatography with cyclohexane and acetone.

Under deprotection conditions B, tert-butyl ((S)-2-((1S,2S,5R)-1-hydroxy-2-isopropyl-5-methylcyclohexane-1-carboxamido)-1-phenylethyl)carbamate (2.26 g) in the presence of TFA (10eq) led to (1S,2S,5R)—N—((S)-2-amino-2-phenylethyl)-1-hydroxy-2-isopropyl-5-methylcyclohexane-1-carboxamide (1.11 g) as ecru solid.

Under coupling conditions F, with (1S,2S,5R)—N—((S)-2-amino-2-phenylethyl)-1-hydroxy-2-isopropyl-5-methylcyclohexane-1-carboxamide (100 mg) and BOC-L-ALA-OH (1.1eq) led to tert-butyl ((S)-1-(((S)-2-((1S,2S,5R)-1-hydroxy-2-isopropyl-5-methylcyclohexane-1-carboxamido)-1-phenylethyl)amino)-1-oxopropan-2-yl)carbamate (156 mg) as ecru solid [no purification by flash chromatography was needed].

Under deprotection conditions B, tert-butyl ((S)-1-(((S)-2-((1S,2S,5R)-1-hydroxy-2-isopropyl-5-methylcyclohexane-1-carboxamido)-1-phenylethyl)amino)-1-oxopropan-2-yl)carbamate (155 mg) in the presence of TFA (10.2eq) led to (1S,2S,5R)—N—((S)-2-((S)-2-aminopropanamido)-2-phenylethyl)-1-hydroxy-2-isopropyl-5-methylcyclohexane-1-carboxamide (103 mg) as white solid.

### Example 64

### methyl 2-(3-(2-((1S,2S,5R)-1-hydroxy-2-isopropyl-5-methylcyclohexane-1-carboxamido)ethyl)phenoxy)acetate

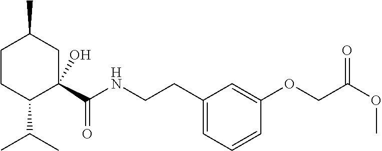 [SMILES: COC(=O)COc1cccc(CCNC(=O)[C@]2(O)C[C@H](C)CC[C@H]2C(C)C)c1]

Under coupling conditions B, (1S,2S,5R)-1-hydroxy-2-isopropyl-5-methylcyclohexane-1-carboxylic acid (750 mg) and 3-hydroxyphenythylamine (0.67eq) [CDI (0.58eq) & pyridine (0.71eq) were added and after amine addition, the reaction mixture was stirred at 50-55° C. during 4 hr] led to (1S,2S,5R)-1-hydroxy-N-(3-hydroxyphenethyl)-2-isopropyl-5-methylcyclohexane-1-carboxamide (450 mg) as white solid after flash chromatography with cyclohexane and EtOAc.

Under alkylation conditions C, (1S,2S,5R)-1-hydroxy-N-(3-hydroxyphenethyl)-2-isopropyl-5-methylcyclohexane-1-carboxamide (90 mg) in the presence of methyl bromoacetate (1.5eq) [NB the reaction mixture was irradiated at 100° C. in a microwave] led to methyl 2-(3-(2-((1S,2S,5R)-1-hydroxy-2-isopropyl-5-methylcyclohexane-1-carboxamido)ethyl)phenoxy)acetate (99 mg) as white solid after flash chromatography with cyclohexane/EtOAc.

### Example 65

### methyl 4-((1S,2S,5R)-1-hydroxy-2-isopropyl-5-methylcyclohexane-1-carboxamido)-3-phenyl butanoate

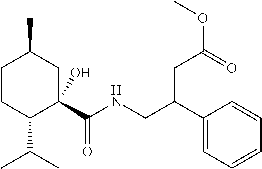 [SMILES: COC(=O)CC(CNC(=O)[C@]1(O)C[C@H](C)CC[C@H]1C(C)C)c1ccccc1]

Under coupling conditions B, (1S,2S,5R)-1-hydroxy-2-isopropyl-5-methylcyclohexane-1-carboxylic acid (1.02 g) and methyl 4-amino-3-phenylbutanoate hydrochloride (1.05eq) [after amine addition, the reaction was heated at 50-55° C. during 16 hr] led to methyl 4-((1S,2S,5R)-1-hydroxy-2-isopropyl-5-methylcyclohexane-1-carboxamido)-3-phenyl butanoate (1.445 g) as white solid after flash chromatography with cyclohexane and EtOAc.

### Example 66

### (1S,2S,5R)-1-hydroxy-N-(4-hydroxy-2-phenylbutyl)-2-isopropyl-5-methylcyclohexane-1-carboxamide

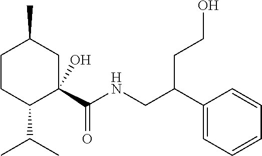 [SMILES: CC(C)[C@@H]1CC[C@@H](C)C[C@@]1(O)C(=O)NCC(CCO)c1ccccc1]

Under coupling conditions B, (1S,2S,5R)-1-hydroxy-2-isopropyl-5-methylcyclohexane-1-carboxylic acid (1.02 g) and methyl 4-amino-3-phenylbutanoate hydrochloride (1.03eq) [after amine addition, the reaction was stirred at 50-55° C. during 5 hr] led to methyl 4-((1S,2S,5R)-1-hydroxy-2-isopropyl-5-methylcyclohexane-1-carboxamido)-3-phenylbutanoate (1.445 g) as white solid after flash chromatography with cyclohexane and EtOAc.

Under reduction conditions B, methyl 4-((1S,2S,5R)-1-hydroxy-2-isopropyl-5-methylcyclohexane-1-carboxamido)-3-phenylbutanoate (200 mg) in the presence of LiBH₄ (11eq) led to (1S,2S,5R)-1-hydroxy-N-(4-hydroxy-2-phenylbutyl)-2-isopropyl-5-methylcyclohexane-1-carboxamide (143 mg) as white solid after flash chromatography with cyclohexane/EtOAc.

### Example 67

### phenyl ((1S,2S,5R)-1-hydroxy-2-isopropyl-5-methylcyclohexane-1-carbonyl)glycinate

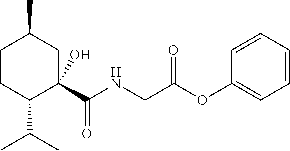 [SMILES: CC(C)[C@@H]1CC[C@@H](C)C[C@@]1(O)C(=O)NCC(=O)Oc1ccccc1]

Under coupling conditions B, (1S,2S,5R)-1-hydroxy-2-isopropyl-5-methylcyclohexane-1-carboxylic acid (1.0 g) and glycine methyl ester hydrochloride (1.02eq) [after amine addition, the reaction was heated at 50-55° C. during 3 hr] led to methyl ((1S,2S,5R)-1-hydroxy-2-isopropyl-5-methylcyclohexane-1-carbonyl)glycinate (1.15 g) as white solid.

Under saponification conditions, methyl ((1S,2S,5R)-1-hydroxy-2-isopropyl-5-methylcyclohexane-1-carbonyl)glycinate (1.15 g) in the presence of sodium hydroxide (1M, 5eq) led to ((1S,2S,5R)-1-hydroxy-2-isopropyl-5-methylcyclohexane-1-carbonyl)glycine (862 mg).

Under coupling conditions C, with ((1S,2S,5R)-1-hydroxy-2-isopropyl-5-methylcyclohexane-1-carbonyl)glycine (100 mg) and phenol as well as DCC (1.05eq) and pyridine (1.8eq) [no addition of DMAP for this reaction] led to phenyl ((1S,2S,5R)-1-hydroxy-2-isopropyl-5-methylcyclohexane-1-carbonyl)glycinate (86 mg) as white solid after flash chromatography with cyclohexane and EtOAc.

### Example 68

### 4-formyl-2-methoxyphenyl ((1S,2S,5R)-1-hydroxy-2-isopropyl-5-methylcyclohexane-1-carbonyl)glycinate

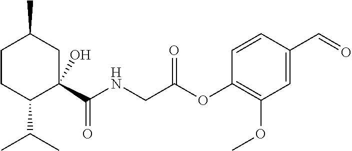 [SMILES: COc1cc(C=O)ccc1OC(=O)CNC(=O)[C@]1(O)C[C@H](C)CC[C@H]1C(C)C]

Under coupling conditions B, (1S,2S,5R)-1-hydroxy-2-isopropyl-5-methylcyclohexane-1-carboxylic acid (1.0 g) and glycine methyl ester hydrochloride (1.02eq) [after amine addition, the reaction was heated at 50-55° C. during 3 hr] led to methyl ((1S,2S,5R)-1-hydroxy-2-isopropyl-5-methylcyclohexane-1-carbonyl)glycinate (1.15 g) as white solid.

Under saponification conditions, methyl ((1S,2S,5R)-1-hydroxy-2-isopropyl-5-methylcyclohexane-1-carbonyl)glycinate (1.15 g) in the presence of sodium hydroxide (1M, 5eq) led to ((1S,2S,5R)-1-hydroxy-2-isopropyl-5-methylcyclohexane-1-carbonyl)glycine (862 mg).

Under coupling conditions D, with ((1S,2S,5R)-1-hydroxy-2-isopropyl-5-methylcyclohexane-1-carbonyl)glycine (90 mg) and vanillin (1.0eq) led to 4-formyl-2-methoxyphenyl ((1S,2S,5R)-1-hydroxy-2-isopropyl-5-methylcyclohexane-1-carbonyl)glycinate (45 mg) as white foam after flash chromatography with cyclohexane and EtOAc.

### Example 69

### methyl 4-((1S,2S,5R)-1-hydroxy-2-isopropyl-5-methylcyclohexane-1-carboxamido)-3-phenyl butanoate

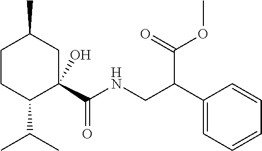 [SMILES: COC(=O)C(CNC(=O)[C@]1(O)C[C@H](C)CC[C@H]1C(C)C)c1ccccc1]

Under coupling conditions B, (1S,2S,5R)-1-hydroxy-2-isopropyl-5-methylcyclohexane-1-carboxylic acid (0.86 g) and methyl 3-amino-2-phenylpropanoate hydrochloride (1.05eq) [after amine addition, the reaction was heated at 50-55° C. during 16 hr] led to methyl 4-((1S,2S,5R)-1-hydroxy-2-isopropyl-5-methylcyclohexane-1-carboxamido)-3-phenyl butanoate (1.107 g) as white solid after flash chromatography with cyclohexane and EtOAc followed by trituration in DCM and n-pentane.

### Example 70

### (1S,2S,5R)-1-hydroxy-N-(3-hydroxy-2-phenylpropyl)-2-isopropyl-5-methylcyclohexane-1-carboxamide

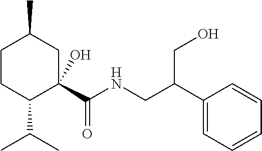 [SMILES: CC(C)[C@@H]1CC[C@@H](C)C[C@@]1(O)C(=O)NCC(CO)c1ccccc1]

Under reduction conditions B, methyl 4-((1S,2S,5R)-1-hydroxy-2-isopropyl-5-methylcyclohexane-1-carboxamido)-3-phenyl butanoate (190 mg) in the presence of LiBH₄ (7eq) led to (1S,2S,5R)-1-hydroxy-N-(3-hydroxy-2-phenylpropyl)-2-isopropyl-5-methylcyclohexane-1-carboxamide (113 mg) as white solid after flash chromatography with cyclohexane/EtOAc followed by preparative HPLC using Xselect CSH Prep C18 5 im OBD 50×250m column with water and acetonitrile containing 0.1% formic acid.

### Example 71

### 2-aminoethyl 3-((1S,2S,5R)-1-hydroxy-2-isopropyl-5-methylcyclohexane-1-carboxamido)-2-phenylpropanoate hydrochloride

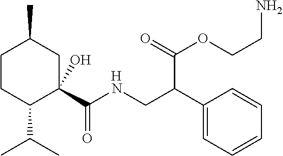

Under saponification conditions, methyl 4-((1S,2S,5R)-1-hydroxy-2-isopropyl-5-methylcyclohexane-1-carboxamido)-3-phenyl butanoate (650 mg) in the presence of sodium hydroxide (12M, 5eq) led to 3-((1S,2S,5R)-1-hydroxy-2-isopropyl-5-methylcyclohexane-1-carboxamido)-2-phenylpropanoic acid (615 mg).

Under coupling conditions D, with 3-((1S,2S,5R)-1-hydroxy-2-isopropyl-5-methylcyclohexane-1-carboxamido)-2-phenylpropanoic acid (150 mg) and tert-butyl N-(2-hydroxyethyl)carbamate (1.2eq) led to 2-((tert-butoxycarbonyl)amino)ethyl 3-((1S,2S,5R)-1-hydroxy-2-isopropyl-5-methylcyclohexane-1-carboxamido)-2-phenylpropanoate (180 mg) as colorless wax after flash chromatography with cyclohexane and EtOAc.

Under deprotection conditions A, 2-((tert-butoxycarbonyl)amino)ethyl 3-((1S,2S,5R)-1-hydroxy-2-isopropyl-5-methylcyclohexane-1-carboxamido)-2-phenylpropanoate (177 mg) in the presence of HCl (2M, 10eq) led to 2-aminoethyl 3-((1S,2S,5R)-1-hydroxy-2-isopropyl-5-methylcyclohexane-1-carboxamido)-2-phenylpropanoate hydrochloride (145 mg).

### Example 72

### (1S,2S,5R)-1-hydroxy-N-(2-hydroxy-2-phenylethyl-2-d)-2-isopropyl-5-methylcyclohexane-1-carboxamide

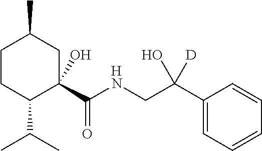

Under coupling conditions B, (1S,2S,5R)-1-hydroxy-2-isopropyl-5-methylcyclohexane-1-carboxylic acid (2.0 g) and 2-aminoacetophenone hydrochloride (1.0eq) [after amine addition, the reaction mixture was heated at 55° C. during 16 hr] led to (1S,2S,5R)-1-hydroxy-2-isopropyl-5-methyl-N-(2-oxo-2-phenylethyl)cyclohexane-1-carboxamide (1.81 g) as pale yellow solid.

Under reduction conditions C, (1S,2S,5R)-1-hydroxy-2-isopropyl-5-methyl-N-(2-oxo-2-phenylethyl)cyclohexane-1-carboxamide (1.80 g) in the presence of NaBD4 (1.25eq) led to (1S,2S,5R)-1-hydroxy-N-(2-hydroxy-2-phenylethyl-2-d)-2-isopropyl-5-methylcyclohexane-1-carboxamide (1.593 g) as ecru solid.

### Example 73

### 2-amino-2-methylpropyl 3-((1S,2S,5R)-1-hydroxy-2-isopropyl-5-methylcyclohexane-1-carboxamido)-2-phenylpropanoate hydrochloride

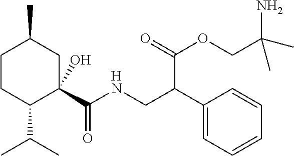

Under saponification conditions, methyl 4-((1S,2S,5R)-1-hydroxy-2-isopropyl-5-methylcyclohexane-1-carboxamido)-3-phenyl butanoate (650 mg) in the presence of sodium hydroxide (12M, 5eq) led to 3-((1S,2S,5R)-1-hydroxy-2-isopropyl-5-methylcyclohexane-1-carboxamido)-2-phenylpropanoic acid (615 mg).

Under coupling conditions D, with 3-((1S,2S,5R)-1-hydroxy-2-isopropyl-5-methylcyclohexane-1-carboxamido)-2-phenylpropanoic acid (150 mg) and N-Boc-2-amino-2-methyl-1-propanol (1.2eq) led to 2-((tert-butoxycarbonyl)amino)-2-methylpropyl 3-((1S,2S,5R)-1-hydroxy-2-isopropyl-5-methylcyclohexane-1-carboxamido)-2-phenylpropanoate (87 mg) as colorless wax after flash chromatography with cyclohexane and EtOAc followed by preparative HPLC using Xselect CSH Prep C18 5 im OBD 50×250m column with water and acetonitrile containing 0.1% formic acid.

Under deprotection conditions A, 2-((tert-butoxycarbonyl)amino)-2-methylpropyl 3-((1S,2S,5R)-1-hydroxy-2-isopropyl-5-methylcyclohexan-1-carboxamido)-2-phenylpropanoate (86 mg) in the presence of HCl (2M, 12eq) led to 2-amino-2-methylpropyl 3-((1S,2S,5R)-1-hydroxy-2-isopropyl-5-methylcyclohexane-1-carboxamido)-2-phenylpropanoate hydrochloride (77 mg) as white solid.

### Example 74

### methyl 2-(3-(2-((1S,2S,5R)-1-hydroxy-2-isopropyl-5-methylcyclohexane-1-carboxamido)ethyl)phenyl)acetate

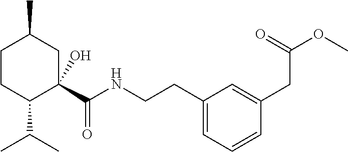

Methyl 2-(3-bromophenyl)acetate (1.04 g) and potassium tert-butyl N-[2-(trifluoroboranuidyl)ethyl]carbamate (1.1eq) as well as cesium carbonate (3.0eq) were dissolved in a 3/1 mixture of toluene and water (0.27M). The resulting solution was degassed using argon and 1,1′-bis(diphenylphosphino)ferrocene-palladium(II) dichloride dichloromethane complex (0.04eq) was added. The reaction mixture was then heated at 80° C. during 16 hrs. Once at room temperature, EtOAc (1V) was added and the resulting mixture was washed with water (1V×2). The organic phase was washed with an 1N aqueous solution of HCl (1V), with brine (1V), dried over Na2SO4, filtrated and concentrated under vacuum. The residue was purified by flash chromatography with cyclohexane and EtOAc to provide methyl 2-(3-(2-((tert-butoxycarbonyl)amino)ethyl)phenyl)acetate (998 mg) as colorless oil.

Under deprotection conditions A, methyl 2-(3-(2-((tert-butoxycarbonyl)amino)ethyl)phenyl)acetate (996 mg) in the presence of HCl (2M, 5.9eq) led to methyl 2-(3-(2-aminoethyl)phenyl)acetate hydrochloride (760 mg) as white solid.

Under coupling conditions B, (1S,2S,5R)-1-hydroxy-2-isopropyl-5-methylcyclohexane-1-carboxylic acid (0.64 g) and methyl 2-(3-(2-aminoethyl)phenyl)acetate hydrochloride (1.05eq) [after amine addition, the reaction was heated at 50-55° C. during 16 hr] led to methyl 2-(3-(2-((1S,2S,5R)-1-hydroxy-2-isopropyl-5-methylcyclohexane-1-carboxamido)ethyl) phenyl)acetate (564 mg) as colorless wax after flash chromatography with cyclohexane and EtOAc.

### Example 75

### methyl 3-(2-(2-((1S,2S,5R)-1-hydroxy-2-isopropyl-5-methylcyclohexane-1-carboxamido)ethyl)phenoxy)propanoate

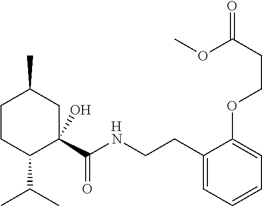

Under coupling conditions A, (1S,2S,5R)-1-hydroxy-2-isopropyl-5-methylcyclohexane-1-carboxylic acid (0.64 g) and 2-(2-aminoethyl)phenol (1.03eq) [after amine addition, the reaction was heated at 55° C. during 16 hr] led to (1S,2S,5R)-1-hydroxy-N-(2-hydroxyphenethyl)-2-isopropyl-5-methylcyclohexane-1-carboxamide (965 mg) as white solid after flash chromatography with cyclohexane and EtOAc.

Under alkylation conditions C, (1S,2S,5R)-1-hydroxy-N-(2-hydroxyphenethyl)-2-isopropyl-5-methylcyclohexane-1-carboxamide (400 mg) in the presence of 3-bromo-1-propanol (1.1eq) led to (1S,2S,5R)-1-hydroxy-N-(2-(3-hydroxypropoxy)phenethyl)-2-isopropyl-5-methylcyclohexane-1-carboxamide (371 mg) as colorless wax after flash chromatography with cyclohexane and EtOAc.

(1S,2S,5R)-1-Hydroxy-N-(2-(3-hydroxypropoxy)phenethyl)-2-isopropyl-5-methylcyclohexane-1-carboxamide (369 mg) was dissolved in acetone (0.15M) and the resulting solution was cooled down to 0° C. Chromium trioxide (3.6eq) was added dropwise during 20 min and the reaction mixture was stirred at 0° C. during 2 hr. Sodium metabisulfite was added and the resulting mixture was stirred at room temperature until the solution turned green. Water (10V) was added and the resulting solution was extracted with diethyl ether (10 v×2). The combined organic phase was washed with an 1N aqueous solution of sodium hydroxide (5V). The combined sodium hydroxide phases were acidified with an 5N aqueous solution of hydrochloric acid and were extracted with diethyl ether (10V×2). All combined diethyl ether phases were washed with brine (10V), dried over Na2SO4, filtrated and concentrated under vacuum to provide 3-(2-(2-((1S,2S,5R)-1-hydroxy-2-isopropyl-5-methylcyclohexane-1-carboxamido)ethyl)phenoxy)propanoic acid (211 mg) as white foam.

3-(2-(2-((1S,2S,5R)-1-Hydroxy-2-isopropyl-5-methylcyclohexane-1-carboxamido)ethyl)phenoxy)propanoic acid (199 mg) was dissolved in DCM/methanol (2/1, 0.1M) and the resulting solution was cooled down to 0° C. A 2M trimethylsilyl-diazomethane solution in hexane (2.2eq) was added and after warming up to room temperature, the reaction mixture was stirred during 26 hr. The reaction was quenched by dropwise addition of acetic acid (2.0eq) and the reaction mixture was then concentrated under vacuum. The residue was dissolved in DCM (5V), washed with a saturated aqueous solution of sodium bicarbonate (5V), with brine (5V), dried over Na2SO4 and concentrated under vacuum. The residue was purified by flash chromatography with cyclohexane and EtOAc to provide methyl 3-(2-(2-((1S,2S,5R)-1-hydroxy-2-isopropyl-5-methylcyclohexane-1-carboxamido)ethyl)phenoxy) propanoate (165 mg).

### Example 76

### 3,5-dihydroxyphenyl ((1S,2S,5R)-1-hydroxy-2-isopropyl-5-methylcyclohexane-1-carbonyl)glycinate

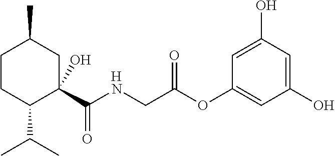

Under coupling conditions B, (1S,2S,5R)-1-hydroxy-2-isopropyl-5-methylcyclohexane-1-carboxylic acid (1.0 g) and glycine methyl ester hydrochloride (1.02eq) [after amine addition, the reaction was heated at 50-55° C. during 3 hr] led to methyl ((1S,2S,5R)-1-hydroxy-2-isopropyl-5-methylcyclohexane-1-carbonyl)glycinate (1.15 g) as white solid.

Under saponification conditions, methyl ((1S,2S,5R)-1-hydroxy-2-isopropyl-5-methylcyclohexane-1-carbonyl)glycinate (1.15 g) in the presence of sodium hydroxide (1M, 5eq) led to ((1S,2S,5R)-1-hydroxy-2-isopropyl-5-methylcyclohexane-1-carbonyl)glycine (862 mg).

Under coupling conditions E, with ((1S,2S,5R)-1-hydroxy-2-isopropyl-5-methylcyclohexane-1-carbonyl)glycine (130 mg) and phloroglucinol (3.0eq) led to 3,5-dihydroxyphenyl ((1S,2S,5R)-1-hydroxy-2-isopropyl-5-methylcyclohexane-1-carbonyl)glycinate (51 mg) as white solid after flash chromatography with cyclohexane and EtOAc followed by preparative HPLC using Xselect CSH Prep C18 5 im OBD 50×250m column with water and acetonitrile containing 0.1% formic acid.

### Example 77

### methyl 2-(2-(2-((1S,2S,5R)-1-hydroxy-2-isopropyl-5-methylcyclohexane-1-carboxamido)ethyl)phenyl)acetate

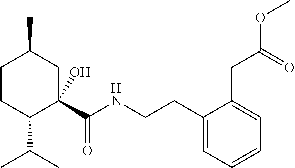

Methyl 2-(2-bromophenyl)acetate (1.008 g) and potassium tert-butyl N-[2-(trifluoroboranuidyl)ethyl]carbamate (1.1eq) as well as cesium carbonate (3.0eq) were dissolved in a 3/1 mixture of toluene and water (0.27M). the resulting solution was degassed using argon and 1,1′bis(diphenylphosphino)ferrocene-palladium(II)dichloride dichloromethane complex (0.04eq) was added. The reaction mixture was then heated at 80° C. during 16 hrs. Once at room temperature, EtOAc (1V) was added and the resulting mixture was washed with water (1V×2). The organic phase was washed with an 1N aqueous solution of HCl (1V), with brine (1V), dried over Na2SO4, filtrated and concentrated under vacuum. The residue was purified by flash chromatography with cyclohexane and EtOAc to provide methyl 2-(2-(2-((tert-butoxycarbonyl)amino)ethyl)phenyl)acetate (1.039 g).

Under deprotection conditions A, methyl 2-(2-(2-((tert-butoxycarbonyl)amino)ethyl)phenyl)acetate (1.036 g) in the presence of HCl (2M, 5.0eq) led to methyl 2-(2-(2-aminoethyl)phenyl)acetate hydrochloride (760 mg) Under coupling conditions B, (1S,2S,5R)-1-hydroxy-2-isopropyl-5-methylcyclohexane-1-carboxylic acid (150 mg) and methyl 2-(2-(2-aminoethyl)phenyl)acetate hydrochloride (1.02eq) [after amine addition, the reaction was heated at 55° C. during 4 hr30 and at room temperature during 72 hrs] led to methyl 2-(2-(2-((1S,2S,5R)-1-hydroxy-2-isopropyl-5-methylcyclohexane-1-carboxamido)ethyl)phenyl)acetate (360 mg) as colorless wax after flash chromatography with cyclohexane and EtOAc followed by preparative HPLC using Xselect CSH Prep C18 5 im OBD 50×250m column with water and acetonitrile containing 0.1% formic acid.

### Example 78

### 2-(3-(2-((1S,2S,5R)-1-hydroxy-2-isopropyl-5-methylcyclohexane-1-carboxamido)ethyl) phenyl)acetic acid

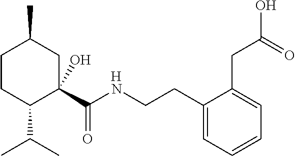

Under saponification condition, methyl 2-(3-(2-((1S,2S,5R)-1-hydroxy-2-isopropyl-5-methylcyclohexane-1-carboxamido)ethyl) phenyl)acetate (460 mg) in the presence of sodium hydroxide (1M, 4.08eq) led to 2-(3-(2-((1S,2S,5R)-1-hydroxy-2-isopropyl-5-methylcyclohexane-1-carboxamido)ethyl) phenyl)acetic acid (436 mg) as a white solid.

### Example 79

### 4-(hydroxymethyl)-2-methoxyphenyl ((1S,2S,5R)-1-hydroxy-2-isopropyl-5-methylcyclohexane-1-carbonyl)glycinate

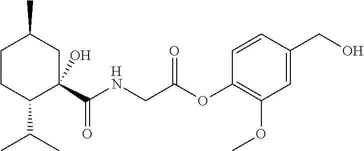

Under coupling conditions B, (1S,2S,5R)-1-hydroxy-2-isopropyl-5-methylcyclohexane-1-carboxylic acid (1.0 g) and glycine methyl ester hydrochloride (1.02eq) [after amine addition, the reaction was heated at 50-55° C. during 3 hr] led to methyl ((1S,2S,5R)-1-hydroxy-2-isopropyl-5-methylcyclohexane-1-carbonyl)glycinate (1.15 g) as white solid.

Under saponification conditions, methyl ((1S,2S,5R)-1-hydroxy-2-isopropyl-5-methylcyclohexane-1-carbonyl)glycinate (1.15 g) in the presence of sodium hydroxide (1M, 5eq) led to ((1S,2S,5R)-1-hydroxy-2-isopropyl-5-methylcyclohexane-1-carbonyl)glycine (862 mg).

Under coupling conditions E, with ((1S,2S,5R)-1-hydroxy-2-isopropyl-5-methylcyclohexane-1-carbonyl)glycine (90 mg) and vanillin (12eq) led to 4-formyl-2-methoxyphenyl ((1S,2S,5R)-1-hydroxy-2-isopropyl-5-methylcyclohexane-1-carbonyl)glycinate (110 mg) as yellow wax after flash chromatography with cyclohexane and EtOAc.

Under reduction conditions C, 4-formyl-2-methoxyphenyl ((1S,2S,5R)-1-hydroxy-2-isopropyl-5-methylcyclohexane-1-carbonyl)glycinate (108 mg) in the presence of NaBH₄ (1.1eq) led to 4-(hydroxymethyl)-2-methoxyphenyl ((1S,2S,5R)-1-hydroxy-2-isopropyl-5-methylcyclohexane-1-carbonyl)glycinate (29 mg) after preparative HPLC using Xselect CSH Prep C18 5 μm OBD 50×250m column with water and acetonitrile containing 0.1% formic acid.

### Example 80

### (1S,2S,5R)-1-hydroxy-N-(2-(3-hydroxypropoxy)phenethyl)-2-isopropyl-5-methylcyclohexane-1-carboxamide

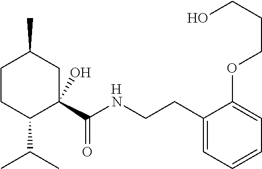

Under coupling conditions A, (1S,2S,5R)-1-hydroxy-2-isopropyl-5-methylcyclohexane-1-carboxylic acid (1.4 g) and 2-(2-aminoethyl)phenol (1.03eq) [after amine addition, the reaction was heated at 65° C. during 4 hr] led to (1S,2S,5R)-1-hydroxy-N-(2-hydroxyphenethyl)-2-isopropyl-5-methylcyclohexane-1-carboxamide (1.296 g) as brown solid after flash chromatography with cyclohexane and EtOAc.

Under alkylation conditions C, (1S,2S,5R)-1-hydroxy-N-(2-hydroxyphenethyl)-2-isopropyl-5-methylcyclohexane-1-carboxamide (100 mg) in the presence of 3-bromo-1-propanol (1.1eq) led to (1S,2S,5R)-1-hydroxy-N-(2-(3-hydroxypropoxy)phenethyl)-2-isopropyl-5-methylcyclohexane-1-carboxamide (77.6 mg) as colorless wax after flash chromatography with cyclohexane and EtOAc.

### Example 81

### (1S,2S,5R)—N-(2-(2-amino-2-oxoethoxy)phenethyl)-1-hydroxy-2-isopropyl-5-methyl cyclohexane-1-carboxamide

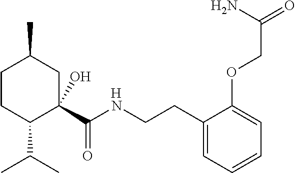

Under coupling conditions A, (1S,2S,5R)-1-hydroxy-2-isopropyl-5-methylcyclohexane-1-carboxylic acid (1.4 g) and 2-(2-aminoethyl)phenol (1.03eq) [after amine addition, the reaction was heated at 65° C. during 4 hr] led to (1S,2S,5R)-1-hydroxy-N-(2-hydroxyphenethyl)-2-isopropyl-5-methylcyclohexane-1-carboxamide (1.296 g) as brown solid after flash chromatography with cyclohexane and EtOAc.

Under alkylation conditions C, (1S,2S,5R)-1-hydroxy-N-(2-hydroxyphenethyl)-2-isopropyl-5-methylcyclohexane-1-carboxamide (100 mg) in the presence of 2-bromoacetamide (1.2eq) led to (1S,2S,5R)—N-(2-(2-amino-2-oxoethoxy)phenethyl)-1-hydroxy-2-isopropyl-5-methylcyclohexane-1-carboxamide (85.2 mg) as white foam after flash chromatography with cyclohexane and EtOAc.

### Examples 82 & 83

### (1S,2S,5R)-1-hydroxy-2-isopropyl-5-methyl-N-((3RS)-3,3,3-trifluoro-2-hydroxy-2-phenylpropyl) cyclohexane-1-carboxamide (82)

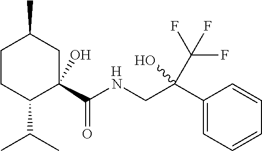

### (1S,2S,5R)-1-hydroxy-2-isopropyl-5-methyl-N-((3RS)-3,3,3-trifluoro-2-hydroxy-2-phenylpropyl) cyclohexane-1-carboxamide(83)

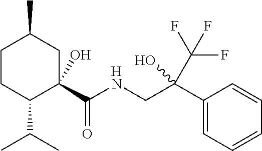

Trifluoroacetophenone (400 μl) and potassium carbonate (0.97eq) were dissolved in nitromethane (8.93eq) at room temperature. The reaction mixture was stirred during 1 hr. Water (10V) and EtOAc (10V) were added. The aqueous phase was extracted with EtOAc (10V). The combined organic phases were washed with an 1N aqueous solution of HCl (10V), with brine (10V), dried over Na₂SO₄, filtrated and concentrated under vacuum to give 1,1,1-Trifluoro-3-nitro-2-phenylpropan-2-ol (648 mg) as colorless oil.

1,1,1-Trifluoro-3-nitro-2-phenylpropan-2-ol (646 mg) was dissolved in methanol (0.27M) and Pd on charcoal (0.08eq) was added. The resulting solution was then put under hydrogen atmosphere and the reaction mixture was stirred at room temperature under hydrogen atmosphere during 8 hrs. The solution was filtrated on GF/F Whatman filter paper and the filter paper was rinsed with methanol: the filtrate was concentrated under vacuum. The residue was dissolved in diethyl ether (1V) and an 1N aqueous solution of HCl (1V) was added. The organic phase was then washed again with 1N aqueous solution of HCl (1V). The combined HCl phases were then brought to pH 12 by addition of a concentrated solution of sodium hydroxide. The resulting aqueous was then extracted with diethyl ether (1V×4). The combined organic phases were dried over Na₂SO₄, filtrated and concentrated under vacuum. The residue was purified by flash chromatography with DCM and methanol to yield 3-amino-1,1,1-trifluoro-2-phenylpropan-2-ol (318 mg) as white solid.

Under coupling conditions A, (1S,2S,5R)-1-hydroxy-2-isopropyl-5-methylcyclohexane-1-carboxylic acid (1.4 g) and the previously isolated amine (1.02eq) [after amine addition, the reaction was heated at 65° C. during 21 hr] led to (1S,2S,5R)-1-hydroxy-2-isopropyl-5-methyl-N-((3RS)-3,3,3-trifluoro-2-hydroxy-2-phenylpropyl) cyclohexane-1-carboxamide (example 82, 77 mg) and (1S,2S,5R)-1-hydroxy-2-isopropyl-5-methyl-N-((3RS)-3,3,3-trifluoro-2-hydroxy-2-phenylpropyl) cyclohexane-1-carboxamide (example 83, 74 mg) as white solid for both after preparative HPLC using Xselect CSH Prep C18 5 μm OBD 50×250m column with water and acetonitrile containing 0.1% formic acid.

### Example 84

### (1S,2S,5R)—N-(2-(2-acetamidoethoxy)phenethyl)-1-hydroxy-2-isopropyl-5-methylcyclohexane-1-carboxamide

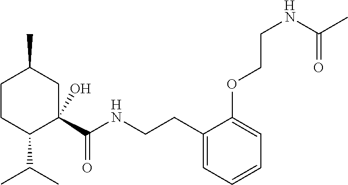

Under coupling conditions A, (1S,2S,5R)-1-hydroxy-2-isopropyl-5-methylcyclohexane-1-carboxylic acid (1.4 g) and 2-(2-aminoethyl)phenol (1.03eq) [after amine addition, the reaction was heated at 65° C. during 4 hr] led to the desired amide (1.296 g) as brown solid after flash chromatography with cyclohexane and EtOAc.

Under alkylation conditions A, the previously amide (150 mg) in the presence of tert-butyl N-(2-bromoethyl)carbamate (1.5eq) led to tert-butyl (2-(2-(2-((1S,2S,5R)-1-hydroxy-2-isopropyl-5-methylcyclohexane-1-carboxamido)ethyl)phenoxy)ethyl)carbamate (196 mg) after flash chromatography with cyclohexane/EtOAc.

Under deprotection conditions B, tert-butyl (2-(2-(2-((1S,2S,5R)-1-hydroxy-2-isopropyl-5-methylcyclohexane-1-carboxamido)ethyl)phenoxy)ethyl)carbamate in the presence of TFA (3.06eq) led to (1S,2S,5R)—N-(2-(2-aminoethoxy)phenethyl)-1-hydroxy-2-isopropyl-5-methylcyclohexane-1-carboxamide (118 mg). (1S,2S,5R)—N-(2-(2-Aminoethoxy)phenethyl)-1-hydroxy-2-isopropyl-5-methylcyclohexane-1-carboxamide (118 mg) was then dissolved in DCM (0.1M) followed by addition of triethylamine (1.15eq). The resulting solution was cooled down to 0° C. and acetic anhydride (0.76eq) was added. The reaction was stirred at room temperature during 16 hrs. After addition of DCM (10V), the resulting solution was washed with water (10V), an 1N aqueous solution of HCl (10V), brine (10V), dried over Na₂SO₄, filtrated and concentrated under vacuum. The residue was purified by flash chromatography with cyclohexane and EtOAc to yield (1S,2S,5R)—N-(2-(2-acetamidoethoxy)phenethyl)-1-hydroxy-2-isopropyl-5-methylcyclohexane-1-carboxamide (113 mg).

### Example 85

### (1S,2S,5R)—N-(2-(2-fluorophenyl)-2-oxoethyl)-1-hydroxy-2-isopropyl-5-methylcyclohexane-1-carboxamide

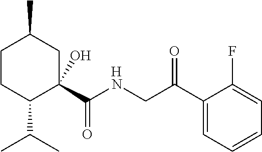

Under coupling conditions B, (1S,2S,5R)-1-hydroxy-2-isopropyl-5-methylcyclohexane-1-carboxylic acid (150 mg) and 2-amino-1-(2-fluorophenyl)ethanone hydrochloride (1.02eq) [after amine addition, the reaction was heated at 65° C. during 16 hr] led to (1S,2S,5R)—N-(2-(2-fluorophenyl)-2-oxoethyl)-1-hydroxy-2-isopropyl-5-methylcyclohexane-1-carboxamide (139 mg) as colorless wax after flash chromatography with cyclohexane and EtOAc.

### Example 86

### (1S,2S,5R)-1-hydroxy-N—((R)-3-hydroxy-1-phenylpropyl)-2-isopropyl-5-methylcyclohexane-1-carboxamide

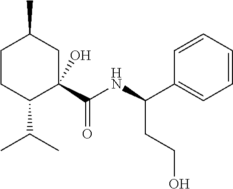 [SMILES: CC(C)[C@@H]1CC[C@@H](C)C[C@@]1(O)C(=O)N[C@H](CCO)c1ccccc1]

Under coupling conditions A, (1S,2S,5R)-1-hydroxy-2-isopropyl-5-methylcyclohexane-1-carboxylic acid (120 mg) and (1R)-1-phenyl-3-propanolamine (1.02eq) [after amine addition, the reaction was heated at 65° C. during 16 hr] led to (1S,2S,5R)-1-hydroxy-N—((R)-3-hydroxy-1-phenylpropyl)-2-isopropyl-5-methylcyclohexane-1-carboxamide (49 mg) as white solid after flash chromatography with cyclohexane and EtOAc.

### Example 87

### 3-(3-(2-((1S,2S,5R)-1-hydroxy-2-isopropyl-5-methylcyclohexane-1-carboxamido)ethyl) phenyl)propanoic acid

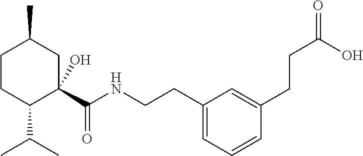 [SMILES: CC(C)[C@@H]1CC[C@@H](C)C[C@@]1(O)C(=O)NCCc1cccc(CCC(=O)O)c1]

Methyl 2-(3-bromophenyl)acetate (977 mg) and potassium tert-butyl N-[2-(trifluoroboranuidyl)ethyl]carbamate (1.1eq) as well as cesium carbonate (3.0eq) were dissolved in a 3/1 mixture of toluene and water (0.27M). The resulting solution was degassed using argon and 1,1′-bis(diphenylphosphino)ferrocene-palladium(II) dichloride dichloromethane complex (0.04eq) was added. The reaction mixture was then heated at 80° C. during 16 hrs. Once at room temperature, EtOAc (1V) was added and the resulting mixture was washed with water (1V×2). The organic phase was washed with an 1N aqueous solution of HCl (1V), with brine (1V), dried over Na₂SO₄, filtrated and concentrated under vacuum. The residue was purified by flash chromatography with cyclohexane and EtOAc to provide methyl 3-(3-(2-((tert-butoxycarbonyl)amino)ethyl)phenyl)propanoate (872 mg) as colorless oil.

Under deprotection conditions A, methyl 3-(3-(2-((tert-butoxycarbonyl)amino)ethyl)phenyl)propanoate (870 mg) in the presence of HCl (1M, 4.95eq) led to methyl 3-(3-(2-aminoethyl)phenyl)propanoate hydrochloride (387 mg).

Under coupling conditions A, (1S,2S,5R)-1-hydroxy-2-isopropyl-5-methylcyclohexane-1-carboxylic acid (200 mg) and methyl 3-(3-(2-aminoethyl)phenyl)propanoate hydrochloride (1.03eq) [after amine addition, the reaction was heated at 55° C. during 16 hr] led to methyl 3-(3-(2-((1S,2S,5R)-1-hydroxy-2-isopropyl-5-methylcyclohexane-1-carboxamido)ethyl)phenyl)propanoate (250 mg) as colorless wax after flash chromatography with cyclohexane and EtOAc.

Under saponification conditions, methyl 3-(3-(2-((1S,2S,5R)-1-hydroxy-2-isopropyl-5-methylcyclohexane-1-carboxamido)ethyl)phenyl)propanoate (170 mg) in the presence of sodium hydroxide (4.6eq) led to 3-(3-(2-((1S,2S,5R)-1-hydroxy-2-isopropyl-5-methylcyclohexane-1-carboxamido)ethyl) phenyl)propanoic acid (149 mg).

### Example 88

### (1S,2S,5R)-1-hydroxy-N-(2-((4-hydroxyphenyl)amino)-2-oxoethyl)-2-isopropyl-5-methylcyclohexane-1-carboxamide

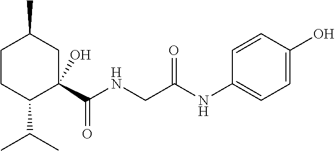 [SMILES: CC(C)[C@@H]1CC[C@@H](C)C[C@@]1(O)C(=O)NCC(=O)Nc1ccc(O)cc1]

Under coupling conditions B, (1S,2S,5R)-1-hydroxy-2-isopropyl-5-methylcyclohexane-1-carboxylic acid (0.5 g) and glycine methyl ester hydrochloride (1.02eq) [after amine addition, the reaction was heated at 65° C. during 16 hr30] led to methyl ((1S,2S,5R)-1-hydroxy-2-isopropyl-5-methylcyclohexane-1-carbonyl)glycinate (577 mg) as white solid.

Under saponification conditions, methyl ((1S,2S,5R)-1-hydroxy-2-isopropyl-5-methylcyclohexane-1-carbonyl)glycinate (577 mg) in the presence of sodium hydroxide (12M, 4.8eq) led to ((1S,2S,5R)-1-hydroxy-2-isopropyl-5-methylcyclohexane-1-carbonyl)glycine (470 mg).

Under coupling conditions E, with ((1S,2S,5R)-1-hydroxy-2-isopropyl-5-methylcyclohexane-1-carbonyl)glycine (90 mg) and 4-aminophenol (1.3eq) led to (1S,2S,5R)-1-hydroxy-N-(2-((4-hydroxyphenyl)amino)-2-oxoethyl)-2-isopropyl-5-methylcyclohexane-1-carboxamide (158 mg) as white solid.

### Examples 89 & 90

### (1S,2S,5R)-1-hydroxy-N-((2RS)-3-hydroxy-2-phenylpropyl)-2-isopropyl-5-methylcyclohexane-1-carboxamide (89)

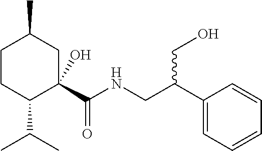 [SMILES: CC(C)[C@@H]1CC[C@@H](C)C[C@@]1(O)C(=O)NCC(CO)c1ccccc1]

### (1S,2S,5R)-1-hydroxy-N-((2RS)-3-hydroxy-2-phenylpropyl)-2-isopropyl-5-methylcyclohexane-1-carboxamide (90)

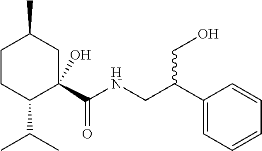 [SMILES: CC(C)[C@@H]1CC[C@@H](C)C[C@@]1(O)C(=O)NCC(CO)c1ccccc1]

Chiral separation of (1S,2S,5R)-1-hydroxy-N-(3-hydroxy-2-phenylpropyl)-2-isopropyl-5-methylcyclohexane-1-carboxamide (103.7 mg) carried out by liquid chromatography using CHIRALPAK IA with heptane and ethanol led to (1S,2S,5R)-1-hydroxy-N-(3-hydroxy-2-phenylpropyl)-2-isopropyl-5-methylcyclohexane-1-carboxamide (example 89, 43 mg) and (1S,2S,5R)-1-hydroxy-N-(3-hydroxy-2-phenylpropyl)-2-isopropyl-5-methylcyclohexane-1-carboxamide (example 90, 45 mg).

### Examples 91 & 92

### (1S,2S,5R)-1-hydroxy-N-((2RS)-2-hydroxy-2-phenylethyl-2-d)-2-isopropyl-5-methylcyclohexane-1-carboxamide (91)

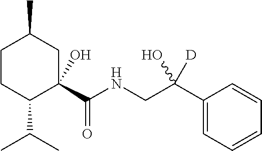 [SMILES: [2H]C(O)(CNC(=O)[C@]1(O)C[C@H](C)CC[C@H]1C(C)C)c1ccccc1]

### (1S,2S,5R)-1-hydroxy-N-((2RS)-2-hydroxy-2-phenylethyl-2-d)-2-isopropyl-5-methylcyclohexane-1-carboxamide (92)

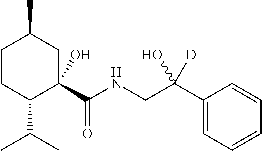 [SMILES: [2H]C(O)(CNC(=O)[C@]1(O)C[C@H](C)CC[C@H]1C(C)C)c1ccccc1]

Chiral separation of (1S,2S,5R)-1-hydroxy-N-(2-hydroxy-2-phenylethyl-2-d)-2-isopropyl-5-methylcyclohexane-1-carboxamide (245.1 mg) carried out by liquid chromatography using CHIRALPAK IA with heptane and ethanol led to (1S,2S,5R)-1-hydroxy-N-(2-hydroxy-2-phenylethyl-2-d)-2-isopropyl-5-methylcyclohexane-1-carboxamide (example 91, 134 mg) and (1S,2S,5R)-1-hydroxy-N-(2-hydroxy-2-phenylethyl-2-d)-2-isopropyl-5-methylcyclohexane-1-carboxamide (example 92, 117 mg).

### Example 93

### methyl 3-(2-(2-((1S,2S,5R)-1-hydroxy-2-isopropyl-5-methylcyclohexane-1-carboxamido) ethyl)phenyl)propanoate

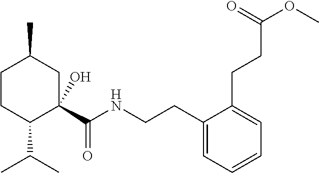 [SMILES: COC(=O)CCc1ccccc1CCNC(=O)[C@]1(O)C[C@H](C)CC[C@H]1C(C)C]

Methyl 2-(3-bromophenyl)acetate (1.01 g) and potassium tert-butyl N-[2-(trifluoroboranuidyl)ethyl]carbamate (1.1eq) as well as cesium carbonate (3.0eq) were dissolved in a 3/1 mixture of toluene and water (0.35M). The resulting solution was degassed using argon and 1,1′-bis(diphenylphosphino)ferrocene-palladium(II) dichloride dichloromethane complex (0.04eq) was added. The reaction mixture was then heated at 80° C. during 16 hrs. Once at room temperature, EtOAc (1V) was added and the resulting mixture was washed with water (1V×2). The organic phase was washed with an 1N aqueous solution of HCl (1V), with brine (1V), dried over Na2SO4, filtrated and concentrated under vacuum. The residue was purified by flash chromatography with cyclohexane and EtOAc to provide methyl 3-(2-(2-((tert-butoxycarbonyl)amino)ethyl)phenyl)propanoate (961 mg) as colorless oil.

Under deprotection conditions A, the tert-butyl N-carbamate (870 mg) in the presence of HCl (1M, 5.0eq) led to methyl 3-(2-(2-aminoethyl)phenyl)propanoate hydrochloride (640 mg).

Under coupling conditions B, (1S,2S,5R)-1-hydroxy-2-isopropyl-5-methylcyclohexane-1-carboxylic acid (170 mg) and methyl 3-(2-(2-aminoethyl)phenyl)propanoate hydrochloride (1.04eq) [after amine addition, the reaction was heated at 65° C. during 16 hr] led to methyl 3-(2-(2-((1S,2S,5R)-1-hydroxy-2-isopropyl-5-methylcyclohexane-1-carboxamido) ethyl)phenyl)propanoate (170 mg) as white solid after flash chromatography with cyclohexane and EtOAc.

### Example 94

### (1S,2S,5R)—N-((3S)-chroman-3-yl)-1-hydroxy-2-isopropyl-5-methylcyclohexane-1-carboxamide

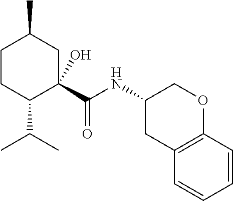 [SMILES: CC(C)[C@@H]1CC[C@@H](C)C[C@@]1(O)C(=O)N[C@@H]1COc2ccccc2C1]

Under coupling conditions B, (1S,2S,5R)-1-hydroxy-2-isopropyl-5-methylcyclohexane-1-carboxylic acid (120 mg) and (3S)-chroman-3-amine hydrochloride (1.03eq) [after amine addition, the reaction was heated at 65° C. during 16 hr] led to (1S,2S,5R)—N-((3S)-chroman-3-yl)-1-hydroxy-2-isopropyl-5-methylcyclohexane-1-carboxamide (65 mg) as white solid after flash chromatography with cyclohexane and EtOAc.

### Example 95

### (1S,2S,5R)—N-((3R)-chroman-3-yl)-1-hydroxy-2-isopropyl-5-methylcyclohexane-1-carboxamide

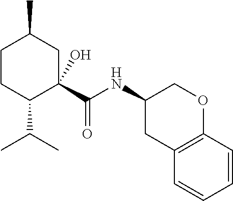 [SMILES: CC(C)[C@@H]1CC[C@@H](C)C[C@@]1(O)C(=O)N[C@H]1COc2ccccc2C1]

Under coupling conditions B, (1S,2S,5R)-1-hydroxy-2-isopropyl-5-methylcyclohexane-1-carboxylic acid (120 mg) and (3R)-chroman-3-amine hydrochloride (1.03eq) [after amine addition, the reaction was heated at 65° C. during 16 hr] led to (1S,2S,5R)—N-((3R)-chroman-3-yl)-1-hydroxy-2-isopropyl-5-methylcyclohexane-1-carboxamide (46 mg) as white solid after flash chromatography with cyclohexane and EtOAc.

### Example 96

### (1S,2S,5R)-1-hydroxy-N-((4-hydroxychroman-4-yl)methyl)-2-isopropyl-5-methylcyclohexane-1-carboxamide

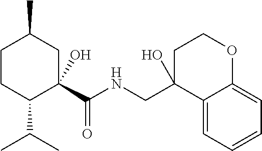 [SMILES: CC(C)[C@@H]1CC[C@@H](C)C[C@@]1(O)C(=O)NCC1(O)CCOc2ccccc21]

Under coupling conditions A, (1S,2S,5R)-1-hydroxy-2-isopropyl-5-methylcyclohexane-1-carboxylic acid (150 mg) and (3R)-chroman-3-amine hydrochloride (1.04eq) [after amine addition, the reaction was heated at 60° C. during 16 hr] led to (1S,2S,5R)-1-hydroxy-N-((4-hydroxychroman-4-yl)methyl)-2-isopropyl-5-methylcyclohexane-1-carboxamide (210 mg) as white solid after flash chromatography with cyclohexane and EtOAc.

### Examples 97 and 98

### (1S,2S,5R)-1-hydroxy-N-((1RS)-isochroman-1-ylmethyl)-2-isopropyl-5-methylcyclohexane-1-carboxamide (97)

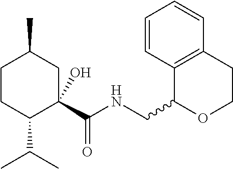 [SMILES: CC(C)[C@@H]1CC[C@@H](C)C[C@@]1(O)C(=O)NCC1OCCc2ccccc21]

### (1S,2S,5R)-1-hydroxy-N-((1RS)-isochroman-1-ylmethyl)-2-isopropyl-5-methylcyclohexane-1-carboxamide (98)

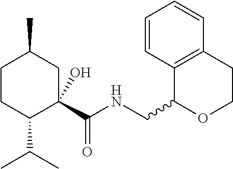 [SMILES: CC(C)[C@@H]1CC[C@@H](C)C[C@@]1(O)C(=O)NCC1OCCc2ccccc21]

Under coupling conditions B, (1S,2S,5R)-1-hydroxy-2-isopropyl-5-methylcyclohexane-1-carboxylic acid (150 mg) and (3,4-dihydro-1H-isochromen-1-ylmethyl)amine hydrochloride (1.04eq) [after amine addition, the reaction was heated at 60° C. during 16 hr] led to (1S,2S,5R)-1-hydroxy-N-((1RS)-isochroman-1-ylmethyl)-2-isopropyl-5-methylcyclohexane-1-carboxamide (example 97, 84 mg) and (1S,2S,5R)-1-hydroxy-N-((1RS)-isochroman-1-ylmethyl)-2-isopropyl-5-methylcyclohexane-1-carboxamide (example 98, 110 mg) as white solid after flash chromatography with cyclohexane and EtOAc.

### Example 99

### (1S,2S,5R)—N-(2-(2,4-dimethoxyphenyl)-2-oxoethyl)-1-hydroxy-2-isopropyl-5-methylcyclohexane-1-carboxamide

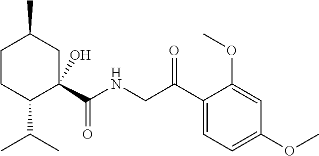 [SMILES: COc1ccc(C(=O)CNC(=O)[C@]2(O)C[C@H](C)CC[C@H]2C(C)C)c(OC)c1]

Under coupling conditions B, (1S,2S,5R)-1-hydroxy-2-isopropyl-5-methylcyclohexane-1-carboxylic acid (350 mg) and 2-(2,4-dimethoxyphenyl)-2-oxoethan-1-aminium chloride (1.04eq) [after amine addition, the reaction was heated at 60° C. during 16 hr] led to (1S,2S,5R)—N-(2-(2,4-dimethoxyphenyl)-2-oxoethyl)-1-hydroxy-2-isopropyl-5-methylcyclohexane-1-carboxamide (549 mg) as white solid after flash chromatography with cyclohexane and EtOAc.

### Example 100

### (1S,2S,5R)—N-(2-(3,4-dihydroxyphenyl)-2-oxoethyl)-1-hydroxy-2-isopropyl-5-methylcyclohexane-1-carboxamide

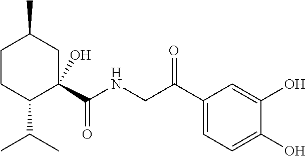 [SMILES: CC(C)[C@@H]1CC[C@@H](C)C[C@@]1(O)C(=O)NCC(=O)c1ccc(O)c(O)c1]

Under coupling conditions B, (1S,2S,5R)-1-hydroxy-2-isopropyl-5-methylcyclohexane-1-carboxylic acid (450 mg) and 2-(3,4-dimethoxyphenyl)-2-oxoethan-1-aminium chloride (1.04eq) [after amine addition, the reaction was heated at 65° C. during 16 hr] led to (1S,2S,5R)—N-(2-(3,4-dimethoxyphenyl)-2-oxoethyl)-1-hydroxy-2-isopropyl-5-methylcyclohexane-1-carboxamide (605 mg) as white solid after flash chromatography with cyclohexane and EtOAc. (1S,2S,5R)—N-(2-(3,4-Dimethoxyphenyl)-2-oxoethyl)-1-hydroxy-2-isopropyl-5-methylcyclohexane-1-carboxamide (445 mg) was dissolved in DCM under argon and cooled down to 0° C. BBr3 (3.31eq) was then added dropwise and the reaction was kept under stirring during 10 min. Once at room temperature, the reaction mixture was stirred during 6 hrs. Before addition of methanol (3 ml), the reaction mixture was cooled to 0° C. The resulting solution was concentrated under vacuum and the residue was dissolved in EtOAc (2V). Water (2V) was added and the aqueous phase was extracted with EtOAc (2V×2). The combined organic phases were washed with brine, dried over Na₂SO₄, filtrated and concentrated under vacuum. The residue was purified by preparative HPLC using Xselect CSH Prep C18 5 μm OBD 50×250m column with water and acetonitrile containing 0.1% formic acid to give (1S,2S,5R)—N-(2-(3,4-dihydroxyphenyl)-2-oxoethyl)-1-hydroxy-2-isopropyl-5-methylcyclohexane-1-carboxamide (194 mg) as pink-orange solid.

### Example 101

### (1S,2S,5R)-1-hydroxy-2-isopropyl-5-methyl-N-((3-(3-(((tetrahydro-2H-pyran-2-yl)oxy)methyl)phenyl)oxetan-3-yl)methyl)cyclohexane-1-carboxamide

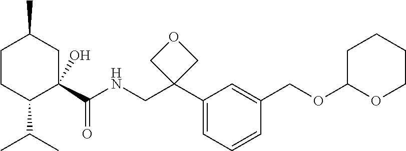 [SMILES: CC(C)[C@@H]1CC[C@@H](C)C[C@@]1(O)C(=O)NCC1(c2cccc(COC3CCCCO3)c2)COC1]

To chloro(1,5-cyclooctadiene)rhodium(I) dimer suspension (0.05eq) in 1,4-dioxane (21 ml) was added potassium hydroxide (1.5M, 1.3eq) at room temperature. Ethyl 2-(oxetan-3-ylidene)acetate (1.0 g) was added dropwise followed by portionwise addition of 3-(hydroxymethyl)phenylboronic acid (1.6eq). The reaction mixture was stirred at room temperature during 4 hrs. EtOAc (1V) was added and the resulting organic solution was washed with water (1V×2), brine (1V), dried over Na2SO4, filtrated and concentrated under vacuum. The residue was purified by flash chromatography with cyclohexane and EtOAc to isolate ethyl 2-(3-(3-(hydroxymethyl)phenyl)oxetan-3-yl)acetate (1.356 g) as pale-yellow oil.

Ethyl 2-(3-(3-(hydroxymethyl)phenyl)oxetan-3-yl)acetate (1.355 g) was dissolved in DCM and 3,4-dihydro-2H-pyran (3.14eq) was added as well as PPTS (0.20eq). The reaction mixture was sonicated and stirred at room temperature during 16 hr30. DCM (1V) was added and the organic phase was washed with water (1V), brine (1V), dried over Na₂SO₄, filtrated and concentrated under vacuum to yield ethyl 2-(3-(3-(((tetrahydro-2H-pyran-2-yl)oxy)methyl)phenyl)oxetan-3-yl)acetate (1.82 g) as pale yellow oil.

Ethyl 2-(3-(3-(((tetrahydro-2H-pyran-2-yl)oxy)methyl)phenyl)oxetan-3-yl)acetate (1.81 g) was dissolved in THF/Ethanol (0.15M) and NaOH (12N, 4.44eq) was added. The reaction mixture was stirred at room temperature during 17 hrs. Solvents were removed and water (5V) was added to the remaining solution: this mixture was extracted with diethyl ether (5V). The aqueous phase was acidified up to pH 1 with HCl 1N and was extracted with diethyl ether (5V×2). The combined organic phases were dried over Na₂SO₄, filtrated and concentrated under vacuum to give 2-(3-(3-(((tetrahydro-2H-pyran-2-yl)oxy)methyl)phenyl)oxetan-3-yl)acetic acid (1.57 g) as yellow wax.

2-(3-(3-(((Tetrahydro-2H-pyran-2-yl)oxy)methyl)phenyl)oxetan-3-yl)acetic acid (1.455 g) was dissolved in toluene and under argon, triethylamine (1.1eq) was added followed by addition of diphenylphosphoryl azide (1.05eq). The reaction mixture was then stirred at 80° C. during 25 min. Once at room temperature, 2-(trimethylsilyl)ethanol was added and after this addition the reaction was stirred at 80° C. during 4 hr45. Once at room temperature, EtOAc (5V) was added and the resulting mixture was washed with an 1N aqueous solution of citric acid (5V), a saturated aqueous solution of sodium bicarbonate (5V), brine (5V), dried over Na₂SO₄, filtrated and concentrated under vacuum. The residue was purified by flash chromatography with cyclohexane and EtOAc to yield 2-(trimethylsilyl)ethyl ((3-(3-(((tetrahydro-2H-pyran-2-yl)oxy)methyl)phenyl)oxetan-3-yl)methyl)carbamate (1.337 g) as colorless oil.

2-(Trimethylsilyl)ethyl ((3-(3-(((tetrahydro-2H-pyran-2-yl)oxy)methyl)phenyl)oxetan-3-yl)methyl)carbamate (404 mg) was dissolved in DMF (0.2M) and CsF (2.89eq) was added. The reaction mixture was stirred at 60° C. during 20 hrs. An 1N aqueous solution of sodium hydroxide (5V) was added and the resulting mixture was extracted with EtOAc (5V×2). The combined organic phases were washed with an 1N aqueous solution of sodium hydroxide (5V), brine (5V), dried over Na₂SO₄, filtrated and concentrated under vacuum to provide (3-(3-(((tetrahydro-2H-pyran-2-yl)oxy)methyl)phenyl)oxetan-3-yl)methanamine (265 mg) as yellow oil.

Under coupling conditions A, (1S,2S,5R)-1-hydroxy-2-isopropyl-5-methylcyclohexane-1-carboxylic acid (170 mg) and (3-(3-(((tetrahydro-2H-pyran-2-yl)oxy)methyl)phenyl)oxetan-3-yl)methanamine (1.12eq) [after amine addition, the reaction was heated at 60° C. during 16 hr] led to (1S,2S,5R)-1-hydroxy-2-isopropyl-5-methyl-N-((3-(3-(((tetrahydro-2H-pyran-2-yl)oxy)methyl)phenyl)oxetan-3-yl)methyl)cyclohexane-1-carboxamide (289 mg) as white solid after flash chromatography with cyclohexane and EtOAc.

### Example 102

### (1S,2S,5R)—N-(2-(2,4-dihydroxyphenyl)-2-oxoethyl)-1-hydroxy-2-isopropyl-5-methylcyclohexane-1-carboxamide

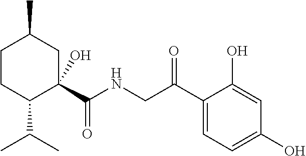 [SMILES: CC(C)[C@@H]1CC[C@@H](C)C[C@@]1(O)C(=O)NCC(=O)c1ccc(O)cc1O]

(1S,2S,5R)—N-(2-(2,4-dimethoxyphenyl)-2-oxoethyl)-1-hydroxy-2-isopropyl-5-methylcyclohexane-1-carboxamide (470 mg) was dissolved in DCM (0.1M) under argon and cooled down to 0° C. BBr₃ (6.6eq) was then added dropwise and the reaction was kept under stirring during 10 min. Once at room temperature, the reaction mixture was stirred during 24 hrs. The reaction was then stirred during 16 hrs at reflux. Before addition of methanol (5 ml), the reaction mixture was cooled to 0° C. The resulting solution was concentrated under vacuum and the residue was dissolved in EtOAc (2V). Water (2V) was added and the aqueous phase was extracted with EtOAc (2V×2). The combined organic phases were washed with brine, dried over Na₂SO₄, filtrated and concentrated under vacuum. The residue was purified by preparative HPLC using Xselect CSH Prep C18 5 μm OBD 50×250m column with water and acetonitrile containing 0.1% formic acid to give (1S,2S,5R)—N-(2-(2,4-dihydroxyphenyl)-2-oxoethyl)-1-hydroxy-2-isopropyl-5-methylcyclohexane-1-carboxamide (7.5 mg) as ecru solid.

### Example 103

### (1S,2S,5R)—N-(chroman-4-ylmethyl)-1-hydroxy-2-isopropyl-5-methylcyclohexane-1-carboxamide

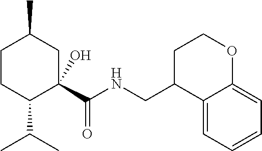 [SMILES: CC(C)[C@@H]1CC[C@@H](C)C[C@@]1(O)C(=O)NCC1CCOc2ccccc21]

Under coupling conditions A, (1S,2S,5R)-1-hydroxy-2-isopropyl-5-methylcyclohexane-1-carboxylic acid (134 mg) and 3,4-dihydro-2H-1-benzopyran-4-ylmethanamine (1.04eq) [after amine addition, the reaction was heated at 60° C. during 16 hr] led to (1S,2S,5R)—N-(chroman-4-ylmethyl)-1-hydroxy-2-isopropyl-5-methylcyclohexane-1-carboxamide (148 mg) as white solid after flash chromatography with cyclohexane and EtOAc.

### Example 104

### (1S,2S,5R)—N-(2-(2,3-dihydroxyphenyl)-2-oxoethyl)-1-hydroxy-2-isopropyl-5-methylcyclohexane-1-carboxamide

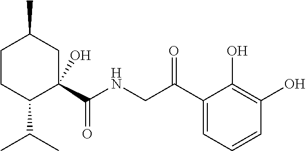 [SMILES: CC(C)[C@@H]1CC[C@@H](C)C[C@@]1(O)C(=O)NCC(=O)c1cccc(O)c1O]

Under coupling conditions A, (1S,2S,5R)-1-hydroxy-2-isopropyl-5-methylcyclohexane-1-carboxylic acid (1.49eq) and 2-amino-1-(2,3-dimethoxyphenyl)ethane-1-ol (322 mg) [after amine addition, the reaction was heated at 60° C. during 21 hr] led to (1S,2S,5R)—N-(2-(2,3-dimethoxyphenyl)-2-hydroxyethyl)-1-hydroxy-2-isopropyl-5-methylcyclohexane-1-carboxamide (122 mg) as white solid after flash chromatography with cyclohexane and EtOAc.

(1S,2S,5R)—N-(2-(2,3-Dimethoxyphenyl)-2-hydroxyethyl)-1-hydroxy-2-isopropyl-5-methylcyclohexane-1-carboxamide (121 mg) was dissolved in DCM (0.15M) and once cooled down to 0° C., the Dess-Martin reagent (1.7eq) was added portionwise. The reaction mixture was stirred at room temperature during 17 hrs. DCM (10V) was added and the resulting organic solution was washed with an 20% aqueous of Na₂S₂O₃ (10V), an 1N aqueous solution of sodium hydroxide (10V), brine (10V), dried over Na₂SO₄, filtrated and concentrated under vacuum. The residue was purified by preparative HPLC using Xselect CSH Prep C18 5 μm OBD 50×250m column with water and acetonitrile containing 0.1% formic acid to give (1S,2S,5R)—N-(2-(2,3-dimethoxyphenyl)-2-oxoethyl)-1-hydroxy-2-isopropyl-5-methylcyclohexane-1-carboxamide (111 mg) as white solid.

(1S,2S,5R)—N-(2-(2,3-Dimethoxyphenyl)-2-oxoethyl)-1-hydroxy-2-isopropyl-5-methylcyclohexane-1-carboxamide (110 mg) was dissolved in DCM (0.1M) under argon and cooled down to 0° C. BBr3 (3.3eq) was then added dropwise and the reaction was kept under stirring during 10 min. Once at room temperature, the reaction mixture was stirred during 4 hrs. Before addition of methanol (2 ml), the reaction mixture was cooled to 0° C. The resulting solution was concentrated under vacuum and the residue was dissolved in EtOAc (2V). Water (2V) was added and the aqueous phase was extracted with EtOAc (2V×2). The combined organic phases were washed with brine, dried over Na₂SO₄, filtrated and concentrated under vacuum. The residue was purified by preparative HPLC using Xselect CSH Prep C18 5 μm OBD 50×250m column with water and acetonitrile containing 0.1% formic acid to give (1S,2S,5R)—N-(2-(2,3-dihydroxyphenyl)-2-oxoethyl)-1-hydroxy-2-isopropyl-5-methylcyclohexane-1-carboxamide (49 mg) as white solid.

### Example 105

### (S)-2-hydroxypropyl 3-(2-((1S,2S,5R)-1-hydroxy-2-isopropyl-5-methylcyclohexane-1-carboxamido)ethyl)benzoate

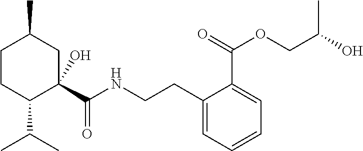 [SMILES: CC(C)[C@@H]1CC[C@@H](C)C[C@@]1(O)C(=O)NCCc1ccccc1C(=O)OC[C@H](C)O]

Under coupling conditions E, with 3-(2-((1S,2S,5R)-1-hydroxy-2-isopropyl-5-methylcyclohexane-1-carboxamido)ethyl)benzoic acid (150 mg) and (S)-2-((tert-butyldimethylsilyl)oxy) propan-1-ol (1.2eq) led to (S)-2-((tert-butyldimethylsilyl)oxy)propyl 3-(2-((1S,2S,5R)-1-hydroxy-2-isopropyl-5-methylcyclohexane-1-carboxamido)ethyl)benzoate (177 mg) as colorless wax after flash chromatography with cyclohexane and EtOAc.

(S)-2-((tert-Butyldimethylsilyl)oxy)propyl 3-(2-((1S,2S,5R)-1-hydroxy-2-isopropyl-5-methylcyclohexane-1-carboxamido)ethyl)benzoate (166 mg) was dissolved in methanol (0.1M) and HCl (1N, 10.33eq) was added followed by addition of THF (0.6V). The reaction mixture was stirred at room temperature during 3 hr. Once the solvent was removed, the remaining aqueous solution was extracted with EtOAc (5V×2). The combined organic phases were washed with brine (5V), dried over Na₂SO₄, filtrated and concentrated under vacuum. The residue was purified by flash chromatography with cyclohexane and EtOAc to yield (S)-2-hydroxypropyl 3-(2-((1S,2S,5R)-1-hydroxy-2-isopropyl-5-methylcyclohexane-1-carboxamido)ethyl)benzoate (94 mg) as white solid.

### Example 106

### methyl 2-(2-((1S,2S,5R)-1-hydroxy-2-isopropyl-5-methylcyclohexane-1-carboxamido)ethyl)benzoate

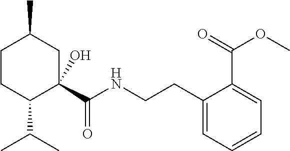 [SMILES: COC(=O)c1ccccc1CCNC(=O)[C@]1(O)C[C@H](C)CC[C@H]1C(C)C]

Under coupling conditions B, (1S,2S,5R)-1-hydroxy-2-isopropyl-5-methylcyclohexane-1-carboxylic acid (507 mg) and methyl 2-(2-aminoethyl)benzoate hydrochloride (478 mg) [after amine addition, the reaction was heated at 50° C. during 16 hr] led to methyl 2-(2-((1S,2S,5R)-1-hydroxy-2-isopropyl-5-methylcyclohexane-1-carboxamido)ethyl)benzoate (358 mg) as white powder after flash chromatography with cyclohexane and EtOAc.

### Example 107

### methyl 3-(2-((1S,2S,5R)-1-hydroxy-2-isopropyl-5-methylcyclohexane-1-carboxamido)ethyl)benzoate

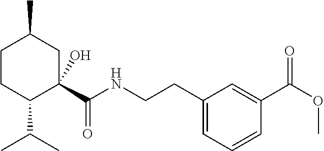 [SMILES: COC(=O)c1cccc(CCNC(=O)[C@]2(O)C[C@H](C)CC[C@H]2C(C)C)c1]

Under coupling conditions B, (1S,2S,5R)-1-hydroxy-2-isopropyl-5-methylcyclohexane-1-carboxylic acid (404 mg) and methyl 3-(2-aminoethyl)benzoate hydrochloride (478 mg) [after amine addition, the reaction was heated at 50° C. during 16 hr] led to methyl 3-(2-((1S,2S,5R)-1-hydroxy-2-isopropyl-5-methylcyclohexane-1-carboxamido)ethyl)benzoate (399 mg) as white powder after flash chromatography with cyclohexane and EtOAc.

### Example 108

### methyl 4-(2-((1S,2S,5R)-1-hydroxy-2-isopropyl-5-methylcyclohexane-1-carboxamido) ethyl)benzoate

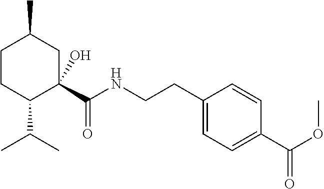 [SMILES: COC(=O)c1ccc(CCNC(=O)[C@]2(O)C[C@H](C)CC[C@H]2C(C)C)cc1]

Under coupling conditions B, (1S,2S,5R)-1-hydroxy-2-isopropyl-5-methylcyclohexane-1-carboxylic acid (408 mg) and methyl 4-(2-aminoethyl)benzoate hydrochloride (1.04eq) [after amine addition, the reaction was heated at 50° C. during 6 hr followed by 72 hrs at room temperature] led to methyl 4-(2-((1S,2S,5R)-1-hydroxy-2-isopropyl-5-methylcyclohexane-1-carboxamido) ethyl)benzoate (389 mg) as white solid after flash chromatography with cyclohexane and EtOAc.

### Example 109

### (1S,2S,5R)-1-hydroxy-N-(4-(hydroxymethyl)phenethyl)-2-isopropyl-5-methylcyclohexane-1-carboxamide

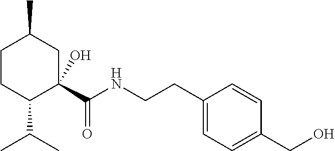 [SMILES: CC(C)[C@@H]1CC[C@@H](C)C[C@@]1(O)C(=O)NCCc1ccc(CO)cc1]

Under reduction condition A, methyl 4-(2-((1S,2S,5R)-1-hydroxy-2-isopropyl-5-methylcyclohexane-1-carboxamido) ethyl)benzoate (173 mg) in the presence of NaBH4 (41.0eq) led to (1S,2S,5R)-1-hydroxy-N-(4-(hydroxymethyl)phenethyl)-2-isopropyl-5-methylcyclohexane-1-carboxamide (108 mg) after flash chromatography with cyclohexane and EtOAc.

### Example 110

### (1S,2S,5R)-1-hydroxy-N-(2-hydroxyethyl)-2-isopropyl-5-methylcyclohexane-1-carboxamide

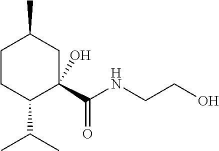 [SMILES: CC(C)[C@@H]1CC[C@@H](C)C[C@@]1(O)C(=O)NCCO]

Under coupling conditions A, (1S,2S,5R)-1-hydroxy-2-isopropyl-5-methylcyclohexane-1-carboxylic acid (125 mg) and ethanolamine (1.05eq) [after amine addition, the reaction was heated at 60° C. during 5 hr] led to (1S,2S,5R)-1-hydroxy-N-(2-hydroxyethyl)-2-isopropyl-5-methylcyclohexane-1-carboxamide (87 mg) as white solid after flash chromatography with cyclohexane and EtOAc.

### Example 111

### 2-((1S,2S,5R)-1-hydroxy-2-isopropyl-5-methylcyclohexane-1-carboxamido)ethyl benzoate

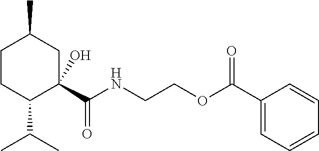 [SMILES: CC(C)[C@@H]1CC[C@@H](C)C[C@@]1(O)C(=O)NCCOC(=O)c1ccccc1]

Under coupling conditions B, benzoic acid (700 mg) and tert-butyl N-(2-hydroxyethyl)carbamate (1.1eq) [acetonitrile was replaced by DCM & after amine addition, the reaction was heated at 60° C. during 2 hr] led to 2-((tert-butoxycarbonyl)amino)ethyl benzoate (1.073 g) as white solid.

Under deprotection conditions A, 2-((tert-butoxycarbonyl)amino)ethyl benzoate (1.073 g) in the presence of HCl (2M, 10eq) led to 2-aminoethyl benzoate hydrochloride (771 mg).

Under coupling conditions B, (1S,2S,5R)-1-hydroxy-2-isopropyl-5-methylcyclohexane-1-carboxylic acid (125 mg) and 2-aminoethyl benzoate hydrochloride (1.1eq) [after amine addition, the reaction mixture was heated at 55° C. during 66 hr] led to 2-((1S,2S,5R)-1-hydroxy-2-isopropyl-5-methylcyclohexane-1-carboxamido)ethyl benzoate (93 mg) as white solid after flash chromatography with cyclohexane and EtOAc.

### Example 112

### (1S,2S,5R)-1-hydroxy-2-isopropyl-5-methyl-N-(2-oxotetrahydrofuran-3-yl)cyclohexane-1-carboxamide

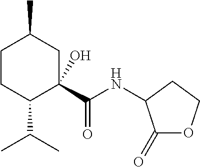 [SMILES: CC(C)[C@@H]1CC[C@@H](C)C[C@@]1(O)C(=O)NC1CCOC1=O]

Under coupling conditions B, (1S,2S,5R)-1-hydroxy-2-isopropyl-5-methylcyclohexane-1-carboxylic acid (100 mg) and alpha-amino-gamma-butyrolactone hydrobromide (1.1eq) [after amine addition, the reaction was heated at 55° C. during 16 hr] led to (1S,2S,5R)-1-hydroxy-2-isopropyl-5-methyl-N-(2-oxotetrahydrofuran-3-yl)cyclohexane-1-carboxamide (54 mg) as white solid after flash chromatography with cyclohexane and EtOAc.

Examples 1 to 112 were characterized by 1H NMR and LC-MS analysis as shown below in Table 1.

TABLE 1

| | LCMS | | |
|---|---|---|---|
| Example | Method | [M + H]⁺ | 1H NMR spectrum (δ ppm, DMSO-d₆) |
| 1 | A | 320 | (400 MHz, DMSO- d₆) 0.67-0.93 (m, 10 H), 1.32-1.43 (m, 4 |
| | | | H), 1.47-1.52 (m, 2 H), 1.67 (br d, J = 13 Hz, 2 H), 3.14-3.20 |
| | | | (m, 1 H), 3.34-3.47 (m, 1 H), 4.63 (br s, 1 H), 4.85 (s, 1 H), |
| | | | 5.50 (br d, J = 3 Hz, 1 H), 7.22-7.36 (m, 5 H), 7.61 (br t, J = 6 Hz, |
| | | | 1 H) |
| 2 | A | 318 | (400 MHz, DMSO- d₆) 0.74-0.93 (m, 10 H), 1.35-1.48 (m, 3 H), |
| | | | 1.48-1.58 (m, 2 H), 1.63-1.80 (m, 3 H), 4.63 (d, J = 5 Hz, 2 H), |
| | | | 4.98 (s, 1 H), 7.54 (t, J = 7 Hz, 2 H), 7.66 (t, J = 6 Hz, 1 H), 7.99 (d, |
| | | | J = 7 Hz, 2 H), 8.06 (br t, J = 5 Hz, 1 H) |
| 3 | A | 362 | (400 MHz, DMSO- d₆) 0.71-0.89 (m, 10 H), 1.31-1.53 (m, 6 H), |
| | | | 1.62-1.76 (m, 2 H), 2.04 (s, 3 H), 3.33-3.47 (m, 1 H), 3.55 (br |
| | | | dd, J = 6, 5 Hz, 1 H), 4.83 (s, 1 H), 5.82 (dd, J = 8, 4 Hz, 1 H), 7.29- |
| | | | 7.41 (m, 5 H), 7.76 (br t, J = 6 Hz, 1 H) |
| 4 | A | 340 | (400 MHz, DMSO- d₆) 0.60-0.95 (m, 10 H), 1.21-1.52 (m, 6 H), |
| | | | 1.61-1.79 (m, 2 H), 3.79-4.04 (m, 2 H), 4.92 (s, 1 H), 7.48- |
| | | | 7.55 (m, 5 H), 7.75 (br t, J = 6 Hz, 1 H) |
| 5 | A | 322 | (400 MHz, DMSO- d₆) 0.71-0.90 (m, 10 H), 1.31-1.54 (m, 6 H), |
| | | | 1.64-1.75 (m, 2 H), 3.33-3.68 (m, 2 H), 4.86 (s, 1 H), 5.53- |
| | | | 5.69 (m, 1 H) ,7.35-7.43 (m, 5 H), 7.85 (br t, J = 6 Hz, 1 H) |
| 6 | A | 363 | (400 MHz, DMSO- d₆) 0.72-0.91 (m, 10 H), 1.31-1.52 (m, 6 H), |
| | | | 1.63-1.73 (m, 2 H), 2.66-2.81 (m, 2 H), 2.92 (t, J = 5 Hz, 2 H), |
| | | | 3.34-3.41 (m, 2 H), 3.93 (t, J = 5 Hz, 2 H), 4.75-4.86 (m, 1 H), |
| | | | 6.84 (td, J = 7, 1 Hz, 1 H), 6.92 (d, J = 8 Hz, 1 H), 7.11 (dd, J = 7, 2 |
| | | | Hz, 1 H), 7.16 (t, J = 7 Hz, 1 H), 7.70 (br t, J = 6 Hz, 1 H) |
| 7 | A | 364 | (400 MHz, DMSO- d₆) 0.71-0.91 (m, 10 H), 1.32-1.54 (m, 6 H), |
| | | | 1.68 (br d, J = 12 Hz, 2 H), 2.68-2.81 (m, 2 H), 3.32-3.43 (m, 2 |
| | | | H), 3.76 (q, J = 5 Hz, 2 H), 4.00 (t, J = 5 Hz, 2 H), 4.75 (s, 1 H), 4.89 |
| | | | (t, J = 6 Hz, 1 H), 6.85 (t, J = 7 Hz, 1 H), 6.94 (d, J = 8 Hz, 1 H), 7.11 |
| | | | (dd, J = 7, 1 Hz, 1 H), 7.17 (t, J = 8 Hz, 1 H), 7.69 (br t, J = 6 Hz, 1 H) |
| 8 | A | 344 | (400 MHz, DMSO- d₆) 0.66 (t, J = 7 Hz, 6 H), 0.74-0.92 (m, 4 H), |
| | | | 1.23-1.49 (m, 6 H), 1.65 (br d, J = 10 Hz, 2 H), 3.01 (t, J = 7 Hz, 2 |
| | | | H), 3.46-3.67 (m, 3 H), 4.78 (s, 1 H), 7.12-7.16 (m, 2 H), 7.46- |
| | | | 7.51 (m, 2 H), 7.96 (t, J = 6 Hz, 1 H) |
| 9 | A | 394 | (500 MHz, DMSO- d₆) 0.74-0.91 (m, 10 H), 1.33-1.54 (m, 6 H), |
| | | | 1.64-1.74 (m, 2 H), 2.67-2.81 (m, 2 H), 3.32-3.45 (m, 2 H), |
| | | | 3.47-3.56 (m, 2 H), 3.79-3.94 (m, 2 H), 3.95-4.07 (m, 1 H), |
| | | | 4.63 (t, J = 5 Hz, 1 H), 4.75 (s, 1 H), 4.96 (t, J = 5 Hz, 1 H), 6.85 (td, |
| | | | J = 7, 1 Hz, 1 H), 6.93 (d, J = 8 Hz, 1 H), 7.11 (dd, J = 7, 2 Hz, 1 H), |
| | | | 7.17 (t, J = 8 Hz, 1 H), 7.65-7.75 (m, 1 H) |
| 10 | A | 394 | (500 MHz, DMSO- d₆) 0.74-0.91 (m, 10 H), 1.33-1.54 (m, 6 H), |
| | | | 1.62-1.75 (m, 2 H), 2.66-2.76 (m, 2 H), 3.31-3.51 (m, 4 H), |
| | | | 3.76-3.87 (m, 2 H), 3.97 (dd, J = 10, 4 Hz, 1 H), 4.62 (t, J = 6 Hz, 1 |
| | | | H), 4.74 (s, 1 H), 4.89 (d, J = 5 Hz, 1 H), 6.75-6.80 (m, 3 H), 7.18 |
| | | | (t, J = 8 Hz, 1 H), 7.67 (br t, J = 6 Hz, 1 H) |
| 11 | A | 406 | (400 MHz, DMSO- d₆) 0.70-0.89 (m, 10 H), 1.30-1.55 (m, 6 H), |
| | | | 1.68 (br d, J = 10 Hz, 2 H), 2.60-2.78 (m, 2 H), 3.22 (s, 11 H), |
| | | | 3.86 (br s, 2 H), 4.46 (br s, 2 H), 4.81 (br s, 1 H), 6.92 (t, J = 7 Hz, |
| | | | 1 H), 7.01 (d, J = 8 Hz, 1 H), 7.12-7.27 (m, 2 H), 7.79 (br t, J = 6 |
| | | | Hz, 1 H) |
| 12 | A | 334 | (400 MHz, DMSO- d₆) 0.69-0.88 (m, 10 H), 1.29-1.54 (m, 6 H), |
| | | | 1.67 (br d, J = 12 Hz, 2 H), 2.29 (s, 3 H), 3.08-3.20 (m, 1 H), 3.33- |
| | | | 3.47 (m, 1 H), 4.59 (br s, 1 H), 4.85 (br s, 1 H), 5.47 (br d, J = 10 |
| | | | Hz, 1 H), 7.05 (d, J = 7 Hz, 1 H), 7.10-7.22 (m, 3 H), 7.51-7.63 |
| | | | (m, 1 H) |
| 13 | B | 391 | (400 MHz, DMSO- d₆) 0.69-0.87 (m, 10 H), 1.23-1.53 (m, 6 H), |
| | | | 1.67 (br d, J = 10 Hz, 2 H), 2.98-3.19 (m, 2 H), 3.20-3.29 (m, 1 |
| | | | H), 3.35-3.54 (m, 2 H), 3.63-3.77 (m, 1 H), 4.46 (t, J = 5 Hz, 2 |
| | | | H), 4.74 (br s, 1 H), 7.32-7.38 (m, 2 H), 7.47-7.55 (m, 1 H), |
| | | | 7.77 (br t, J = 6 Hz, 1 H), 8.00 (d, J = 8 Hz, 1 H), 8.11 (br s, 3 H) |
| 14 | A | 334 | (400 MHz, DMSO- d₆) 0.73-0.92 (m, 10 H), 1.31-1.54 (m, 6 H), |
| | | | 1.67 (br d, J = 10 Hz, 2 H), 2.66-2.81 (m, 2 H), 3.27-3.31 (m, 2 |
| | | | H), 4.56 (d, J = 4 Hz, 2 H), 4.73 (s, 1 H), 5.08 (br s, 1 H), 7.13- |
| | | | 7.21 (m, 3 H), 7.34-7.39 (m, 1 H), 7.79 (br t, J = 6 Hz, 1 H) |
| 15 | A | 334 | (400 MHz, DMSO- d₆) 0.72-0.91 (m, 10 H), 1.32-1.54 (m, 6 H), |
| | | | 1.63-1.74 (m, 2 H), 2.67-2.76 (m, 2 H), 3.33-3.42 (m, 2 H), |
| | | | 4.45-4.49 (m, 2 H), 4.73 (br s, 1 H), 5.11 (br s, 1 H), 7.06 (d, J = 7 |
| | | | Hz, 1 H), 7.13-7.17 (m, 2 H), 7.19-7.26 (m, 1 H), 7.70 (br t, J = 6 |
| | | | Hz, 1 H) |
| 16 | A | 391 | (400 MHz, DMSO- d₆) 0.69-0.89 (m, 10 H), 1.28-1.51 (m, 6 H), |
| | | | 1.66 (br d, J = 10 Hz, 2 H), 2.80-2.90 (m, 2 H), 3.20-3.29 (m, 2 |
| | | | H), 3.33-3.39 (m, 2 H), 4.42-4.46 (m, 2 H), 7.43-7.48 (m, 1 H), |
| | | | 7.53 (d, J = 7 Hz, 1 H), 7.76 (t, J = 6 Hz, 1 H), 7.92-7.95 (m, 2 H), |
| | | | 8.16 (br s, 3 H) |
| 17 | A | 346 | (400 MHz, DMSO- d₆) 0.53 (d, J = 7 Hz, 3 H), 0.58 (br d, J = 7 Hz, |
| | | | 3 H), 0.68-0.87 (m, 5 H), 1.21-1.41 (m, 5 H), 1.63 (br d, J = 12 |
| | | | Hz, 2 H), 3.65 (dt, J = 14, 4 Hz, 1 H), 3.87-3.96 (m, 1 H), 4.82 (s, |
| | | | 1 H), 5.74 (t, J = 4 Hz, 1 H), 7.56-7.83 (m, 5 H) |
| 18 | A | 346 | (500 MHz, DMSO- d₆) 0.68-0.85 (m, 10 H), 1.09-1.21 (m, 2 H), |
| | | | 1.26-1.51 (m, 4 H), 1.63 (br d, J = 10 Hz, 2 H), 3.69-3.79 (m, 2 |
| | | | H), 4.77 (s, 1 H), 5.73 (t, J = 4 Hz, 1 H), 7.60 (t, J = 8 Hz, 1 H), 7.63- |
| | | | 7.85 (m, 4 H) |
| 19 | A | 405 | (400 MHz, DMSO- d₆) 0.71-0.88 (m, 10 H), 1.29-1.55 (m, 6 H), |
| | | | 1.68 (br d, J = 12 Hz, 2 H), 2.67-2.83 (m, 2 H), 3.08-3.17 (m, 1 |
| | | | H), 3.19-3.35 (m, 3 H), 3.61 (s, 3 H), 4.20 (br s, 1 H), 7.14-7.24 |
| | | | (m, 4 H), 7.81 (t, J = 6 Hz, 1 H), 8.62 (br s, 3 H) |
| 20 | B | 405 | (400 MHz, DMSO- d₆) 0.72-0.88 (m, 10 H), 1.28-1.54 (m, 6 H), |
| | | | 1.67 (br d, J = 10 Hz, 2 H), 2.60-2.77 (m, 2 H), 3.04-3.27 (m, 2 |
| | | | H), 3.36-3.48 (m, 2 H), 3.70 (s, 3 H), 4.29 (br s, 1 H), 4.62-4.93 |
| | | | (m, 1 H), 7.03-7.15 (m, 3 H), 7.25 (t, J = 8 Hz, 1 H), 7.78 (br t, |
| | | | J = 6 Hz, 1 H), 8.49 (br s, 3 H) |
| 21 | A | 391 | (400 MHz, DMSO- d₆) 0.68-0.90 (m, 10 H), 1.27-1.51 (m, 6 H), |
| | | | 1.67 (br d, J = 13 Hz, 2 H), 2.78-2.90 (m, 2 H), 3.32-3.51 (m, 2 |
| | | | H), 3.68-3.74 (m, 2 H), 4.25-4.30 (m, 2 H), 4.71 (s, 1 H), 4.91 |
| | | | (t, J = 6 Hz, 1 H), 7.42-7.53 (m, 2 H), 7.73 (br t, J = 6 Hz, 1 H), |
| | | | 7.82-7.86 (m, 2 H) |
| 22 | A | 392 | (400 MHz, DMSO- d₆) 0.69-0.91 (m, 10 H), 1.27-1.51 (m, 6 H), |
| | | | 1.67 (br d, J = 12 Hz, 2 H), 3.00-3.16 (m, 2 H), 3.31-3.48 (m, 2 |
| | | | H), 3.70-3.75 (m, 2 H), 4.26-4.32 (m, 2 H), 4.71 (s, 1 H), 4.92 |
| | | | (t, J = 6 Hz, 1 H), 7.30-7.38 (m, 2 H), 7.50 (t, J = 8 Hz, 1 H), 7.73 |
| | | | (br t, J = 6 Hz, 1 H), 7.85 (d, J = 7 Hz, 1 H) |
| 23 | B | 344 | (400 MHz, DMSO- d₆) 0.71-0.88 (m, 10 H), 1.32-1.56 (m, 6 H), |
| | | | 1.69 (br d, J = 12 Hz, 2 H), 1.91-2.00 (m, 1 H), 2.16 (td, J = 9, 4 |
| | | | Hz, 1 H), 2.70-2.79 (m, 1 H), 2.87 (br d, J = 9 Hz, 1 H), 3.31- |
| | | | 3.43 (m, 2 H), 5.00 (d, J = 3 Hz, 1 H), 5.46 (d, J = 14 Hz, 1 H), 7.16- |
| | | | 7.24 (m, 3 H), 7.30-7.37 (m, 1 H), 7.60 (br t, J = 6 Hz, 1 H) |
| 24 | A | 394 | (400 MHz, DMSO- d₆) 0.63-0.97 (m, 10 H), 1.22-1.58 (m, 6 H), |
| | | | 1.67 (br d, J = 12 Hz, 2 H), 2.68-2.79 (m, 2 H), 3.23-3.28 (m, 1 |
| | | | H), 3.36-3.43 (m, 1 H), 3.50 (br t, J = 5 Hz, 2 H), 3.82-3.89 (m, 2 |
| | | | H), 3.99-4.02 (m, 1 H), 4.63 (t, J = 6 Hz, 1 H), 4.74 (s, 1 H), 4.94- |
| | | | 5.02 (m, 1 H), 6.84 (t, J = 7 Hz, 1 H), 6.92 (d, J = 8 Hz, 1 H), 7.10 |
| | | | (dd, J = 7, 2 Hz, 1 H), 7.14-7.19 (m, 1 H), 7.68 (br t, J = 6 Hz, 1 H) |
| 25 | A | 394 | (400 MHz, DMSO- d₆) 0.73-0.91 (m, 10 H), 1.34-1.53 (m, 6 H), |
| | | | 1.64-1.72 (m, 2 H), 2.66-2.78 (m, 2 H), 3.32-3.41 (m, 2 H), |
| | | | 3.50 (br t, J = 4 Hz, 2 H), 3.82-3.90 (m, 2 H), 3.98-4.02 (m, 1 H), |
| | | | 4.62 (br t, J = 6 Hz, 1 H), 4.74 (s, 1 H), 4.93-4.97 (m, 1 H), 6.84 |
| | | | (t, J = 7 Hz, 1 H), 6.92 (d, J = 8 Hz, 1 H), 7.10 (dd, J = 7, 2 Hz, 1 H), |
| | | | 7.16 (t, J = 8 Hz, 1 H), 7.68 (br t, J = 6 Hz, 1 H) |
| 26 | A | 421 | (400 MHz, DMSO- d₆) 0.70-0.88 (m, 10 H), 1.29-1.53 (m, 6 H), |
| | | | 1.67 (br d, J = 12 Hz, 2 H), 1.94 (br s, 2 H), 2.64-2.76 (m, 2 H), |
| | | | 3.33-3.44 (m, 2 H), 3.65 (s, 3 H), 3.72 (br s, 1 H), 4.02-4.12 (m, |
| | | | 2 H), 4.73 (s, 1 H), 6.72-6.80 (m, 3 H), 7.18 (t, J = 8 Hz, 1 H), |
| | | | 7.66 (br t, J = 6 Hz, 1 H) |
| 27 | A | 421 | (500 MHz, DMSO- d₆) 0.69-0.88 (m, 10 H), 1.32-1.44 (m, 4 H), |
| | | | 1.44-1.53 (m, 2 H), 1.58-1.79 (m, 2 H), 2.08 (s, 2 H), 2.64- |
| | | | 2.80 (m, 2 H), 3.27-3.34 (m, 1 H), 3.35-3.51 (m, 1 H), 3.66 (s, 3 |
| | | | H), 3.75 (t, J = 5 Hz, 1 H), 4.03-4.16 (m, 2 H), 4.73 (s, 1 H), 6.74- |
| | | | 6.83 (m, 3 H), 7.19 (t, J = 8 Hz, 1 H), 7.64-7.75 (m, 1 H) |
| 28 | A | 376 | (400 MHz, DMSO- d₆) 0.67-0.90 (m, 10 H), 1.24-1.49 (m, 9 H), |
| | | | 1.67 (br d, J = 13 Hz, 2 H), 2.78-2.89 (m, 2 H), 3.31-3.50 (m, 2 |
| | | | H), 4.31 (q, J = 7 Hz, 2 H), 4.70 (s, 1 H), 7.41-7.53 (m, 2 H), 7.70 |
| | | | (br t, J = 6 Hz, 1 H), 7.80 (dt, J = 4, 2 Hz, 2 H) |
| 29 | A | 460 | (400 MHz, DMSO- d₆) 0.65-0.90 (m, 10 H), 1.26-1.49 (m, 6 H), |
| | | | 1.66 (br d, J = 11 Hz, 2 H), 2.23 (s, 3 H), 2.79-2.90 (m, 2 H), 3.31- |
| | | | 3.37 (m, 1 H), 3.43-3.52 (m, 1 H), 4.69 (s, 1 H), 5.22 (s, 2 H), |
| | | | 7.45 (t, J = 8 Hz, 1 H), 7.54 (d, J = 8 Hz, 1 H), 7.70 (t, J = 6 Hz, 1 H), |
| | | | 7.80-7.86 (m, 2 H) |
| 30 | A | 347 | (400 MHz, DMSO- d₆) 0.72-0.90 (m, 10 H), 1.33-1.62 (m, 6 H), |
| | | | 1.68 (br d, J = 11 Hz, 2 H), 3.72-3.84 (m, 2 H), 4.31 (d, J = 6 Hz, 2 |
| | | | H), 4.91 (br s, 1 H), 7.20-7.35 (m, 5 H), 7.95 (br t, J = 5 Hz, 1 H), |
| | | | 8.25 (br t, J = 6 Hz, 1 H) |
| 31 | A | 346 | (400 MHz, DMSO- d₆) 0.71-0.91 (m, 10 H), 1.30-1.55 (m, 6 H), |
| | | | 1.67 (br d, J = 11 Hz, 2 H), 3.56 (dd, J = 13, 6 Hz, 1 H), 3.69 (dd, |
| | | | J = 13, 7 Hz, 1 H), 4.66-4.79 (m, 4 H), 4.85 (s, 1 H), 7.14 (d, J = 8 |
| | | | Hz, 2 H), 7.24-7.30 (m, 1 H), 7.33-7.40 (m, 2 H), 7.59 (br t, J = 6 |
| | | | Hz, 1 H) |
| 32 | B | 336 | (400 MHz, DMSO- d₆) 0.69-0.88 (m, 10 H), 1.30-1.56 (m, 6 H), |
| | | | 1.67 (br d, J = 11 Hz, 2 H), 3.03-3.48 (m, 2 H), 4.49-4.56 (m, 1 |
| | | | H), 4.89 (s, 1 H), 5.47 (dd, J = 10, 4 Hz, 1 H), 6.63 (dd, J = 8, 2 Hz, |
| | | | 1 H), 6.72-6.79 (m, 2 H), 7.10 (td, J = 8, 2 Hz, 1 H), 7.52-7.65 |
| | | | (m, 1 H), 9.29 (s, 1 H) |
| 33 | B | 408 | (400 MHz, DMSO- d₆) 0.69-0.88 (m, 10 H), 1.26-1.55 (m, 6 H), |
| | | | 1.67 (br d, J = 10 Hz, 2 H), 3.09-3.21 (m, 1 H), 3.37-3.51 (m, 1 |
| | | | H), 3.71 (s, 3 H), 4.58-4.64 (m, 1 H), 4.77 (d, J = 2 Hz, 2 H), 4.86 |
| | | | (s, 1 H), 5.54 (dd, J = 12, 4 Hz, 1 H), 6.80 (dd, J = 8, 2 Hz, 1 H), |
| | | | 6.89-7.01 (m, 2 H), 7.24 (t, J = 8 Hz, 1 H), 7.57 (s, 1 H) |
| 34 | A | 378 | (400 MHz, DMSO- d₆) 0.62-0.85 (m, 10 H), 1.20-1.49 (m, 6 H), |
| | | | 1.65 (br d, J = 11 Hz, 2 H), 3.18-3.28 (m, 1 H), 3.44 (br dd, J = 13, |
| | | | 7 Hz, 1 H), 3.85 (s, 3 H), 4.73-4.82 (m, 2 H), 5.63-5.72 (m, 1 |
| | | | H), 7.46 (t, J = 8 Hz, 1 H), 7.55-7.67 (m, 2 H), 7.84 (d, J = 7 Hz, 1 |
| | | | H), 7.95 (d, J = 2 Hz, 1 H) |
| 35 | A | 336 | (400 MHz, DMSO- d₆) 0.62-0.96 (m, 10 H), 1.29-1.59 (m, 6 H), |
| | | | 1.67 (br d, J = 11 Hz, 2 H), 3.00-3.24 (m, 1 H), 3.35-3.54 (m, 1 |
| | | | H), 4.86-4.94 (m, 2 H), 5.13-5.60 (m, 1 H), 6.74-6.79 (m, 2 H), |
| | | | 7.04 (t, J = 8 Hz, 1 H), 7.29-7.34 (m, 1 H), 7.58 (q, J = 5 Hz, 1 H), |
| | | | 9.13-9.56 (m, 1 H) |
| 36 | A | 408 | (500 MHz, DMSO- d₆) 0.59-0.91 (m, 10 H), 1.27-1.56 (m, 6 H), |
| | | | 1.63-1.73 (m, 2 H), 3.13-3.28 (m, 1 H), 3.42-3.56 (m, 1 H), |
| | | | 3.71 (d, J = 1 Hz, 3 H), 4.83 (d, J = 6 Hz, 2 H), 4.88 (d, J = 11 Hz, 1 |
| | | | H), 4.96-5.14 (m, 1 H), 5.38-5.47 (m, 1 H), 6.87 (d, J = 8 Hz, 1 |
| | | | H), 6.97 (t, J = 7 Hz, 1 H), 7.20 (t, J = 8 Hz, 1 H), 7.45 (ddd, J = 7, 6, |
| | | | 1 Hz, 1 H), 7.50-7.58 (m, 1 H) |
| 37 | A | 350 | (400 MHz, DMSO- d₆) 0.71-0.89 (m, 10 H), 1.27-1.58 (m, 6 H), |
| | | | 1.68 (br d, J = 10 Hz, 2 H), 3.08-3.54 (m, 2 H), 4.50 (d, J = 6 Hz, 2 |
| | | | H), 4.62 (br d, J = 5 Hz, 1 H), 4.86 (s, 1 H), 5.14 (t, J = 6 Hz, 1 H), |
| | | | 5.50 (dd, J = 8, 4 Hz, 1 H), 7.18-7.32 (m, 4 H), 7.51-7.74 (m, 1 |
| | | | H) |
| 38 | A | 334 | (400 MHz, DMSO- d₆) 0.75-0.93 (m, 10 H), 1.35-1.48 (m, 3 H), |
| | | | 1.48-1.58 (m, 2 H), 1.62-1.80 (m, 3 H), 4.62 (d, J = 6 Hz, 2 H), |
| | | | 4.99 (s, 1 H), 6.95 (t, J = 8 Hz, 1 H), 7.00 (dd, J = 8, 1 Hz, 1 H), 7.51 |
| | | | (ddd, J = 8, 7, 2 Hz, 1 H), 7.87 (dd, J = 8, 2 Hz, 1 H), 8.06 (t, J = 5 |
| | | | Hz, 1 H), 11.42 (s, 1 H) |
| 39 | A | 334 | (400 MHz, DMSO- d₆) 0.74-0.93 (m, 10 H), 1.35-1.48 (m, 3 H), |
| | | | 1.48-1.58 (m, 2 H), 1.62-1.80 (m, 3 H), 4.57 (d, J = 5 Hz, 2 H), |
| | | | 4.98 (s, 1 H), 7.04 (dd, J = 8, 2 Hz, 1 H), 7.28-7.37 (m, 2 H), 7.44 |
| | | | (d, J = 8 Hz, 1 H), 8.02 (t, J = 5 Hz, 1 H), 9.78 (s, 1 H) |
| 40 | A | 320 | (400 MHz, DMSO- d₆) 0.72-0.91 (m, 10 H), 1.30-1.54 (m, 6 H), |
| | | | 1.63-1.75 (m, 2 H), 2.58-2.69 (m, 2 H), 3.21-3.29 (m, 1 H), |
| | | | 3.32-3.39 (m, 1 H), 4.73 (s, 1 H), 6.57-6.63 (m, 3 H), 7.06 (t, |
| | | | J = 8 Hz, 1 H), 7.66 (br t, J = 6 Hz, 1 H), 9.21 (s, 1 H) |
| 41 | A | 336 | (400 MHz, DMSO- d₆) 0.67-0.95 (m, 10 H), 1.21-1.54 (m, 6 H), |
| | | | 1.61-1.74 (m, 2 H), 3.19-3.28 (m, 2 H), 4.53 (br s, 1 H), 4.87 (s, |
| | | | 1 H), 5.41-5.49 (m, 1 H), 6.62 (dd, J = 8, 2 Hz, 1 H), 6.69-6.85 |
| | | | (m, 2 H), 7.09 (t, J = 8 Hz, 1 H), 7.55 (br t, J = 5 Hz, 1 H), 9.28 (br s, |
| | | | 1 H) |
| 42 | A | 336 | (400 MHz, DMSO- d₆) 0.71-0.90 (m, 10 H), 1.28-1.59 (m, 6 H), |
| | | | 1.67 (br d, J = 12 Hz, 2 H), 3.05-3.15 (m, 1 H), 3.36-3.42 (m, 1 |
| | | | H), 4.49-4.55 (m, 1 H), 4.85 (s, 1 H), 5.42 (d, J = 4 Hz, 1 H), 6.62 |
| | | | (dd, J = 8, 2 Hz, 1 H), 6.71-6.80 (m, 2 H), 7.09 (t, J = 8 Hz, 1 H), |
| | | | 7.60 (t, J = 6 Hz, 1 H), 9.25 (s, 1 H) |
| 43 | A | 350 | (400 MHz, DMSO- d₆) 0.70-0.90 (m, 10 H), 1.31-1.52 (m, 6 H), |
| | | | 1.62-1.74 (m, 2 H), 3.21-3.38 (m, 2 H), 4.49 (d, J = 6 Hz, 2 H), |
| | | | 4.59-4.64 (m, 1 H), 4.86 (s, 1 H), 5.14 (t, J = 6 Hz, 1 H), 5.52 (d, |
| | | | J = 4 Hz, 1 H), 7.18-7.30 (m, 4 H), 7.58 (t, J = 6 Hz, 1 H) |
| 44 | A | 350 | (400 MHz, DMSO- d₆) 0.71-0.90 (m, 10 H), 1.29-1.59 (m, 6 H), |
| | | | 1.63-1.74 (m, 2 H), 3.09-3.25 (m, 1 H), 3.42-3.48 (m, 1 H), |
| | | | 4.49 (d, J = 6 Hz, 2 H), 4.58-4.65 (m, 1 H), 4.86 (s, 1 H), 5.14 (t, |
| | | | J = 6 Hz, 1 H), 5.49 (d, J = 4 Hz, 1 H), 7.17-7.31 (m, 4 H), 7.63 (br |
| | | | t, J = 6 Hz, 1 H) |
| 45 | A | 334 | (400 MHz, DMSO- d₆) 0.69-0.87 (m, 10 H), 1.28-1.52 (m, 6 H), |
| | | | 1.59-1.74 (m, 2 H), 2.29 (s, 3 H), 3.30-3.33 (m, 2 H), 4.56- |
| | | | 4.61 (m, 1 H), 4.85 (s, 1 H), 5.49 (d, J = 4 Hz, 1 H), 7.05 (d, J = 8 |
| | | | Hz, 1 H), 7.11-7.22 (m, 3 H), 7.54 (br t, J = 6 Hz, 1 H) |
| 46 | A | 334 | (400 MHz, DMSO- d₆) 0.71-0.88 (m, 10 H), 1.27-1.56 (m, 6 H), |
| | | | 1.60-1.74 (m, 2 H), 2.29 (s, 3 H), 3.11-3.25 (m, 1 H), 3.39- |
| | | | 3.48 (m, 1 H), 4.57-4.62 (m, 1 H), 4.86 (s, 1 H), 5.45 (d, J = 4 Hz, |
| | | | 1 H), 7.05 (d, J = 7 Hz, 1 H), 7.11-7.22 (m, 3 H), 7.60 (br t, J = 6 |
| | | | Hz, 1 H) |
| 47 | A | 334 | (400 MHz, DMSO- d₆) 0.67-0.95 (m, 10 H), 1.18-1.54 (m, 6 H), |
| | | | 1.63-1.70 (m, 2 H), 2.70-2.74 (m, 1 H), 2.86 (dd, J = 14, 6 Hz, 1 |
| | | | H), 3.34-3.40 (m, 1 H), 3.95 (br d, J = 5 Hz, 1 H), 4.78 (s, 1 H), |
| | | | 4.85 (br s, 1 H), 7.15-7.27 (m, 6 H), 7.48 (d, J = 9 Hz, 1 H) |
| 48 | A | 329 | (400 MHz, DMSO- d₆) 0.67-0.89 (m, 10 H), 1.26-1.48 (m, 6 H), |
| | | | 1.62-1.73 (m, 2 H), 2.79-2.87 (m, 2 H), 3.32-3.51 (m, 2 H), |
| | | | 4.71 (s, 1 H), 7.46-7.51 (m, 1 H), 7.57 (d, J = 7 Hz, 1 H), 7.63- |
| | | | 7.73 (m, 3 H) |
| 49 | A | 347 | (400 MHz, DMSO- d₆) 0.68-0.90 (m, 10 H), 1.30-1.52 (m, 6 H), |
| | | | 1.67 (br d, J = 12 Hz, 2 H), 2.80 (br t, J = 7 Hz, 2 H), 3.36-3.49 (m, |
| | | | 2 H), 4.73 (br s, 1 H), 7.26-7.40 (m, 3 H), 7.67-7.77 (m, 3 H), |
| | | | 7.90 (br s, 1 H) |
| 50 | A | 319 | (500 MHz, DMSO- d₆) 0.71-0.89 (m, 10 H), 1.29-1.51 (m, 6 H), |
| | | | 1.63-1.73 (m, 2 H), 1.88 (br s, 2 H), 3.14 (ddd, J = 13, 8, 5 Hz, 1 |
| | | | H), 3.31-3.39 (m, 1 H), 3.92 (dd, J = 7, 6 Hz, 1 H), 4.78 (s, 1 H), |
| | | | 7.19-7.23 (m, 1 H), 7.27-7.38 (m, 4 H), 7.63 (br t, J = 6 Hz, 1 H) |
| 51 | A | 304 | (500 MHz, DMSO- d₆) 0.71-0.90 (m, 10 H), 1.30-1.52 (m, 6 H), |
| | | | 1.64-1.73 (m, 2 H), 2.69-2.78 (m, 2 H), 3.31-3.43 (m, 2 H), |
| | | | 4.73 (s, 1 H), 7.17-7.22 (m, 3 H), 7.24-7.32 (m, 2 H), 7.67 (br t, |
| | | | J = 6 Hz, 1 H) |
| 52 | A | 348 | (400 MHz, DMSO- d₆) 0.74-0.93 (m, 10 H), 1.33-1.56 (m, 6 H), |
| | | | 1.64-1.77 (m, 2 H), 2.73-2.84 (m, 4 H), 3.23-3.39 (m, 2 H), |
| | | | 3.49-3.69 (m, 2 H), 4.64 (br t, J = 5 Hz, 1 H), 4.75 (s, 1 H), 7.10- |
| | | | 7.19 (m, 4 H), 7.80 (br t, J = 6 Hz, 1 H) |
| 53 | A | 347 | (400 MHz, DMSO- d₆) 0.74-0.91 (m, 10 H), 1.33-1.54 (m, 6 H), |
| | | | 1.64-1.75 (m, 2 H), 2.67-2.79 (m, 6 H), 3.27-3.37 (m, 2 H), |
| | | | 4.77 (br s, 1 H), 7.09-7.17 (m, 4 H), 7.80 (br t, J = 6 Hz, 1 H) |
| 54 | A | 300 | (400 MHz, DMSO- d₆) 0.74-0.92 (m, 10 H), 1.18 (dd, J = 6, 1 Hz, |
| | | | 6 H), 1.30-1.57 (m, 5 H), 1.63-1.78 (m, 3 H), 3.72-3.86 (m, 2 |
| | | | H), 4.86-4.94 (m, 2 H), 8.04 (t, J = 6 Hz, 1 H) |
| 55 | A | 378 | (400 MHz, DMSO- d₆) 0.72-0.90 (m, 10 H), 1.24-1.52 (m, 6 H), |
| | | | 1.63-1.74 (m, 2 H), 3.27-3.33 (m, 1 H), 3.44-3.52 (m, 1 H), |
| | | | 3.58 (s, 1 H), 3.79 (d, J = 16 Hz, 1 H), 3.95 (d, J = 16 Hz, 1 H), 4.57 |
| | | | (dd, J = 7, 5 Hz, 1 H), 4.80 (s, 1 H), 7.30-7.39 (m, 5 H), 7.82 (br s, |
| | | | 1 H) |
| 56 | A | 364 | (400 MHz, DMSO- d₆) 0.73-0.90 (m, 10 H), 1.32-1.56 (m, 6 H), |
| | | | 1.67 (br d, J = 12 Hz, 2 H), 3.15-3.29 (m, 3 H), 3.40-3.55 (m, 3 |
| | | | H), 4.42 (dd, J = 8, 5 Hz, 1 H), 4.54 (t, J = 5 Hz, 1 H), 4.84 (s, 1 H), |
| | | | 7.27-7.38 (m, 5 H), 7.57-7.65 (m, 1 H) |
| 57 | A | 392 | (400 MHz, DMSO- d₆) 0.64-0.90 (m, 10 H), 1.28-1.52 (m, 6 H), |
| | | | 1.66 (br d, J = 10 Hz, 2 H), 3.29-3.36 (m, 1 H), 3.43-3.57 (m, 1 |
| | | | H), 3.63 (s, 3 H), 3.91 (d, J = 16 Hz, 1 H), 4.06 (d, J = 16 Hz, 1 H), |
| | | | 4.57 (dd, J = 7, 5 Hz, 1 H), 4.84 (s, 1 H), 7.28-7.40 (m, 5 H), 7.69 |
| | | | (br d, J = 5 Hz, 1 H) |
| 58 | A | 318 | (400 MHz, DMSO- d₆) 0.69-0.92 (m, 10 H), 1.29-1.54 (m, 6 H), |
| | | | 1.63-1.74 (m, 2 H), 2.27 (s, 3 H), 2.69 (td, J = 7, 4 Hz, 2 H), 3.22- |
| | | | 3.46 (m, 2 H), 4.73 (s, 1 H), 6.97-7.02 (m, 3 H), 7.16 (t, J = 7 Hz, |
| | | | 1 H), 7.66 (br t, J = 6 Hz, 1 H) |
| 59 | A | 318 | (400 MHz, DMSO- d₆) 0.73-0.89 (m, 10 H), 1.29-1.57 (m, 6 H), |
| | | | 1.63-1.76 (m, 2 H), 2.30 (s, 3 H), 2.68-2.77 (m, 2 H), 3.25- |
| | | | 3.36 (m, 2 H), 4.74 (s, 1 H), 7.07-7.15 (m, 4 H), 7.77 (br t, J = 6 |
| | | | Hz, 1 H) |
| 60 | A | 318 | (400 MHz, DMSO- d₆) 0.71-0.88 (m, 10 H), 1.29-1.54 (m, 4 H), |
| | | | 1.62-1.74 (m, 2 H), 2.26 (s, 3 H), 2.68 (td, J = 7, 3 Hz, 2 H), 3.22- |
| | | | 3.28 (m, 1 H), 3.33-3.42 (m, 1 H), 4.73 (s, 1 H), 7.08 (s, 4 H), |
| | | | 7.64 (t, J = 6 Hz, 1 H) |
| 61 | A | 392 | (400 MHz, DMSO- d₆) 0.70-0.88 (m, 10 H), 1.29-1.56 (m, 6 H), |
| | | | 1.67 (br d, J = 10 Hz, 2 H), 2.78 (dt, J = 11, 7 Hz, 2 H), 3.33-3.43 |
| | | | (m, 2 H), 3.70 (s, 3 H), 4.72 (br s, 1 H), 4.82 (s, 2 H), 6.85-6.92 |
| | | | (m, 2 H), 7.12-7.18 (m, 2 H), 7.61 (br t, J = 6 Hz, 1 H) |
| 62 | A | 390 | (400 MHz, DMSO- d₆) 0.68-0.89 (m, 10 H), 1.14 (d, J = 7 Hz, 3 |
| | | | H), 1.24-1.53 (m, 6 H), 1.62-1.75 (m, 2 H), 1.81 (br s, 2 H), |
| | | | 3.17-3.30 (m, 2 H), 3.38-3.50 (m, 1 H), 4.82 (s, 1 H), 4.90 (br d, |
| | | | J = 5 Hz, 1 H), 7.21-7.34 (m, 5 H), 7.83 (t, J = 6 Hz, 1 H), 8.22 (br |
| | | | d, J = 7 Hz, 1 H) |
| 63 | A | 390 | (400 MHz, DMSO- d₆) 0.68-0.88 (m, 10 H), 1.11 (d, J = 7 Hz, 3 |
| | | | H), 1.27-1.53 (m, 6 H), 1.62-1.71 (m, 2 H), 1.74 (br s, 2 H), |
| | | | 3.22-3.28 (m, 1 H), 3.32-3.37 (m, 1 H), 3.38-3.50 (m, 1 H), |
| | | | 4.80 (s, 1 H), 4.86-4.94 (m, 1 H), 7.21-7.34 (m, 5 H), 7.81 (t, |
| | | | J = 6 Hz, 1 H), 8.18 (br d, J = 8 Hz, 1 H) |
| 64 | A | 392 | (400 MHz, DMSO- d₆) 0.71-0.88 (m, 10 H), 1.29-1.53 (m, 6 H), |
| | | | 1.63-1.74 (m, 2 H), 2.65-2.76 (m, 2 H), 3.33-3.43 (m, 2 H), |
| | | | 3.70 (s, 3 H), 4.76 (s, 3 H), 6.73-6.83 (m, 3 H), 7.19 (t, J = 8 Hz, 1 |
| | | | H), 7.68 (br t, J = 6 Hz, 1 H) |
| 65 | A | 376 | (400 MHz, DMSO- d₆) 0.62-0.94 (m, 10 H), 1.22-1.55 (m, 6 H), |
| | | | 1.66 (br d, J = 11 Hz, 2 H), 2.51-2.61 (m, 2 H), 2.64-2.78 (m, 1 |
| | | | H), 3.28-3,32 (m, 2 H), 3.47 (s, 3 H), 4.72 (br d, J = 8 Hz, 1 H), |
| | | | 7.15-7.36 (m, 5 H), 7.53-7.67 (m, 1 H) |
| 66 | A | 348 | (400 MHz, DMSO- d₆) 0.63-0.87 (m, 10 H), 1.22-1.51 (m, 6 H), |
| | | | 1.58-1.70 (m, 3 H), 1.74-1.85 (m, 1 H), 2.87-2.96 (m, 1 H), |
| | | | 3.16-3.28 (m, 3 H), 3.32-3.45 (m, 1 H), 4.34-4.38 (m, 1 H), |
| | | | 4.69 (d, J = 5 Hz, 1 H), 7.16-7.22 (m, 3 H), 7.25-7.43 (m, 3 H) |
| 67 | A | 334 | (400 MHz, DMSO- d₆) 0.72-0.93 (m, 10 H), 1.34-1.58 (m, 5 H), |
| | | | 1.65-1.79 (m, 3 H), 4.05-4.17 (m, 2 H), 4.95 (s, 1 H), 7.10 (d, |
| | | | J = 8 Hz, 2 H), 7.23-7.31 (m, 1 H), 7.43 (t, J = 7 Hz, 2 H), 8.26 (t, |
| | | | J = 6 Hz, 1 H) |
| 68 | A | 392 | (400 MHz, DMSO- d₆) 0.70-0.90 (m, 10 H), 1.34-1.59 (m, 5 H), |
| | | | 1.63-1.78 (m, 3 H), 3.87 (s, 3 H), 4.16 (dd, J = 6, 3 Hz, 2 H), 4.94 |
| | | | (s, 1 H), 7.32 (d, J = 8 Hz, 1 H), 7.57-7.64 (m, 2 H), 8.28 (t, J = 6 |
| | | | Hz, 1 H), 9.98 (s, 1 H) |
| 69 | A | 362 | (400 MHz, DMSO- d₆) 0.66-0.86 (m, 10 H), 1.24-1.49 (m, 6 H), |
| | | | 1.61-1.72 (m, 2 H), 3.41-3.76 (m, 5 H), 3.95-4.02 (m, 1 H), |
| | | | 4.76 (br d, J = 13 Hz, 1 H), 7.25-7.36 (m, 5 H), 7.72 (br s, 1 H) |
| 70 | A | 334 | (400 MHz, DMSO- d₆) 0.65-0.88 (m, 10 H), 1.23-1.50 (m, 6 H), |
| | | | 1.65 (br d, J = 11 Hz, 2 H), 2.87-2.95 (m, 1 H), 3.30 (s, 1 H), 3.38- |
| | | | 3.47 (m, 1 H), 3.52-3.61 (m, 2 H), 4.69-4.75 (m, 2 H), 7.17- |
| | | | 7.30 (m, 5 H), 7.53 (dt, J = 12, 6 Hz, 1 H) |
| 71 | A | 391 | (400 MHz, DMSO- d₆) 0.66-0.88 (m, 10 H), 1.20-1.52 (m, 6 H), |
| | | | 1.58-1.70 (m, 2 H), 3.07 (br s, 2 H), 3.36-3.60 (m, 2 H), 3.69- |
| | | | 3.90 (m, 1 H), 4.03 (q, J = 7 Hz, 1 H), 4.11-4.29 (m, 2 H), 4.78 (br |
| | | | s, 1 H), 7.26-7.37 (m, 5 H), 7.78 (dt, J = 18, 6 Hz, 1 H), 8.04 (br s, |
| | | | 3 H) |
| 72 | A | 321 | (400 MHz, DMSO- d₆) 0.68-0.89 (m, 10 H), 1.28-1.53 (m, 6 H), |
| | | | 1.66 (br d, J = 10 Hz, 2 H), 3.10-3.52 (m, 2 H), 4.85 (s, 1 H), 5.48 |
| | | | (br d, J = 11 Hz, 1 H), 7.22-7.37 (m, 5 H), 7.51-7.65 (m, 1 H) |
| 73 | A | 419 | (400 MHz, DMSO- d₆) 0.65-0.88 (m, 10 H), 1.16-1.49 (m, 12 |
| | | | H), 1.65 (br d, J = 10 Hz, 2 H), 3.51-3.65 (m, 1 H), 3.71-3.84 (m, |
| | | | 1 H), 3.96-4.13 (m, 3 H), 4.78 (br d, J = 5 Hz, 1 H), 7.27-7.37 |
| | | | (m, 5 H), 7.75 (dt, J = 18, 6 Hz, 1 H), 8.15 (br s, 3 H) |
| 74 | A | 376 | (400 MHz, DMSO- d₆) 0.71-0.89 (m, 10 H), 1.30-1.54 (m, 6 H), |
| | | | 1.67 (br d, J = 13 Hz, 2 H), 2.60-2.77 (m, 2 H), 3.34-3.43 (m, 2 |
| | | | H), 3.58-3.66 (m, 5 H), 4.73 (br s, 1 H), 7.07-7.13 (m, 3 H), |
| | | | 7.23 (t, J = 7 Hz, 1 H), 7.71 (br t, J = 6 Hz, 1 H) |
| 75 | A | 406 | (400 MHz, DMSO- d₆) 0.68-0.88 (m, 10 H), 1.24-1.53 (m, 6 H), |
| | | | 1.67 (br d, J = 11 Hz, 2 H), 2.60-2.74 (m, 2 H), 2.83 (t, J = 6 Hz, 2 |
| | | | H), 3.18-3.44 (m, 2 H), 3.64 (s, 3 H), 4.21 (t, J = 6 Hz, 2 H), 4.73 |
| | | | (br s, 1 H), 6.83-6.90 (m, 1 H), 6.96 (d, J = 8 Hz, 1 H), 7.11 (br d, |
| | | | J = 7 Hz, 1 H), 7.17 (t, J = 7 Hz, 1 H), 7.56 (br t, J = 5 Hz, 1 H) |
| 76 | A | 366 | (400 MHz, DMSO- d6) 0.72-0.91 (m, 10 H), 1.33-1.57 (m, 5 H), |
| | | | 1.65-1.78 (m, 3 H), 3.97-4.10 (m, 2 H), 4.92 (s, 1 H), 5.94 (d, |
| | | | J = 2 Hz, 2 H), 6.09 (t, J = 2 Hz, 1 H), 8.21 (t, J = 6 Hz, 1 H), 9.48 (s, |
| | | | 2 H) |
| 77 | A | 376 | (400 MHz, DMSO- d₆) 0.74-0.89 (m, 10 H), 1.31-1.56 (m, 6 H), |
| | | | 1.68 (br d, J = 11 Hz, 2 H), 2.67-2.76 (m, 2 H), 3.19-3.28 (m, 2 |
| | | | H), 3.61 (s, 3 H), 3.77 (d, J = 3 Hz, 2 H), 4.75 (s, 1 H), 7.14-7.21 |
| | | | (m, 4 H), 7.81 (t, J = 6 Hz, 1 H) |
| 78 | A | 362 | (400 MHz, DMSO- d₆) 0.73-0.88 (m, 10 H), 1.32-1.54 (m, 6 H), |
| | | | 1.63-1.74 (m, 2 H), 2.66-2.76 (m, 2 H), 3.34-3.41 (m, 2 H), |
| | | | 3.52 (s, 2 H), 4.73 (s, 1 H), 7.07-7.11 (m, 3 H), 7.20-7.25 (m, 1 |
| | | | H), 7.71 (t, J = 6 Hz, 1 H), 12.00-12.64 (br s, 1H ) |
| 79 | A | 394 | (400 MHz, DMSO- d₆) 0.72-0.90 (m, 10 H), 1.33-1.58 (m, 5 H), |
| | | | 1.65-1.78 (m, 3 H), 3.76 (s, 3 H), 4.11 (dd, J = 6, 3 Hz, 2 H), 4.48 |
| | | | (br d, J = 4 Hz, 2 H), 4.92 (s, 1 H), 5.21 (br s, 1 H), 6.89 (d, J = 8 |
| | | | Hz, 1 H), 6.98 (d, J = 8 Hz, 1 H), 7.08 (s, 1 H), 8.22 (br t, J = 6 Hz, 1 |
| | | | H) |
| 80 | A | 378 | (400 MHz, DMSO- d₆) 0.70-0.90 (m, 10 H), 1.30-1.52 (m, 6 H), |
| | | | 1.67 (br d, J = 11 Hz, 2 H), 1.89 (quin, J = 6 Hz, 2 H), 2.65-2.79 |
| | | | (m, 2 H), 3.33-3.43 (m, 2 H), 3.57-3.63 (m, 2 H), 4.04 (t, J = 6 |
| | | | Hz, 2 H), 4.46-4.56 (m, 1 H), 4.71 (br s, 1 H), 6.83 (t, J = 7 Hz, 1 |
| | | | H), 6.93 (d, J = 7 Hz, 1 H), 7.10 (dd, J = 7, 2 Hz, 1 H), 7.16 (t, J = 8 |
| | | | Hz, 1 H), 7.61 (br t, J = 6 Hz, 1 H) |
| 81 | A | 377 | (400 MHz, DMSO- d₆) 0.68-0.97 (m, 10 H), 1.28-1.57 (m, 6 H), 1.61- |
| | | | 1.78 (m, 2 H), 2.72-2.88 (m, 2 H), 3.22-3.48 (m, 2 H), 4.32-4.52 (m, 2 |
| | | | H), 4.75 (s, 1 H), 6.79-6.94 (m, 2 H), 7.10-7.25 (m, 2 H), 7.39-7.62 (m, |
| | | | 2 H), 7.82 (t, J = 6 Hz, 1 H) |
| 82 | A | 388 | (400 MHz, DMSO- d₆) 0.49 (br dd, J = 10, 7 Hz, 7 H), 0.76 (br d, |
| | | | J = 6 Hz, 5 H), 1.30 (br s, 4 H), 1.62 (br d, J = 11 Hz, 2 H), 3.50- |
| | | | 3.58 (m, 1 H), 4.25 (br dd, J = 14, 8 Hz, 1 H), 4.90 (br s, 1 H), 7.12 |
| | | | (br s, 1 H), 7.35 (br d, J = 6 Hz, 3 H), 7.51-7.64 (m, 3 H) |
| 83 | A | 388 | (400 MHz, DMSO- d₆) 0.52-0.90 (m, 10 H), 1.04-1.42 (m, 6 H), |
| | | | 1.60 (br d, J = 10 Hz, 2 H), 3.67 (dd, J = 14, 4 Hz, 1 H), 4.14 (dd, |
| | | | J = 14, 8 Hz, 1 H), 4.91 (br s, 1 H), 7.27-7.41 (m, 4 H), 7.56 (br d, |
| | | | J = 7 Hz, 2 H), 7.81 (br dd, J = 7, 4 Hz, 1 H) |
| 84 | A | 405 | (400 MHz, DMSO- d₆) 0.72-0.89 (m, 10 H), 1.34-1.44 (m, 4 H), |
| | | | 1.44-1.55 (m, 2 H), 1.68 (br d, J = 10 Hz, 2 H), 1.83 (s, 3 H), 2.66- |
| | | | 2.78 (m, 2 H), 3.32-3.40 (m, 2 H), 3.42-3.51 (m, 2 H), 3.97 (t, |
| | | | J = 5 Hz, 2 H), 4.79 (s, 1 H), 6.85 (t, J = 7 Hz, 1 H), 6.92 (d, J = 8 Hz, |
| | | | 1 H), 7.11 (dd, J = 7, 2 Hz, 1 H), 7.16 (t, J = 8 Hz, 1 H), 7.83 (br t, |
| | | | J = 6 Hz, 1 H), 8.23 (br t, J = 6 Hz, 1 H) |
| 85 | A | 336 | (400 MHz, DMSO- d₆) 0.71-0.93 (m, 10 H), 1.29-1.83 (m, 8 H), |
| | | | 4.48 (dd, J = 5, 3 Hz, 2 H), 4.98 (s, 1 H), 7.33-7.42 (m, 2 H), 7.65- |
| | | | 7.75 (m, 1 H), 7.87 (td, J = 8, 2 Hz, 1 H), 8.11 (t, J = 5 Hz, 1 H) |
| 86 | A | 334 | (400 MHz, DMSO- d₆) 0.61-0.95 (m, 10 H), 1.31-1.54 (m, 6 H), |
| | | | 1.64-1.79 (m, 2 H), 1.80-1.97 (m, 2 H), 3.32-3.43 (m, 2 H), |
| | | | 4.51 (t, J = 5 Hz, 1 H), 4.77 (s, 1 H), 4.95 (td, J = 8, 6 Hz, 1 H), 7.15- |
| | | | 7.37 (m, 5 H), 8.06 (d, J = 9 Hz, 1 H) |
| 87 | A | 376 | (400 MHz, DMSO- d₆) 0.73-0.91 (m, 10 H), 1.31-1.54 (m, 6 H), |
| | | | 1.63-1.74 (m, 2 H), 2.49-2.56 (m, 2 H), 2.64-2.90 (m, 4 H), |
| | | | 3.34-3.42 (m, 2 H), 4.58-4.92 (m, 1 H), 7.01-7.07 (m, 3 H), |
| | | | 7.16-7.21 (m, 1 H), 7.68 (t, J = 6 Hz, 1 H), 11.73-12.65 (br s, |
| | | | 1H ) |
| 88 | A | 349 | (400 MHz, DMSO- d₆) 0.74-0.93 (m, 10 H), 1.27-1.81 (m, 8 H), |
| | | | 3.87 (d, J = 6 Hz, 2 H), 4.96 (s, 1 H), 6.66-6.73 (m, 2 H), 7.33 (m, |
| | | | J = 9 Hz, 2 H), 7.94 (t, J = 5 Hz, 1 H), 9.16 (br s, 1 H), 9.66 (s, 1 H) |
| 89 | B | 334 | (400 MHz, DMSO- d₆) 0.68-0.90 (m, 10 H), 1.25-1.51 (m, 6 H), |
| | | | 1.66 (br d, J = 10 Hz, 2 H), 2.86-2.99 (m, 1 H), 3.26-3.36 (m, 1 |
| | | | H), 3.53-3.62 (m, 3 H), 4.71-4.78 (m, 2 H), 7.18-7.32 (m, 5 H), |
| | | | 7.54 (br t, J = 6 Hz, 1 H) |
| 90 | B | 334 | (400 MHz, DMSO- d₆) 0.65-0.91 (m, 10 H), 1.24-1.48 (m, 6 H), |
| | | | 1.57-1.72 (m, 2 H), 2.92 (quin, J = 7 Hz, 1 H), 3.38-3.49 (m, 2 |
| | | | H), 3.53-3.62 (m, 2 H), 4.70-4.78 (m, 2 H), 7.18-7.30 (m, 5 H), |
| | | | 7.56 (br t, J = 6 Hz, 1 H) |
| 91 | B | 321 | (400 MHz, DMSO- d₆) 0.68-0.90 (m, 10 H), 1.28-1.52 (m, 6 H), |
| | | | 1.62-1.75 (m, 2 H), 3.32-3.48 (m, 2 H), 4.86 (s, 1 H), 5.51 (s, 1 |
| | | | H), 7.22-7.36 (m, 5 H), 7.57 (br t, J = 6 Hz, 1 H) |
| 92 | B | 321 | (400 MHz, DMSO- d₆) 0.73-0.84 (m, 10 H), 1.30-1.53 (m, 6 H), |
| | | | 1.67 (br d, J = 13 Hz, 2 H), 3.13-3.22 (m, 1 H), 3.44 (dd, J = 13, 7 |
| | | | Hz, 1 H), 4.86 (s, 1 H), 5.48 (s, 1 H), 7.19-7.40 (m, 5 H), 7.62 (br |
| | | | t, J = 6 Hz, 1 H ) |
| 93 | A | 390 | (400 MHz, DMSO- d₆) 0.72-0.89 (m, 10 H), 1.29-1.56 (m, 6 H), |
| | | | 1.68 (br d, J = 10 Hz, 2 H), 2.59 (t, J = 8 Hz, 2 H), 2.67-2.80 (m, 2 |
| | | | H), 2.87-2.94 (m, 2 H), 3.20-3.39 (m, 2 H), 3.59 (s, 3 H), 4.73 |
| | | | (s, 1 H), 7.11-7.18 (m, 4 H), 7.82 (br t, J = 6 Hz, 1 H) |
| 94 | A | 332 | (400 MHz, DMSO- d₆) 0.56-0.95 (m, 10 H), 1.28-1.54 (m, 6 H), |
| | | | 1.66 (br d, J = 12 Hz, 2 H), 2.82 (br dd, J = 16, 6 Hz, 1 H), 2.95 (br |
| | | | dd, J = 16, 5 Hz, 1 H), 3.98 (dd, J = 10, 7 Hz, 1 H), 4.08 (br d, J = 10 |
| | | | Hz, 1 H), 4.18 (br s, 1 H), 4.90 (br s, 1 H), 6.78 (d, J = 8 Hz, 1 H), |
| | | | 6.85 (t, J = 7 Hz, 1 H), 7.02-7.12 (m, 2 H), 7.60 (br d, J = 7.91 Hz, |
| | | | 1 H) |
| 95 | A | 332 | (400 MHz, DMSO- d₆) 0.73-0.93 (m, 10 H), 1.29-1.61 (m, 6 H), |
| | | | 1.63-1.73 (m, 2 H), 2.80 (dd, J = 16, 7 Hz, 1 H), 2.96 (dd, J = 16, 5 |
| | | | Hz, 1 H), 3.93 (dd, J = 10, 7 Hz, 1 H), 4.05 (dd, J = 10, 2 Hz, 1 H), |
| | | | 4.14-4.23 (m, 1 H), 4.89 (s, 1 H), 6.79 (d, J = 8 Hz, 1 H), 6.85 (td, |
| | | | J = 7, 1 Hz, 1 H), 7.06-7.11 (m, 2 H), 7.61 (br d, J = 8 Hz, 1 H) |
| 96 | A | | (400 MHz, DMSO- d₆) 0.73-0.90 (m, 10 H), 1.32-1.55 (m, 6 H), |
| | | | 1.64-1.74 (m, 2 H), 1.82-1.97 (m, 2 H), 3.31-3.35 (m, 1 H), |
| | | | 3.60 (dd, J = 14, 7 Hz, 1 H), 4.15-4.20 (m, 2 H), 4.99 (d, J = 8 Hz, |
| | | | 1 H), 5.52 (d, J = 11 Hz, 1 H), 6.74 (d, J = 8 Hz, 1 H), 6.88 (t, J = 7 |
| | | | Hz, 1 H), 7.14 (t, J = 8 Hz, 1 H), 7.46 (ddd, J = 8, 4, 2 Hz, 1 H), 7.57- |
| | | | 7.62 (m, 1 H) |
| 97 | A | 346 | (400 MHz, DMSO- d₆) 0.72-0.90 (m, 10 H), 1.24-1.46 (m, 4 H), |
| | | | 1.50-1.72 (m, 4 H), 2.66-2.73 (m, 1 H), 2.81-2.90 (m, 1 H), |
| | | | 3.44 (ddd, J = 14, 8, 6 Hz, 1 H), 3.62-3.73 (m, 2 H), 4.06 (dt, |
| | | | J = 11, 5 Hz, 1 H), 4.77 (dd, J = 8, 3 Hz, 1 H), 4.86 (s, 1 H), 7.12- |
| | | | 7.19 (m, 4 H), 7.56 (br t, J = 6 Hz, 1 H) |
| 98 | A | 346 | (400 MHz, DMSO- d₆) 0.60 (t, J = 7 Hz, 6 H), 0.80 (d, J = 6 Hz, 4 |
| | | | H), 0.96-1.09 (m, 1 H), 1.24-1.48 (m, 5 H), 1.66 (br d, J = 11 Hz, |
| | | | 2 H), 2.65-2.72 (m, 1 H), 2.82-2.91 (m, 1 H), 3.39-3.50 (m, 1 |
| | | | H), 3.66-3.77 (m, 2 H), 4.06-4.13 (m, 1 H), 4.81-4.90 (m, 2 H), |
| | | | 7.11-7.19 (m, 4 H), 7.47-7.54 (m, 1 H) |
| 99 | A | 378 | (400 MHz, DMSO- d₆) 0.73-0.90 (m, 10 H), 1.30-1.82 (m, 8 H), |
| | | | 3.86 (s, 3 H), 3.94 (s, 3 H), 4.42 (s, 2 H), 4.96 (s, 1 H), 6.61-6.72 |
| | | | (m, 2 H), 7.76 (d, J = 9 Hz, 1 H), 7.99 (t, J = 5 Hz, 1 H) |
| 100 | A | 350 | (400 MHz, DMSO- d₆) 0.73-0.94 (m, 10 H), 1.35-1.57 (m, 6 H), |
| | | | 1.62-1.79 (m, 2 H), 4.51 (d, J = 5 Hz, 2 H), 4.98 (br s, 1 H), 6.82 |
| | | | (d, J = 8 Hz, 1 H), 7.35 (d, J = 2 Hz, 1 H), 7.40 (dd, J = 8, 2 Hz, 1 H), |
| | | | 7.96 (t, J = 5 Hz, 1 H), 9.34 (br s, 1 H), 9.89 (s, 1 H) |
| 101 | A | 460 | (400 MHz, DMSO- d₆) 0.69-0.88 (m, 10 H), 1.30-1.56 (m, 10 |
| | | | H), 1.58-1.80 (m, 4 H), 3.44-3.60 (m, 2 H), 3.64-3.73 (m, 1 H), |
| | | | 3.75-3.86 (m, 1 H), 4.45 (d, J = 12 Hz, 1 H), 4.65-4.76 (m, 6 H), |
| | | | 4.83 (s, 1 H), 7.06 (d, J = 8 Hz, 1 H), 7.09 (s, 1 H), 7.25 (d, J = 8 Hz, |
| | | | 1 H), 7.35 (t, J = 8 Hz, 1 H), 7.63 (br t, J = 6 Hz, 1 H) |
| 102 | A | 350 | (400 MHz, DMSO- d₆) 0.72-0.94 (m, 10 H), 1.32-1.61 (m, 5 H), |
| | | | 1.64-1.80 (m, 3 H), 4.55 (d, J = 6 Hz, 2 H), 4.99 (s, 1 H), 6.31 (d, |
| | | | J = 2 Hz, 1 H), 6.38 (dd, J = 9, 2 Hz, 1 H), 7.80 (d, J = 9 Hz, 1 H), |
| | | | 8.03 (t, J = 5 Hz, 1 H), 10.57 (s, 1 H), 11.85 (s, 1 H) |
| 103 | A | 346 | (400 MHz, DMSO- d₆) 0.75-0.93 (m, 10 H), 1.32-1.63 (m, 6 H), |
| | | | 1.66-1.92 (m, 4 H), 2.95 (dq, J = 9, 4 Hz, 1 H), 3.27-3.48 (m, 2 |
| | | | H), 4.07-4.17 (m, 2 H), 4.82 (d, J = 9 Hz, 1 H), 6.74 (d, J = 8 Hz, 1 |
| | | | H), 6.84 (t, J = 7 Hz, 1 H), 7.08 (t, J = 8 Hz, 1 H), 7.19 (d, J = 8 Hz, 1 |
| | | | H), 7.95 (q, J = 6 Hz, 1 H) |
| 104 | A | 350 | (400 MHz, DMSO- d₆) 0.77-0.94 (m, 10 H), 1.36-1.48 (m, 3 H), |
| | | | 1.50-1.58 (m, 2 H), 1.64-1.80 (m, 3 H), 4.62 (d, J = 6 Hz, 2 H), |
| | | | 4.98 (br s, 1 H), 6.76 (t, J = 8 Hz, 1 H), 7.04 (dd, J = 8, 1 Hz, 1 H), |
| | | | 7.34 (dd, J = 8, 1 Hz, 1 H), 8.06 (t, J = 5 Hz, 1 H) |
| 105 | A | 406 | (400 MHz, DMSO- d₆) 0.62-0.92 (m, 10 H), 1.14 (d, J = 6 Hz, 3 H), 1.21- |
| | | | 1.55 (m, 6 H), 1.59-1.77 (m, 2 H), 2.76-2.93 (m, 2 H), 3.32-3.51 (m, 2 |
| | | | H), 3.89-4.00 (m, 1 H), 4.04-4.21 (m, 2 H), 4.70 (s, 1 H), 4.93 (d, J = 5 |
| | | | Hz, 1 H), 7.37-7.56 (m, 2 H), 7.71 (t, J = 6 Hz, 1 H), 7.80-7.88 (m, 2 H) |
| 106 | A | 362 | (400 MHz, DMSO- d₆) 0.70-0.96 (m, 10 H), 1.27-1.51 (m, 6 H), |
| | | | 1.62-1.74 (m, 2 H), 2.99-3.16 (m, 2 H), 3.32-3.47 (m, 2 H), |
| | | | 3.85 (s, 3 H), 4.71 (s, 1 H), 7.30-7.38 (m, 2 H), 7.46-7.54 (m, 1 |
| | | | H), 7.71 (br t, J = 5.65 Hz, 1 H), 7.80 (d, J = 8.17 Hz, 1 H) |
| 107 | A | 362 | (400 MHz, DMSO- d₆) 0.68-0.87 (m, 10 H), 1.27-1.42 (m, 5 H), |
| | | | 1.43-1.50 (m, 1 H), 1.63-1.73 (m, 2 H), 2.78-2.89 (m, 2 H), |
| | | | 3.32-3.39 (m, 1 H), 3.41-3.50 (m, 1 H), 3.85 (s, 3 H), 4.70 (s, 1 |
| | | | H), 7.42-7.53 (m, 2 H), 7.70 (br t, J = 6 Hz, 1 H), 7.78-7.83 (m, 2 |
| | | | H) d6 |
| 108 | A | 362 | (400 MHz, DMSO- d₆) 0.65-0.88 (m, 10 H), 1.24-1.53 (m, 6 H), |
| | | | 1.60-1.75 (m, 2 H), 2.84 (br t, J = 6.96 Hz, 2 H), 3.32-3.40 (m, 1 |
| | | | H), 3.41-3.50 (m, 1 H), 3.84 (s, 3 H), 4.72 (s, 1 H), 7.37 (m, |
| | | | J = 8.28 Hz, 2 H), 7.71 (br t, J = 5.77 Hz, 1 H), 7.88 (m, J = 8.28 Hz, |
| | | | 2 H) |
| 109 | A | 334 | (400 MHz, DMSO- d₆) 0.71-0.92 (m, 10 H), 1.32-1.55 (m, 6 H), |
| | | | 1.62-1.76 (m, 2 H), 2.66-2.77 (m, 2 H), 3.22-3.47 (m, 2 H), |
| | | | 4.46 (d, J = 6 Hz, 2 H), 4.75 (s, 1 H), 5.08 (t, J = 6 Hz, 1 H), 7.16 (m, |
| | | | J = 8 Hz, 2 H), 7.23 (m, J = 8 Hz, 2 H), 7.67 (br t, J = 6 Hz, 1 H) |
| 110 | A | 244 | (400 MHz, DMSO- d₆) 0.68-0.97 (m, 10 H), 1.30-1.60 (m, 6 H), |
| | | | 1.63-1.80 (m, 2 H), 3.16 (qd, J = 6, 2 Hz, 2 H), 3.37-3.45 (m, 2 |
| | | | H), 4.68 (t, J = 5 Hz, 1 H), 4.81 (s, 1 H), 7.66 (br t, J = 6 Hz, 1 H) |
| 111 | A | 348 | (400 MHz, DMSO- d₆) 0.59-0.95 (m, 10 H), 1.29-1.80 (m, 8 H), |
| | | | 3.31 (s, 2 H), 3.53-3.61 (m, 1 H), 4.31 (t, J = 5 Hz, 2 H), 4.77 (s, 1 |
| | | | H), 7.45-7.55 (m, 2 H), 7.59-7.74 (m, 1 H), 7.87-8.09 (m, 2 H) |
| 112 | A | 284 | (400 MHz, DMSO- d₆) 0.70-1.00 (m, 10 H), 1.29-1.58 (m, 6 H), |
| | | | 1.61-1.81 (m, 2 H), 2.19-2.39 (m, 2 H), 4.20 (ddd, J = 10, 9, 7 |
| | | | Hz, 1 H), 4.33 (td, J = 9, 2 Hz, 1 H), 4.49-4.67 (m, 1 H), 4.88 (s, 1 |
| | | | H), 8.26 (d, J = 8 Hz, 1 H) |

Examples 113 to 174 were synthesized by parallel synthesis by set of 20 to 40 amines from a solution A and purified by RP-HPLC chromatography as described above in Experiment procedures.

(1S,2S,5R)-1-Hydroxy-2-isopropyl-5-methyl-cyclohexanecarboxylic acid (1.24 g) was dissolved in EtOAc (0.1M) and CDI (1.05eq) was added. The resulting solution (solution A) was allowed to stir at room temperature for one hour and said solution (“solution A”), was used for coupling reactions with 31 amines.

### Example 113: (1S,2S,5R)—N-[2-(3,4-dimethoxyphenyl)ethyl]-1-hydroxy-2-isopropyl-5-methyl-cyclohexanecarboxamide

The solution A (2 ml, 0.2 mmol) was added to 2-(3,4-dimethoxyphenyl)ethanamine (0.2 mmol, 1.0eq) at room temperature and the resulting reaction mixture was then heated at 80° C. btw 150 and 210 min. The solvent was removed in vacuo and the residue was dissolved in 2 ml of a 9:1 DMF/TFA mixture (9/1, 2 ml). The resulting crude solution was purified by RP-HPLC chromatography under conditions E below to yield (1S,2S,5R)—N-[2-(3,4-dimethoxyphenyl)ethyl]-1-hydroxy-2-isopropyl-5-methyl-cyclohexanecarboxamide.

Examples 114 to 152 and examples 156 to 174 were synthesized under same experimental conditions as for example 113 and purified by RP-HPLC chromatography Method G as described above.

The compound structure and compound name for Examples, 113 to 152 and 156 to 174, the amine used for synthesis and the RP-HPLC purification method are shown in Table 2.

TABLE 2

| | | | | RP-HPLC |
|---|---|---|---|---|
| | | | | Purification |
| Example | Structure | Compound Name | Amine used | method |
| 113 | 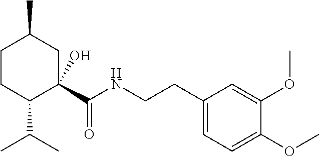 [SMILES: COc1ccc(CCNC(=O)[C@]2(O)C[C@H](C)CC[C@H]2C(C)C)cc1OC] | (1S,2S,5R)-N-[2-(3,4- dimethoxyphenyl)ethyl]- 1-hydroxy-2-isopropyl-5- methyl- cyclohexanecarboxamide | 2-(3,4- dimethoxyphenyl) ethanamine | G |
| 114 | 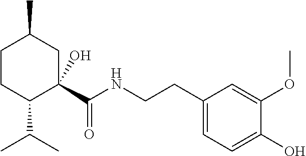 [SMILES: COc1cc(CCNC(=O)[C@]2(O)C[C@H](C)CC[C@H]2C(C)C)ccc1O] | (1S,2S,5R)-1-hydroxy- N-[2-(4-hydroxy-3- methoxy-phenyl)ethyl]- 2-isopropyl-5-methyl- cyclohexanecarboxamide | 4-(2-aminoethyl)- 2-methoxy-phenol | G |
| 115 | 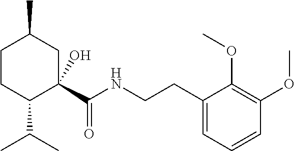 [SMILES: COc1cccc(CCNC(=O)[C@]2(O)C[C@H](C)CC[C@H]2C(C)C)c1OC] | (1S,2S,5R)-N-[2-(2,3- dimethoxyphenyl)ethyl]- 1-hydroxy-2-isopropyl-5- methyl- cyclohexanecarboxamide | 2-(2,3- dimethoxyphenyl) ethanamine | G |
| 116 | 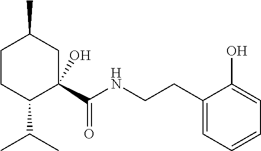 [SMILES: CC(C)[C@@H]1CC[C@@H](C)C[C@@]1(O)C(=O)NCCc1ccccc1O] | (1S,2S,5R)-1-hydroxy- N-[2-(2- hydroxyphenyl)ethyl]-2- isopropyl-5-methyl- cyclohexanecarboxamide | 2-(2- aminoethyl)phenol | G |
| 117 | 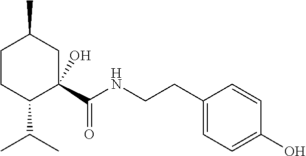 [SMILES: CC(C)[C@@H]1CC[C@@H](C)C[C@@]1(O)C(=O)NCCc1ccc(O)cc1] | (1S,2S,5R)-1-hydroxy- N-[2-(4- hydroxyphenyl)ethyl]-2- isopropyl-5-methyl- cyclohexanecarboxamide | 4-(2- aminoethyl)phenol | G |
| 118 | 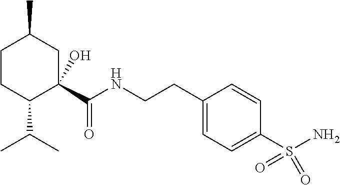 [SMILES: CC(C)[C@@H]1CC[C@@H](C)C[C@@]1(O)C(=O)NCCc1ccc(S(N)(=O)=O)cc1] | (1S,2S,5R)-1-hydroxy-2- isopropyl-5-methyl-N-[2- (4- sulfamoylphenyl)ethyl] cyclohexanecarboxamide | 4-(2-aminoethyl) benzene- sulfonamide | G |
| 119 | 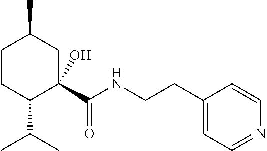 [SMILES: CC(C)[C@@H]1CC[C@@H](C)C[C@@]1(O)C(=O)NCCc1ccncc1] | (1S,2S,5R)-1-hydroxy-2- isopropyl-5-methyl-N-[2- (4-pyridyl)ethyl] cyclohexanecarboxamide | 2-(4-pyridyl) ethanamine | G |
| 120 | 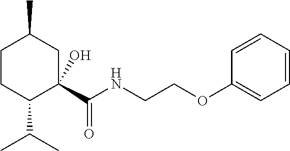 [SMILES: CC(C)[C@@H]1CC[C@@H](C)C[C@@]1(O)C(=O)NCCOc1ccccc1] | (1S,2S,5R)-1-hydroxy-2- isopropyl-5-methyl-N-(2- phenoxyethyl) cyclohexanecarboxamide | 2-phenoxy- ethanamine | G |
| 121 | 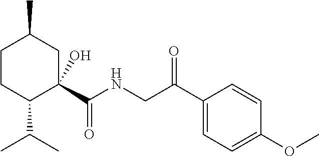 [SMILES: COc1ccc(C(=O)CNC(=O)[C@]2(O)C[C@H](C)CC[C@H]2C(C)C)cc1] | (1S,2S,5R)-1-hydroxy-2- isopropyl-N-[2-(4- methoxyphenyl)-2-oxo- ethyl]-5-methyl- cyclohexanecarboxamide | 2-amino-1-(4- methoxyphenyl) ethanone | G |
| 122 | 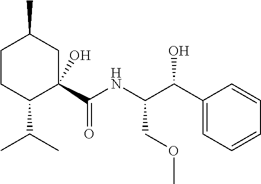 [SMILES: COC[C@H](NC(=O)[C@]1(O)C[C@H](C)CC[C@H]1C(C)C)[C@H](O)c1ccccc1] | (1S,2S,5R)-1-hydroxy- N-[(1S,2S)-2-hydroxy-1- (methoxymethyl)-2- phenyl-ethyl]-2- isopropyl-5-methyl- cyclohexanecarboxamide | (1S,2S)-2-amino- 3-methoxy-1- phenyl-propan-1-ol | G |
| 123 | 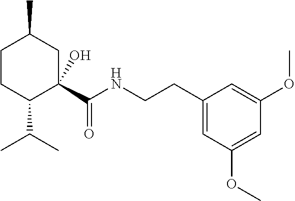 [SMILES: COc1cc(CCNC(=O)[C@]2(O)C[C@H](C)CC[C@H]2C(C)C)cc(OC)c1] | (1S,2S,5R)-N-[2-(3,5- dimethoxyphenyl)ethyl]- 1-hydroxy-2-isopropyl-5- methyl- cyclohexanecarboxamide | 2-(3,5- dimethoxyphenyl) ethanamine | G |
| 124 | 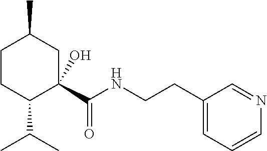 [SMILES: CC(C)[C@@H]1CC[C@@H](C)C[C@@]1(O)C(=O)NCCc1cccnc1] | (1S,2S,5R)-1-hydroxy-2- isopropyl-5-methyl-N-[2- (3-pyridyl)ethyl] cyclohexanecarboxamide | 2-(3-pyridyl) ethanamine | G |
| 125 | 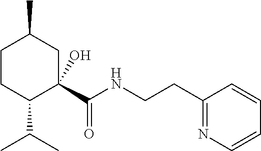 [SMILES: CC(C)[C@@H]1CC[C@@H](C)C[C@@]1(O)C(=O)NCCc1ccccn1] | (1S,2S,5R)-1-hydroxy-2- isopropyl-5-methyl-N-[2- (2-pyridyl)ethyl] cyclohexanecarboxamide | 2-(2-pyridyl) ethanamine | G |
| 126 | 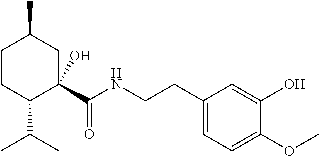 [SMILES: COc1ccc(CCNC(=O)[C@]2(O)C[C@H](C)CC[C@H]2C(C)C)cc1O] | (1S,2S,5R)-1-hydroxy- N-[2-(3-hydroxy-4- methoxy-phenyl)ethyl]- 2-isopropyl-5-methyl- cyclohexanecarboxamide | 5-(2-aminoethyl)- 2-methoxy-phenol | G |
| 127 | 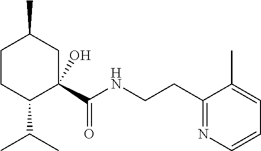 [SMILES: Cc1cccnc1CCNC(=O)[C@]1(O)C[C@H](C)CC[C@H]1C(C)C] | (1S,2S,5R)-1-hydroxy-2- isopropyl-5-methyl-N-[2- (3-methyl-2- pyridyl)ethyl] cyclohexanecarboxamide | 2-(3-methyl-2- pyridyl) ethanamine | G |
| 128 | 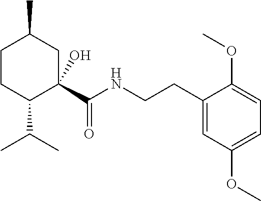 [SMILES: COc1ccc(OC)c(CCNC(=O)[C@]2(O)C[C@H](C)CC[C@H]2C(C)C)c1] | (1S,2S,5R)-N-[2-(2,5- dimethoxyphenyl)ethyl]- 1-hydroxy-2-isopropyl-5- methyl- cyclohexanecarboxamide | 2-(2,5- dimethoxyphenyl) ethanamine | G |
| 129 | 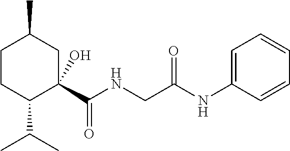 [SMILES: CC(C)[C@@H]1CC[C@@H](C)C[C@@]1(O)C(=O)NCC(=O)Nc1ccccc1] | (1S,2S,5R)-N-(2-anilino- 2-oxo-ethyl)-1-hydroxy- 2-isopropyl-5-methyl- cyclohexanecarboxamide | 2-amino-N-phenyl- acetamide | G |
| 130 | 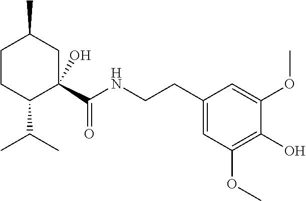 [SMILES: COc1cc(CCNC(=O)[C@]2(O)C[C@H](C)CC[C@H]2C(C)C)cc(OC)c1O] | (1S,2S,5R)-1-hydroxy- N-[2-(4-hydroxy-3,5- dimethoxy- phenyl)ethyl]-2- isopropyl-5-methyl- cyclohexanecarboxamide | 4-(2-aminoethyl)- 2,6-dimethoxy- phenol | G |
| 131 | 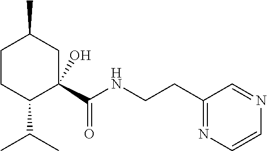 [SMILES: CC(C)[C@@H]1CC[C@@H](C)C[C@@]1(O)C(=O)NCCc1cnccn1] | (1S,2S,5R)-1-hydroxy-2- isopropyl-5-methyl-N-(2- pyrazin-2-ylethyl) cyclohexanecarboxamide | 2-pyrazin-2- ylethanamine | G |
| 132 | 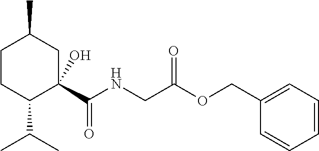 [SMILES: CC(C)[C@@H]1CC[C@@H](C)C[C@@]1(O)C(=O)NCC(=O)OCc1ccccc1] | benzyl 2-[[(1S,2S,5R)-1- hydroxy-2-isopropyl-5- methyl- cyclohexanecarbonyl] amino]acetate | benzyl 2- aminoacetate | G |
| 133 | 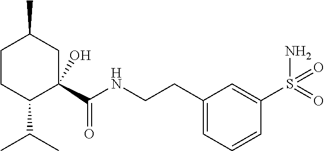 [SMILES: CC(C)[C@@H]1CC[C@@H](C)C[C@@]1(O)C(=O)NCCc1cccc(S(N)(=O)=O)c1] | (1S,2S,5R)-1-hydroxy-2- isopropyl-5-methyl-N-[2- (3- sulfamoylphenyl)ethyl] cyclohexanecarboxamide | 3-(2-aminoethyl) benzene- sulfonamide | G |
| 134 | 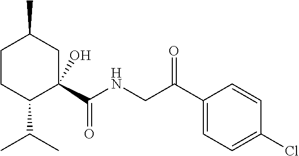 [SMILES: CC(C)[C@@H]1CC[C@@H](C)C[C@@]1(O)C(=O)NCC(=O)c1ccc(Cl)cc1] | (1S,2S,5R)-N-[2-(4- chlorophenyl)-2-oxo- ethyl]-1-hydroxy-2- isopropyl-5-methyl- cyclohexanecarboxamide | 2-amino-1-(4- chlorophenyl) ethanone | G |
| 135 | 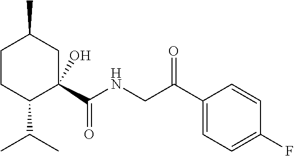 [SMILES: CC(C)[C@@H]1CC[C@@H](C)C[C@@]1(O)C(=O)NCC(=O)c1ccc(F)cc1] | (1S,2S,5R)-N-[2-(4- fluorophenyl)-2-oxo- ethyl]-1-hydroxy-2- isopropyl-5-methyl- cyclohexanecarboxamide | 2-amino-1-(4- fluorophenyl) ethanone | G |
| 136 | 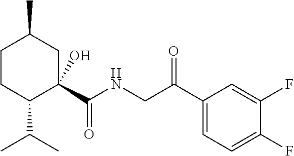 [SMILES: CC(C)[C@@H]1CC[C@@H](C)C[C@@]1(O)C(=O)NCC(=O)c1ccc(F)c(F)c1] | (1S,2S,5R)-N-[2-(3,4- difluorophenyl)-2-oxo- ethyl]-1-hydroxy-2- isopropyl-5-methyl- cyclohexanecarboxamide | 2-amino-1-(3,4- difluorophenyl) ethanone | G |
| 137 | 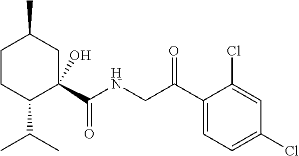 [SMILES: CC(C)[C@@H]1CC[C@@H](C)C[C@@]1(O)C(=O)NCC(=O)c1ccc(Cl)cc1Cl] | (1S,2S,5R)-N-[2-(2,4- dichlorophenyl)-2-oxo- ethyl]-1-hydroxy-2- isopropyl-5-methyl- cyclohexanecarboxamide | 2-amino-1-(2,4- dichlorophenyl) ethanone | G |
| 138 | 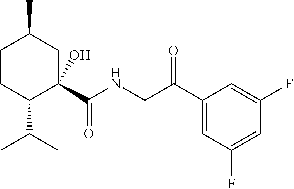 [SMILES: CC(C)[C@@H]1CC[C@@H](C)C[C@@]1(O)C(=O)NCC(=O)c1cc(F)cc(F)c1] | (1S,2S,5R)-N-[2-(3,5- difluorophenyl)-2-oxo- ethyl]-1-hydroxy-2- isopropyl-5-methyl- cyclohexanecarboxamide | 2-amino-1-(3,5- difluorophenyl) ethanone | G |
| 139 | 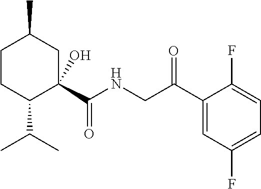 [SMILES: CC(C)[C@@H]1CC[C@@H](C)C[C@@]1(O)C(=O)NCC(=O)c1cc(F)ccc1F] | (1S,2S,5R)-N-[2-(2,5- difluorophenyl)-2-oxo- ethyl]-1-hydroxy-2- isopropyl-5-methyl- cyclohexanecarboxamide | 2-amino-1-(2,5- difluorophenyl) ethanone | G |
| 140 | 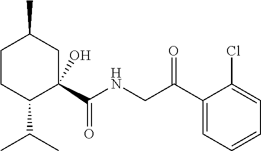 [SMILES: CC(C)[C@@H]1CC[C@@H](C)C[C@@]1(O)C(=O)NCC(=O)c1ccccc1Cl] | (1S,2S,5R)-N-[2-(2- chlorophenyl)-2-oxo- ethyl]-1-hydroxy-2- isopropyl-5-methyl- cyclohexanecarboxamide | 2-amino-1-(2- chlorophenyl) ethanone | G |
| 141 | 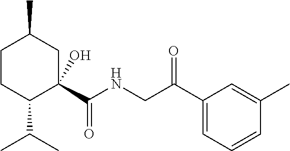 [SMILES: Cc1cccc(C(=O)CNC(=O)[C@]2(O)C[C@H](C)CC[C@H]2C(C)C)c1] | (1S,2S,5R)-1-hydroxy-2- isopropyl-5-methyl-N-[2- (m-tolyl)-2-oxo-ethyl] cyclohexanecarboxamide | 2-amino-1-(m- tolyl)ethanone | G |
| 142 | 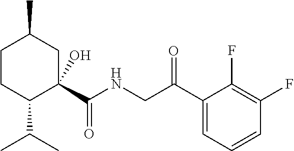 [SMILES: CC(C)[C@@H]1CC[C@@H](C)C[C@@]1(O)C(=O)NCC(=O)c1cccc(F)c1F] | (1S,2S,5R)-N-[2-(2,3- difluorophenyl)-2-oxo- ethyl]-1-hydroxy-2- isopropyl-5-methyl- cyclohexanecarboxamide | 2-amino-1-(2,3- difluorophenyl) ethanone | G |
| 143 | 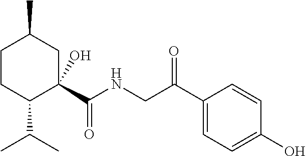 [SMILES: CC(C)[C@@H]1CC[C@@H](C)C[C@@]1(O)C(=O)NCC(=O)c1ccc(O)cc1] | (1S,2S,5R)-1-hydroxy- N-[2-(4-hydroxyphenyl)- 2-oxo-ethyl]-2-isopropyl- 5-methyl- cyclohexanecarboxamide | 2-amino-1-(4- hydroxyphenyl) ethanone | G |
| 144 | 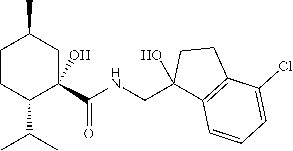 [SMILES: CC(C)[C@@H]1CC[C@@H](C)C[C@@]1(O)C(=O)NCC1(O)CCc2c(Cl)cccc21] | (1S,2S,5R)-N-[(4-chloro- 1-hydroxy-indan-1- yl)methyl]-1-hydroxy-2- isopropyl-5-methyl- cyclohexanecarboxamide | 1-(aminomethyl)- 4-chloro-indan-1- ol | G |
| 145 | 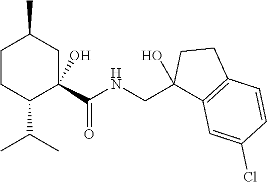 [SMILES: CC(C)[C@@H]1CC[C@@H](C)C[C@@]1(O)C(=O)NCC1(O)CCc2ccc(Cl)cc21] | (1S,2S,5R)-N-[(6-chloro- 1-hydroxy-indan-1- yl)methyl]-1-hydroxy-2- isopropyl-5-methyl- cyclohexanecarboxamide | 1-(aminomethyl)- 6-chloro-indan-1- ol | G |
| 146 | 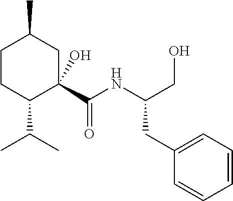 [SMILES: CC(C)[C@@H]1CC[C@@H](C)C[C@@]1(O)C(=O)N[C@H](CO)Cc1ccccc1] | (1S,2S,5R)-N-[(1S)-1- benzyl-2-hydroxy-ethyl]- 1-hydroxy-2-isopropyl-5- methyl- cyclohexanecarboxamide | (2S)-2-amino-3- phenyl-propan-1-ol | G |
| 147 | 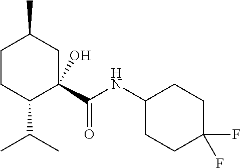 [SMILES: CC(C)[C@@H]1CC[C@@H](C)C[C@@]1(O)C(=O)NC1CCC(F)(F)CC1] | (1S,2S,5R)-N-(4,4- difluorocyclohexyl)-1- hydroxy-2-isopropyl-5- methyl- cyclohexanecarboxamide | 4,4-difluorocyclo- hexanamine | G |
| 148 | 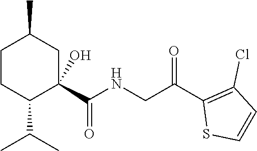 [SMILES: CC(C)[C@@H]1CC[C@@H](C)C[C@@]1(O)C(=O)NCC(=O)c1sccc1Cl] | (1S,2S,5R)-N-[2-(3- chloro-2-thienyl)-2-oxo- ethyl]-1-hydroxy-2- isopropyl-5-methyl- cyclohexanecarboxamide | 2-amino-1-(3- chloro-2- thienyl)ethanone | G |
| 149 | 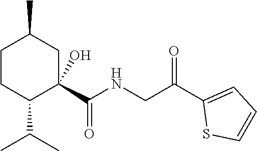 [SMILES: CC(C)[C@@H]1CC[C@@H](C)C[C@@]1(O)C(=O)NCC(=O)c1cccs1] | (1S,2S,5R)-1-hydroxy-2- isopropyl-5-methyl-N-[2- oxo-2-(2-thienyl)ethyl] cyclohexanecarboxamide | 2-amino-1-(2- thienyl)ethanone | G |
| 150 | 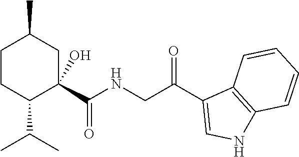 [SMILES: CC(C)[C@@H]1CC[C@@H](C)C[C@@]1(O)C(=O)NCC(=O)c1c[nH]c2ccccc12] | (1S,2S,5R)-1-hydroxy- N-[2-(1H-indol-3-yl)-2- oxo-ethyl]-2-isopropyl-5- methyl- cyclohexanecarboxamide | 2-amino-1-(1H- indol-3- yl)ethanone | G |
| 151 | 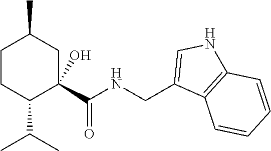 [SMILES: CC(C)[C@@H]1CC[C@@H](C)C[C@@]1(O)C(=O)NCc1c[nH]c2ccccc12] | (1S,2S,5R)-1-hydroxy- N-[(1R)-3-hydroxy-1-(2- thienylmethyl)propyl]-2- isopropyl-5-methyl- cyclohexanecarboxamide | (3R)-3-amino-4- (2-thienyl)butan-1- ol | G |
| 155 | 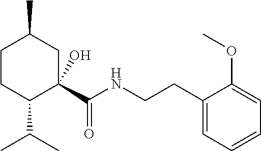 [SMILES: COc1ccccc1CCNC(=O)[C@]1(O)C[C@H](C)CC[C@H]1C(C)C] | (1S,2S,5R)-1-hydroxy-2- isopropyl-N-[2-(2- methoxyphenyl)ethyl]-5- methyl- cyclohexanecarboxamide | 2-(2- methoxyphenyl) ethanamine | G |
| 156 | 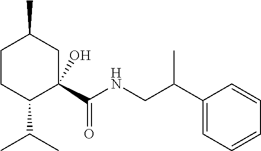 [SMILES: CC(CNC(=O)[C@]1(O)C[C@H](C)CC[C@H]1C(C)C)c1ccccc1] | (1S,2S,5R)-1-hydroxy-2- isopropyl-5-methyl-N-(2- phenylpropyl) cyclohexanecarboxamide | 2-phenylpropan-1- amine | G |
| 157 | 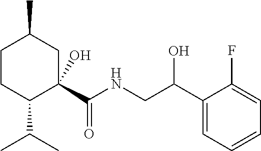 [SMILES: CC(C)[C@@H]1CC[C@@H](C)C[C@@]1(O)C(=O)NCC(O)c1ccccc1F] | (1S,2S,5R)-N-[2-(2- fluorophenyl)-2- hydroxy-ethyl]-1- hydroxy-2-isopropyl-5- methyl- cyclohexanecarboxamide | 2-amino-1-(2- fluorophenyl) ethanol | G |
| 158 | 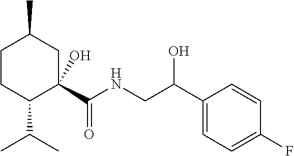 [SMILES: CC(C)[C@@H]1CC[C@@H](C)C[C@@]1(O)C(=O)NCC(O)c1ccc(F)cc1] | (1S,2S,5R)-N-[2-(4- fluorophenyl)-2- hydroxy-ethyl]-1- hydroxy-2-isopropyl-5- methyl- cyclohexanecarboxamide | 2-amino-1-(4- fluorophenyl) ethanol | G |
| 159 | 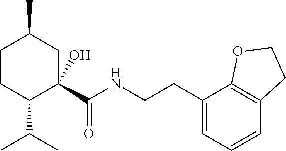 [SMILES: CC(C)[C@@H]1CC[C@@H](C)C[C@@]1(O)C(=O)NCCc1cccc2c1OCC2] | (1S,2S,5R)-N-[2-(2,3- dihydrobenzofuran-7- yl)ethyl]-1-hydroxy-2- isopropyl-5-methyl- cyclohexanecarboxamide | 2-(2,3- dihydrobenzo- furan-7- yl)ethanamine | G |
| 160 | 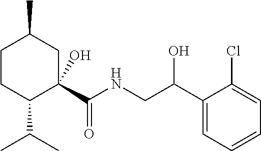 [SMILES: CC(C)[C@@H]1CC[C@@H](C)C[C@@]1(O)C(=O)NCC(O)c1ccccc1Cl] | (1S,2S,5R)-N-[2-(2- chlorophenyl)-2- hydroxy-ethyl]-1- hydroxy-2-isopropyl-5- methyl- cyclohexanecarboxamide | 2-amino-1-(2- chlorophenyl) ethanol | G |
| 161 | 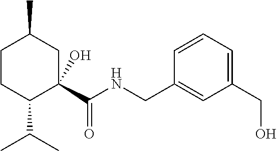 [SMILES: CC(C)[C@@H]1CC[C@@H](C)C[C@@]1(O)C(=O)NCc1cccc(CO)c1] | (1S,2S,5R)-1-hydroxy- N-[[3- (hydroxymethyl)phenyl] methyl]-2-isopropyl-5- methyl- cyclohexanecarboxamide | [3-(aminomethyl) phenyl]methanol | G |
| 162 | 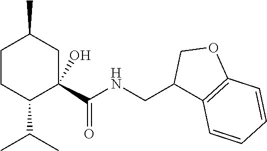 [SMILES: CC(C)[C@@H]1CC[C@@H](C)C[C@@]1(O)C(=O)NCC1COc2ccccc21] | (1S,2S,5R)-N-(2,3- dihydrobenzofuran-3- ylmethyl)-1-hydroxy-2- isopropyl-5-methyl- cyclohexanecarboxamide | 2,3-dihydrobenzo- furan-3- ylmethanamine | G |
| 163 | 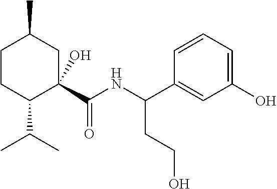 [SMILES: CC(C)[C@@H]1CC[C@@H](C)C[C@@]1(O)C(=O)NC(CCO)c1cccc(O)c1] | (1S,2S,5R)-1-hydroxy- N-[3-hydroxy-1-(3- hydroxyphenyl)propyl]- 2-isopropyl-5-methyl- cyclohexanecarboxamide | 3-(1-amino-3- hydroxy- propyl)phenol | G |
| 164 | 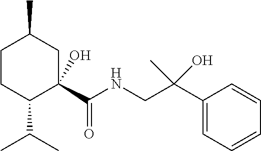 [SMILES: CC(C)[C@@H]1CC[C@@H](C)C[C@@]1(O)C(=O)NCC(C)(O)c1ccccc1] | (1S,2S,5R)-1-hydroxy- N-(2-hydroxy-2-phenyl- propyl)-2-isopropyl-5- methyl- cyclohexanecarboxamide | 1-amino-2-phenyl- propan-2-ol | G |
| 165 | 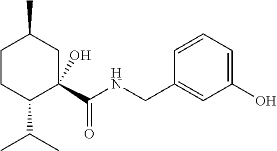 [SMILES: CC(C)[C@@H]1CC[C@@H](C)C[C@@]1(O)C(=O)NCc1cccc(O)c1] | (1S,2S,5R)-1-hydroxy- N-[(3- hydroxyphenyl)methyl]- 2-isopropyl-5-methyl- cyclohexanecarboxamide | 3-(aminomethyl) phenol | G |
| 166 | 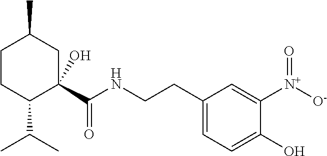 [SMILES: CC(C)[C@@H]1CC[C@@H](C)C[C@@]1(O)C(=O)NCCc1ccc(O)c([N+](=O)[O-])c1] | (1S,2S,5R)-1-hydroxy- N-[2-(4-hydroxy-3-nitro- phenyl)ethyl]-2- isopropyl-5-methyl- cyclohexanecarboxamide | 4-(2-aminoethyl)- 2-nitro-phenol | G |
| 167 | 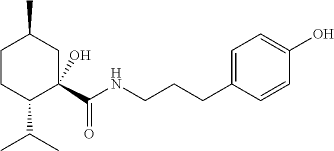 [SMILES: CC(C)[C@@H]1CC[C@@H](C)C[C@@]1(O)C(=O)NCCCc1ccc(O)cc1] | (1S,2S,5R)-1-hydroxy- N-[3-(4- hydroxyphenyl)propyl]- 2-isopropyl-5-methyl- cyclohexanecarboxamide | 4-(3-aminopropyl) phenol | G |
| 168 | 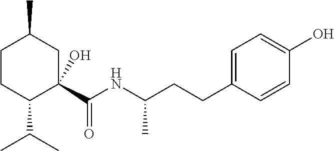 [SMILES: CC(C)[C@@H]1CC[C@@H](C)C[C@@]1(O)C(=O)N[C@@H](C)CCc1ccc(O)cc1] | (1S,2S,5R)-1-hydroxy- N-[(1S)-3-(4- hydroxyphenyl)-1- methyl-propyl]-2- isopropyl-5-methyl- cyclohexanecarboxamide | 4-[(3S)-3- aminobutyl]phenol | G |
| 169 | 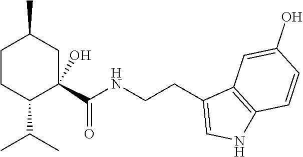 [SMILES: CC(C)[C@@H]1CC[C@@H](C)C[C@@]1(O)C(=O)NCCc1c[nH]c2ccc(O)cc12] | (1S,2S,5R)-1-hydroxy- N-[2-(5-hydroxy-1H- indol-3-yl)ethyl]-2- isopropyl-5-methyl- cyclohexanecarboxamide | 3-(2-aminoethyl)- 1H-indol-5-ol | G |
| 170 | 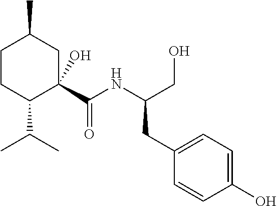 [SMILES: CC(C)[C@@H]1CC[C@@H](C)C[C@@]1(O)C(=O)N[C@@H](CO)Cc1ccc(O)cc1] | (1S,2S,5R)-1-hydroxy- N-[(1R)-1- (hydroxymethyl)-2-(4- hydroxyphenyl)ethyl]-2- isopropyl-5-methyl- cyclohexanecarboxamide | 4-[(2R)-2-amino- 3-hydroxy- propyl]phenol | G |
| 171 | 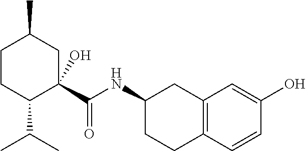 [SMILES: CC(C)[C@@H]1CC[C@@H](C)C[C@@]1(O)C(=O)N[C@@H]1CCc2ccc(O)cc2C1] | (1S,2S,5R)-1-hydroxy- N-[(2R)-7- hydroxytetralin-2-yl]-2- isopropyl-5-methyl- cyclohexanecarboxamide | (3R)-3- aminotetralin-6-ol | G |
| 172 | 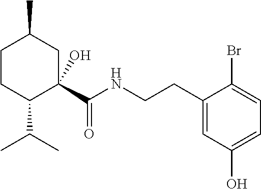 [SMILES: CC(C)[C@@H]1CC[C@@H](C)C[C@@]1(O)C(=O)NCCc1cc(O)ccc1Br] | (1S,2S,5R)-N-[2-(2- bromo-5-hydroxy- phenyl)ethyl]-1-hydroxy- 2-isopropyl-5-methyl- cyclohexanecarboxamide | 3-(2-aminoethyl)- 4-bromo-phenol | G |
| 173 | 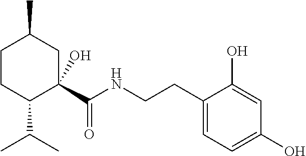 [SMILES: CC(C)[C@@H]1CC[C@@H](C)C[C@@]1(O)C(=O)NCCc1ccc(O)cc1O] | (1S,2S,5R)-N-[2-(2,4- dihydroxyphenyl)ethyl]- 1-hydroxy-2-isopropyl-5- methyl- cyclohexanecarboxamide | 4-(2-aminoethyl) benzene-1,3-diol | G |
| 174 | 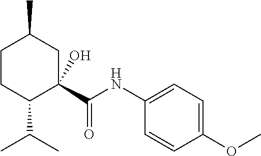 [SMILES: COc1ccc(NC(=O)[C@]2(O)C[C@H](C)CC[C@H]2C(C)C)cc1] | (1S,2S,5R)-1-hydroxy- N-(4-methoxyphenyl)-5- methyl-2-propan-2- ylcyclohexane-1- carboxamide | 4-Methoxyphenyl- amine | G |

### Example 152

### (1S,2S,5R)-1-hydroxy-2-isopropyl-N-[(5-methoxyindan-1-yl)methyl]-5-methyl-cyclohexanecarboxamide

Solution A (2 ml, 0.2 mmol) was added to (5-methoxyindan-1-yl)methanamine (0.2 mmol, 1.0eq) at room temperature and the resulting reaction mixture was then heated at 80° C. btw 150 and 210 min. The solvent was removed in vacuo and the residue was dissolved in 2 ml of a 9:1 DMF/TFA mixture (9/1, 2 ml). The resulting crude solution was subjected to RP-HPLC chromatography under conditions F.

Examples, 153 and 154, were synthesized and purified under same experimental conditions as for example 152 as shown in Table 3.

TABLE 3

| | | Compound | | Purification |
|---|---|---|---|---|
| Example | Structure | Name | Amine used | Method |
| 152 | 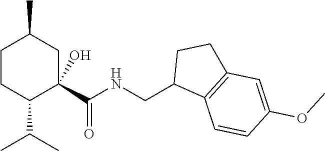 [SMILES: COc1ccc2c(c1)CCC2CNC(=O)[C@]1(O)C[C@H](C)CC[C@H]1C(C)C] | (1S,2S,5R)-1-hydroxy-2- isopropyl-N-[(5-methoxyindan-1- yl)methyl]-5-methyl- cyclohexanecarboxamide | (5-methoxyindan-1- yl)methanamine | H |
| 153 | 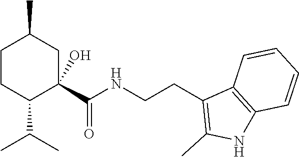 [SMILES: Cc1[nH]c2ccccc2c1CCNC(=O)[C@]1(O)C[C@H](C)CC[C@H]1C(C)C] | (1S,2S,5R)-1-hydroxy-2- isopropyl-5-methyl-N-[2-(2- methyl-1H-indol-3- yl)ethyl]cyclohexanecarboxamide | 2-(2-methyl-1H- indol-3- yl)ethanamine | H |
| 154 | 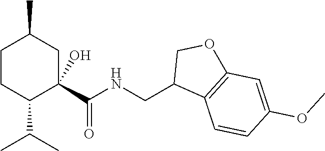 [SMILES: COc1ccc2c(c1)OCC2CNC(=O)[C@]1(O)C[C@H](C)CC[C@H]1C(C)C] | (1S,2S,5R)-1-hydroxy-2- isopropyl-N-[(6-methoxy-2,3- dihydrobenzofuran-3-yl)methyl]- 5-methyl- cyclohexanecarboxamide | (6-methoxy-2,3- dihydrobenzofuran- 3-yl)methanamine | H |

Examples 113 to 174, were characterized by 1H NMR and LC-MS analysis as shown below in Table 4:

TABLE 4

| | | 1H NMR spectrum (δ ppm, DMSO- | LCMS | |
|---|---|---|---|---|
| Example | Compound Name | d₆) | Method | [M + H]+ |
| 113 | (1S,2S,5R)-N-[2-(3,4- | (400.23 MHz, DMSO-d₆) 7.63 (br t, | C | 364 |
| | dimethoxyphenyl)ethyl]-1- | J = 5.69, 5.69 Hz, 1 H), 6.84 (d, | | |
| | hydroxy-2-isopropyl-5- | J = 8.15 Hz, 1 H), 6.80 (d, J = 1.86 | | |
| | methyl- | Hz, 1 H), 6.71 (dd, J = 8.19, 1.96 Hz, | | |
| | cyclohexanecarboxamide | 1 H), 4.74 (br s), 3.68-3.77 (m, 6 | | |
| | | H), 3.42 (br u), 3.39-3.33 (m), 3.33- | | |
| | | 3.22 (m), 2.73-2.60 (m, 2 H), 2.50 | | |
| | | (u), 2.08 (u), 2.07 (u), 1.74-1.62 (m, | | |
| | | 2 H), 1.54-1.30 (m, 6 H), 0.92-0.70 | | |
| | | (m) | | |
| 114 | (1S,2S,5R)-1-hydroxy-N- | (400.23 MHz, DMSO-d₆) 8.67 (br | C | 350 |
| | [2-(4-hydroxy-3-methoxy- | s), 7.61 (br t, J = 5.62, 5.62 Hz, 1 H), | | |
| | phenyl)ethyl]-2-isopropyl- | 6.75 (d, J = 1.83 Hz, 1 H), 6.67 (d, | | |
| | 5-methyl- | J = 7.95 Hz, 1 H), 6.58 (dd, J = 7.95, | | |
| | cyclohexanecarboxamide | 1.83 Hz, 1 H), 4.75 (br s), 3.75 (s, 3 | | |
| | | H), 3.42 (br u), 3.38-3.31 (m), 3.31- | | |
| | | 3.20 (m), 2.67-2.57 (m, 2 H), 2.50 | | |
| | | (u), 2.09 (u), 1.74-1.62 (m, 2 H), | | |
| | | 1.54-1.32 (m, 6 H), 0.89-0.72 (m) | | |
| 115 | (1S,2S,5R)-N-[2-(2,3- | (400.23 MHz, DMSO-d₆) 7.72 (br t, | C | 364 |
| | dimethoxyphenyl)ethyl]-1- | J = 5.75, 5.75 Hz, 1 H), 6.99-6.94 | | |
| | hydroxy-2-isopropyl-5- | (m, 1 H), 6.90 (dd, J = 8.29, 1.55 Hz, | | |
| | methyl- | 1 H), 6.76 (dd, J = 7.52, 1.53 Hz, 1 | | |
| | cyclohexanecarboxamide | H), 4.72 (br s), 3.80-3.70 (m, 6 H), | | |
| | | 3.41 (br u), 3.37-3.21 (m), 2.78- | | |
| | | 2.65 (m, 2 H), 2.50 (u), 1.74-1.62 | | |
| | | (m, 2 H), 1.53-1.30 (m, 6 H), 0.93- | | |
| | | 0.71 (m, 10 H) | | |
| 116 | (1S,2S,5R)-1-hydroxy-N- | (400.23 MHz, DMSO-d₆) 7.64-7.53 | C | 320 |
| | [2-(2- | (m, 1 H), 7.36-7.20 (m, 5 H), 5.63- | | |
| | hydroxyphenyl)ethyl]-2- | 5.43 (br m), 4.86 (br s, 1 H), 4.67- | | |
| | isopropyl-5-methyl- | 4.60 (m, 1 H), 3.48-3.43 (m), 3.32 | | |
| | cyclohexanecarboxamide | (u), 3.20-3.13 (m), 2.50 (u), 1.74- | | |
| | | 1.62 (m, 2 H), 1.55-1.27 (m), 0.91- | | |
| | | 0.67 (m) | | |
| 117 | (1S,2S,5R)-1-hydroxy-N- | (400.23 MHz, DMSO-d₆) 9.13 (br | C | 320 |
| | [2-(4- | s, 1 H), 7.62 (br t, J = 5.93, 5.93 Hz, | | |
| | hydroxyphenyl)ethyl]-2- | 1 H), 7.00-6.96 (m, 2 H), 6.68-6.64 | | |
| | isopropyl-5-methyl- | (m, 2 H), 4.72 (br s, 1 H), 3.33 (br | | |
| | cyclohexanecarboxamide | u), 3.30-3.17 (m), 2.64-2.56 (m), | | |
| | | 2.50 (u), 2.09 (u), 1.74-1.63 (m, 2 | | |
| | | H), 1.54-1.30 (m, 6 H), 0.91-0.72 | | |
| | | (m) | | |
| 118 | (1S,2S,5R)-1-hydroxy-2- | (400.23 MHz, DMSO-d₆) 7.78-7.70 | C | 383 |
| | isopropyl-5-methyl-N-[2- | (m, 3 H), 7.60 (u), 7.42-7.36 (m, 2 | | |
| | (4- | H), 7.27 (s, 2 H), 4.74 (br s), 3.47- | | |
| | sulfamoylphenyl)ethyl]cyc | 3.40 (m), 3.39 (br u) 3.36-3.28 (m), | | |
| | lohexanecarboxamide | 2.87-2.76 (m, 2 H), 2.50 (u), 2.08 | | |
| | | (u), 1.75-1.63 (m, 2 H), 1.53-1.30 | | |
| | | (m, 6 H), 0.92-0.70 (m, 10 H) | | |
| 119 | (1S,2S,5R)-1-hydroxy-2- | (400.23 MHz, DMSO-d₆) 8.72-8.68 | C | 305 |
| | isopropyl-5-methyl-N-[2- | (m, 2 H), 7.85 (br t, J = 5.81, 5.81 | | |
| | (4- | Hz, 1 H), 7.72-7.68 (m, 2 H), 4.75 | | |
| | pyridyl)ethyl]cyclohexane | (br s, 1 H), 3.57 (br u), 3.58-3.48 | | |
| | carboxamide | (m), 3.48-3.39 (m), 3.02-2.94 (m, 2 | | |
| | | H), 2.50 (u), 1.73-1.62 (m, 2 H), | | |
| | | 1.47-1.22 (m, 6 H), 0.89-0.76 (m, 4 | | |
| | | H), 0.73 (d, J = 6.74 Hz, 3 H), 0.67 | | |
| | | (d, J = 6.87 Hz, 3 H) | | |
| 120 | (1S,2S,5R)-1-hydroxy-2- | (400.23 MHz, DMSO-d₆) 7.86 (br | C | 320 |
| | isopropyl-5-methyl-N-(2- | t, J = 5.81, 5.81 Hz, 1 H), 7.32-7.24 | | |
| | phenoxyethyl)cyclohexan | (m, 2 H), 6.95-6.89 (m, 3 H), 4.85 | | |
| | ecarboxamide | (s, 1 H), 4.04-3.95 (m, 2 H), 3.55- | | |
| | | 3.40 (m), 3.31 (u), 2.50 (u), 1.76- | | |
| | | 1.33 (m, 8H), 0.93-0.69 (m, 10 H) | | |
| 121 | (1S,2S,5R)-1-hydroxy-2- | (400.23 MHz, DMSO-d₆) 8.05- | C | 348 |
| | isopropyl-N-[2-(4- | 7.95 (m, 3 H), 7.08-7.02 (m, 2 H), | | |
| | methoxyphenyl)-2-oxo- | 4.99 (s, 1 H), 4.59 (d, J = 5.38 Hz, 2 | | |
| | ethyl]-5-methyl- | H), 3.85 (s, 3 H), 3.30 (u), 2.50 (u), | | |
| | cyclohexanecarboxamide | 1.80-1.63 (m, 3 H), 1.58-1.35 (m, 5 | | |
| | | H), 0.94-0.74 (m, 10 H) | | |
| 122 | (1S,2S,5R)-1-hydroxy-N- | — | C | 364 |
| | [(1S,2S)-2-hydroxy-1- | | | |
| | (methoxymethyl)-2- | | | |
| | phenyl-ethyl]-2-isopropyl- | | | |
| | 5-methyl- | | | |
| | cyclohexanecarboxamide | | | |
| 123 | (1S,2S,5R)-N-[2-(3,5- | (400.23 MHz, DMSO-d₆) 7.65 (br | C | 364 |
| | dimethoxyphenyl)ethyl]-1- | t, J = 6.01, 6.01 Hz, 1 H), 6.40-6.30 | | |
| | hydroxy-2-isopropyl-5- | (m, 3 H), 4.75 (br s), 3.71 (s, 6 H), | | |
| | methyl- | 3.45-3.36 (m), 3.33 (br u), 3.30- | | |
| | cyclohexanecarboxamide | 3.23 (m), 2.73-2.61 (m, 2 H), 2.50 | | |
| | | (u), 1.73-1.63 (m, 2 H), 1.53-1.29 | | |
| | | (m, 6 H), 0.92-0.71 (m, 10 H) | | |
| 124 | (1S,2S,5R)-1-hydroxy-2- | (400.23 MHz, DMSO-d₆) 8.69- | C | 305 |
| | isopropyl-5-methyl-N-[2- | 8.64 (m, 2 H), 8.18-8.13 (m, 1 H), | | |
| | (3- | 7.84 (br t, J = 5.87, 5.87 Hz, 1 H), | | |
| | pyridyl)ethyl]cyclohexane | 7.74 (dd, J = 7.82, 5.38 Hz, 1 H), | | |
| | carboxamide | 4.64 (br s), 3.64 (br u), 3.55-3.45 | | |
| | | (m), 3.45-3.36 (m), 2.97-2.87 (m, | | |
| | | 2H), 2.50 (u), 1.73-1.62 (m, 2 H), | | |
| | | 1.46-1.21 (m, 6 H), 0.89-0.65 (m, | | |
| | | 10 H) | | |
| 125 | (1S,2S,5R)-1-hydroxy-2- | (400.23 MHz, DMSO-d₆) 8.68- | C | 305 |
| | isopropyl-5-methyl-N-[2- | 8.63 (m, 2 H), 8.17-8.12 (m, 1 H), | | |
| | (2- | 7.83 (br t, J = 5.87, 5.87 Hz, 1 H), | | |
| | pyridyl)ethyl]cyclohexane | 7.74 (dd, J = 7.95, 5.38 Hz, 1 H), | | |
| | carboxamide | 4.74 (br s), 3.65 (br u), 3.55-3.45 | | |
| | | (m), 3.45-3.36 (m), 2.97-2.86 (m, | | |
| | | 2H), 2.50 (u), 1.73-1.62 (m, 2 H), | | |
| | | 1.46-1.21 (m, 6 H), 0.88-0.64 (m, | | |
| | | 10 H) | | |
| 126 | (1S,2S,5R)-1-hydroxy-N- | (400.23 MHz, DMSO-d₆) 8.77 (br | C | 350 |
| | [2-(3-hydroxy-4-methoxy- | s), 7.63 (br t, J = 5.62, 5.62 Hz, 1 H), | | |
| | phenyl)ethyl]-2-isopropyl- | 6.80 (d, J = 8.19 Hz, 1 H), 6.62 (d, | | |
| | 5-methyl- | J = 1.96 Hz, 1 H), 6.56 (dd, J = 8.07, | | |
| | cyclohexanecarboxamide | 1.96 Hz, 1 H), 4.70 (br s), 3.72 (s, 3 | | |
| | | H), 3.46 (br u), 3.37-3.27 (m), 3.27- | | |
| | | 3.16 (m), 2.62-2.53 (m), 2.50 (u), | | |
| | | 1.75-1.61 (m, 2 H), 1.54-1.29 (m, 6 | | |
| | | H), 0.92-0.70 (m) | | |
| 127 | (1S,2S,5R)-1-hydroxy-2- | (400.23 MHz, DMSO-d₆) 8.78 (br | C | 319 |
| | isopropyl-5-methyl-N-[2- | s), 7.63 (br t, J = 5.78, 5.78 Hz, 1 H), | | |
| | (3-methyl-2- | 6.80 (d, J = 8.13 Hz, 1 H), 6.62 (d, | | |
| | pyridyl)ethyl]cyclohexane | J = 2.00 Hz, 1 H), 6.56 (dd, J = 8.13, | | |
| | carboxamide | 2.00 Hz, 1 H), 4.71 (br s), 3.71 (s, 3 | | |
| | | H), 3.45 (br u), 3.37-3.27 (m), 3.27- | | |
| | | 3.16 (m), 2.61-2.54 (m), 2.50 (u), | | |
| | | 2.08 (u), 1.74-1.62 (m, 2 H), 1.54- | | |
| | | 1.30 (m, 6 H), 0.92-0.72 (m) | | |
| 128 | (1S,2S,5R)-N-[2-(2,5- | (400.23 MHz, DMSO-d₆) 7.65 (br | C | 364 |
| | dimethoxyphenyl)ethyl]-1- | t, J = 5.56, 5.56 Hz, 1 H), 6.88-6.84 | | |
| | hydroxy-2-isopropyl-5- | (m, 1 H), 6.75-6.71 (m, 2 H), 4.70 | | |
| | methyl- | (br s), 3.75-3.66 (m, 6 H), 3.40 (br | | |
| | cyclohexanecarboxamide | u), 3.36-3.22 (m), 2.77-2.62 (m, 2 | | |
| | | H), 2.50 (u), 1.73-1.62 (m, 2 H), | | |
| | | 1.53-1.29 (m, 6 H), 0.91-0.69 (m, | | |
| | | 10 H) | | |
| 129 | (1S,2S,5R)-N-(2-anilino- | (400.23 MHz, DMSO-d₆) 9.94 (s, | C | 333 |
| | 2-oxo-ethyl)-1-hydroxy-2- | 1 H), 7.98 (t, J = 5.56, 5.56 Hz, 1 H), | | |
| | isopropyl-5-methyl- | 7.56 (d, J = 7.58 Hz, 2 H), 7.34-7.27 | | |
| | cyclohexanecarboxamide | (m, 2 H), 7.07-7.02 (m, 1 H), 4.98 | | |
| | | (s, 1 H), 3.92 (d, J = 5.62 Hz, 2 H), | | |
| | | 3.31 (u), 2.50 (u), 1.79-1.34 (m, 8 | | |
| | | H), 0.94-0.74 (m, 10 H) | | |
| 130 | (1S,2S,5R)-1-hydroxy-N- | (400.23 MHz, DMSO-d₆) 8.05 (br | C | 380 |
| | [2-(4-hydroxy-3,5- | s), 7.61 (br t, J = 5.91, 5.91 Hz, 1 H), | | |
| | dimethoxy-phenyl)ethyl]- | 6.45 (s, 2 H), 4.75 (br s), 3.73 (s, 6 | | |
| | 2-isopropyl-5-methyl- | H), 3.47 (br u), 3.43-3.34 (m), 3.32- | | |
| | cyclohexanecarboxamide | 3.22 (m), 2.69-2.56 (m, 2 H), 2.50 | | |
| | | (u), 2.09 (u), 1.74-1.63 (m, 2 H), | | |
| | | 1.54-1.31 (m, 6 H), 0.92-0.71 (m, | | |
| | | 10 H) | | |
| 131 | (1S,2S,5R)-1-hydroxy-2- | (400.23 MHz, DMSO-d₆) 8.56- | C | 306 |
| | isopropyl-5-methyl-N-(2- | 8.52 (m, 2 H), 8.47 (d, J = 2.57 Hz, 1 | | |
| | pyrazin-2- | H), 7.82 (br t, J = 5.69, 5.69 Hz, 1 | | |
| | ylethyl)cyclohexanecarbo | H), 4.73 (s, 1 H), 3.60-3.42 (m, 2 | | |
| | xamide | H), 3.31 (u), 2.96 (t, J = 6.79, 6.79 | | |
| | | Hz, 2 H), 2.50 (u), 1.72-1.62 (m, 2 | | |
| | | H), 1.48-1.27 (m, 6 H), 0.90-0.67 | | |
| | | (m, 10 H) | | |
| 132 | benzyl 2-[[(1S,2S,5R)-1- | (400.23 MHz, DMSO-d₆) 8.13 (t, | C | 348 |
| | hydroxy-2-isopropyl-5- | J = 6.05, 6.05 Hz, 1 H), 7.40-7.30 | | |
| | methyl- | (m, 5 H), 5.16-5.07 (m, 2 H), 4.89 | | |
| | cyclohexanecarbonyl]ami | (s, 1 H), 3.97-3.84 (m, 2 H), 3.31 | | |
| | no]acetate | (u), 2.50 (u), 2.09 (u), 1.76-1.63 (m, | | |
| | | 3 H), 1.55-1.30 (m, 5 H), 0.92-0.71 | | |
| | | (m, 10 H) | | |
| 133 | (1S,2S,5R)-1-hydroxy-2- | (400.23 MHz, DMSO-d₆) 7.81 (br | C | 383 |
| | isopropyl-5-methyl-N-[2- | t, J = 5.93, 5.93 Hz, 1 H), 7.70-7.64 | | |
| | (3- | (m, 2 H), 7.50-7.41 (m, 2 H), 7.28 | | |
| | sulfamoylphenyl)ethyl]cyc | (s, 2 H), 4.74 (br s), 3.39 (br u), | | |
| | lohexanecarboxamide | 3.35-3.30 (m), 2.86-2.76 (m, 2 H), | | |
| | | 2.50 (u), 2.09 (u), 1.75-1.63 (m, 2 | | |
| | | H), 1.54-1.31 (m, 6 H), 0.92-0.72 | | |
| | | (m, 10 H) | | |
| 134 | (1S,2S,5R)-N-[2-(4- | (600.05 MHz, DMSO-d₆) 8.09 (t, | C | 352 |
| | chlorophenyl)-2-oxo- | J = 5.41, 5.41 Hz, 1 H), 8.01-7.98 | | |
| | ethyl]-1-hydroxy-2- | (m, 2 H), 7.61-7.58 (m, 2 H), 4.98 | | |
| | isopropyl-5-methyl- | (s, 1 H), 4.63-4.54 (m, 2 H), 3.31 | | |
| | cyclohexanecarboxamide | (u), 2.50 (u), 1.77-1.65 (m, 2 H), | | |
| | | 1.63-1.58 (m, 1 H), 1.54-1.47 (m, 2 | | |
| | | H), 1.45-1.33 (m, 3 H), 0.92-0.79 | | |
| | | (m, 7 H), 0.75 (d, J = 6.79 Hz, 3 H) | | |
| 135 | (1S,2S,5R)-N-[2-(4- | (600.05 MHz, DMSO-d₆) 8.10- | C | 336 |
| | fluorophenyl)-2-oxo- | 8.06 (m, 3 H), 7.38-7.33 (m, 2 H), | | |
| | ethyl]-1-hydroxy-2- | 4.99 (s, 1 H), 4.64-4.56 (m, 2 H), | | |
| | isopropyl-5-methyl- | 3.31 (u), 2.50 (u), 1.78-1.61 (m, 3 | | |
| | cyclohexanecarboxamide | H), 1.55-1.48 (m, 2 H), 1.46-1.35 | | |
| | | (m, 3 H), 0.93-0.79 (m, 7 H), 0.76 | | |
| | | (d, J = 6.79 Hz, 3 H) | | |
| 136 | (1S,2S,5R)-N-[2-(3,4- | (600.05 MHz, DMSO-d₆) 8.12 (t, | C | 354 |
| | difluorophenyl)-2-oxo- | J = 5.41, 5.41 Hz, 1 H), 8.07-8.02 | | |
| | ethyl]-1-hydroxy-2- | (m, 1 H), 7.90-7.86 (m, 1 H), 7.63- | | |
| | isopropyl-5-methyl- | 7.58 (m, 1 H), 4.98 (br s, 1 H), | | |
| | cyclohexanecarboxamide | 4.62-4.53 (m, 2 H), 3.31 (u), 2.50 | | |
| | | (u), 1.77-1.66 (m, 2 H), 1.62-1.56 | | |
| | | (m, 1 H), 1.54-1.47 (m, 2 H), 1.45- | | |
| | | 1.33 (m, 3 H), 0.92-0.79 (m, 7 H), | | |
| | | 0.74 (d, J = 6.97 Hz, 3 H) | | |
| 137 | (1S,2S,5R)-N-[2-(2,4- | (600.05 MHz, DMSO-d₆) 8.24 (t, | C | 386 |
| | dichlorophenyl)-2-oxo- | J = 5.59, 5.59 Hz, 1 H), 7.72-7.67 | | |
| | ethyl]-1-hydroxy-2- | (m, 2 H), 7.53 (dd, J = 8.44, 2.02 Hz, | | |
| | isopropyl-5-methyl- | 1 H), 4.90 (s, 1 H), 4.36-4.27 (m, 2 | | |
| | cyclohexanecarboxamide | H), 3.31 (u), 2.50 (u), 2.09 (u), | | |
| | | 1.72-1.62 (m, 2 H), 1.46 (br s, 1 H), | | |
| | | 1.47-1.42 (m, 1 H), 1.41-1.27 (m, 3 | | |
| | | H), 1.25-1.19 (m, 2 H), 0.88-0.76 | | |
| | | (m, 4 H), 0.74 (d, J = 6.79 Hz, 3 H), | | |
| | | 0.67 (d, J = 6.97 Hz, 3 H) | | |
| 138 | (1S,2S,5R)-N-[2-(3,5- | (600.05 MHz, DMSO-d₆) 8.16 (t, | C | 354 |
| | difluorophenyl)-2-oxo- | J = 5.41, 5.41 Hz, 1 H), 7.70-7.65 | | |
| | ethyl]-1-hydroxy-2- | (m, 2 H), 7.61-7.56 (m, 1 H), 4.97 | | |
| | isopropyl-5-methyl- | (br s, 1 H), 4.61-4.51 (m, 2 H), 3.31 | | |
| | cyclohexanecarboxamide | (u), 2.50 (u), 2.09 (u), 1.77-1.65 (m, | | |
| | | 2 H), 1.59-1.46 (m, 3 H), 1.44-1.32 | | |
| | | (m, 3 H), 0.91-0.78 (m, 7 H), 0.73 | | |
| | | (d, J = 6.79 Hz, 3 H) | | |
| 139 | (1S,2S,5R)-N-[2-(2,5- | (600.05 MHz, DMSO-d₆) 8.14 (br | C | 354 |
| | difluorophenyl)-2-oxo- | t, J = 5.41, 5.41 Hz, 1 H), 7.63-7.58 | | |
| | ethyl]-1-hydroxy-2- | (m, 1 H), 7.58-7.52 (m, 1 H), 7.48- | | |
| | isopropyl-5-methyl- | 7.42 (m, 1 H), 4.98 (br s, 1 H), | | |
| | cyclohexanecarboxamide | 4.46-4.42 (m, 2 H), 3.29 (u), 2.50 | | |
| | | (u), 2.09 (u), 1.76-1.65 (m, 2 H), | | |
| | | 1.55-1.48 (m, 2 H), 1.48-1.30 (m, 4 | | |
| | | H), 0.92-0.78 (m, 7 H), 0.73 (d, | | |
| | | J = 6.79 Hz, 3 H) | | |
| 140 | (1S,2S,5R)-N-[2-(2- | (600.05 MHz, DMSO-d₆) 8.18 (t, | C | 352 |
| | chlorophenyl)-2-oxo- | J = 5.50, 5.50 Hz, 1 H), 7.70-7.67 | | |
| | ethyl]-1-hydroxy-2- | (m, 1 H), 7.54-7.50 (m, 2 H), 7.46- | | |
| | isopropyl-5-methyl- | 7.42 (m, 1 H), 4.91 (br s, 1 H), | | |
| | cyclohexanecarboxamide | 4.39-4.36 (m, 2 H), 3.40 (u), 2.50 | | |
| | | (u), 2.08 (u), 1.74-1.64 (m, 2 H), | | |
| | | 1.51-1.46 (m, 1 H), 1.45-1.32 (m, 4 | | |
| | | H), 1.30-1.25 (m, 1 H), 0.89-0.74 | | |
| | | (m, 7 H), 0.70 (d, J = 6.79 Hz, 3 H) | | |
| 141 | (1S,2S,5R)-1-hydroxy-2- | (600.05 MHz, DMSO-d₆) 8.06 (t, | C | 332 |
| | isopropyl-5-methyl-N-[2- | J = 5.41, 5.41 Hz, 1 H), 7.81 (s, 1 | | |
| | (m-tolyl)-2-oxo- | H), 7.79 (d, J = 7.70 Hz, 1 H), 7.47 | | |
| | ethyl]cyclohexanecarboxa | (d, J = 7.52 Hz, 1 H), 7.42 (t, J = 7.61, | | |
| | mide | 7.61 Hz, 1 H), 4.99 (br s, 1 H), | | |
| | | 4.65-4.56 (m, 2 H), 3.31 (u), 2.50 | | |
| | | (u), 2.41-2.35 (m, 3 H), 2.07 (u), | | |
| | | 1.78-1.63 (m, 3 H), 1.56-1.49 (m, 2 | | |
| | | H), 1.46-1.36 (m, 3H), 0.92-0.80 | | |
| | | (m, 7 H), 0.76 (d, J = 6.97 Hz, 3 H) | | |
| 142 | (1S,2S,5R)-N-[2-(2,3- | (600.05 MHz, DMSO-d₆) 8.23 (br | C | 354 |
| | difluorophenyl)-2-oxo- | t, J = 5.41, 5.41 Hz, 1 H), 7.63-7.57 | | |
| | ethyl]-1-hydroxy-2- | (m, 1 H), 7.22-7.17 (m, 2 H), 4.88 | | |
| | isopropyl-5-methyl- | (s, 1 H), 4.28-4.25 (m, 2 H), 3.31 | | |
| | cyclohexanecarboxamide | (u), 2.50 (u), 1.73-1.63 (m, 2 H), | | |
| | | 1.50-1.-45 (m, 1 H), 1.42-1.28 (m, | | |
| | | 4 H), 1.25-1.19 (m, 1 H), 0.88-0.73 | | |
| | | (m, 7 H), 0.70 (d, J = 6.79 Hz, 3 H) | | |
| 143 | (1S,2S,5R)-1-hydroxy-N- | (600.05 MHz, DMSO-d₆) 12.47 | C | 334 |
| | [2-(4-hydroxyphenyl)-2- | (u), 10.39 (s, 1 H), 8.00 (t, J = 5.32, | | |
| | oxo-ethyl]-2-isopropyl-5- | 5.32 Hz, 1 H), 7.89-7.85 (m, 2 H), | | |
| | methyl- | 6.87-6.84 (m, 2 H), 4.99 (br s, 1 H), | | |
| | cyclohexanecarboxamide | 4.55 (d, J = 5.32 Hz, 2 H), 4.38 (u), | | |
| | | 3.31 (u), 2.50 (u), 1.78-1.64 (m), | | |
| | | 1.57-1.26 (m), 0.92-0.74 (m) | | |
| 144 | (1S,2S,5R)-N-[(4-chloro- | (600.05 MHz, DMSO-d₆) 7.61- | C | 378 |
| | 1-hydroxy-indan-1- | 7.55 (m, 1 H), 7.32-7.26 (m, 2 H), | | |
| | yl)methyl]-1-hydroxy-2- | 7.25-7.20 (m, 1 H), 5.63 (br s), 4.95 | | |
| | isopropyl-5-methyl- | (br s), 3.45-3.41 (m), 3.37-3.35 (m), | | |
| | cyclohexanecarboxamide | 3.34 (u), 3.32-3.28 (m), 2.94-2.87 | | |
| | | (m, 1 H), 2.78-2.71 (m, 1 H), 2.50 | | |
| | | (u), 2.26-2.21 (m, 1 H), 2.08 (u), | | |
| | | 2.00-1.95 (m, 1 H), 1.73-1.64 (m, 2 | | |
| | | H), 1.51-1.28 (m, 6 H), 0.88-0.75 | | |
| | | (m, 7 H), 0.73-0.69 (m, 3 H) | | |
| 145 | (1S,2S,5R)-N-[(6-chloro- | (600.05 MHz, DMSO-d₆) 7.62 (m, | C | 378 |
| | 1-hydroxy-indan-1- | 1 H), 7.31 (m, 1 H), 7.24 (m, 2 H), | | |
| | yl)methyl]-1-hydroxy-2- | 5.59 (br s), 5.00 (br s), 3.37 (u), | | |
| | isopropyl-5-methyl- | 3.36-3.33 (m), 2.88-2.82 (m, 1 H), | | |
| | cyclohexanecarboxamide | 2.76-2.68 (m, 1 H), 2.50 (u), 2.22- | | |
| | | 2.16 (m, 1 H), 2.08 (u), 2.00-1.92 | | |
| | | (m, 1 H), 1.76-1.64 (m, 2 H), 1.55- | | |
| | | 1.26 (m, 6 H), 0.91-0.77 (m, 7 H), | | |
| | | 0.72 (d, J = 6.90 Hz, 3 H) | | |
| 146 | (1S,2S,5R)-N-[(1S)-1- | (600.05 MHz, DMSO-d₆) 7.40- | C | 334 |
| | benzyl-2-hydroxy-ethyl]-1- | 7.36 (m, 1 H), 7.26-7.22 (m, 2 H), | | |
| | hydroxy-2-isopropyl-5- | 7.21-7.18 (m, 2 H), 7.18-7.14 (m, 1 | | |
| | methyl- | H), 4.72 (br s), 4.04-3.96 (m, 1 H), | | |
| | cyclohexanecarboxamide | 3.43 (u), 3.39-3.35 (m), 3.31-3.27 | | |
| | | (m), 2.89-2.85 (m, 1 H), 2.72-2.67 | | |
| | | (m, 1 H), 2.50 (u), 1.71-1.62 (m, 2 | | |
| | | H), 1.48-1.16 (m), 0.89-0.80 (m), | | |
| | | 0.78 (d, J = 6.45 Hz, 3 H), 0.69 (d, | | |
| | | J = 6.77 Hz, 3 H), 0.61 (d, J = 6.94 | | |
| | | Hz, 3 H) | | |
| 147 | (1S,2S,5R)-N-(4,4- | (600.05 MHz, DMSO-d₆) 7.54 (br | C | 318 |
| | difluorocyclohexyl)-1- | d, J = 7.89 Hz, 1 H), 4.73 (br s, 1 H), | | |
| | hydroxy-2-isopropyl-5- | 3.80-3.72 (m, 1 H), 3.33 (u), 2.50 | | |
| | methyl- | (u), 2.04-1.84 (m, 4 H), 1.78-1.65 | | |
| | cyclohexanecarboxamide | (m, 4 H), 1.63-1.49 (m, 4 H), 1.46- | | |
| | | 1.33 (m, 4 H), 0.92-0.76 (m, 10 H) | | |
| 148 | (1S,2S,5R)-N-[2-(3- | (600.05 MHz, DMSO-d₆) 8.10 (d, | C | 358 |
| | chloro-2-thienyl)-2-oxo- | J = 5.32 Hz, 1 H), 8.06 (br t, J = 5.50, | | |
| | ethyl]-1-hydroxy-2- | 5.50 Hz, 1 H), 7.29 (d, J = 5.14 Hz, 1 | | |
| | isopropyl-5-methyl- | H), 5.01 (s, 1 H), 4.57-4.54 (m, 2 | | |
| | cyclohexanecarboxamide | H), 3.30 (u), 2.50 (u), 2.07 (u), | | |
| | | 1.78-1.66 (m, 3 H), 1.56-1.50 (m, 2 | | |
| | | H), 1.47-1.35 (m, 3 H), 0.91-0.80 | | |
| | | (m, 7 H), 0.78 (d, J = 7.12 Hz, 3H) | | |
| 149 | (1S,2S,5R)-1-hydroxy-2- | (600.05 MHz, DMSO-d₆) 8.11- | C | 324 |
| | isopropyl-5-methyl-N-[2- | 8.01 (m, 3 H), 7.27-7.24 (m, 1 H), | | |
| | oxo-2-(2- | 4.97 (s, 1 H), 4.60-4.51 (m, 2 H), | | |
| | thienyl)ethyl]cyclohexane | 3.30 (u), 2.50 (u), 2.07 (u), 1.78- | | |
| | carboxamide | 1.65 (m, 3 H), 1.56-1.49 (m, 2 H), | | |
| | | 1.46-1.34 (m, 3 H), 0.92-0.73 (m, | | |
| | | 10 H) | | |
| 150 | (1S,2S,5R)-1-hydroxy-N- | (600.05 MHz, DMSO-d₆) 12.01 (br | C | 357 |
| | [2-(1H-indol-3-yl)-2-oxo- | s, 1 H), 8.45 (d, J = 3.12 Hz, 1 H), | | |
| | ethyl]-2-isopropyl-5- | 8.34 (u), 8.17-8.14 (m, 1 H), 8.05 | | |
| | methyl- | (t, J = 5.14, 5.14 Hz, 1 H), 7.49-7.46 | | |
| | cyclohexanecarboxamide | (m, 1 H), 7.24-7.17 (m, 2 H), 5.02 | | |
| | | (s, 1 H), 4.57-4.48 (m, 2 H), 3.30 | | |
| | | (u), 2.50 (u), 1.79-1.63 (m, 3 H), | | |
| | | 1.59-1.51 (m, 2 H), 1.47-1.28 (m, 3 | | |
| | | H), 0.93-0.81 (m, 7 H), 0.78 (d, | | |
| | | J = 6.97 Hz, 3 H) | | |
| 151 | (1S,2S,5R)-1-hydroxy-N- | (600.05 MHz, DMSO-d₆) 10.88 (br | C | 329 |
| | [(1R)-3-hydroxy-1-(2- | s, 1 H), 7.71 (br t, J = 5.50, 5.50 Hz, | | |
| | thienylmethyl)propyl]-2- | 1 H), 7.56-7.53 (m, 1 H), 7.35-7.33 | | |
| | isopropyl-5-methyl- | (m, 1 H), 7.23 (d, J = 2.20 Hz, 1 H), | | |
| | cyclohexanecarboxamide | 7.09-7.05 (m, 1 H), 6.98-6.94 (m, 1 | | |
| | | H), 4.79 (br s), 4.49-4.39 (m, 2 H), | | |
| | | 3.33 (u), 2.50 (u), 2.07 (u), 1.72- | | |
| | | 1.64 (m, 2 H), 1.60-1.54 (m, 2 H), | | |
| | | 1.46-1.33 (m), 0.92-0.73 (m) | | |
| 152 | (1S,2S,5R)-1-hydroxy-2- | (600.05 MHz, DMSO-d6) 7.69- | C | 360 |
| | isopropyl-N-[(5- | 6.65 (m, 1 H), 7.15-7.12 (m, 1 H), | | |
| | methoxyindan-1- | 6.80-6.77 (m, 1 H), 6.70-6.67 (m, 1 | | |
| | yl)methyl]-5-methyl- | H), 4.80 (br s), 3.72-3.69 (m, 3 H), | | |
| | cyclohexanecarboxamide | 3.40 (u), 3.37-3.33 (m), 3.27-.312 | | |
| | | (m, 2 H), 2.88-2.80 (m, 1 H), 2.78- | | |
| | | 2.71 (m, 1 H), 2.50 (u), 2.15-2.08 | | |
| | | (m, 1 H), 2.07 (u), 1.78-1.65 (m, 3 | | |
| | | H), 1.55-1.47 (m, 2 H), 1.44-1.34 | | |
| | | (m, 3 H), 0.93-0.74 (m, 10 H) | | |
| 153 | (1S,2S,5R)-1-hydroxy-2- | (600.05 MHz, DMSO-d₆) 10.69 (s, | C | 357 |
| | isopropyl-5-methyl-N-[2- | 1 H), 7.70 (br t, J = 5.78, 5.78 Hz, 1 | | |
| | (2-methyl-1H-indol-3- | H), 7.46 (d, J = 7.70 Hz, 1 H), 7.21 | | |
| | yl)ethyl]cyclohexanecarbo | (d, J = 8.07 Hz, 1 H), 6.98-6.94 (m, 1 | | |
| | xamide | H), 6.93-6.89 (m, 1 H), 4.74 (br s, 1 | | |
| | | H), 3.38 (u), 3.34-3.29 (m), 3.26- | | |
| | | 3.19 (m, 1 H), 2.82-2.70 (m, 2 H), | | |
| | | 2.50 (u), 2.33 (s, 3 H), 1.72-1.65 | | |
| | | (m, 2 H), 1.56-1.50 (m, 2 H), 1.45- | | |
| | | 1.33 (m, 4 H), 0.91-0.74 (m, 10 H) | | |
| 154 | (1S,2S,5R)-1-hydroxy-2- | — | C | 362 |
| | isopropyl-N-[(6-methoxy- | | | |
| | 2,3-dihydrobenzofuran-3- | | | |
| | yl)methyl]-5-methyl- | | | |
| | cyclohexanecarboxamide | | | |
| 155 | (1S,2S,5R)-1-hydroxy-2- | (400.23 MHz, DMSO-d₆) 7.66 (br | C | 334 |
| | isopropyl-N-[2-(2- | t, J = 5.50, 5.50 Hz, 1 H), 7.22-7.15 | | |
| | methoxyphenyl)ethyl]-5- | (m, 1 H), 7.13-7.09 (m, 1 H), 6.97- | | |
| | methyl- | 6.92 (m, 1 H), 6.88-6.82 (m, 1 H), | | |
| | cyclohexanecarboxamide | 4.71 (br s, 1 H), 3.78 (s, 3 H), 3.38 | | |
| | | (u), 3.35-3.22 (m), 2.78-2.65 (m, 2 | | |
| | | H), 2.50 (u), 1.74-1.62 (m, 2 H), | | |
| | | 1.55-1.26 (m, 6 H), 1.06 (u), 0.94- | | |
| | | 0.69 (m, 10 H) | | |
| 156 | (1S,2S,5R)-1-hydroxy-2- | (400.23 MHz, DMSO-d₆) 7.55- | C | 318 |
| | isopropyl-5-methyl-N-(2- | 7.47 (m, 1 H), 7.32-7.25 (m, 2 H), | | |
| | phenylpropyl)cyclohexane | 7.24-7.16 (m, 3 H), 4.72 (br s, 1 H), | | |
| | carboxamide | 3.38 (u), 3.34-3.28 (m), 3.27-3.18 | | |
| | | (m, 1 H), 2.50 (u), 1.71-1.61 (m, 2 | | |
| | | H), 1,53-1,28 (m, 6 H), 1.16 (dd, | | |
| | | J = 6.97, 1.22 Hz, 3 H), 0.89-0.66 | | |
| | | (m, 10 H) | | |
| 157 | (1S,2S,5R)-N-[2-(2- | (400.23 MHz, DMSO-d₆) 7.67- | C | 338 |
| | fluorophenyl)-2-hydroxy- | 7.57 (m, 1 H), 7.53-7.46 (m, 1 H), | | |
| | ethyl]-1-hydroxy-2- | 7.33-7.26 (m, 1 H), 7.21-7.08 (m, 2 | | |
| | isopropyl-5-methyl- | H), 5.62 (br s), 4.97-4.90 (m, 1 H), | | |
| | cyclohexanecarboxamide | 4.84 (br s, 1 H), 3.54-3.46 (m), 3.38 | | |
| | | (u), 3.28-3.18 (m), 2.50 (u), 2.08 | | |
| | | (u), 1.72-1.61 (m, 2 H), 1.52-1.22 | | |
| | | (m, 6 H), 1.06 (u), 0.92-0.62 (m, 10 | | |
| | | H) | | |
| 158 | (1S,2S,5R)-N-[2-(4- | (400.23 MHz, DMSO-d₆) 8.35 (u), | C | 338 |
| | fluorophenyl)-2-hydroxy- | 7.64-7.51 (m, 1 H), 7.42-7.32 (m, 2 | | |
| | ethyl]-1-hydroxy-2- | H), 7.18-7.08 (m, 2 H), 5.57 (br s), | | |
| | isopropyl-5-methyl- | 4.84 (br s, 1 H), 4.70-4.60 (m, 1 H), | | |
| | cyclohexanecarboxamide | 3.46-3.38 (m), 3.33 (u), 3.22-3-14 | | |
| | | (m, 1 H), 2.50 (u), 2.07 (u), 1.76- | | |
| | | 1.23 (m, 8 H), 1.05 (u), 0.89-0.64 | | |
| | | (m, 10 H) | | |
| 159 | (1S,2S,5R)-N-[2-(2,3- | (400.23 MHz, DMSO-d₆) 7.69 (br | C | 346 |
| | dihydrobenzofuran-7- | t, J = 5.26, 5.26 Hz, 1 H), 7.09-7.04 | | |
| | yl)ethyl]-1-hydroxy-2- | (m, 1 H), 6.94-6.89 (m, 1 H), 6.76- | | |
| | isopropyl-5-methyl- | 6.70 (m, 1 H), 4.69 (br s), 4.51 (t, | | |
| | cyclohexanecarboxamide | J = 8.74, 8.74 Hz, 2 H), 3.42 (u), | | |
| | | 3.38-3.22 (m), 3.16 (t, J = 8.64, 8.64 | | |
| | | Hz, 2 H), 2.73-2.59 (m, 2 H), 2.50 | | |
| | | (u), 1.74-1.62 (m, 2 H), 1.53-1.29 | | |
| | | (m, 6 H), 0.92-0.70 (m, 10 H) | | |
| 160 | (1S,2S,5R)-N-[2-(2- | (400.23 MHz, DMSO-d₆) 7.66- | C | 354 |
| | chlorophenyl)-2-hydroxy- | 7.55 (m, 2 H), 7.40-7.24 (m, 3 H), | | |
| | ethyl]-1-hydroxy-2- | 5.70 (br s), 5.03-4.98 (m, 1 H), 4.89 | | |
| | isopropyl-5-methyl- | (br s, 1 H), 3.57-3.48 (m), 3.42 (u), | | |
| | cyclohexanecarboxamide | 3.35-3.27 (m), 3.22-3.14 (m, 1 H), | | |
| | | 2.50 (u), 2.08 (u), 1.74-1.61 (m, 2 | | |
| | | H), 1.55-1.22 (m, 6 H), 1.06 (u), | | |
| | | 0.94-0.59 (m, 10 H) | | |
| 161 | (1S,2S,5R)-1-hydroxy-N- | (400.23 MHz, DMSO-d₆) 8.21- | C | 320 |
| | [[3- | 8.14 (m, 1 H), 7.37-7.08 (m, 4 H), | | |
| | (hydroxymethyl)phenyl]m | 5.39 (u), 4.94-4.65 (br m), 4.46 (s, | | |
| | ethyl]-2-isopropyl-5- | 2 H), 4.36-4.22 (m, 2 H), 3.41 (u), | | |
| | methyl- | 2.50 (u), 1.77-1.34 (m, 8 H), 0.97- | | |
| | cyclohexanecarboxamide | 0.71 (m, 10 H) | | |
| 162 | (1S,2S,5R)-N-(2,3- | (400.23 MHz, DMSO-d₆) 7.97-7.88 | C | 332 |
| | dihydrobenzofuran-3- | (m, 1 H), 7.28-7.23 (m, 1 H), 7.14- | | |
| | ylmethyl)-1-hydroxy-2- | 7.08 (m, 1 H), 7.86-7.73 (m, 2 H), | | |
| | isopropyl-5-methyl- | 4.91-4.74 (m), 4.53-4.44 (m, 1 H), | | |
| | cyclohexanecarboxamide | 4.35-4.29 (m, 1 H), 3.72-3.60 (m), | | |
| | | 3.36 (u), 3.32-3.21 (m), 2.50 (u), | | |
| | | 2.07 (u), 1.77-1.64 (m), 1.56-1.31 | | |
| | | (m), 0.94-0.70 (m) | | |
| 163 | (1S,2S,5R)-1-hydroxy-N- | (400.23 MHz, DMSO-d₆) 9.34- | C | 350 |
| | [3-hydroxy-1-(3- | 9.20 (m, 1 H), 8.01-7.91 (m, 1 H), | | |
| | hydroxyphenyl)propyl]-2- | 7.12-7.04 (m, 1 H), 6.76-6.67 (m, 2 | | |
| | isopropyl-5-methyl- | H), 6.65-6.57 (m, 1 H), 4.90-4.81 | | |
| | cyclohexanecarboxamide | (m, 1 H), 4.80-4.64 (m), 4.40-4.28 | | |
| | | (m), 3.37 (u), 2.50 (u), 2.09 (u), | | |
| | | 1.92-1.30 (m), 0.95-0.63 (m) | | |
| 164 | (1S,2S,5R)-1-hydroxy-N- | (400.23 MHz, DMSO-d₆) 7.52- | C | 332 |
| | (2-hydroxy-2-phenyl- | 7.37 (m, 3 H), 7.33-7.25 (m, 2 H), | | |
| | propyl)-2-isopropyl-5- | 7.23-7.16 (m, 1 H), 5,59-5.24 (br | | |
| | methyl- | m), 4.88 (br s), 3.56-3.49 (m), 3.46- | | |
| | cyclohexanecarboxamide | 3.40 (m), 3.36 (u), 3.33-3.23 (m), | | |
| | | 2.50 (u), 2.08 (u), 1.71-1.20 (m), | | |
| | | 1.05 (u), 0.92-0.70 (m), 0.65-0.54 | | |
| | | (m) | | |
| 165 | (1S,2S,5R)-1-hydroxy-N- | (600.05 MHz, DMSO-d₆) 9.27 (br | C | 306 |
| | [(3- | s, 1 H), 8.11-8.07 (m, 1 H), 7.10- | | |
| | hydroxyphenyl)methyl]-2- | 7.05 (m, 1 H), 6.68-6.64 (m, 2 H), | | |
| | isopropyl-5-methyl- | 6.62-6.59 (m, 1 H), 4.80 (br s), | | |
| | cyclohexanecarboxamide | 4.29-4.23 (m, 1 H), 4.18-4.13 (m, 1 | | |
| | | H), 3.61 (u), 2.50 (u), 2.08 (u), | | |
| | | 1.76-1.65 (m, 2 H), 1.61-1.35 (m), | | |
| | | 1.08 (u), 0.95-0.70 (m) | | |
| 166 | (1S,2S,5R)-1-hydroxy-N- | (600.05 MHz, DMSO-d₆) 10.69 (br | C | 365 |
| | [2-(4-hydroxy-3-nitro- | s, 1 H), 7.73-7-62 (m, 2 H), 7.41- | | |
| | phenyl)ethyl]-2-isopropyl- | 7.38 (m, 1 H), 7.05-7.02 (m, 1 H), | | |
| | 5-methyl- | 4.66 (br s), 3.85 (u), 3.45-3.38 (m, | | |
| | cyclohexanecarboxamide | 1 H), 3.32-3.25 (m, 1 H), 2.78-2.69 | | |
| | | (m, 2 H), 2.50 (u), 2.08 (u), 1.71- | | |
| | | 1.61 (m, 2 H), 1.51-1.22 (m, 6 H), | | |
| | | 1.05 (u), 0.89-0.64 (m, 10 H) | | |
| 167 | (1S,2S,5R)-1-hydroxy-N- | (600.05 MHz, DMSO-d₆) 9.09 (br | C | 334 |
| | [3-(4- | s, 1 H), 7.70 (br t, J = 5.78, 5.78 Hz, | | |
| | hydroxyphenyl)propyl]-2- | 1 H), 6.98-6.93 (m, 2 H), 6.67-6.63 | | |
| | isopropyl-5-methyl- | (m, 2 H), 4.72 (br s), 3.68 (u), 3.11- | | |
| | cyclohexanecarboxamide | 3.05 (m, 2 H), 2.50 (u), 2.46-2.41 | | |
| | | (m, 2 H), 2.08 (u), 1.74-1.61 (m, 4 | | |
| | | H), 1.59-1.51 (m, 2 H), 1.46-1.34 | | |
| | | (m, 4 H), 0.91-0.76 (m, 10 H) | | |
| 168 | (1S,2S,5R)-1-hydroxy-N- | (600.05 MHz, DMSO-d₆) 9.08 (br | C | 348 |
| | [(1S)-3-(4- | s, 1 H), 7.40 (br d, J = 8.62 Hz, 1 H), | | |
| | hydroxyphenyl)-1-methyl- | 6.98-6.91 (m, 2 H), 6.67-6.61 (m, 2 | | |
| | propyl]-2-isopropyl-5- | H), 4.74 (br s, 1 H), 3.80-3.73 (m, 1 | | |
| | methyl- | H), 3.34 (u), 2.50 (u), 2.48-2.43 | | |
| | cyclohexanecarboxamide | (m), 2.41-2.35 (m, 1 H), 1.75-1.53 | | |
| | | (m, 6 H), 1.47-1.35 (m, 4 H), 1.10- | | |
| | | 1.01 (m, 3 H), 0.95-0.77 (m, 10 H) | | |
| 169 | (1S,2S,5R)-1-hydroxy-N- | (600.05 MHz, DMSO-d₆) 10.47 (s, | C | 359 |
| | [2-(5-hydroxy-1H-indol-3- | 1 H), 8.72-8.39 (br m), 7.75-7.71 | | |
| | yl)ethyl]-2-isopropyl-5- | (m, 1 H), 7.12-7.09 (m, 1 H), 7.03- | | |
| | methyl- | 7.01 (m, 1 H), 6.86-6.85 (m, 1 H), | | |
| | cyclohexanecarboxamide | 6.59-6.56 (m, 1 H), 4.85-4.48 (br | | |
| | | m), 3.95 (u), 3.41-3.30 (m, 2 H), | | |
| | | 2.78-2.68 (m, 2 H), 2.50 (u), 2.08 | | |
| | | (u), 1.83 (u), 1.74-1.27 (m), 0.91- | | |
| | | 0.68 (m) | | |
| 170 | (1S,2S,5R)-1-hydroxy-N- | (600.05 MHz, DMSO-d₆) 9.08 (br | C | 350 |
| | [(1R)-1-(hydroxymethyl)- | s, 1 H), 7.31 (d, J = 8.80 Hz, 1 H), | | |
| | 2-(4- | 6.98-6.95 (m, 2 H), 6.64-6.61 (m, 2 | | |
| | hydroxyphenyl)ethyl]-2- | H), 4.73 (br s), 4.45-4.28 (br m), | | |
| | isopropyl-5-methyl- | 3.93-3.87 (m, 1 H), 3.52 (u), 3.37- | | |
| | cyclohexanecarboxamide | 3.32 (m), 3.28-3.24 (m), 2.75-2.70 | | |
| | | (m), 2.60-2.55 (m), 2.50 (u), 2.08 | | |
| | | (u), 1.70-1.61 (m, 2 H), 1.44-1.22 | | |
| | | (m, 6 H), 0.87-0.59 (m) | | |
| 171 | (1S,2S,5R)-1-hydroxy-N- | (600.05 MHz, DMSO-d₆) 9.01 (br | C | 346 |
| | [(2R)-7-hydroxytetralin-2- | s, 1 H), 7.51 (br d, J = 8.07 Hz, 1 H), | | |
| | yl]-2-isopropyl-5-methyl- | 6.86 (d, J = 8.44 Hz, 1 H), 6.52 (dd, | | |
| | cyclohexanecarboxamide | J = 8.16, 2.48 Hz, 1 H), 6.44 (d, | | |
| | | J = 2.38 Hz, 1 H), 4.79 (br s, 1 H), | | |
| | | 3.96 (m, 1 H), 2.79 (dd, J = 16.23, | | |
| | | 4.86 Hz, 1 H), 2.69 (br t, J = 6.51, | | |
| | | 6.51 Hz, 2 H), 2.62 (br dd, J = 16.32, | | |
| | | 8.99 Hz, 1 H), 2.50 (u), 1.83 (br dd, | | |
| | | J = 11.92, 4.03 Hz, 1 H), 1.69 (m, 3 | | |
| | | H), 1.56 (m, 2 H), 1.42 (m, 4 H), | | |
| | | 0.89 (br dd, J = 12.84, 3.48 Hz, 1 H), | | |
| | | 0.80 (m, 9 H) | | |
| 172 | (1S,2S,5R)-N-[2-(2- | (600.05 MHz, DMSO-d₆) 9.56 (br | C | 398 |
| | bromo-5-hydroxy- | s, 1 H), 7.76 (br t, J = 5.78, 5.78 Hz, | | |
| | phenyl)ethyl]-1-hydroxy- | 1 H), 7.32 (d, J = 8.62 Hz, 1 H), 6.72 | | |
| | 2-isopropyl-5-methyl- | (d, J = 2.75 Hz, 1 H), 6.58 (dd, | | |
| | cyclohexanecarboxamide | J = 8.62, 2.93 Hz, 1 H), 4.71 (br s), | | |
| | | 3.60 (u), 3.39-3.25 (m, 2 H), 2.80- | | |
| | | 2.69 (m, 2 H), 2.50 (u), 2.08 (u), | | |
| | | 1.73-1.63 (m, 2 H), 1.52-1.45 (m, 2 | | |
| | | H), 1.44-1.28 (m, 4 H), 1.07 (u), | | |
| | | 0.91-0.71 10 H) | | |
| 173 | (1S,2S,5R)-N-[2-(2,4- | (600.05 MHz, DMSO-d₆) 9.15 (br | C | 336 |
| | dihydroxyphenyl)ethyl]-1- | s, 1 H), 8.95 (br s, 1 H), 7.62-7.58 | | |
| | hydroxy-2-isopropyl-5- | (br m, 1 H), 6.80-6.76 (m, 1 H), | | |
| | methyl- | 6.27-6.25 (m, 1 H), 6.13-6.09 (m, 1 | | |
| | cyclohexanecarboxamide | H), 4.70 (br s), 3.71 (u), 3.30-3.27 | | |
| | | (m, 1 H), 3.22-3.15 (m, 1 H), 2.62- | | |
| | | 2.51 (m), 2.50 (u), 2.08 (u), 1.73- | | |
| | | 1.63 (m, 2 H), 1.55-1.47 (m, 2 H), | | |
| | | 1.44-1.32 (m, 4 H), 0.91-0.72 (m, | | |
| | | 10 H) | | |
| 174 | (1S,2S,5R)-1-hydroxy-N- | — | C | 306 |
| | (4-methoxyphenyl)-5- | | | |
| | methyl-2-propan-2- | | | |
| | ylcyclohexane-1- | | | |
| | carboxamide | | | |

### Examples 175 & 176

### (1S,2S,5R)-1-hydroxy-N-(((1RS)-hydroxy-2,3-dihydro-1H-inden-1-yl)methyl)-2-isopropyl-5-meth ylcyclohexane-1-carboxamide (175)

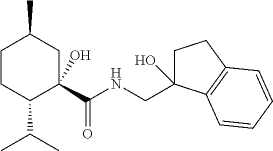 [SMILES: CC(C)[C@@H]1CC[C@@H](C)C[C@@]1(O)C(=O)NCC1(O)CCc2ccccc21]

### (1S,2S,5R)-1-hydroxy-N-(((1RS)-hydroxy-2,3-dihydro-1H-inden-1-yl)methyl)-2-isopropyl-5-methylcyclohexane-1-carboxamide (176)

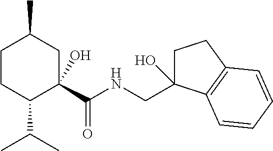 [SMILES: CC(C)[C@@H]1CC[C@@H](C)C[C@@]1(O)C(=O)NCC1(O)CCc2ccccc21]

Chiral separation of (1S,2S,5R)-1-hydroxy-N-((1-hydroxy-2,3-dihydro-1H-inden-1-yl)methyl)-2-isopropyl-5-methylcyclohexane-1-carboxamide (230 mg) carried out by liquid chromatography using Chiralcel OZ with heptane and ethanol led to (1S,2S,5R)-1-hydroxy-N-(((1RS)-hydroxy-2,3-dihydro-1H-inden-1-yl)methyl)-2-isopropyl-5-methylcyclohexane-1-carboxamide (example 175, 124 mg) and (1S,2S,5R)-1-hydroxy-N-(((1RS)-hydroxy-2,3-dihydro-1H-inden-1-yl)methyl)-2-isopropyl-5-methylcyclohexane-1-carboxamide (example 176, 103 mg).

### Example 177

### (1S,2S,5R)-1-hydroxy-N-(((1RS)-hydroxy-1,2,3,4-tetrahydronaphthalen-1-yl)methyl)-2-isopropyl-5-methylcyclohexane-1-carboxamide (177)

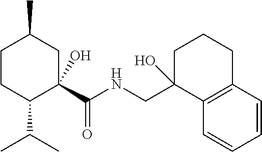 [SMILES: CC(C)[C@@H]1CC[C@@H](C)C[C@@]1(O)C(=O)NCC1(O)CCCc2ccccc21]

Chiral separation of (1S,2S,5R)-1-hydroxy-N-((1-hydroxy-1,2,3,4-tetrahydronaphthalen-1-yl)methyl)-2-isopropyl-5-methylcyclohexane-1-carboxamide (230 mg) carried out by liquid chromatography using Chiralcel OZ with heptane and ethanol led to (1S,2S,5R)-1-hydroxy-N-(((1RS)-hydroxy-1,2,3,4-tetrahydronaphthalen-1-yl)methyl)-2-isopropyl-5-methylcyclohexane-1-carboxamide (example 177,126 mg) and its diastereoisomer (121 mg).

### Example 178

### (1S,2S,5R)-1-hydroxy-N-((2R)-2-hydroxy-2-phenylethyl)-2-isopropyl-5-methylcyclohexane-1-carboxamide (178)

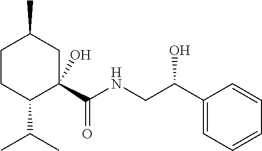 [SMILES: CC(C)[C@@H]1CC[C@@H](C)C[C@@]1(O)C(=O)NC[C@H](O)c1ccccc1]

Chiral separation of (1S,2S,5R)-1-hydroxy-N-(2-hydroxy-2-phenylethyl)-2-isopropyl-5-methylcyclohexane-1-carboxamide (262 mg) carried out by liquid chromatography using Chirlapak AD with heptane and ethanol led to 1S,2S,5R)-1-hydroxy-N-((2R)-2-hydroxy-2-phenylethyl)-2-isopropyl-5-methylcyclohexane-1-carboxamide (example 178, 23 mg) and (1S,2S,5R)-1-hydroxy-N-((2S)-2-hydroxy-2-phenylethyl)-2-isopropyl-5-methylcyclohexane-1-carboxamide (example 1, 173 mg).

### Examples 179 & 180

### (1S,2S,5R)—N-((2R)-2-fluoro-2-phenylethyl)-1-hydroxy-2-isopropyl-5-methylcyclohexane-1-carboxamide (179)

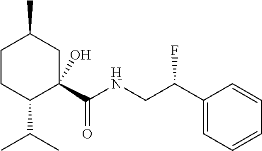 [SMILES: CC(C)[C@@H]1CC[C@@H](C)C[C@@]1(O)C(=O)NC[C@H](F)c1ccccc1]

### (1S,2S,5R)—N-((2S)-2-fluoro-2-phenylethyl)-1-hydroxy-2-isopropyl-5-methylcyclohexane-1-carboxamide (180)

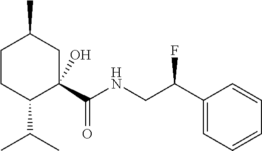 [SMILES: CC(C)[C@@H]1CC[C@@H](C)C[C@@]1(O)C(=O)NC[C@@H](F)c1ccccc1]

Chiral separation of (1S,2S,5R)—N-(2-fluoro-2-phenylethyl)-1-hydroxy-2-isopropyl-5-methylcyclohexane-1-carboxamide (144 mg) carried out by SFC using Cellulose with CO2 90% MeOH 0.1% TEA 10% led to (1S,2S,5R)—N-((2R)-2-fluoro-2-phenylethyl)-1-hydroxy-2-isopropyl-5-methylcyclohexane-1-carboxamide (example 179, 66 mg) and (1S,2S,5R)—N-((2S)-2-fluoro-2-phenylethyl)-1-hydroxy-2-isopropyl-5-methylcyclohexane-1-carboxamide (example 180, 57 mg).

### Examples 181 & 182

### (1S,2S,5R)—N-((2RS)-2-((S)-2-aminopropanamido)-2-phenylethyl)-1-hydroxy-2-isopropyl-5-methylcyclohexane-1-carboxamide (181)

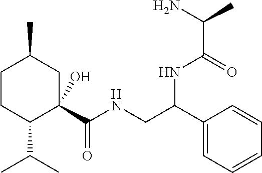 [SMILES: CC(C)[C@@H]1CC[C@@H](C)C[C@@]1(O)C(=O)NCC(NC(=O)[C@H](C)N)c1ccccc1]

### (1S,2S,5R)—N-((2RS)-2-((S)-2-aminopropanamido)-2-phenylethyl)-1-hydroxy-2-isopropyl-5-methylcyclohexane-1-carboxamide (182)

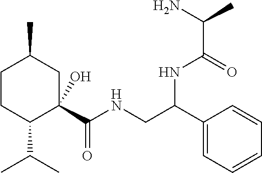 [SMILES: CC(C)[C@@H]1CC[C@@H](C)C[C@@]1(O)C(=O)NCC(NC(=O)[C@H](C)N)c1ccccc1]

Under coupling conditions F, with (1S,2S,5R)—N-((2S)-amino-2-phenylethyl)-1-hydroxy-2-isopropyl-5-methylcyclohexane-1-carboxamide (100 mg) and BOC-ALA-OH (1.1eq) [NB THF was used as solvent instead of DMF] led to tert-butyl ((S)-1-(((S)-2-((1S,2S,5R)-1-hydroxy-2-isopropyl-5-methylcyclohexane-1-carboxamido)-1-phenylethyl)amino)-1-oxopropan-2-yl)carbamate (127 mg).

Under deprotection conditions B, to tert-butyl ((S)-1-(((S)-2-((1S,2S,5R)-1-hydroxy-2-isopropyl-5-methylcyclohexane-1-carboxamido)-1-phenylethyl)amino)-1-oxopropan-2-yl)carbamate (124.4 mg) in the presence of TFA (9.7eq) led to (1S,2S,5R)—N—((S)-2-((S)-2-aminopropanamido)-2-phenylethyl)-1-hydroxy-2-isopropyl-5-methylcyclohexane-1-carboxamide (95.5 mg) as white solid.

Chiral separation of (1S,2S,5R)—N-(2-((S)-2-aminopropanamido)-2-phenylethyl)-1-hydroxy-2-isopropyl-5-methylcyclohexane-1-carboxamide (91 mg) carried out by liquid chromatography using Chiralpak AD-H with heptane and ethanol led to (1S,2S,5R)—N-((2RS)-2-((S)-2-aminopropanamido)-2-phenylethyl)-1-hydroxy-2-isopropyl-5-methylcyclohexane-1-carboxamide (example 181, 6.7 mg) and (1S,2S,5R)—N-((2RS)-2-((S)-2-aminopropanamido)-2-phenylethyl)-1-hydroxy-2-isopropyl-5-methylcyclohexane-1-carboxamide (example 182, 47.1 mg).

### Examples 183 & 184

### (1S,2S,5R)—N-((2RS)-(2-aminoacetamido)-2-phenylethyl)-1-hydroxy-2-isopropyl-5-methylcyclohexane-1-carboxamide (183)

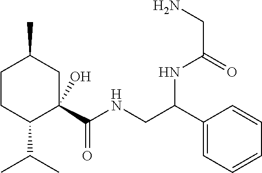 [SMILES: CC(C)[C@@H]1CC[C@@H](C)C[C@@]1(O)C(=O)NCC(NC(=O)CN)c1ccccc1]

### (1S,2S,5R)—N-((2RS)-2-((S)-2-aminopropanamido)-2-phenylethyl)-1-hydroxy-2-isopropyl-5-methylcyclohexane-1-carboxamide (184)

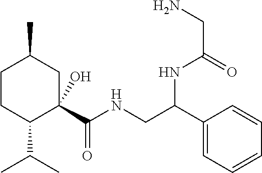 [SMILES: CC(C)[C@@H]1CC[C@@H](C)C[C@@]1(O)C(=O)NCC(NC(=O)CN)c1ccccc1]

Under coupling conditions F, with (1S,2S,5R)—N-((2S)-amino-2-phenylethyl)-1-hydroxy-2-isopropyl-5-methylcyclohexane-1-carboxamide (100 mg) and BOC-GLY-OH (1.1eq) [NB THF was used as solvent instead of DMF] led to tert-butyl (2-(((S)-2-((1S,2S,5R)-1-hydroxy-2-isopropyl-5-methylcyclohexane-1-carboxamido)-1-phenylethyl)amino)-2-oxoethyl)carbamate (142 mg).

Under deprotection conditions B, tert-butyl (2-(((S)-2-((1S,2S,5R)-1-hydroxy-2-isopropyl-5-methylcyclohexane-1-carboxamido)-1-phenylethyl)amino)-2-oxoethyl)carbamate (140 mg) in the presence of TFA (9.7eq) led to (1S,2S,5R)—N—((S)-2-(2-aminoacetamido)-2-phenylethyl)-1-hydroxy-2-isopropyl-5-methylcyclohexane-1-carboxamide (81.9 mg) as white solid.

Chiral separation of (1S,2S,5R)—N-(2-(2-aminoacetamido)-2-phenylethyl)-1-hydroxy-2-isopropyl-5-methylcyclohexane-1-carboxamide (32 mg) carried out by SFC using Chiralpak AD-H with mobile phase CO2 85% MeOH 15% TEA 0.1% led to (1S,2S,5R)—N-((2RS)-(2-aminoacetamido)-2-phenylethyl)-1-hydroxy-2-isopropyl-5-methylcyclohexane-1-carboxamide (example 183, 14.8 mg) and (1S,2S,5R)—N-((2RS)-(2-aminoacetamido)-2-phenylethyl)-1-hydroxy-2-isopropyl-5-methylcyclohexane-1-carboxamide (example 184, 2.3 mg).

### Example 185

### (1S,2S,5R)-1-hydroxy-2-isopropyl-N-[2-[3-[2-[2-(2-methoxyethoxy)ethoxy]ethoxy]phenyl]ethyl]-5-methyl-cyclohexanecarboxamide

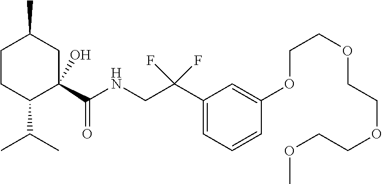 [SMILES: COCCOCCOCCOc1cccc(C(F)(F)CNC(=O)[C@]2(O)C[C@H](C)CC[C@H]2C(C)C)c1]

Under alkylation conditions C, (1S,2S,5R)-1-hydroxy-N-(3-hydroxyphenethyl)-2-isopropyl-5-methylcyclohexane-1-carboxamide (50 mg) in the presence of 1-bromo-2-[2-(2-methoxyethoxy)ethoxy]ethane (1.2eq) led to (1S,2S,5R)-1-hydroxy-2-isopropyl-N-[2-[3-[2-[2-(2-methoxyethoxy)ethoxy]ethoxy]phenyl]ethyl]-5-methyl-cyclohexanecarboxamide (32 mg) after flash chromatography with heptane and EtOAc.

### Example 186

### (1S,2S,5R)—N-[2,2-difluoro-2-(2-methoxyphenyl)ethyl]-1-hydroxy-2-isopropyl-5-methyl-cyclohexanecarboxamide

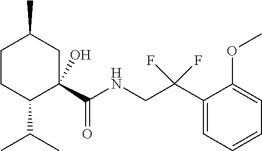 [SMILES: COc1ccccc1C(F)(F)CNC(=O)[C@]1(O)C[C@H](C)CC[C@H]1C(C)C]

Under coupling conditions B, (1S,2S,5R)-1-hydroxy-2-isopropyl-5-methylcyclohexane-1-carboxylic acid (200 mg) and 2,2-difluoro-2-(2-methoxyphenyl)ethan-1-amine hydrochloride (1.1eq) [after amine addition, the reaction was heated at 70° C. during 17 hr] led to (1S,2S,5R)—N-[2,2-difluoro-2-(2-methoxyphenyl)ethyl]-1-hydroxy-2-isopropyl-5-methyl-cyclohexanecarboxamide (150.6 mg) after flash chromatography with heptane and EtOAc.

### Example 187

### (1S,2S,5R)—N-[2,2-difluoro-2-(3-methylphenyl)ethyl]-1-hydroxy-2-isopropyl-5-methyl-cyclohexanecarboxamide

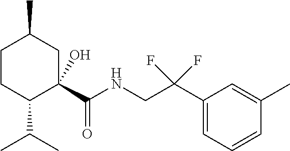 [SMILES: Cc1cccc(C(F)(F)CNC(=O)[C@]2(O)C[C@H](C)CC[C@H]2C(C)C)c1]

Under coupling conditions B, (1S,2S,5R)-1-hydroxy-2-isopropyl-5-methylcyclohexane-1-carboxylic acid (90 mg) and 2,2-difluoro-2-(3-methylphenyl)ethan-1-amine (1.1eq) [after amine addition, the reaction was heated at 70° C. during 17 hr] led to (1S,2S,5R)—N-[2,2-difluoro-2-(m-tolyl)ethyl]-1-hydroxy-2-isopropyl-5-methyl-cyclohexanecarboxamide (52.5 mg) after flash chromatography with heptane and EtOAc.

### Example 188

### (1S,2S,5R)—N-[2,2-difluoro-2-(3-methoxyphenyl)ethyl]-1-hydroxy-2-isopropyl-5-methyl-cyclohexanecarboxamide

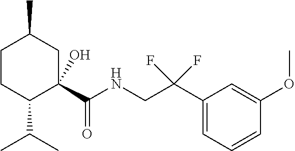 [SMILES: COc1cccc(C(F)(F)CNC(=O)[C@]2(O)C[C@H](C)CC[C@H]2C(C)C)c1]

Under coupling conditions A, (1S,2S,5R)-1-hydroxy-2-isopropyl-5-methylcyclohexane-1-carboxylic acid (90 mg) and 2,2-difluoro-2-(3-methoxyphenyl)ethan-1-amine (1.1eq) [after amine addition, the reaction was heated at 70° C. during 17 hr] led to (1S,2S,5R)—N-[2,2-difluoro-2-(3-methoxyphenyl)ethyl]-1-hydroxy-2-isopropyl-5-methyl-cyclohexanecarboxamide (102.5 mg) after flash chromatography with heptane and EtOAc.

Examples 175 to 188 were characterized by 1H NMR and LCMS analysis as shown below in Table 5:

TABLE 5

| | LCMS | | | |
|---|---|---|---|---|
| Example | Method | [M + H]⁺ | [M − H]⁻ | 1H NMR spectrum (400 MHZ, DMSO-d₆) δ ppm |
| 175 | D | — | 344 | 0.72 (d, J = 7 Hz, 3 H), 0.80 (d, J = 7 Hz, 6 H), 0.86 |
| | | | | (m, 1 H), 1.21-1.54 (m, 6 H), 1.62-1.78(m, 2 H), |
| | | | | 1.94 (m, 1 H), 2.15 (m, 1 H), 2.75 (m, 1 H), 2.87 (m, |
| | | | | 1 H), 3.31 (m , 2 H), 5.05 (m, 1 H), 5.49 (s, 1 H), |
| | | | | 7.09-7.25 (m, 3 H), 7.31 (d, J = 7 Hz, 1 H), 7.60 (t, |
| | | | | J = 6 Hz, 1 H) |
| 176 | D | — | 344 | 0.75 (d, J = 7 Hz, 3 H), 0.80 (d, J = 7 Hz, 6 H), 0.87 |
| | | | | (m, 1 H), 1.28-1.58 (m, 6 H), 1.61-1.79 (m, 2 H), |
| | | | | 1.93 (dt, J = 13, 8 Hz, 1 H), 2.15 (m, 1 H), 2.75 (m, 1 |
| | | | | H), 2.87 (br dd, J = 9, 4 Hz, 1 H), 3.27 (m, 1 H), 3.37 |
| | | | | (m, 1 H), 5.01 (s, 1 H), 5.46 (s, 1 H), 7.14-7.23 (m, |
| | | | | 3 H), 7.34 (d, J = 7 Hz, 1 H), 7.60 (t, J = 6 Hz, 1 H) |
| 177 | D | — | 358 | 0.77 (d, J = 7 Hz, 3 H), 0.81 (dd, J = 7, 5 Hz, 6 H), |
| | | | | 0.89 (m, 1 H), 1.29-1.48 (m, 4 H), 1.49-1.82 (m, 7 |
| | | | | H), 1.89 (br dd, J = 13, 5 Hz, 1 H), 2.7 (m, 2 H), 3.09 |
| | | | | (dd, J = 13, 4 Hz, 1 H), 3.55 (dd, J = 13, 8 Hz, 1 H), |
| | | | | 5.02 (s, 1 H), 5.32 (s, 1 H), 7.04 (m, 1 H), 7.10-7.20 |
| | | | | (m, 2 H), 7.51 (m, 1 H), 7.58 (m, 1 H) |
| 178 | D | 320 | 318 | 0.70 (d, J = 7 Hz, 3 H), 0.74 (d, J = 7 Hz, 3 H), 0.79 (d, |
| | | | | J = 6 Hz, 3 H), 0.86 (m, 1 H), 1.20-1.51 (m, 6 H), |
| | | | | 1.59-1.76 (m, 2 H), 3.33 (m, 2 H), 4.64 (q, J = 6 Hz, |
| | | | | 1 H), 4.87 (s, 1 H), 5.54 (d, J = 4 Hz, 1 H), 7.22 (m, 1 |
| | | | | H), 7.27-7.40 (m, 4 H), 7.56 (t, J = 6 Hz, 1 H) |
| 179 | D | 322 | 320 | 0.73 (d, J = 7 Hz, 3 H), 0.75 (d, J = 7 Hz, 3 H), 0.80 (d, |
| | | | | J = 6 Hz, 3 H), 0.88 (m, 1 H), 1.30-1.54 (m, 6 H), |
| | | | | 1.63-1.76 (m, 2 H), 3.38-3.70 (m, 2 H), 4.88 (s, 1 |
| | | | | H), 5.61 (ddd, J = 48, 8, 4 Hz, 1 H), 7.35-7.45 (m, 5 |
| | | | | H), 7.86 (br t, J = 6 Hz, 1 H) |
| 180 | D | 322 | — | — |
| 181 | D | 390 | 388 | 0.71-0.89 (m, 10 H), 1.12 (d, J = 7 Hz, 3 H), 1.22 - |
| | | | | 1.56 (m, 6 H), 1.59-1.75 (m, 2 H), 2.08 (br s, 2 H), |
| | | | | 3.25 (m, 1 H), 3.34-3.47 (m, 2 H), 4.81 (s, 1 H), |
| | | | | 4.91 (m, 1 H), 7.13-7.43 (m, 5 H), 7.79 (t, J = 6 Hz, 1 |
| | | | | H), 8.25 (br d, J = 8 Hz, 1 H) |
| 182 | D | 390 | 388 | 0.71 (d, J = 7 Hz, 3 H), 0.75 (d, J = 7 Hz, 3 H), 0.79 (d, |
| | | | | J = 6 Hz, 3 H), 0.85 (m, 1 H), 1.13 (d, J = 7 Hz, 3 H), |
| | | | | 1.29-1.55 (m, 6 H), 1.56-1.85 (m, 2 H), 3.25-3.30 |
| | | | | (m, 2 H), 3.45 (m, 1 H), 4.83 (s, 1 H), 4.91 (m, 1 H), |
| | | | | 7.23 (m, 1 H), 7.27-7.36 (m, 4 H), 7.83 (t, J = 6 Hz, 1 |
| | | | | H), 8.23 (br d, J = 7 Hz, 1 H) |
| 183 | D | 376 | 374 | 0.70 (d, J = 7 Hz, 3 H), 0.75 (d, J = 7 Hz, 3 H), 0.80 (d, |
| | | | | J = 6 Hz, 3 H), 0.87 (m, 1 H), 1.22-1.51 (m, 6 H), |
| | | | | 1.60-1.74 (m, 2 H), 1.81 (br s, 2 H), 3.09 (s, 2 H), |
| | | | | 3.28 (m, 1 H), 3.47 (m, 1 H), 4.82 (s, 1 H), 4.96 (m, 1 |
| | | | | H), 7.24 (m, 1 H), 7.27-7.35 (m, 4 H), 7.83 (t, J = 6 |
| | | | | Hz, 1 H), 8.21 (br d, J = 8 Hz, 1 H) |
| 184 | D | 376 | 374 | 0.74 (d, J = 4 Hz, 3 H), 0.75 (d, J = 4 Hz, 3 H), 0.79 (d, |
| | | | | J = 6 Hz, 3 H), 0.87 (m, 1 H), 1.30-1.52 (m, 6 H), |
| | | | | 1.61-1.72 (m, 2 H), 1.73-1.94 (m, 2 H), 3.09 (s, 2 |
| | | | | H), 3.38 (m, 1 H), 3.46 (m, 1 H), 4.81 (s, 1 H), 4.92- |
| | | | | 5.02 (m, 1 H), 7.23 (m, 1 H), 7.28-7.35 (m, 4 H), |
| | | | | 7.79 (t, J = 6 Hz, 1 H), 8.24 (br d, J = 8 Hz, 1 H) |
| 185 | E | — | 466 | — |
| 186 | F | — | 370 | — |
| 187 | F | — | 354 | — |
| 188 | F | — | 370 | — |

Some compounds of formula (I) were subjected to pharmacological tests for determining their activation effects on TRPM8 receptor.

### In Vitro Pig and Human TRPM8 Fluorescence Calcium Flux Assays

For functional expression of TRPM8, the full-length cDNA encoding human (NM_024080) and pig (XM_001927892.1) TRPM8 sequences were subcloned into p658 and pcDNA5/FRT/TO mammalian expression vectors, respectively. Engineered recombinant CHO cell lines expressing human or pig TRPM8 were generated. Culture media was Ham-F12 with 10% FCS for both lines, and for the pig cell line only 400 μg/ml hygromycin B and 30 μg/mL blasticidin were added as selection antibiotics (all reagents were obtained from Invitrogen, Fisher Scientific). Cells were grown in culture flasks up to 80% confluence where they were harvested using accutase (Sigma, MO, USA) to detach the cells by enzymatic dissociation and either seeded directly into assay plates or cryopreserved for future use.

For calcium flux assays, cells were plated into clear base poly-D-lysine coated 384-well plates (BD Biosciences, NJ USA) at a density of 15,000 cells per well in appropriate culture medium and grown overnight. The following day, all medium was removed then the cells were incubated with 2 μM of Fluo4-AM Dye (Molecular Probes) prepared in complete HBSS assay buffer containing 20 mM HEPES, 0.1% BSA, and 2.5 mM probenecid at room temperature for one hour. Following incubation, plates were inserted into a FDSS6000 instrument (Hamamatsu, Photonics, Japan) instrument, where cells were challenged with compounds of the formula (at varying concentrations) and intracellular calcium was measured kinetically for 3 min after addition.

This protocol allowed the determination of an EC50 value, from the sixteen-point dose response data, for each compound of the formula tested. Maximal fluorescence intensity achieved upon addition of 100 μM menthol (stock solution prepared in ethanol from solid, Sigma-Aldrich) was exported from the FDSS and further analyzed using IDBS XLFit 5. Data were normalized to the average 100 μM menthol response (maximum control wells) included in each plate. The dose response curves from the average of wells for each data point were analyzed by using nonlinear regression of sigmoidal dose response (formula 205). Finally, the (menthol-related) EC50 values (Half maximal effective concentration) were calculated with the best-fit dose curve determined by IDBS XLFit 5 software.

### In Vitro Pig and Human TRPM8 Automated Patch-Clamp Assays

Automated whole-cell recordings of compound effects were performed on the SyncroPatch 384PE (Nanion Technologies, Munich, Germany) incorporated into a Biomek FX pipetting robot (Beckman Coulter, Jersey City, NJ, USA) using the same engineered CHO cell lines expressing human or pig TRPM8 channels. Data acquisition and analysis were performed with the proprietary software PatchControl 384 and DataControl 384, respectively (Nanion Technologies, Munich, Germany). All recordings were carried out using planar borosilicate glass patch clamp chips in a 384-microtiter plate format with patch hole resistances of 2-4 MO. For recordings, standard intracellular solution was used containing (in mM): 130 KF, 4 NaCl, 1 MgCl₂, 0.5 CaCl₂, 10 HEPES and 10 EGTA/KOH (pH 7.2) and standard extracellular solution contained in mM: 150 NaCl, 4 KCl, 0.5 CaCl₂, 1 MgCl₂ and 10 HEPES (pH 7.3).

Prior to the electrophysiological measurements, cells were harvested using Accutase (GIBCO, Fisher Scientific) and resuspended in warmed extracellular solution. The cell suspension was kept in the dedicated cell reservoir at 28° C. at a 1 million/ml cell density and shaken at 500 rounds per min (rpm). Experiments were performed at 28° C. throughout. Cells were distributed in high resistance 4-holes-per-well Nanion chips where they were caught on the patch holes by application of a −80 mbar pressure. The cell membrane was ruptured to obtain the whole-cell configuration with a pressure pulse of −250 mbar for 2 seconds. Voltage protocols were constructed using PatchControl 384. The cells were held at a holding potential of −60 mV and TRPM8 currents were recorded using a ramp protocol from −80 to +80 mV every 5 seconds before and after compound addition. Resulting currents were recorded at both −80 and +80 mV and current kinetics were exported and analyzed. From each plate, control wells with 200 μM menthol were recorded to validate the assay. One concentration of compound was applied to each of the other wells. For each concentration, compound response was determined by subtracting the baseline current recorded before compound addition from the maximum peak elicited currents obtained after compound addition. Then, each compound response was normalized to control condition without compound and maximum current (Emax) obtained for each compound. Nanion DataControl 384 software plotted current response for each compound concentration and was able to calculate an EC50 value for each active compound.

The Table 6 below indicates the in vitro results of pig and human TRPM8 fluorescence calcium flux assays and of pig TRPM8 automated patch-clamp assay for compounds of formula (I), and demonstrates that the compounds tested have an agonist activity regarding TRPM8 receptor

TABLE 6

| | | | Pig TRPM8 | Human TRPM8 |
|---|---|---|---|---|
| | Pig TRPM8 | Human TRPM8 | Automated | Automated |
| | Calcium Flux | Calcium Flux | Patch-Clamp | Patch-Clamp |
| Example | EC50 (nM) | EC50 (nM) | EC50 (nM) | EC50 (nM) |
| 1 | 1.2 | 1.7 | 733 | 810 |
| 2 | 0.7 | 1.2 | 596 | 450 |
| 3 | 35.5 | 36.8 | 9912 | 1120 |
| 4 | 1.2 | 2.8 | 467 | 541 |
| 5 | 0.7 | 1.3 | 478 | 950 |
| 6 | 0.5 | 0.3 | 237 | 72 |
| 7 | 8.1 | 8.5 | 6193 | 2800 |
| 8 | 712.9 | 1514.9 | — | — |
| 9 | 6.3 | 8.8 | 4095 | 2400 |
| 10 | 9.7 | 21.4 | 4799 | 4130 |
| 11 | 298.5 | 148.0 | 4901 | 3700 |
| 12 | 2.0 | 3.0 | — | — |
| 13 | 2.0 | 1.0 | 782 | 150 |
| 14 | 6.7 | 10.3 | 3296 | 2720 |
| 15 | 2.1 | 3.0 | 1505 | 1430 |
| 16 | 13.0 | 16.0 | 2366 | 1290 |
| 17 | 191.0 | 410.0 | — | — |
| 18 | 242.1 | 757.3 | — | — |
| 19 | 19.9 | 7.1 | 1522 | 1700 |
| 20 | 6.6 | 3.2 | 1416 | 1400 |
| 21 | 56.3 | 65.1 | 5293 | 5500 |
| 22 | 34.5 | 25.2 | 3838 | 3600 |
| 23 | 6.0 | 7.0 | 1068 | 500 |
| 24 | 13.4 | 7.8 | 1700 | 2580 |
| 25 | 32.8 | 14.5 | 3400 | 2150 |
| 26 | 5.5 | 3.7 | 1100 | 1800 |
| 27 | 10.3 | 3.1 | 4000 | 2400 |
| 28 | 311.3 | 249.7 | 5400 | 1440 |
| 29 | 221.3 | 210.7 | — | — |
| 30 | 766.2 | 3250.6 | >10000 | — |
| 31 | 9.3 | 10.4 | 3155 | 2078 |
| 32 | 3.0 | 3.0 | 507 | 1230 |
| 33 | 38.0 | 65.0 | 4309 | 4200 |
| 34 | 80.0 | 97.0 | 3207 | 4590 |
| 35 | 3.0 | 3.0 | 1328 | 1010 |
| 36 | 36.0 | 47.0 | 3808 | 1880 |
| 37 | 11.0 | 17.0 | 2357 | 3100 |
| 38 | 1.8 | 2.4 | 276 | 470 |
| 39 | 0.9 | 1.2 | 430 | 460 |
| 40 | 1.4 | 2.5 | 1755 | 1445 |
| 41 | 74.5 | 194.7 | 5114 | 1100 |
| 42 | 3.8 | 12.4 | 921 | 910 |
| 43 | 56.9 | 191.3 | 3967 | 4000 |
| 44 | 10.6 | 34.9 | 711 | 1800 |
| 45 | 18.2 | 48.5 | — | — |
| 46 | 4.6 | 11.9 | — | — |
| 47 | 585.7 | 640.7 | — | — |
| 48 | 5.0 | 13.5 | 4600 | 2630 |
| 49 | 24.2 | 67.0 | 1400 | 1940 |
| 50 | 0.4 | 0.4 | 71 | 140 |
| 51 | 4.5 | 7.8 | 823 | 3030 |
| 52 | 24.3 | 26.0 | 864 | 1300 |
| 53 | 2.6 | 2.4 | 326 | 200 |
| 54 | 91.1 | 225.6 | 8728 | 5650 |
| 55 | 54.3 | 102.3 | 3548 | 4470 |
| 56 | 11.2 | 19.6 | 6156 | >10000 |
| 57 | 219.5 | 253.8 | >10000 | — |
| 58 | 2.2 | 5.2 | 887 | 1030 |
| 59 | 4.3 | 6.2 | — | — |
| 60 | 7.1 | 43.5 | — | — |
| 61 | 7.4 | 11.1 | 5045 | 2915 |
| 62 | 2.3 | 1.6 | — | — |
| 63 | 5.7 | 2.6 | 57 | 53 |
| 64 | 8.3 | 16.6 | 3866 | 5821 |
| 65 | 729.0 | 353.0 | — | — |
| 66 | 75.0 | 51.0 | 6058 | 3700 |
| 67 | 5.0 | 9.0 | 3360 | 2700 |
| 68 | 32.0 | 44.0 | 3641 | 2690 |
| 69 | 42.0 | 46.0 | 1579 | 1930 |
| 70 | 8.0 | 9.0 | 3742 | 3990 |
| 71 | 2.0 | 1.0 | 862 | 183 |
| 72 | 2.0 | 3.0 | 790 | 1390 |
| 73 | 8.0 | 4.0 | 1122 | 690 |
| 74 | 9.0 | 41.0 | 5186 | 2900 |
| 75 | 109.0 | 84.0 | — | — |
| 76 | 364.6 | 501.3 | — | — |
| 77 | 93.0 | 90.0 | — | — |
| 78 | 327.0 | 575.0 | — | — |
| 79 | 57.6 | 84.5 | 5159 | 5500 |
| 80 | 82.0 | 40.2 | 3966 | 1800 |
| 81 | 25.2 | 27.7 | 4246 | 1500 |
| 82 | 486.4 | 332.2 | — | — |
| 83 | 136.4 | 167.9 | — | — |
| 84 | 140.7 | 143.2 | — | — |
| 85 | 2.4 | 3.3 | 967 | 700 |
| 86 | 749.1 | 546.7 | >10000 | — |
| 87 | 258.6 | 982.3 | — | — |
| 88 | 20.0 | 56.7 | 1092 | 1500 |
| 89 | 47.6 | 90.3 | 3252 | 3300 |
| 90 | 14.4 | 41.8 | 2003 | 3000 |
| 91 | 5.2 | 19.0 | — | — |
| 92 | 2.3 | 4.1 | — | — |
| 93 | 67.8 | 49.3 | 2000 | 2800 |
| 94 | 7.7 | 6.4 | 1500 | 1070 |
| 95 | 3.1 | 3.6 | — | — |
| 96 | 25.2 | 10.3 | 1100 | 630 |
| 97 | 29.2 | 11.1 | — | — |
| 98 | 64.7 | 52.8 | — | — |
| 99 | 99.1 | 110.3 | — | — |
| 100 | 2.9 | 2.2 | — | — |
| 101 | 173.8 | 376.3 | — | — |
| 102 | 69.0 | 42.0 | — | — |
| 103 | 14.9 | 18.8 | — | — |
| 104 | 0.6 | 1.1 | — | — |
| 105 | 28.4 | 30.8 | — | — |
| 106 | 23.2 | 29.3 | >10000 | — |
| 107 | 21.7 | 42.8 | 2439 | 3800 |
| 108 | 73.0 | 211.5 | >10000 | — |
| 109 | 11.9 | 22.4 | 1769 | 2200 |
| 110 | 639.2 | 1639.1 | >10000 | — |
| 111 | 25.1 | 72.7 | 5070 | 5100 |
| 112 | 892.4 | 1580.5 | — | — |
| 113 | 93.5 | 99.4 | 9635 | 1400 |
| 114 | 28.2 | 59.8 | — | — |
| 115 | 362.4 | 786.0 | — | — |
| 116 | 2.5 | 3.3 | 504 | 1300 |
| 117 | 4.5 | 17.8 | 2334 | 2500 |
| 118 | 264.1 | 557.4 | — | — |
| 119 | 6.3 | 37.4 | 7209 | 3200 |
| 120 | 13.9 | 31.1 | 5597 | 3600 |
| 121 | 4.9 | 18.7 | — | — |
| 122 | 748.6 | 7512.2 | — | — |
| 123 | 229.0 | 334.3 | — | — |
| 124 | 11.7 | 36.6 | 6757 | 6500 |
| 125 | 15.2 | 42.3 | 7740 | 5900 |
| 126 | 25.7 | 92.5 | — | — |
| 127 | 151.5 | 359.3 | — | — |
| 128 | 212.2 | 303.1 | — | — |
| 129 | 2.6 | 6.9 | 1250 | 3000 |
| 130 | 185.4 | 433.4 | — | — |
| 131 | 266.8 | 602.7 | — | — |
| 132 | 79.1 | 303.5 | — | — |
| 133 | 72.2 | 140.3 | — | — |
| 134 | 1.3 | 3.4 | — | — |
| 135 | 1.1 | 2.1 | — | — |
| 136 | 1.3 | 2.7 | 510 | 610 |
| 137 | 41.6 | 125.1 | — | — |
| 138 | 1.7 | 3.8 | — | — |
| 139 | 1.7 | 5.3 | — | — |
| 140 | 3.3 | 9.4 | — | — |
| 141 | 1.2 | 2.7 | — | — |
| 142 | 4.0 | 12.0 | 3288 | 1410 |
| 143 | 7.3 | 5.6 | 985 | 1100 |
| 144 | 1.8 | 4.8 | — | — |
| 145 | 2.7 | 2.9 | 515 | 230 |
| 146 | 16.0 | 16.0 | 2386 | 1200 |
| 147 | 901.0 | 1160.0 | — | — |
| 148 | 4.0 | 4.0 | — | — |
| 149 | 2.0 | 6.0 | 1556 | 2300 |
| 150 | 4.0 | 4.0 | — | — |
| 151 | 126.0 | 188.0 | — | — |
| 152 | 47.0 | 310.0 | — | — |
| 153 | 76.0 | 130.0 | — | — |
| 154 | 46.0 | 85.0 | — | — |
| 155 | 17.4 | 23.8 | 3500 | 1200 |
| 156 | 21.5 | 42.4 | 1900 | 1800 |
| 157 | 4.7 | 8.4 | — | — |
| 158 | 3.0 | 7.0 | 940 | 1900 |
| 159 | 4.9 | 9.2 | 1030 | 1000 |
| 160 | 16.4 | 16.1 | 480 | 620 |
| 161 | 516.2 | 881.7 | — | — |
| 162 | 4.0 | 5.3 | 1400 | — |
| 163 | 939.7 | 658.3 | — | — |
| 164 | 21.2 | 34.3 | 1040 | 2200 |
| 165 | 368.7 | 330.3 | — | — |
| 166 | 15.2 | 16.6 | — | — |
| 167 | 91.8 | 58.4 | — | — |
| 168 | 343.0 | 333.9 | — | — |
| 169 | 27.4 | 20.9 | — | — |
| 170 | 60.3 | 37.1 | — | — |
| 171 | 6.1 | 3.2 | — | — |
| 172 | 19.0 | 9.7 | — | — |
| 173 | 21.7 | 24.6 | — | — |
| 174 | 33.4 | 118.7 | 2167 | — |
| 175 | 534.6 | 43.0 | — | — |
| 176 | 1.3 | 1.1 | — | — |
| 177 | 3.4 | 3.6 | — | — |
| 178 | 357.6 | 1258.8 | — | — |
| 179 | 0.6 | 1.1 | 598 | 590 |
| 180 | 1.2 | 3.0 | 1007 | 1700 |
| 181 | 61.4 | 22.5 | — | — |
| 182 | 3.8 | 3.7 | 52 | 66 |
| 183 | 2.8 | 2.7 | 20 | 120 |
| 184 | 28.1 | 14.5 | 567 | 600 |
| 185 | 68.1 | 110.0 | — | — |
| 186 | 26.1 | 43.9 | 2100 | 1000 |
| 187 | 6.2 | 33.8 | 550 | 510 |
| 188 | 24.1 | 76.2 | — | — |

For testing the efficacy of TRPM8 agonists for enhancing swallowing, experiment using the pig model can be conducted according to assay described below. The efficacy can be compared to a vehicle control.

### In Vivo Assay

Male castrated German Landrace pigs (weight range of 20 to 35 kg) were used. Anesthesia was induced by injecting 20 ml of a urethane solution (20 g/100 mL dissolved in saline) into an ear vein corresponding to a dose of around 16.8 mg/Kg. Anesthesia was maintained by continuous infusion of 15-20 mL per hour of the urethane solution, which was infused into an epigastric vein, and additionally by infusion of Zoletil and Rompun (500 mg Zoletil is disssovled in 10 ml of Rompun 2%, then diluted 1:10 with saline and 3-5 mL per hour of this diluted solution. Bupivacain 0.5% JENAPHARM@ was injected for additional infiltration anesthesia. Anesthesia was monitored via pulsoximetry (ear) and regular reflex testing for pain. Body temperature was monitored and maintained by an infrared lamp.

Preparation of pigs: Swallowing responses were assessed by manometry with a pressure probe placed into the mouth of the pig. The pressure probe consisted of a plastic tube (diameter 3.3 mm) to which a small balloon was attached. The tube was advanced about 12-14 cm into the mouth related to the snout. The balloon was then inflated with air to yield a pressure of 20-30mbar (vehicle). Ideally a swallowing response with 1 ml of fluid raised this pressure by 20-50mbar which was then referred to as the swallowing pressure (the increment of this pre-set pressure in the inflated balloon after swallowing) to be further enhanced by an effective test drug.

The free end of the tube was connected to a differential pressure transducer MPX Type 399/2 (Hugo Sachs Elektronik-Harvard Apparatus) and a Hugo Sachs Plugsys-amplifier system. The biological signals were recorded by a Hugo Sachs Plugsys-amplifier system and continuously stored on a computer hard disk by an on line data acquisition and analysis system (Hem 4.2 Notocord Systems, Croissy-sur-Seine, France).

A second tube referred to as injection tube was placed dorsally to the manometer tube to enable the administration of the vehicle that induced the baseline swallowing activity, or of this same vehicle containing the test compound.

Induction and assessment of swallowing activity: 0.5h after anesthesia induction 1 ml of a fluid or the vehicle for the test drug was injected into the injection tube placed into the oral cavity. The number of swallows was counted and the highest-pressure increment was registered. Challenges with vehicle were repeated at intervals of 30 min until two consecutive vehicle challenges show an about equal swallowing response. The next challenge was the vehicle loaded with the test drug (otherwise same procedure). The efficacy of a compound to enhance swallowing as shown in Table 7 was expressed as the % increase in pressure and frequency of swallowing after administration of the compound compared to the pressure and frequency recorded for its vehicle administered just before.

TABLE 7
Efficacy of tested compound

| | dose per pig | % increase pressure | % increase |
|---|---|---|---|
| Example | (mg) | vs vehicle | frequence vs vehicle |
| 1 | 10 | 274 | 231 |
| 50 | 10 | 63 | 100 |
| 51 | 10 | 186 | 250 |
| 52 | 10 | 202 | 172 |
| 4 | 10 | 221 | 417 |
| 40 | 10 | 393 | 62 |
| 54 | 10 | 64 | 75 |
| 108 | 10 | 169 | 114 |
| 14 | 10 | 73 | 62 |
| 31 | 10 | 253 | 119 |
| 63 | 10 | 186 | 191 |
| 13 | 10 | 53 | 100 |
| 80 | 10 | 86 | 91 |
| 21 | 10 | 181 | 171 |
| 22 | 10 | 182 | 500 |
| 81 | 10 | 315 | 247 |
| 49 | 10 | 133 | 300 |
| 25 | 10 | 168 | 173 |

It is therefore apparent that the compounds of formula (I), or a pharmaceutically acceptable salt thereof can activate TRPM8 receptors. The compounds of formula (I), or a pharmaceutically acceptable salt thereof, can therefore be used for preparing medicaments, especially medicaments which are agonists or openers of TRPM8 receptor.

Accordingly, also provided herein is medicament which comprises a compound of the formula (I), or a pharmaceutically acceptable salt thereof.

In one embodiment, provided herein is a compound of formula (I) as defined above, or a pharmaceutically acceptable salt thereof, for use in therapy, especially as agonist of TRPM8 receptor.

In one embodiment, provided herein is a compound of formula (I) as defined above, or a pharmaceutically acceptable salt thereof, for use in the treatment of oropharyngeal dysphagia, chronic cough, pharyngeal irritation, chronic itch, dry and pruritic skin.

In one embodiment, provided herein is a compound of formula (I) defined above, or a pharmaceutically acceptable salt thereof, for use in the treatment of oropharyngeal dysphagia.

In one embodiment, provided is a method of treating the pathological conditions indicated above, comprising administering to a subject in need thereof a therapeutically effective amount of a compound of formula (I), or a pharmaceutically acceptable salt thereof. In an embodiment of this method of treatment, the subject is a human.

In one embodiment, provided is the use of a compound of the formula (I), or a pharmaceutically acceptable salt thereof, for the manufacture of a medicament useful in treating any of the pathological conditions indicated above, more particularly the use in treating oropharyngeal dysphagia.

In one embodiment, provided is a pharmaceutical composition comprising a compound of formula (I), or a pharmaceutically acceptable salt thereof, and also at least one pharmaceutically acceptable excipient. The said excipients are selected, in accordance with the pharmaceutical form and method of administration desired, from the customary excipients, which are known to a person skilled in the art.

In one embodiment, provided is a pharmaceutical composition for oral administration including an effective amount of a compound of formula (I) or a pharmaceutically acceptable salt thereof and excipients in the form of orally disintegrating tablet, liquid, lozenge, film, oral solution, suspension, drop, droplet, dropper, spray, emulsion or syrup. Compositions intended for oral use are prepared according to any method known in the art for the manufacture of pharmaceutical compositions.

In one embodiment, provided is a pharmaceutical composition comprising a compound of formula (I) or a pharmaceutically acceptable salt thereof being administered to a patient via topical, sublingual, buccal, pharynx, oropharyngeal, throat administration. In certain embodiment, pharmaceutical composition is administered directly to the oral cavity or the oropharyngeal surface of the patient (e.g., in the form of a spray or drops).

In one embodiment, provided is a delivery device for delivering a pharmaceutical composition comprising a compound of formula (I) or a pharmaceutically acceptable salt thereof to a patient.

In one embodiment, the delivery device may be a spraying or atomising device, such as a pump-action spray or an aerosol spray.

In one embodiment, the delivery device may be spray containers, bottles, vials, or small portable devices such as pumps, atomizers.

In one embodiment, the delivery device is a standard dropper mounted into a cap for closing a bottle containing the pharmaceutical composition.

In one embodiment, the delivery device is a syringe.

In one embodiment, the delivery device is an oral catheter inserted in the oral cavity. The pharmaceutical composition is administered through this catheter reaching the back of the oral cavity or the oropharyngeal surface of a patient.

In one embodiment, provided is a kit, the kit comprising: i) a pharmaceutical composition comprising a compound of formula (I) and ii) a delivery device for delivering the pharmaceutical composition to a patient. The pharmaceutical composition may be separate from the delivery device.

One embodiment is a pharmaceutical composition comprising a compound of formula (I) in an amount of 0.3 mg/mL to 20 mg/mL in a form of a spray, drop, dropper, buccal spray, pharynx spray or throat spray.

In one embodiment, the composition further comprises a PEG 400, polyethylene glycol (15)-hydroxystearate, ethanol.

In one embodiment, provided is use of a compound of formula (I) or a pharmaceutically acceptable salt thereof in the manufacture of a medicament for use in a method of treatment.

In one embodiment, provided is a method of treatment of a condition, comprising: contacting a pharmaceutical composition comprising as active principle a compound of formula (I) or a pharmaceutically acceptable salt thereof and a excipient with, e.g., the oral cavity, or the oropharyngeal surface of the human, thereby delivering an amount of the compound therapeutically effective for treatment of (e.g., alleviation of) the condition.

In one embodiment, provided is a method of treatment of oropharyngeal dysphagia comprising: contacting a pharmaceutical composition comprising a compound of formula (I) and a pharmaceutically acceptable excipient with oral cavity, or the oropharyngeal surface of the human, thereby delivering an effective amount of the compound to the mucous membranes of a human.

In one embodiment, provided is a method of treatment of (e.g., alleviation of) oropharyngeal dysphagia.

There may be particular cases in which higher or lower dosages are appropriate. According to usual practice, the dosage that is appropriate for each patient is determined by the doctor according to the mode of administration and the weight and response of the said patient.

What is claimed is:

1. A compound of formula (I): 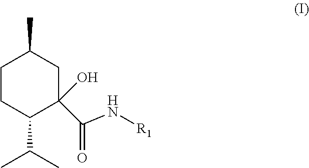
wherein:
R₁ is —C(R₂)(R₃)—[C(R₄)(R₅)]ₘ-L-R₆ or —R₇
m represents 0, 1, 2 or 3;
R₂ and R₃ independently represent a hydrogen atom, a deuterium atom, a —(C₁-C₆)-alkyl group, a (C₁-C₆)-alkyl-OH group, a —C(═O)NH₂ group, a —(C₁-C₆)-alkoxyl group, or a —C(═O)O(C₁-C₆)-alkyl group;
R₄ and R₅ independently represent a hydrogen atom, a deuterium atom, a fluorine atom, a —NH₂ group, a —OH group, a —(C₁-C₆)-alkyl group, a —CF₃ group, a carboxyl group, or a —R₈—(C₁-C₆)-alkyl-R₉ group wherein:
R₈ represents a bond, an —O—, a —OC(═O)— group, a —N(H)C(═O)— group, a —C(═O)O— group, or a —C(═O)N(H)— group;
R₉ represents a hydrogen atom, a —C(═O)—OH group, a —C(═O)O(C₁-C₃)-alkyl group, a —OH group, an —O—(C₁-C₃)-alkyl group, or a —NH₂ group;
or R₄ and R₅ can form, together with the carbon atom to which they are attached, a heterocycloalkyl group comprising 3 to 5 carbon atoms and comprising from 1 or 2 heteroatoms selected from oxygen and nitrogen;
L represents a bond, a —(C₁-C₆)-alkylene- group, an —O—(C₁-C₆)-alkylene- group, an —O—, a —OC(═O)— group, a —N(H)— group, a —C(═O)— group, a —C(═O)O— group, a —C(═O)—O—(C₁-C₃)-alkyl- group, a —C(═O)—N(H)— or a —CONH(C₁-C₆)-alkyl- group;
R₆ is selected from the group consisting of a —OH group; a —(C₁-C₆)-alkyl group; a phenyl group, a monocyclic heteroaryl group comprising 3 to 5 carbon atoms and comprising from 1 to 2 heteroatoms independently selected from oxygen, nitrogen and sulfur, an ortho-fused bicyclic heteroaryl group comprising 7 to 10 carbon atoms and comprising from 1 to 4 heteroatoms independently selected from oxygen, nitrogen and sulfur, an ortho-fused bicyclic cycloalkyl group comprising 8 to 11 carbon atoms, and an ortho-fused bicyclic heterocycloalkyl group comprising 8 to 9 carbon atoms and comprising 1 or 2 heteroatoms independently selected from oxygen and nitrogen; said phenyl, monocyclic heteroaryl, ortho-fused bicyclic heteroaryl, ortho-fused bicyclic cycloalkyl, ortho-fused bicyclic heterocycloalkyl groups being unsubstituted or substituted with 1 to 3 substituents independently selected from the group consisting of:
a halogen atom, a —OH group, an oxo group, an —O—(C₁-C₆)-alkyl group, a —(C₁-C₆)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₆)-alkyl group, a —C(═O)N(C₁-C₃)-alkyl group, a —(OCH₂CH₂)ₙ—R₁₀ group, and a —R₁₁—(C₁-C₆)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents an —O—(C₁-C₄)-alkyl group, a —N⁺—(CH₃)₃ group, or —N⁺H—(CH₃)₂ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, a —C(═O)H group, a heterocyclic group or an —O-heterocyclic group, said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group;
R₇ represents a phenyl group, a monocyclic cycloalkyl group comprising 4 to 7 carbon atoms, a monocyclic heterocycloalkyl group comprising 3 to 6 carbon atoms and comprising from 1 to 2 heteroatoms independently selected from oxygen and nitrogen, an ortho-fused bicyclic cycloalkyl group comprising 8 to 11 carbon atoms, or an ortho-fused bicyclic heterocycloalkyl group comprising 8 to 9 carbon atoms and comprising 1 or 2 heteroatoms independently selected from oxygen and nitrogen; wherein said phenyl group being unsubstituted or substituted with 1 to 3 substituents independently selected from a halogen atom, a —(C₁-C₃)-alkyl group, an —O—(C₁-C₃)-alkyl group and a morpholine group; said monocyclic cycloalkyl, monocyclic heterocycloalkyl, an ortho-fused bicyclic heterocycloalkyl groups being unsubstituted or substituted with 1 to 3 substituents independently selected from the group consisting of: a halogen atom, an oxo group, a —(C₁-C₆)-alkyl group, a phenyl group, an —O— group, a benzyl group, a —OH group, and an —O—(C₁-C₆)-alkyl group;
or a pharmaceutically acceptable salt thereof.

2. The compound of formula (I) according to claim 1, wherein R₁ represents a —C(R₂)(R₃)—[C(R₄)(R₅)]ₘ-L-R₆ group; m represents 0 or 1; or a pharmaceutically acceptable salt thereof.

3. The compound of formula (I) according to claim 1 having the absolute configuration corresponding to a compound of formula (Ia), 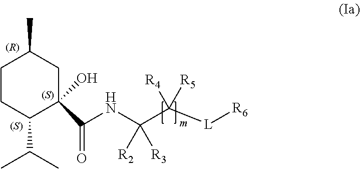
wherein:
m represents 0 or 1;
R₆ represents a phenyl group, wherein said phenyl group being unsubstituted or substituted with 1 to 3 substituents independently selected from the group consisting of:
a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, a —C(═O)N(C₁-C₃)-alkyl group, a —(OCH₂CH₂)ₙ—R₁₀ group, and a —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents an —O—(C₁-C₃)-alkyl group, a —N⁺—(CH₃)₃ group, or —N⁺H—(CH₃)₂ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, a —C(═O)H group, a heterocyclic group or an —O-heterocyclic group, said heterocyclic group and said —O-heterocyclic group comprising 3 to 9 carbon atoms and comprising from 1 to 3 heteroatoms independently selected from oxygen, nitrogen and sulfur being unsubstituted or substituted with 1 to 3 substituents independently selected from an oxo group and a —(C₁-C₃)-alkyl group;
or a pharmaceutically acceptable salt thereof.

4. The compound of formula (Ia) according to claim 3, wherein
R₂ and R₃ independently represent a hydrogen atom, a —(C₁-C₃)-alkyl group, a (C₁-C₃)-alkyl-OH group, a —(C₁-C₃)-alkoxyl group;
R₄ and R₅ independently represent a hydrogen atom, a deuterium atom, a fluorine atom, a —NH₂ group, a —OH group, a —(C₁-C₃)-alkyl group, a —CF₃ group, or a —R₈—(C₁-C₄)-alkyl-R₉ group wherein:
R₈ represents a bond, an —O—, a —OC(═O)— group, a —N(H)C(═O)— group, a —C(═O)O— group, or a —C(═O)N(H)— group;
R₉ represents a hydrogen atom, a —C(═O)—OH group, a —C(═O)O(C₁-C₃)-alkyl group,
a —OH group, an —O—(C₁-C₃)-alkyl group, or a —NH₂ group;
L represents a bond, an —O—, a —OC(═O)— group, a —C(═O)— group, a —C(═O)O— group, a —C(═O)—O—(C₁-C₃)-alkyl- group, or a —C(═O)—N(H)—;
R₆ represents a phenyl group, wherein said phenyl group being unsubstituted or substituted with 1 to 3 substituents independently selected from the group consisting of:
a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, a —C(═O)N(C₁-C₃)-alkyl group, a —(OCH₂CH₂)ₙ—R₁₀ group, and a —R₁₁—(C₁-C₃)-alkyl-R₁₂ group which is unsubstituted or substituted on the (C₁-C₆)-alkyl with 1 to 3 substituents independently selected from a —OH group, a —NH₂ group and —OCH₃ group; wherein n represents 1, 2 or 3; R₁₀ represents an —O—(C₁-C₃)-alkyl group, or a —N⁺—(CH₃)₃ group, or —N⁺H—(CH₃)₂ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, a —C(═O)H group;
or a pharmaceutically acceptable salt thereof.

5. The compound of formula (Ia) according to claim 3, wherein:
m represents 1;
R₂ and R₃ independently represent a hydrogen atom, a —(C₁-C₃)-alkyl group, a —(C₁-C₃)-alkyl-OH group;
R₄ and R₅ independently represent a hydrogen atom, a deuterium atom, a fluorine atom, a —NH₂ group, a —OH group, a —(C₁-C₃)-alkyl group, or a —CF₃ group,
L represents a bond or a —C(═O)— group;
R₆ represents a phenyl group, wherein said phenyl group being unsubstituted or substituted with 1 to 2 substituents independently selected from the group consisting of:
a halogen atom, a —OH group, an —O—(C₁-C₃)-alkyl group, a —(C₁-C₃)-alkyl group, a —NO₂ group, a —CN group, a —C(═O)H group, a —SO₂NH₂ group, a —C(═O)NH₂ group, a —OCH₂C(═O)NH₂ group, a —C(═O)O(C₁-C₃)-alkyl group, a —C(═O)N(C₁-C₃)-alkyl group, a —(OCH₂CH₂)ₙ—R₁₀ group, and a —R₁₁—(C₁-C₃)-alkyl-R₁₂ group which is unsubstituted or substituted on the (C₁-C₆)-alkyl with a —OH group or a —NH₂ group; wherein n represents 1, 2 or 3; R₁₀ represents an —O—(C₁-C₃)-alkyl group or a —N⁺—(CH₃)₃ group; R₁₁ represents a bond, an —O—, or a —C(═O)O group; R₁₂ represents a —OH group, a —C(═O)OH group, a —C(═O)O(C₁-C₃)-alkyl group, a —C(═O)N(C₁-C₃)-alkyl group, a —NH₂ group, a —NH—C(═O)(C₁-C₃)-alkyl group, or a —C(═O)H group;
or a pharmaceutically acceptable salt thereof.

6. The compound of formula (I) according to claim 1, which is (1S,2S, 5R)-1-hydroxy-N-(3-hydroxyphenethyl)-2-isopropyl-5-methylcyclohexanecarboxamide; or a pharmaceutically acceptable salt thereof.

7. The compound of formula (I) according to claim 1, which is 2-hydroxyethyl 2-(2-((1S,2S,5R)-1-hydroxy-2-isopropyl-5-methylcyclohexane-1-carboxamido)ethyl) benzoate; or a pharmaceutically acceptable salt thereof.

8. The compound of formula (I) according to claim 1, which is (1S,2S,5R)-1-hydroxy-N-(2-(2-hydroxyethyl)phenethyl)-2-isopropyl-5-methylcyclohexane-1-carboxamide; or a pharmaceutically acceptable salt thereof.

9. The compound of formula (I) according to claim 1, which is Methyl-3-(2-((1S,2S,5R)-1-hydroxy-2-isopropyl-5-methylcyclohexane-1-carboxamido)ethyl)benzoate; or a pharmaceutically acceptable salt thereof.

10. The compound of formula (I) according to claim 1, which is (1S,2S,5R)—N-(2-(2-amino-2-oxoethoxy)phenethyl)-1-hydroxy-2-isopropyl-5-methylcyclohexane-1-carboxamide; or a pharmaceutically acceptable salt thereof.

11. The compound of formula (I) according to claim 1, which is (1S,2S,5R)-1-hydroxy-N—((S)-2-hydroxy-2-phenylethyl)-2-isopropyl-5-methylcyclohexane-1-carboxamide; or a pharmaceutically acceptable salt thereof.

12. The compound of formula (I) according to claim 1, which is (2-hydroxyethyl 3-(2-((1S,2S,5R)-1-hydroxy-2-isopropyl-5-methylcyclohexane-1-carboxamido)ethyl) benzoate; or a pharmaceutically acceptable salt thereof.

13. The compound of formula (I) according to claim 1, which is (1S,2S,5R)-1-hydroxy-2-isopropyl-5-methyl-N-((3-phenyloxetan-3-yl)methyl)cyclohexane-1-carboxamide; or a pharmaceutically acceptable salt thereof.

14. A process for preparing a compound of formula (I) according to claim 1, comprising submitting a compound (intermediate 4) 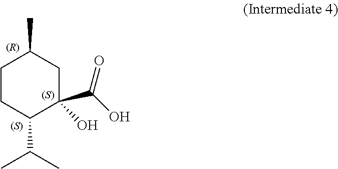
to a coupling reaction with a compound of formula (Ic) or a compound of formula (Id) 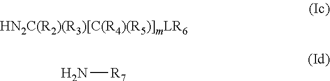
wherein m, R₂, R₃, R₄, R₅, R₆, L, R₇ are as defined in claim 1.

15. A compound selected from the group consisting of:
(1S,2S,5R)-1-hydroxy-N—((S)-2-hydroxy-2-phenylethyl)-2-isopropyl-5-methylcyclohexane-1-carboxamide;
2-hydroxyethyl 2-(2-((1S,2S,5R)-1-hydroxy-2-isopropyl-5-methylcyclohexane-1-carboxamido)ethyl) benzoate;
(1S,2S,5R)-1-hydroxy-N-(3-hydroxyphenethyl)-2-isopropyl-5-methylcyclohexane-1-carboxamide;
2-hydroxyethyl 3-(2-((1S,2S,5R)-1-hydroxy-2-isopropyl-5-methylcyclohexane-1-carboxamido)ethyl) benzoate;
(1S,2S,5R)-1-hydroxy-N-(2-(2-hydroxyethyl)phenethyl)-2-isopropyl-5-methylcyclohexane-1-carboxamide;
Methyl-3-(2-((1S,2S,5R)-1-hydroxy-2-isopropyl-5-methylcyclohexane-1-carboxamido)ethyl)benzoate;
(1S,2S,5R)-1-hydroxy-2-isopropyl-5-methyl-N-((3-phenyloxetan-3-yl)methyl)cyclohexane-1-carboxamide; and
(1S,2S,5R)—N-(2-(2-amino-2-oxoethoxy)phenethyl)-1-hydroxy-2-isopropyl-5-methylcyclohexane-1-carboxamide;
or a pharmaceutically acceptable salt thereof.

16. A pharmaceutical composition comprising a compound of formula (I) according to claim 1 or a pharmaceutically acceptable salt and at least one pharmaceutically acceptable excipient.

17. The pharmaceutical composition according to claim 16, wherein the compound of formula (I) is (1S,2S,5R)-1-hydroxy-N—((S)-2-hydroxy-2-phenylethyl)-2-isopropyl-5-methylcyclohexane-1-carboxamide; or a pharmaceutically acceptable salt thereof.

18. The pharmaceutical composition according to claim 16, wherein the compound of formula (I) is 2-hydroxyethyl 2-(2-((1S,2S,5R)-1-hydroxy-2-isopropyl-5-methylcyclohexane-1-carboxamido)ethyl) benzoate; or a pharmaceutically acceptable salt thereof.

19. The pharmaceutical composition according to claim 16, wherein the compound of formula (I) is (1S,2S,5R)-1-hydroxy-N-(3-hydroxyphenethyl)-2-isopropyl-5-methylcyclohexane-1-carboxamide; or a pharmaceutically acceptable salt thereof.

20. The pharmaceutical composition according to claim 16, wherein the compound of formula (I) is 2-hydroxyethyl 3-(2-((1S,2S,5R)-1-hydroxy-2-isopropyl-5-methylcyclohexane-1-carboxamido)ethyl) benzoate; or a pharmaceutically acceptable salt thereof.

21. The pharmaceutical composition according to claim 8, wherein the compound of formula (I) is (1S,2S,5R)-1-hydroxy-N-(2-(2-hydroxyethyl)phenethyl)-2-isopropyl-5-methylcyclohexane-1-carboxamide; or a pharmaceutically acceptable salt thereof.

22. The pharmaceutical composition according to claim 16, wherein the compound of formula (I) is Methyl-3-(2-((1S,2S,5R)-1-hydroxy-2-isopropyl-5-methylcyclohexane-1-carboxamido)ethyl)benzoate; or a pharmaceutically acceptable salt thereof.

23. The pharmaceutical composition according to claim 16, wherein the compound of formula (I) is (1S,2S,5R)-1-hydroxy-2-isopropyl-5-methyl-N-((3-phenyloxetan-3-yl)methyl)cyclohexane-1-carboxamide; or a pharmaceutically acceptable salt thereof.

24. The pharmaceutical composition according to claim 16, wherein the compound of formula (I) is (1S,2S,5R)—N-(2-(2-amino-2-oxoethoxy)phenethyl)-1-hydroxy-2-isopropyl-5-methylcyclohexane-1-carboxamide; or a pharmaceutically acceptable salt thereof.

25. A method of treating a disease involving activation of TRPM8 receptors, comprising administering to a subject in need thereof a therapeutically effective amount of a compound of formula (I) according to claim 1, or a pharmaceutically acceptable salt thereof.

26. A method of treating oropharyngeal dysphagia, comprising administering to a subject in need thereof a therapeutically effective amount of a compound of formula (I) according to claim 1, or a pharmaceutically acceptable salt thereof.

27. A medicament, characterized in that it comprises a compound of formula (I) according to any of claims 1 to 5, or a pharmaceutically acceptable salt thereof.

28. A compound of formula (I) according to any of claims 1 to 5, or a pharmaceutically acceptable salt thereof, for use in the treatment of oropharyngeal dysphagia.
